(19) United States
(12) Patent Application Publication
(10) Pub. No.: US 2002/0182675 A1
(43) Pub. Date: Dec. 5, 2002

(54) Novel genes encoding proteins having diagnostic, preventive, therapeutic and other uses

(75) Inventors: Sean A. McCarthy, San Diego, CA (US); Christopher C. Fraser, Lexington, MA (US); John D. Sharp, Arlington, MA (US); Thomas M. Barnes, Brookline, MA (US)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: Millennium Pharmaceuticals, Inc., Cambridge, MA

(21) Appl. No.: 10/042,431

(22) Filed: Oct. 25, 2001

Related U.S. Application Data
(63) Continuation-in-part of application No. 09/578,063, filed on May 24, 2000.
Application No. 09/578,063 is a continuation-in-part of application No. 09/333,159, filed on Jun. 14, 1999.

(51) Int. Cl. C12P 21/02; C12N 5/06; C07K 14/435; C07H 21/04; C12N 9/00

(52) U.S. Cl. 435/69.1; 435/183; 435/320.1; 435/325; 530/350; 536/23.2

(57) ABSTRACT

The invention provides isolated nucleic acids encoding a variety of proteins having diagnostic, preventive, therapeutic, and other uses. These nucleic and proteins are useful for diagnosis, prevention, and therapy of a number of human and other animal disorders. The invention also provides antisense nucleic acid molecules, expression vectors containing the nucleic acid molecules of the invention, host cells into which the expression vectors have been introduced, and non-human transgenic animals in which a nucleic acid molecule of the invention has been introduced or disrupted. The invention still further provides isolated polypeptides, fusion polypeptides, antigenic peptides and antibodies. Diagnostic, screening, and therapeutic methods utilizing compositions of the invention are also provided. The nucleic acids and polypeptides of the present invention are useful as modulating agents in regulating a variety of cellular processes.

### CROSS-REFERENCE TO RELATED APPLICATIONS

This application is a continuation-in-part of co-pending U.S. patent application Ser. No 09/578,063, filed May 24, 2000, which is a continuation-in-part of co-pending U.S. patent application Ser. No. 09/333,159, filed Jun. 14, 1999.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND OF THE INVENTION

The molecular bases underlying many human and animal physiological states (e.g., diseased and homeostatic states of various tissues) remain unknown. Nonetheless, it is well understood that these states result from interactions among the proteins and nucleic acids present in the cells of the relevant tissues. In the past, the complexity of biological systems overwhelmed the ability of practitioners to understand the molecular interactions giving rise to normal and abnormal physiological states. More recently, though, the techniques of molecular biology, transgenic and null mutant animal production, computational biology, pharmacogenomics, and the like have enabled practitioners to discern the role and importance of individual genes and proteins in particular physiological states.

Knowledge of the sequences and other properties of genes (particularly including the portions of genes encoding proteins) and the proteins encoded thereby enables the practitioner to design and screen agents which will affect, prospectively or retrospectively, the physiological state of an animal tissue in a favorable way. Such knowledge also enables the practitioner, by detecting the levels of gene expression and protein production, to diagnose the current physiological state of a tissue or animal and to predict such physiological states in the future. This knowledge furthermore enables the practitioner to identify and design molecules which bind with the polynucleotides and proteins, in vitro, in vivo, or both.

The present invention provides sequence information for polynucleotides derived from human and murine genes and for proteins encoded thereby, and thus enables the practitioner to assess, predict, and affect the physiological state of various human and murine tissues.

### BRIEF SUMMARY OF THE INVENTION

The present invention is based, at least in part, on the discovery of a variety of human and murine cDNA molecules which encode proteins which are herein designated TANGO 202, TANGO 234, TANGO 265, TANGO 273, TANGO 286, TANGO 294, and INTERCEPT 296. These seven proteins, fragments thereof, derivatives thereof, and variants thereof are collectively referred to herein as the polypeptides of the invention or the proteins of the invention. Nucleic acid molecules encoding polypeptides of the invention are collectively referred to as nucleic acids of the invention.

The nucleic acids and polypeptides of the present invention are useful as modulating agents in regulating a variety of cellular processes. Accordingly, in one aspect, the present invention provides isolated nucleic acid molecules encoding a polypeptide of the invention or a biologically active portion thereof. The present invention also provides nucleic acid molecules which are suitable as primers or hybridization probes for the detection of nucleic acids encoding a polypeptide of the invention.

The invention also features nucleic acid molecules which are at least 40% (or 50%, 60%, 70%, 80%, 90%, 95%, or 98%) identical to the nucleotide sequence of any of SEQ ID NOs: 1, 2, 9, 10, 17, 18, 25, 26, 33, 34, 45, 46, 53, 54, 67, 68, 72, and 73, the nucleotide sequence of a cDNA clone deposited with ATCC® as one of Accession numbers 207219, 207184, 207228, 207185, 207220, and 207221 (“a cDNA of a clone deposited as one of ATCC® 207219, 207184, 207228, 207185, 207220, and 207221”), or a complement thereof.

The invention features nucleic acid molecules which include a fragment of at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or 4928) consecutive nucleotide residues of any of SEQ ID NOs: 1, 2, 9, 10, 17, 18, 25, 26, 33, 34, 45, 46, 53, 54, 67, 68, 72, and 73, the nucleotide sequence of a cDNA of a clone deposited as one of ATCC® 207219, 207184, 207228, 207185, 207220, and 207221, or a complement thereof.

The invention also features nucleic acid molecules which include a nucleotide sequence encoding a protein having an amino acid sequence that is at least 50% (or 60%, 70%, 80%, 90%, 95%, or 98%) identical to the amino acid sequence of any of SEQ ID NOs: 3-8, 11-16, 19-24, 27-32, 35-44, 47-52, 55-66, 69, and 74, or the amino acid sequence encoded by a cDNA of a clone deposited as one of ATCC® 207219, 207184, 207228, 207185, 207220, and 207221, or a complement thereof.

In preferred embodiments, the nucleic acid molecules have the nucleotide sequence of any of SEQ ID NOs: 1, 2, 9, 10, 17, 18, 25, 26, 33, 34, 45, 46, 53, 54, 67, 68, 72, and 73, or the nucleotide sequence of a cDNA of a clone deposited as one of ATCC® 207219, 207184, 207228, 207185, 207220, and 207221.

Also within the invention are nucleic acid molecules which encode a fragment of a polypeptide having the amino acid sequence of any of SEQ ID NOs: 3-8, 11-16, 19-24, 27-32, 35-44, 47-52, 55-66, 69, and 74, or the amino acid sequence encoded by a cDNA of a clone deposited as one of ATCC® 207219, 207184, 207228, 207185, 207220, and 207221, the fragment including at least 8 (10, 15, 20, 25, 30, 40, 50, 75, 100, 125, 150, or 200) consecutive amino acids of any of SEQ ID NOs: 3-8, 11-16, 19-24, 27-32, 35-44, 47-52, 55-66, 69, and 74, or the amino acid sequence encoded by a cDNA of a clone deposited as one of ATCC® 207219, 207184, 207228, 207185, 207220, and 207221.

The invention includes nucleic acid molecules which encode a naturally occurring allelic variant of a polypeptide comprising the amino acid sequence of any of SEQ ID NOs: 3-8, 11-16, 19-24, 27-32, 35-44, 47-52, 55-66, 69, and 74, or the amino acid sequence encoded by a cDNA of a clone deposited as one of ATCC® 207219, 207184, 207228, 207185, 207220, and 207221, wherein the nucleic acid molecule hybridizes under stringent conditions to a nucleic acid molecule having a nucleic acid sequence encoding any of SEQ ID NOs: 1, 2, 9, 10, 17, 18, 25, 26, 33, 34, 45, 46, 53, 54, 67, 68, 72, and 73, the nucleotide sequence of a cDNA of a clone deposited as one of ATCC® 207219, 207184, 207228, 207185, 207220, and 207221, or a complement thereof.

Also within the invention are isolated polypeptides or proteins having an amino acid sequence that is at least about 50%, preferably 60%, 75%, 90%, 95%, or 98% identical to the amino acid sequence of any of SEQ ID NOs: 3-8, 11-16, 19-24, 27-32, 35-44, 47-52, 55-66, 69, and 74.

Also within the invention are isolated polypeptides or proteins which are encoded by a nucleic acid molecule having a nucleotide sequence that is at least about 40%, preferably 50%, 75%, 85%, or 95% identical the nucleic acid sequence encoding any of SEQ ID NOs: 3-8, 11-16, 19-24, 27-32, 35-44, 47-52, 55-66, 69, and 74, and isolated polypeptides or proteins which are encoded by a nucleic acid molecule consisting of the nucleotide sequence which hybridizes under stringent hybridization conditions to a nucleic acid molecule having the nucleotide sequence of any of SEQ ID NOs: 1, 2, 9, 10, 17, 18, 25, 26, 33, 34, 45, 46, 53, 54, 67, 68, 72, and 73.

Also within the invention are polypeptides which are naturally occurring allelic variants of a polypeptide that includes the amino acid sequence of any of SEQ ID NOs: 3-8, 11-16, 19-24, 27-32, 35-44, 47-52, 55-66, 69, and 74, or the amino acid sequence encoded by a cDNA of a clone deposited as one of ATCC® 207219, 207184, 207228, 207185, 207220, and 207221, wherein the polypeptide is encoded by a nucleic acid molecule which hybridizes under stringent conditions to a nucleic acid molecule having the nucleotide sequence of any of SEQ ID NOs: 1, 2, 9, 10, 17, 18, 25, 26, 33, 34, 45, 46, 53, 54, 67, 68, 72, and 73, or a complement thereof.

The invention also features nucleic acid molecules that hybridize under stringent conditions to a nucleic acid molecule having the nucleotide sequence of any of SEQ ID NOs: 1, 2, 9, 10, 17, 18, 25, 26, 33, 34, 45, 46, 53, 54, 67, 68, 72, and 73, the nucleotide sequence of a cDNA of a clone deposited as one of ATCC® 207219, 207184, 207228, 207185, 207220, and 207221, or a complement thereof. In other embodiments, the nucleic acid molecules are at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or 4928) nucleotides in length and hybridize under stringent conditions to a nucleic acid molecule having the nucleotide sequence of any of SEQ ID NOs: 1, 2, 9, 10, 17, 18, 25, 26, 33, 34, 45, 46, 53, 54, 67, 68, 72, and 73, the nucleotide sequence of a cDNA of a clone deposited as one of ATCC® 207219, 207184, 207228, 207185, 207220, and 207221, or a complement thereof. In some embodiments, the isolated nucleic acid molecules encode a cytoplasmic, transmembrane, extracellular, or other domain of a polypeptide of the invention. In other embodiments, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention. In another embodiment, the invention provides isolated host cells, e.g., mammalian and non-mammalian cells, containing such a vector or a nucleic acid of the invention. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention. Preferred proteins and polypeptides possess at least one biological activity possessed by the corresponding naturally-occurring human polypeptide. An activity, a biological activity, and a functional activity of a polypeptide of the invention refers to an activity exerted by a protein or polypeptide of the invention on a responsive cell as determined in vivo, or in vitro, according to standard techniques.

Such activities can be a direct activity, such as an association with or an enzymatic activity on a second protein, or an indirect activity, such as a cellular process (e.g., signaling activity) mediated by interaction of the protein with a second protein. Such activities include, by way of example, formation of protein-protein interactions with proteins of one or more signaling pathways (e.g., with a protein with which the naturally-occurring polypeptide interacts); binding with a ligand of the naturally-occurring protein; and binding with an intracellular target of the naturally-occurring protein. Other activities include modulation of one or more of cellular proliferation, of cellular differentiation, of chemotaxis, of cellular migration, and of cell death (e.g., apoptosis).

By way of example, TANGO 202 exhibits the ability to affect growth, proliferation, survival, differentiation, and activity of human hematopoietic cells (e.g., bone marrow stromal cells) and fetal cells. TANGO 202 modulates cellular binding to one or more mediators, modulates proteolytic activity in vivo, modulates developmental processes, and modulates cell growth, proliferation, survival, differentiation, and activity. Thus, TANGO 202 can be used to prevent, diagnose, or treat disorders relating to aberrant cellular protease activity, inappropriate interaction (or non-interaction) of cells with mediators, inappropriate development, and blood and hematopoietic cell-related disorders. Exemplary disorders for which TANGO 202 is useful include immune disorders, infectious diseases, auto-immune disorders, vascular and cardiovascular disorders, disorders related to mal-expression of growth factors, cancers, hematological disorders, various cancers, birth defects, developmental defects, and the like.

Further by way of example, TANGO 234 exhibits the ability to affect growth, proliferation, survival, differentiation, and activity of human lung, hematopoietic, and fetal cells and of (e.g., bacterial or fungal) cells and viruses which infect humans. TANGO 234 modulates growth, proliferation, survival, differentiation, and activity of gamma delta T cells, for example. Furthermore, TANGO 234 modulates cholesterol deposition on human arterial walls, and is involved in uptake and metabolism of low density lipoprotein and regulation of serum cholesterol levels.

Thus, TANGO 234 can be used to affect development and persistence of atherogenesis and arteriosclerosis, as well as other vascular and cardiovascular disorders. Other exemplary disorders for which TANGO 234 is useful include immune development disorders and disorders involving generation and persistence of an immune response to bacterial, fungal, and viral infections.

Still further by way of example, TANGO 265 modulates growth and regeneration of neuronal and epithelial tissues, and guides neuronal axon development. TANGO 265 is a transmembrane protein which mediates cellular interaction with cells, molecules and structures (e.g., extracellular matrix) in the extracellular environment. TANGO 265 is therefore involved in growth, organization, and adhesion of tissues and the cells which constitute those tissues. Furthermore, TANGO 265 modulates growth, proliferation, survival, differentiation, and activity of neuronal cells and immune system cells. Thus, TANGO 265 can be used, for example, to prevent, diagnose, or treat disorders characterized by aberrant organization or development of a tissue or organ, for guiding neural axon development, for modulating differentiation of cells of the immune system, for modulating cytokine production by cells of the immune system, for modulating reactivity of cells of the immune system toward cytokines, for modulating initiation and persistence of an inflammatory response, and for modulating proliferation of epithelial cells.

Yet further by way of example, TANGO 273 protein mediates one or more physiological responses of cells to bacterial infection, e.g., by mediating one or more of detection of bacteria in a tissue in which it is expressed, movement of cells with relation to sites of bacterial infection, production of biological molecules which inhibit bacterial infection, and production of biological molecules which alleviate cellular or other physiological damage wrought by bacterial infection. TANGO 273, a transmembrane protein, is also involved in transmembrane signal transduction, and therefore mediates transmission of signals between the extracellular and intracellular environments of cells. TANGO 273 mediates regulation of cell growth and proliferation, endocytosis, activation of respiratory burst, and other physiological processes triggered by transmission of a signal via a protein with which TANGO 273 interacts. The compositions and methods of the invention can therefore be used to prevent, diagnose, and treat disorders involving one or more physiological activities mediated by TANGO 273 protein. Such disorders include, for example, various bone-related disorders such as metabolic, homeostatic, and developmental bone disorders (e.g., osteoporosis, various cancers, skeletal development disorders, bone fragility and the like), disorders caused by or related to bacterial infection, and disorders characterized by aberrant transmembrane signal transduction by TANGO 273.

As an additional example, TANGO 286 protein is involved in lipid-binding physiological processes such as lipid transport, metabolism, serum lipid particle regulation, host anti-microbial defensive mechanisms, and the like. Thus, the compositions and methods of the invention can therefore be used to prevent, diagnose, and treat disorders involving one or more physiological activities mediated by TANGO 286 protein. Such disorders include, for example, lipid transport disorders, lipid metabolism disorders, obesity, disorders of serum lipid particle regulation, disorders involving insufficient or inappropriate host anti-microbial defensive mechanisms, vasculitis, bronchiectasis, LPS-related disorders such as shock, disseminated intravascular coagulation, anemia, thrombocytopenia, adult respiratory distress syndrome, renal failure, liver disease, and disorders associated with Gram negative bacterial infections, such as bacteremia, endotoxemia, sepsis, and the like.

Further by way of example, TANGO 294 protein is involved in facilitating absorption and metabolism of fat. Thus, the compositions and methods of the invention can therefore be used to prevent, diagnose, and treat disorders involving one or more physiological activities mediated by TANGO 294 protein. Such disorders include, for example, inadequate expression of gastric/pancreatic lipase, cystic fibrosis, exocrine pancreatic insufficiency, medical treatments which alter fat absorption, obesity, and the like.

As another example, INTERCEPT 296 protein is involved in physiological processes related to disorders of the human lung and esophagus. Thus, the compositions and methods of the invention can be used to prevent, diagnose, and treat these disorders. Such disorders include, for example, various cancers, bronchitis, cystic fibrosis, respiratory infections (e.g., influenza, bronchiolitis, pneumonia, and tuberculosis), asthma, emphysema, chronic bronchitis, bronchiectasis, pulmonary edema, pleural effusion, pulmonary embolus, adult and infant respiratory distress syndromes, heartburn, and gastric reflux esophageal disease.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

In one embodiment, the isolated polypeptide of the invention lacks both a transmembrane and a cytoplasmic domain. In another embodiment, the polypeptide lacks both a transmembrane domain and a cytoplasmic domain and is soluble under physiological conditions.

The polypeptides of the present invention, or biologically active portions thereof, can be operably linked to a heterologous amino acid sequence to form fusion proteins. The invention further features antibody substances that specifically bind a polypeptide of the invention such as monoclonal or polyclonal antibodies, antibody fragments, single-chain antibodies, and the like. In addition, the polypeptides of the invention or biologically active portions thereof can be incorporated into pharmaceutical compositions, which optionally include pharmaceutically acceptable carriers. These antibody substances can be made, for example, by providing the polypeptide of the invention to an immunocompetent vertebrate and thereafter harvesting blood or serum from the vertebrate.

In another aspect, the present invention provides methods for detecting the presence of the activity or expression of a polypeptide of the invention in a biological sample by contacting the biological sample with an agent capable of detecting an indicator of activity such that the presence of activity is detected in the biological sample.

In another aspect, the invention provides methods for modulating activity of a polypeptide of the invention comprising contacting a cell with an agent that modulates (inhibits or enhances) the activity or expression of a polypeptide of the invention such that activity or expression in the cell is modulated. In one embodiment, the agent is an antibody that specifically binds to a polypeptide of the invention.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense with respect to the coding strand of an mRNA encoding a polypeptide of the invention.

The present invention also provides methods to treat a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention. In other embodiments, the modulator is a peptide, peptidominetic, or other small molecule (e.g., a small organic molecule).

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

In yet a further aspect, the invention provides substantially purified antibodies or fragments thereof (i.e., antibody substances), including non-human antibodies or fragments thereof, which specifically bind with a polypeptide of the invention or with a portion thereof. In various embodiments, these substantially purified antibodies/fragments can be human, non-human, chimeric, and/or humanized antibodies. Non-human antibodies included in the invention include, by way of example, goat, mouse, sheep, horse, chicken, rabbit, and rat antibodies. In addition, the antibodies of the invention can be polyclonal antibodies or monoclonal antibodies.

In a particularly preferred embodiment, the antibody substance of the invention specifically binds with an extracellular domain of one of TANGO 202, TANGO 234, TANGO 265, TANGO 273, TANGO 286, TANGO 294, and INTERCEPT 296. Preferably, the extracellular domain with which the antibody substance binds has an amino acid sequence selected from the group consisting of SEQ ID NOs: 5, 6, 14, 22, 30, 37, 49, 50, and 56-58.

Any of the antibody substances of the invention can be conjugated with a therapeutic moiety or with a detectable substance. Non-limiting examples of detectable substances that can be conjugated with the antibody substances of the invention include an enzyme, a prosthetic group, a fluorescent material (i.e., a fluorophore), a luminescent material, a bioluminescent material, and a radioactive material (e.g., a radionuclide or a substituent comprising a radionuclide).

The invention also provides a kit containing an antibody substance of the invention conjugated with a detectable substance, and instructions for use. Still another aspect of the invention is a pharmaceutical composition comprising an antibody substance of the invention and a pharmaceutically acceptable carrier. In preferred embodiments, the pharmaceutical composition contains an antibody substance of the invention, a therapeutic moiety (preferably conjugated with the antibody substance), and a pharmaceutically acceptable carrier.

The invention includes a method of assessing whether a first human patient is afflicted with an epithelial or endothelial tumor (e.g., a colon, prostate, pancreatic, lung, or breast tumor). The method comprises comparing:

a) occurrence of a nucleic acid molecule of claim 1 in a sample obtained from the first patient and

b) occurrence of the nucleic acid molecule in a control sample selected from the group consisting of

i) a control sample obtained from a tissue that is obtained from the first patient and that is known not to comprise the tumor; and

ii) a control sample obtained from a second patient who is known not to be afflicted with the tumor.

A difference between the first sample and the control sample is an indication that the patient is afflicted with the tumor.

The invention also includes a method of assessing whether a first human patient is afflicted with an epithelial or endothelial tumor. This method comprises comparing

a) occurrence of a nucleic acid molecule of claim 1 in a sample obtained from the first patient and

b) occurrence of the nucleic acid molecule in a control sample selected from the group consisting of

i) a control sample obtained from a tissue that is obtained from the first patient and that is known to comprise the tumor; and

ii) a control sample obtained from a second patient who is known to be afflicted with the tumor.

A difference between the first sample and the control sample is an indication that the patient is not afflicted with the tumor.

In another embodiment, the invention includes a method of screening for agents which decrease the activity of a TANGO-294-like lipase protein. The method comprises:

contacting a test compound with a TANGO 294-like lipase polypeptide encoded by an isolated nucleic acid molecule of claim 1 and

detecting binding between the test compound and the TANGO 294-like lipase polypeptide,

Binding between the test compound and the TANGO 294-like lipase polypeptide is an indication that the test compound is an agent which decreases the activity of the TANGO 294-like lipase protein.

A pharmaceutical composition can be made by identifying an agent according to this method and combining the agent and a pharmaceutically acceptable carrier to form the pharmaceutical composition. This pharmaceutical composition can be administered to a human afflicted with a disorder in order to modulate the activity of a TANGO 294-like lipase protein in the disorder. The disorder can, for example be a tumor, a disorder of fat absorption, a disorder of fat metabolism, a blood flow disorder, a blood pressure disorder, an inflammatory disorder, an immune disorder, a thrombotic disorder, or a disorder involving inappropriate platelet adherence. Specific examples of these disorders include tumors of endothelial or epithelial origin, colon tumors, pancreatic tumors, inadequate expression of gastric lipase, inadequate expression of pancreatic lipase, cystic fibrosis, exocrine pancreatic insufficiency, obesity, arterial hypertension, renovascular hypertension, syncope, orthostatic hypotension, shock, gastritis, gastric ulcer, colitis, irritable bowel syndrome, inflammatory bowel syndrome, dermatitis, pancreatitis, rheumatoid arthritis, psoriasis, myasthenia gravis, an allergy, insulin resistance, systemic lupus erythematosus, scleroderma, and autoimmune diabetes mellitus, an infection of a human by an infectious agent (e.g., human immunodeficiency virus), hemophilia, stroke, myocardial infarction, coronary artery disease, and atherosclerosis.

In still another embodiment, the invention includes a method of screening for agents which modulate the activity of a TANGO 294-like lipase protein. This method comprises:

contacting a test compound with a TANGO 294-like lipase polypeptide encoded by an isolated nucleic acid molecule of claim 1 and

detecting a TANGO 294-like lipase activity of the polypeptide,

Increased TANGO 294-like lipase activity in the presence of the test compound is an indication that the test compound is an agent useful for increasing the activity of the TANGO 294-like lipase protein. Decreased TANGO 294-like lipase activity in the presence of the test compound is an indication that the test compound is an agent useful for decreasing the activity of the TANGO 294-like lipase protein.

A pharmaceutical composition can be made by identifying an agent according to this method and combining the agent and a pharmaceutically acceptable carrier to form the pharmaceutical composition. This pharmaceutical composition can be administered to a human afflicted with a disorder in order to modulate the activity of a TANGO 294-like lipase protein in the disorder. The disorder can, for example be a tumor, a disorder of fat absorption, a disorder of fat metabolism, a blood flow disorder, a blood pressure disorder, an inflammatory disorder, an immune disorder, a thrombotic disorder, or a disorder involving inappropriate platelet adherence. Specific examples of these disorders include tumors of endothelial or epithelial origin, colon tumors, pancreatic tumors, inadequate expression of gastric lipase, inadequate expression of pancreatic lipase, cystic fibrosis, exocrine pancreatic insufficiency, obesity, arterial hypertension, renovascular hypertension, syncope, orthostatic hypotension, shock, gastritis, gastric ulcer, colitis, irritable bowel syndrome, inflammatory bowel syndrome, dermatitis, pancreatitis, rheumatoid arthritis, psoriasis, myasthenia gravis, an allergy, insulin resistance, systemic lupus erythematosus, scleroderna, and autoimmune diabetes mellitus, an infection of a human by an infectious agent (e.g., human immunodeficiency virus), hemophilia, stroke, myocardial infarction, coronary artery disease, and atherosclerosis.

In yet another embodiment, the invention includes a method of screening for agents which decrease the activity of a TANGO 294-like lipase protein. This method comprises:

contacting a test compound with an isolated nucleic acid molecule of claim 1 and

detecting binding of the test compound with the isolated nucleic acid molecule,

Binding between the test compound and the isolated nucleic acid molecule is an indication that the test compound is a agent useful for decreasing the activity of the TANGO 294-like lipase protein.

A pharmaceutical composition can be made by identifying an agent according to this method and combining the agent and a pharmaceutically acceptable carrier to form the pharmaceutical composition. This pharmaceutical composition can be administered to a human afflicted with a disorder in order to modulate the activity of a TANGO 294-like lipase protein in the disorder. The disorder can, for example be a tumor, a disorder of fat absorption, a disorder of fat metabolism, a blood flow disorder, a blood pressure disorder, an inflammatory disorder, an immune disorder, a thrombotic disorder, or a disorder involving inappropriate platelet adherence. Specific examples of these disorders include tumors of endothelial or epithelial origin, colon tumors, pancreatic tumors, inadequate expression of gastric lipase, inadequate expression of pancreatic lipase, cystic fibrosis, exocrine pancreatic insufficiency, obesity, arterial hypertension, renovascular hypertension, syncope, orthostatic hypotension, shock, gastritis, gastric ulcer, colitis, irritable bowel syndrome, inflammatory bowel syndrome, dermatitis, pancreatitis, rheumatoid arthritis, psoriasis, myasthenia gravis, an allergy, insulin resistance, systemic lupus erythematosus, scleroderma, and autoimmune diabetes mellitus, an infection of a human by an infectious agent (e.g., human immunodeficiency virus), hemophilia, stroke, myocardial infarction, coronary artery disease, and atherosclerosis.

The invention also includes a method of reducing the activity of a TANGO 294-like lipase protein (i.e., TANGO 294, or a variant or alternative form thereof as disclosed herein) of a cell. The method comprises contacting the cell with a reagent which specifically binds with an isolated TANGO 294 nucleic acid molecule or with an isolated TANGO 294 polypeptide.

Other features and advantages of the invention will be apparent from the following detailed description and claims.

### BRIEF DESCRIPTION OF THE SEVERAL VIEWS OF THE DRAWINGS

FIG. 1 comprises FIGS. 1A-1M. The nucleotide sequence (SEQ ID NO: 1) of a cDNA encoding the human TANGO 202 protein described herein is listed in FIGS 1A-1D. The open reading frame (ORF; residues 34 to 1458; SEQ ID NO: 2) of the cDNA is indicated by nucleotide triplets, above which the amino acid sequence (SEQ ID NO: 3) of human TANGO 202 is listed. The nucleotide sequence (SEQ ID NO: 67) of a cDNA encoding the murine TANGO 202 protein described herein is listed in FIGS. 1E-1I. The ORF (residues 81 to 1490; SEQ ID NO: 68) of the cDNA is indicated by nucleotide triplets, above which the amino acid sequence (SEQ ID NO: 69) of murine TANGO 202 is listed. An alignment of the amino acid sequences of human (“Hum.”; SEQ ID NO: 3) and murine (“Mur.”; SEQ ID NO: 69) TANGO 202 protein is shown in FIGS. 1J and 1K, wherein identical amino acid residues are indicated by “:” and similar amino acid residues are indicated by “.”.

FIG. 1L is a hydrophilicity plot of human TANGO 202 protein, in which the locations of cysteine residues (“Cys”) and potential N-glycosylation sites (“Ngly”) are indicated by vertical bars and the predicted extracellular (“out”), intracellular (“ins”), or transmembrane (“TM”) locations of the protein backbone is indicated by a horizontal bar.

FIG. 1M is a hydrophilicity plot of murine TANGO 202 protein.

FIG. 2 comprises FIGS. 2A to 2Q-17. The nucleotide sequence (SEQ ID NO: 9) of a cDNA encoding the human TANGO 234 protein described herein is listed in FIGS. 2A-2I. The ORF (residues 28 to 4386; SEQ ID NO: 10) of the cDNA is indicated by nucleotide triplets, above which the amino acid sequence (SEQ ID NO: 11) of human TANGO 234 is listed.

FIG. 2J is a hydrophilicity plot of human TANGO 234 protein. An alignment of the amino acid sequences of human TANGO 234 (“Hum”; SEQ ID NO: 11) and bovine WC1 (“WC1”; SEQ ID NO: 78) proteins is shown in FIGS. 2K-2P, wherein identical amino acid residues are indicated by “:” and similar amino acid residues are indicated by “.”. An alignment of the nucleotide sequences of an ORF encoding human TANGO 234 (“Hum”; SEQ ID NO: 10) and an ORF encoding bovine WC1 (“WC1”; SEQ ID NO: 79) proteins is shown in FIGS. 2Q-1 to 2Q-17, wherein identical nucleotide residues are indicated by “:”.

FIG. 3 comprises FIGS. 3A-3U. The nucleotide sequence (SEQ ID NO: 17) of a cDNA encoding the human TANGO 265 protein described herein is listed in FIGS. 3A-3E. The ORF (residues 32 to 2314; SEQ ID NO: 18) of the cDNA is indicated by nucleotide triplets, above which the amino acid sequence (SEQ ID NO: 19) of human TANGO 265 is listed. An alignment of the amino acid sequences of human TANGO 265 protein (“Hum.”; SEQ ID NO: 19) and murine semaphorin B protein (“Mur.”; SEQ ID NO: 70; GenBank Accession No. X85991) is shown in FIGS. 3F-3H, wherein identical amino acid residues are indicated by “:” and similar amino acid residues are indicated by “.”.

In FIGS. 31-3T, an alignment of the nucleotide sequences of the cDNA encoding human TANGO 265 protein (“Hum.”; SEQ ID NO: 17) and the nucleotide sequences of the cDNA encoding murine semaphorin B protein (“Mur.”; SEQ ID NO: 71; GenBank Accession No. X85991) is shown.

FIG. 3U is a hydrophilicity plot of TANGO 265 protein.

FIG. 4 comprises FIGS. 4A-4J. The nucleotide sequence (SEQ ID NO: 25) of a cDNA encoding the human TANGO 273 protein described herein is listed in FIGS. 4A-4C. The ORF (residues 135 to 650; SEQ ID NO: 26) of the cDNA is indicated by nucleotide triplets, above which the amino acid sequence (SEQ ID NO: 27) of human TANGO 273 is listed. The nucleotide sequence (SEQ ID NO: 72) of a cDNA encoding the murine TANGO 273 protein described herein is listed in FIGS. 4D-4G. The ORF (residues 137 to 652; SEQ ID NO: 73) of the cDNA is indicated by nucleotide triplets, above which the amino acid sequence (SEQ ID NO: 74) of murine TANGO 273 is listed. An alignment of the amino acid sequences of human (“Hum.”; SEQ ID NO: 27) and murine (“Mur.”; SEQ ID NO: 74) TANGO 273 protein is shown in FIG. 4H, wherein identical amino acid residues are indicated by “:” and similar amino acid residues are indicated by “.”.

FIG. 4J is a hydrophilicity plot of human TANGO 273 protein, and

FIG. 4J is a hydrophilicity plot of murine TANGO 273 protein.

FIG. 5 comprises FIGS. 5A-5I. The nucleotide sequence (SEQ ID NO: 33) of a cDNA encoding the human TANGO 286 protein described herein is listed in FIGS. 5A-5D. The ORF (residues 133 to 1497; SEQ ID NO: 34) of the cDNA is indicated by nucleotide triplets, above which the amino acid sequence (SEQ ID NO: 35) of human TANGO 286 is listed.

FIG. 5E is a hydrophilicity plot of TANGO 286 protein. An alignment of the amino acid sequences of human TANGO 286 (“286”; SEQ ID NO: 35) and BPI protein (“BPI”; SEQ ID NO: 38) protein is shown in FIGS. 5F and 5G, wherein identical amino acid residues are indicated by “:” and similar amino acid residues are indicated by “.”. An alignment of the amino acid sequences of human TANGO 286 (“286”; SEQ ID NO: 35) and RENP protein (“RENP”; SEQ ID NO: 39) is shown in FIGS. 5H and 5I, wherein identical amino acid residues are indicated by “:” and similar amino acid residues are indicated by “.”.

FIG. 6 comprises FIGS. 6A-6H. The nucleotide sequence (SEQ ID NO: 45) of a cDNA encoding the human TANGO 294 protein described herein is listed in FIGS. 6A-6C. The ORF (residues 126 to 1394; SEQ ID NO: 46) of the cDNA is indicated by nucleotide triplets, above which the amino acid sequence (SEQ ID NO: 47) of human TANGO 294 is listed. An alignment of the amino acid sequences of human TANGO 294 protein (“294”; SEQ ID NO: 47) and a known human lipase protein (“HLP”; SEQ ID NO: 75; GenBank Accession No. NP_{—}004181) is shown in FIGS. 6D and 6E, wherein identical amino acid residues are indicated by “:” and similar amino acid residues are indicated by “.”.

FIG. 6F is a hydrophilicity plot of TANGO 294 protein. An alignment of the amino acid sequences of human TANGO 294 protein (“294”; SEQ ID NO: 47) and a known human lysosomal acid lipase protein (“LAL”; SEQ ID NO: 41) is shown in FIGS. 6G and 6H, wherein identical amino acid residues are indicated by and similar amino acid residues are indicated by “.”

FIG. 7 comprises FIGS. 7A-7F. The nucleotide sequence (SEQ ID NO: 53) of a cDNA encoding the human INTERCEPT 296 protein described herein is listed in FIGS. 7A-7C. The ORF (residues 70 to 1098; SEQ ID NO: 54) of the cDNA is indicated by nucleotide triplets, above which the amino acid sequence (SEQ ID NO: 55) of human INTERCEPT 296 protein is listed.

FIG. 7D is a hydrophilicity plot of INTERCEPT 296 protein. An alignment of the amino acid sequences of human INTERCEPT 296 protein (“296”; SEQ ID NO: 55) and C. elegans C06E1.3 related protein (“CRP”; SEQ ID NO: 40) is shown in FIGS. 7E and 7F, wherein identical amino acid residues are indicated by “:” and similar amino acid residues are indicated by “.”.

### DETAILED DESCRIPTION OF THE INVENTION

The present invention is based, at least in part, on the discovery of a variety of human and murine cDNA molecules which encode proteins which are herein designated TANGO 202, TANGO 234, TANGO 265, TANGO 273, TANGO 286, TANGO 294, and INTERCEPT 296. These proteins exhibit a variety of physiological activities, and are included in a single application for the sake of convenience. It is understood that the allowability or non-allowability of claims directed to one of these proteins has no bearing on the allowability of claims directed to the others. The characteristics of each of these proteins and the cDNAs encoding them are now described separately.

TANGO 202

A cDNA clone (designated jthke096b05) encoding at least a portion of human TANGO 202 protein was isolated from a human fetal skin cDNA library. The corresponding murine cDNA was isolated as a clone (designated jtmMa044f07) from a bone marrow stromal cell cDNA library. The human TANGO 202 protein is predicted by structural analysis to be a type I membrane protein, although it can exist in a secreted form as well. The murine TANGO 202 protein is predicted by structural analysis to be a secreted protein.

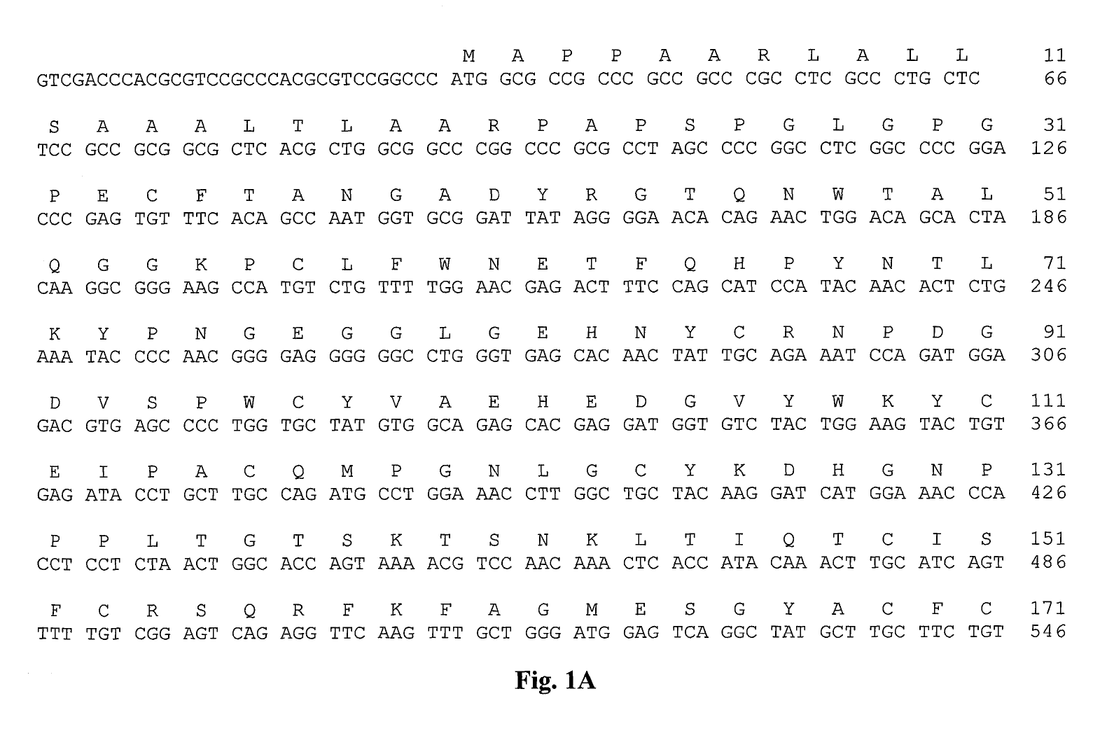

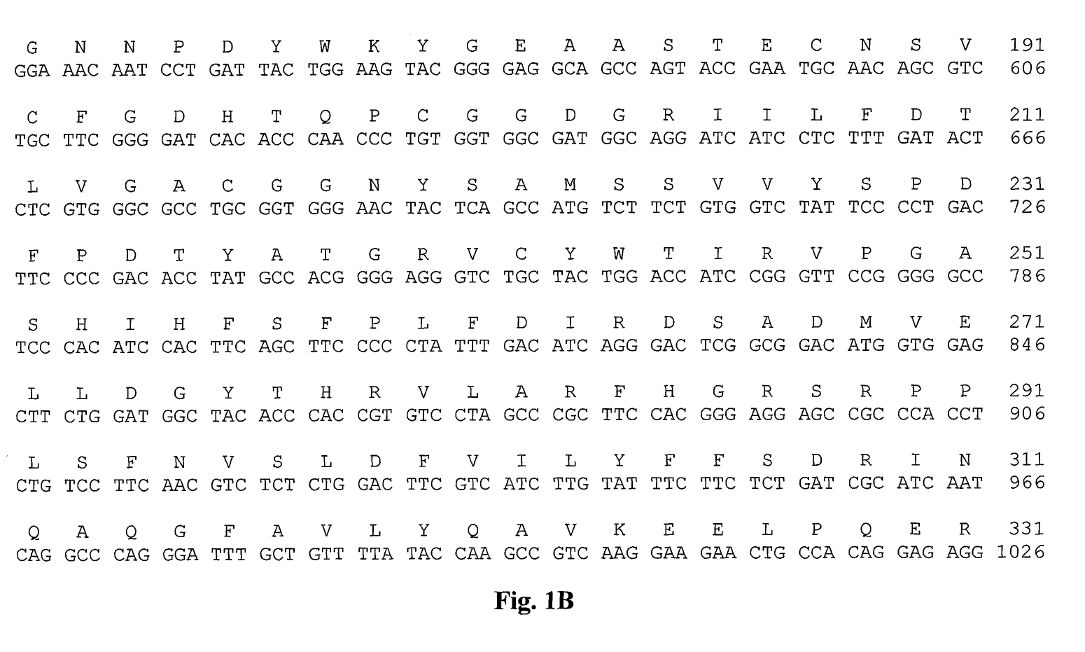

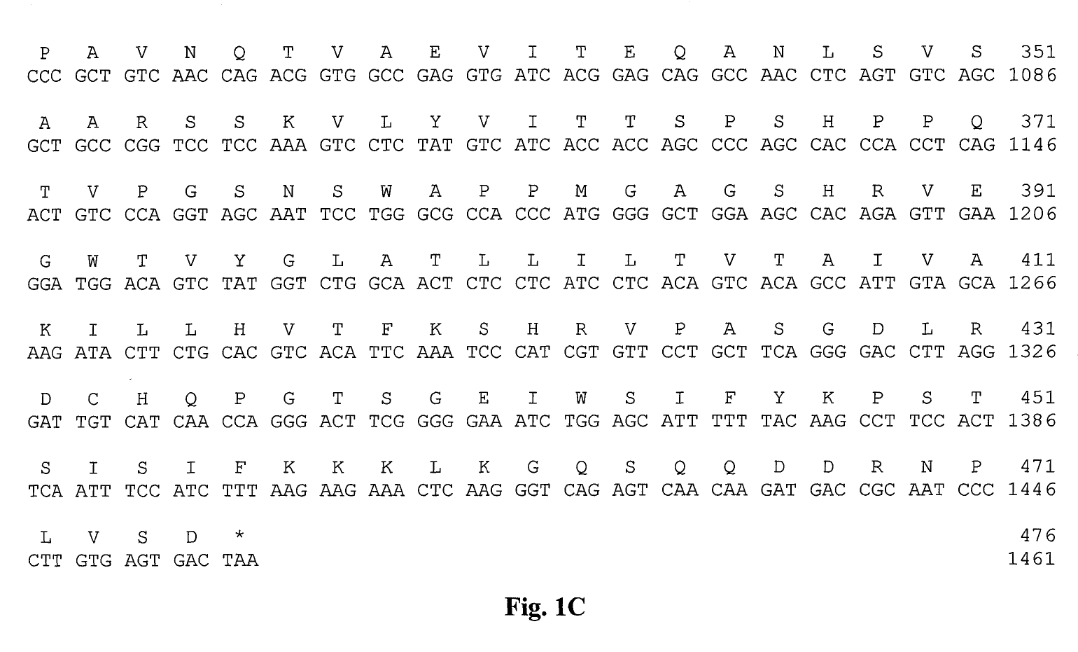

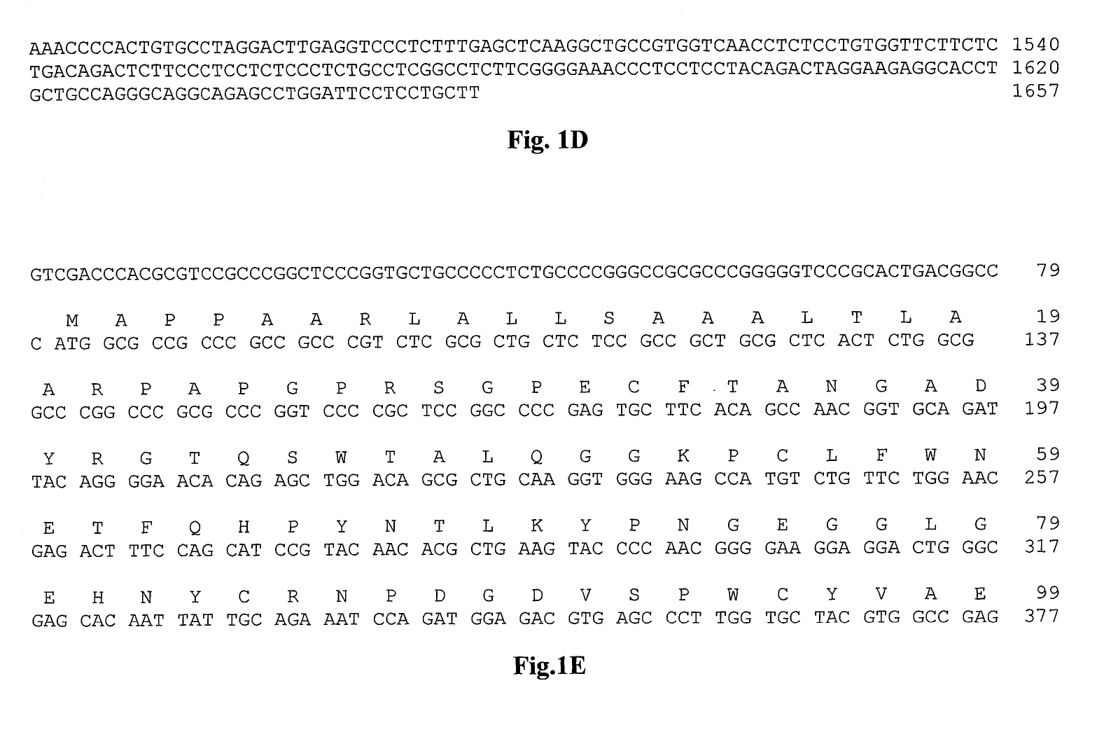

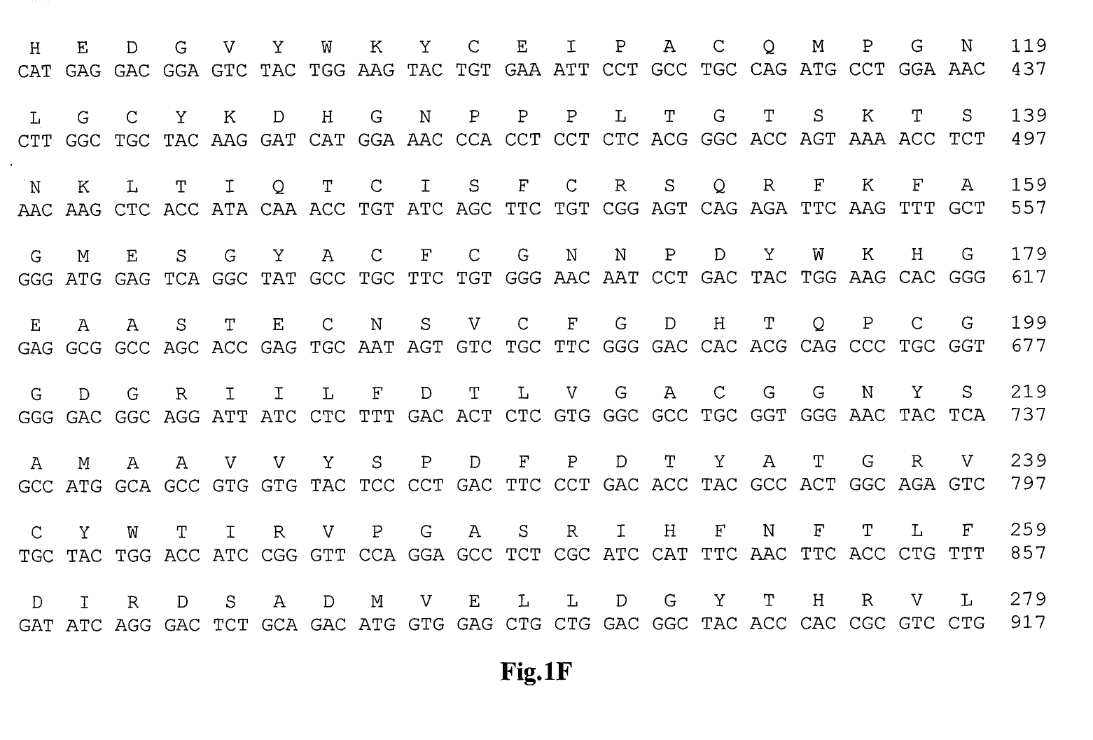

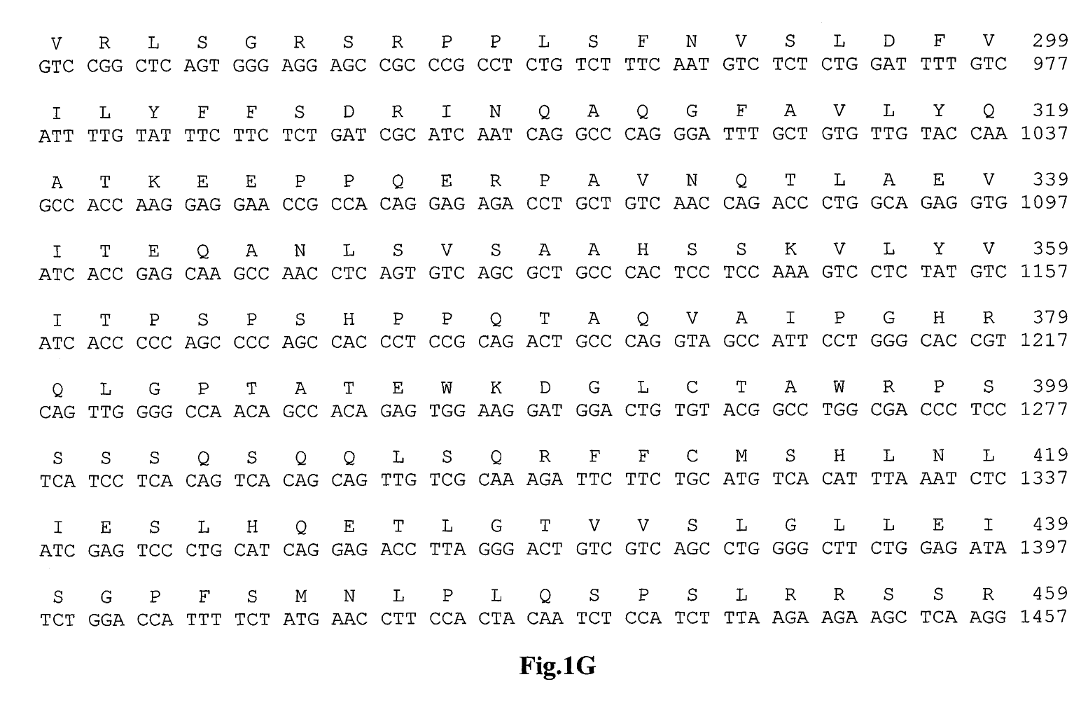

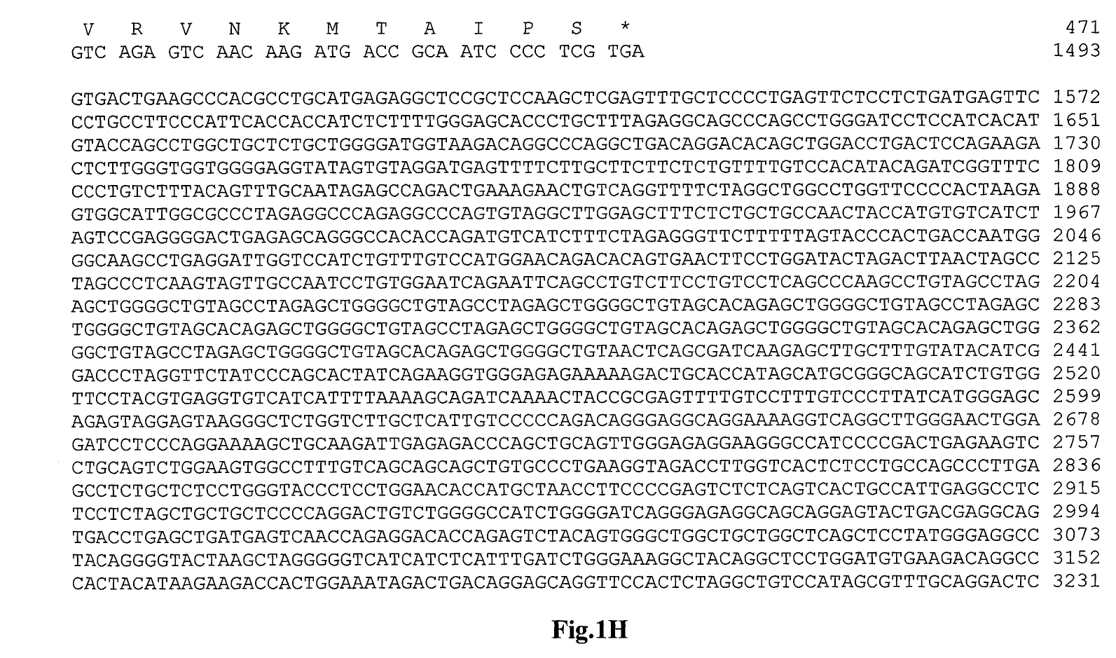

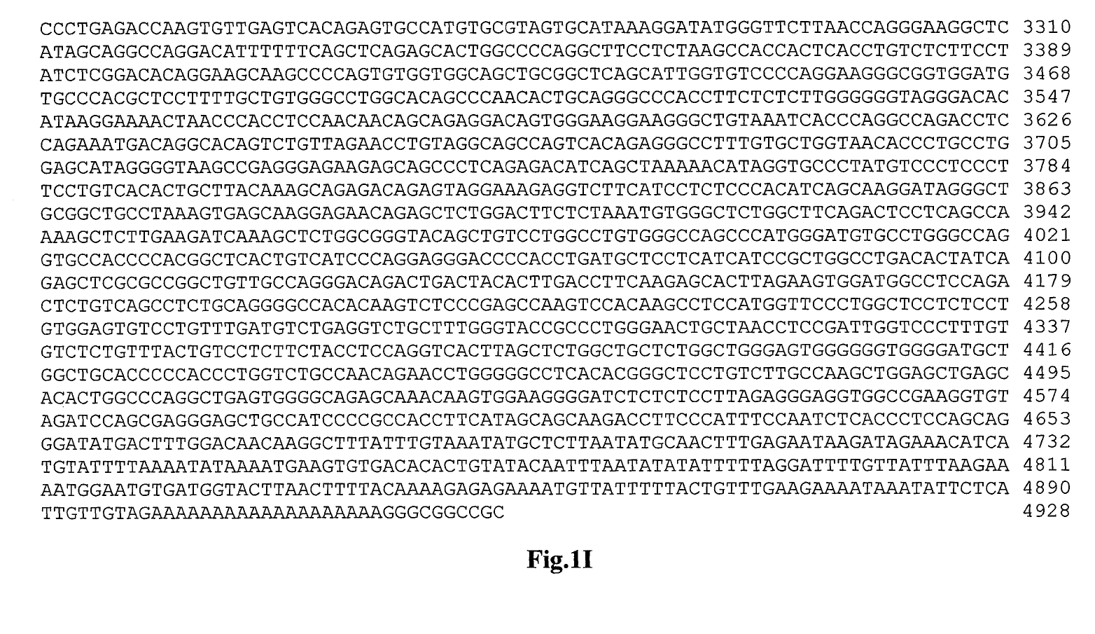

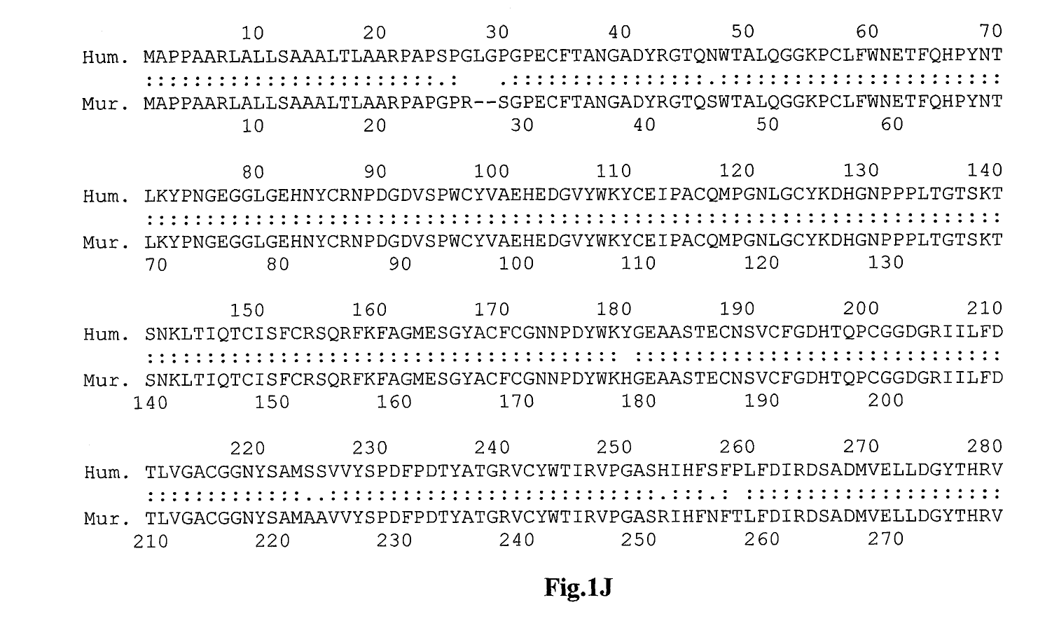

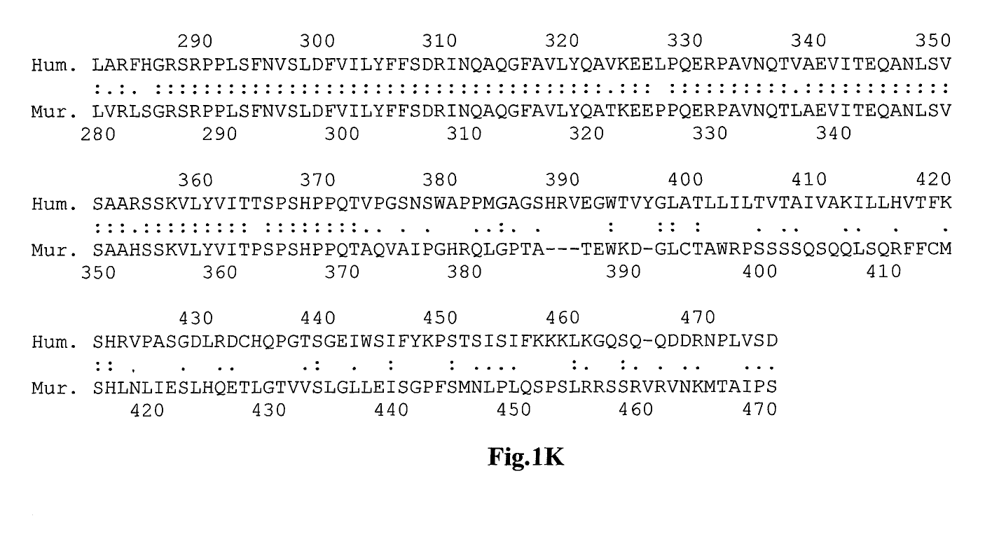

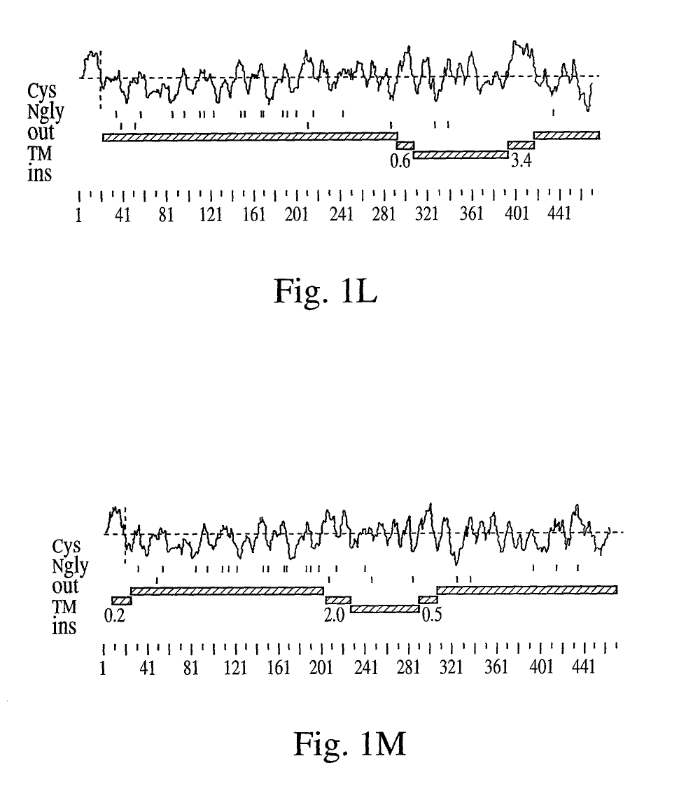

The full length of the cDNA encoding human TANGO 202 protein (FIG. 1; SEQ ID NO: 1) is 1656 nucleotide residues. The open reading frame (ORF) of this cDNA, nucleotide residues 34 to 1458 of SEQ ID NO: 1 (i.e., SEQ ID NO: 2), encodes a 475-amino acid transmembrane protein (FIG. 1; SEQ ID NO: 3).

The invention thus includes purified human TANGO 202 protein, both in the form of the immature 475 amino acid residue protein (SEQ ID NO: 3) and in the form of the mature 456 amino acid residue protein (SEQ ID NO: 5). The invention also includes purified murine TANGO 202 protein, both in the form of the immature 470 amino acid residue protein (SEQ ID NO: 67) and in the form of the mature 451 amino acid residue protein (SEQ ID NO: 43). Mature human or murine TANGO 202 proteins can be synthesized without the signal sequence polypeptide at the amino terminus thereof, or they can be synthesized by generating immature TANGO 202 protein and cleaving the signal sequence therefrom.

In addition to full length mature and immature human and murine TANGO 202 proteins, the invention includes fragments, derivatives, and variants of these TANGO 202 proteins, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 1 or some portion thereof or SEQ ID NO: 67 or some portion thereof, such as the portion which encodes mature human or murine TANGO 202 protein, immature human or murine TANGO 202 protein, or a domain of human or murine TANGO 202 protein. These nucleic acids are collectively referred to as nucleic acids of the invention.

TANGO 202 proteins and nucleic acid molecules encoding them comprise a family of molecules having certain conserved structural and functional features. As used herein, the term “family” is intended to mean two or more proteins or nucleic acid molecules having a common or similar domain structure and having sufficient amino acid or nucleotide sequence identity as defined herein. Family members can be from either the same or different species (e.g., human and mouse, as described herein). For example, a family can comprise two or more proteins of human origin, or can comprise one or more proteins of human origin and one or more of non-human origin.

A common domain present in TANGO 202 proteins is a signal sequence. As used herein, a signal sequence includes a peptide of at least about 10 amino acid residues in length which occurs at the amino terminus of membrane-bound and secreted proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 202 protein contains a signal sequence corresponding to amino acid residues 1 to 19 of SEQ ID NO: 3 (SEQ ID NO: 4) or to amino acid residues 1 to 19 of SEQ ID NO: 69 (SEQ ID NO: 42). The signal sequence is cleaved during processing of the mature protein.

TANGO 202 proteins can also include an extracellular domain. As used herein, an “extracellular domain” refers to a portion of a protein which is localized to the non-cytoplasmic side of a lipid bilayer of a cell when a nucleic acid encoding the protein is expressed in the cell. The human TANGO 202 protein extracellular domain is located from about amino acid residue 20 to about amino acid residue 392 of SEQ ID NO: 3 in the non-secreted form, and from about amino acid residue 20 to amino acid residue 475 of SEQ ID NO: 3 (i.e., the entire mature human protein). The murine TANGO 202 protein extracellular domain is located from about amino acid residue 20 to amino acid residue 470 of SEQ ID NO: 69 (i.e., the entire mature murine protein).

TANGO 202 proteins of the invention can also include a transmembrane domain. As used herein, a “transmembrane domain” refers to an amino acid sequence having at least about 20 to 25 amino acid residues in length and which contains at least about 65-70% hydrophobic amino acid residues such as alanine, leucine, phenylalanine, protein, tyrosine, tryptophan, or valine. In a preferred embodiment, a transmembrane domain contains at least about 15 to 30 amino acid residues, preferably about 20-25 amino acid residues, and has at least about 60-80%, more preferably 65-75%, and more preferably at least about 70% hydrophobic residues. Thus, in one embodiment, a TANGO 202 protein of the invention contains a transmembrane domain corresponding to about amino acid residues 393 to 415 of SEQ ID NO: 3 (SEQ ID NO: 7).

In addition, TANGO 202 proteins of the invention can include a cytoplasmic domain, particularly including a carboxyl-terminal cytoplasmic domain. As used herein, a “cytoplasmic domain” refers to a portion of a protein which is localized to the cytoplasmic side of a lipid bilayer of a cell when a nucleic acid encoding the protein is expressed in the cell. The cytoplasmic domain is located from about amino acid residue 416 to amino acid residue 475 of SEQ ID NO: 3 (SEQ ID NO: 8) in the non-secreted form of human TANGO 202 protein.

TANGO 202 proteins typically comprise a variety of potential post-translational modification sites (often within an extracellular domain), such as those described herein in Tables I (for human TANGO 202) and II (for murine TANGO 202), as predicted by computerized sequence analysis of TANGO 202 proteins using amino acid sequence comparison software (comparing the amino acid sequence of TANGO 202 with the information in the PROSITE database {rel. 12.2; February, 1995} and the Hidden Markov Models database {Rel. PFAM 3.3}).

TABLE I

| | Amino Acid | |
|---|---|---|
| Type of Potential Modification Site or | Residues of | Amino Acid |
| Domain | SEQ ID NO: 3 | Sequence |
| N-glycosylation site | 47 to 50 | NWTA |
| | 61 to 64 | NETF |
| | 219 to 222 | NYSA |
| | 295 to 298 | NVSL |
| | 335 to 338 | NQTV |
| | 347 to 350 | NLSV |
| Protein kinase C phosphorylation site | 70 to 72 | TLK |
| | 137 to 139 | TSK |
| | 141 to 143 | SNK |
| | 155 to 157 | SQR |
| | 238 to 240 | TGR |
| | 245 to 247 | TIR |
| | 277 to 279 | THR |
| | 307 to 309 | SDR |
| | 355 to 357 | SSK |
| | 387 to 389 | SHR |
| | 418 to 420 | TFK |
| | 421 to 423 | SHR |
| Casein kinase II phosphorylation site | 337 to 340 | TVAE |
| | 438 to 441 | TSGE |
| | 464 to 467 | SQQD |
| N-myristoylation site | 53 to 58 | GGKPCL |
| | 120 to 125 | GNLGCY |
| | 136 to 141 | GTSKTS |
| | 162 to 167 | GMESGY |
| | 214 to 219 | GACGGN |
| Kringle domain signature | 85 to 90 | YCRNPD |
| Kringle Domain | 34 to 116 | See FIG. 1 |
| CUB domain | 216 to 320 | See FIG. 1 |

TABLE II

| | Amino Acid | |
|---|---|---|
| Type of Potential Modification | Residues of | Amino Acid |
| Site or Domain | SEQ ID NO: 69 | Sequence |
| N-glycosylation site | 59 to 62 | NETF |
| | 217 to 220 | NYSA |
| | 255 to 258 | NFTL |
| | 293 to 296 | NVSL |
| | 333 to 336 | NQTL |
| | 345 to 348 | NLSV |
| cAMP- or cGMP-dependent protein | 455 to 458 | RRSS |
| kinase phosphorylation site | | |
| Protein kinase C phosphorylation site | 68 to 70 | TLK |
| | 135 to 137 | TSK |
| | 139 to 141 | SNK |
| | 153 to 155 | SQR |
| | 236 to 238 | TGR |
| | 243 to 245 | TIR |
| | 275 to 277 | THR |
| | 283 to 285 | SGR |
| | 305 to 307 | SDR |
| | 353 to 355 | SSK |
| | 408 to 410 | SQR |
| | 453 to 455 | SLR |
| | 457 to 459 | SSR |
| Casein kinase II phosphorylation site | 28 to 31 | SGPE |
| | 257 to 260 | TLFD |
| | 321 to 324 | TKEE |
| | 335 to 338 | TLAE |
| | 384 to 387 | TATE |
| N-myristoylation site | 51 TO 56 | GGKPCL |
| | 118 TO 123 | GNLGCY |
| | 134 TO 139 | GTSKTS |
| | 160 TO 165 | GMESGY |
| | 212 TO 217 | GACGGN |
| | 391 TO 396 | GLCTAW |
| | 429 TO 434 | GTVVSL |
| Kringle domain signature | 83 to 88 | YCRNPD |
| Kringle Domain | 32 to 114 | See FIG. 1 |
| CUB domain | 214 to 318 | See FIG. 1 |

As used herein, the term “post-translational modification site” refers to a protein domain that includes about 3 to 10 amino acid residues, more preferably about 3 to 6 amino acid residues wherein the domain has an amino acid sequence which comprises a consensus sequence which is recognized and modified by a protein-modifying enzyme. Exemplary protein-modifying enzymes include amino acid glycosylases, cAMP- and cGMP-dependent protein kinases, protein kinase C, casein kinase II, myristoylases, and prenyl transferases. In various embodiments, the protein of the invention has at least 1, 2, 4, 6, 10, 15, or 20 or more of the post-translational modification sites described herein in Tables I and II.

Exemplary additional domains present in human and murine TANGO 202 protein include Kringle domains and CUB domains. In one embodiment, the protein of the invention has at least one domain that is at least 55%, preferably at least about 65%, more preferably at least about 75%, yet more preferably at least about 85%, and most preferably at least about 95% identical to one of the domains described herein in Tables I and II. Preferably, the protein of the invention has at least one Kringle domain and one CUB domain.

A Kringle domain has a characteristic profile that has been described in the art (Castellino and Beals (1987) J. Mol. Evol. 26:358-369; Patthy (1985) Cell 41:657-663; Ikeo et al. (1991) FEBS Lett. 287:146-148). Many, but not all, Kringle domains comprise a conserved hexapeptide signature sequence, namely

(F or Y)-C-R-N-P-(D or N or R).

The cysteine residue is involved in a disulfide bond.

Kringle domains are triple-looped, disulfide cross-linked domains found in a varying number of copies in, for example, some serine proteases and plasma proteins. Kringle domains have a role in binding mediators (e.g., membranes, other proteins, or phospholipids) and in regulation of proteolytic activity. Kringle domains have been identified in the following proteins, for example: apolipoprotein A, blood coagulation factor XII (Hageman factor), hepatocyte growth factor (HGF), HGF-like protein (Friezner Degen et al., (1991) Biochemistry 30:9781-9791), HGF activator (Miyazawa et al., (1993) J. Biol. Chem. 268:10024-10028), plasminogen, thrombin, tissue plasminogen activator, urokinase-type plasminogen activator, and four influenza neuraminidases. The presence of a Kringle domain in each of human and murine TANGO 202 protein indicates that TANGO 202 is involved in one or more physiological processes in which these other Kringle domain-containing proteins are involved, has biological activity in common with one or more of these other Kringle domain-containing proteins, or both.

CUB domains are extracellular domains of about 110 amino acid residues which occur in functionally diverse, mostly developmentally regulated proteins (Bork and Beckmann (1993) J. Mol. Biol. 231:539-545; Bork (1991) FEBS Lett. 282:9-12). Many CUB domains contain four conserved cysteine residues, although some, like that of TANGO 202, contain only two of the conserved cysteine residues. The structure of the CUB domain has been predicted to assume a beta-barrel configuration, similar to that of immunoglobulins. Other proteins which have been found to comprise one or more CUB domains include, for example, mammalian complement sub-components Cls and Clr, hamster serine protease Casp, mammalian complement activating component of Ra-reactive factor, vertebrate enteropeptidase, vertebrate bone morphogenic protein 1, sea urchin blastula proteins BP10 and SpAN, Caenorhabditis elegans hypothetical proteins F42A10.8 and R151.5, neuropilin (A5 antigen), sea urchin fibropellins I and III, mammalian hyaluronate-binding protein TSG-6 (PS4), mammalian spermadhesins, and Xenopus embryonic protein UVS.2. The presence of a CUB domain in each of human and murine TANGO 202 protein indicates that TANGO 202 is involved in one or more physiological processes in which these other CUB domain-containing proteins are involved, has biological activity in common with one or more of these other CUB domain-containing proteins, or both.

The signal peptide prediction program SIGNALP (Nielsen et al. (1997) Protein Engineering 10:1-6) predicted that human TANGO 202 protein includes a 19 amino acid signal peptide (amino acid residues 1 to 19 of SEQ ID NO: 3; SEQ ID NO: 4) preceding the mature TANGO 202 protein (amino acid residues 20 to 475 of SEQ ID NO: 3; SEQ ID NO: 5). Human TANGO 202 protein includes an extracellular domain (amino acid residues 20 to 392 of SEQ ID NO: 3; SEQ ID NO: 6); a transmembrane domain (amino acid residues 393 to 415 of SEQ ID NO: 3; SEQ ID NO: 7); and a cytoplasmic domain (amino acid residues 416 to 475 of SEQ ID NO: 3; SEQ ID NO: 8). The murine homolog of TANGO 202 protein is predicted to be a secreted protein. Thus, it is recognized that human TANGO 202 can also exist in the form of a secreted protein, likely being translated from an alternatively spliced TANGO 202 mRNA. In a variant form of the protein, an extracellular portion of TANGO 202 protein (e.g., amino acid residues 20 to 392 of SEQ ID NO: 3) can be cleaved from the mature protein to generate a soluble fragment of TANGO 202.

FIG. 1L depicts a hydrophilicity plot of human TANGO 202 protein. Relatively hydrophobic regions are above the dashed horizontal line, and relatively hydrophilic regions are below the dashed horizontal line. The hydrophobic region which corresponds to amino acid residues 1 to 19 of SEQ ID NO: 3 is the signal sequence of human TANGO 202 (SEQ ID NO: 4). The hydrophobic region which corresponds to amino acid residues 393 to 415 of SEQ ID NO: 3 is the transmembrane domain of human TANGO 202 (SEQ ID NO: 7). As described elsewhere herein, relatively hydrophilic regions are generally located at or near the surface of a protein, and are more frequently effective immunogenic epitopes than are relatively hydrophobic regions. For example, the region of human TANGO 202 protein from about amino acid residue 61 to about amino acid residue 95 appears to be located at or near the surface of the protein, while the region from about amino acid residue 395 to about amino acid residue 420 appears not to be located at or near the surface.

The predicted molecular weight of human TANGO 202 protein without modification and prior to cleavage of the signal sequence is about 51.9 kilodaltons. The predicted molecular weight of the mature human TANGO 202 protein without modification and after cleavage of the signal sequence is about 50.1 kilodaltons.

The full length of the cDNA encoding murine TANGO 202 protein (FIG. 1; SEQ ID NO: 67) is 4928 nucleotide residues. The ORF of this cDNA, nucleotide residues 81 to 1490 of SEQ ID NO: 67 (i.e., SEQ ID NO: 68), encodes a 470-amino acid secreted protein (FIG. 1; SEQ ID NO: 69).

The signal peptide prediction program SIGNALP (Nielsen et al. (1997) Protein Engineering 10:1-6) predicted that murine TANGO 202 protein includes a 19 amino acid signal peptide (amino acid residues 1 to 19 of SEQ ID NO: 69; SEQ ID NO: 42) preceding the mature TANGO 202 protein (amino acid residues 20 to 470 of SEQ ID NO: 69; SEQ ID NO: 43). Murine TANGO 202 protein is a secreted protein.

FIG. 1M depicts a hydrophilicity plot of murine TANGO 202 protein. Relatively hydrophobic regions are above the dashed horizontal line, and relatively hydrophilic regions are below the dashed horizontal line. The hydrophobic region which corresponds to amino acid residues 1 to 19 of SEQ ID NO: 69 is the signal sequence of murine TANGO 202 (SEQ ID NO: 42). As described elsewhere herein, relatively hydrophilic regions are generally located at or near the surface of a protein, and are more frequently effective immunogenic epitopes than are relatively hydrophobic regions. For example, the region of murine TANGO 202 protein from about amino acid residue 61 to about amino acid residue 95 appears to be located at or near the surface of the protein, while the region from about amino acid residue 295 to about amino acid residue 305 appears not to be located at or near the surface

The predicted molecular weight of murine TANGO 202 protein without modification and prior to cleavage of the signal sequence is about 51.5 kilodaltons. The predicted molecular weight of the mature murine TANGO 202 protein without modification and after cleavage of the signal sequence is about 49.7 kilodaltons.

Human and murine TANGO 202 proteins exhibit considerable sequence similarity, as indicated herein in FIGS. 1J and 1K. FIGS. 1J and 1K depict an aligmnent of human and murine TANGO 202 amino acid sequences (SEQ ID NOs: 3 and 69, respectively). In this alignment (made using the ALIGN software {Myers and Miller (1989) CABIOS, ver. 2.0}; pam120.mat scoring matrix; gap penalties −12/−4), the proteins are 76.5% identical. The human and murine ORFs encoding TANGO 202 are 87.4% identical, as assessed using the same software and parameters.

In situ hybridization experiments in mouse tissues indicated that mRNA corresponding to the cDNA encoding TANGO 202 is expressed in the tissues listed in Table III, wherein “+” indicates detectable expression and “++” indicates a greater level of expression than “+”.

TABLE III

| | | Relative Level of |
|---|---|---|
| Animal | Tissue | Expression |
| Mouse | bladder, especially in | ++ |
| (Adult) | transitional | |
| | epithelium | |
| | renal glomeruli | + |
| | brain | + |
| | heart | + |
| | liver | + |
| | spleen | + |
| | placenta | + |
| Mouse | ubiquitous | + |
| (Embryo) | | |

Biological Function of TANGO 202 Proteins, Nucleic Acids, and Modulators Thereof

TANGO 202 proteins are involved in disorders which affect both tissues in which they are normally expressed and tissues in which they are normally not expressed. Based on the observation that TANGO 202 is expressed in human fetal skin, ubiquitously in fetal mouse tissues, in adult murine bone marrow stromal cells, and in cells of adult murine bladder, renal glomeruli, brain, heart, liver, spleen and placenta, TANGO 202 protein is involved in one or more biological processes which occur in these tissues. In particular, TANGO 202 is involved in modulating growth, proliferation, survival, differentiation, and activity of cells of these tissues including, but not limited to, hematopoietic and fetal cells. Thus, TANGO 202 has a role in disorders which affect these cells and their growth, proliferation, survival, differentiation, and activity. Ubiquitous expression of TANGO 202 in fetal murine tissues, contrasted with limited expression in adult murine tissues further indicates that TANGO 202 is involved in disorders in which it is inappropriately expressed (e.g., disorders in which TANGO 202 is expressed in adult murine tissues other than bone marrow stromal cells and disorders in which TANGO 202 is not expressed in one or more developing fetal tissues).

The presence of a Kringle domain in both the murine and human TANGO 202 proteins indicates that this protein is involved in modulating cellular binding to one or more mediators (e.g., proteins, phospholipids, intracellular organelles, or other cells), in modulating proteolytic activity, or both. The presence of a Kringle domain in other proteins (e.g., growth factors) indicates activities that these proteins share with TANGO 202 protein (e.g., modulating cell dissociation and migration into and through extracellular matrices). The presence of Kringle domains in numerous plasma proteins, particularly coupled with the observation that TANGO 202 is expressed in adult murine bone marrow stromal cells, indicates a role for TANGO 202 protein in modulating binding of blood or hematopoietic cells (or both) to one or more mediators. Thus, TANGO 202 is involved in disorders relating to aberrant cellular protease activity, inappropriate interaction or non-interaction of cells with mediators, and in blood and hematopoietic cell-related disorders. Such disorders include, by way of example and not limitation, immune disorders, infectious diseases, auto-immune disorders, vascular and cardiovascular disorders, disorders related to mal-expression of growth factors, cancers, hematological disorders, and the like.

The cDNA encoding TANGO 202 exhibits significant nucleotide sequence similarity with a polynucleotide encoding a kringle-domain-containing protein (designated HTHBZ47) described in the European Patent Application No. EP 0 911 399 A2 (published Apr. 28, 1999). Thus, the TANGO 202 protein can exhibit one or more of the activities exhibited by HTHBZ47, and can be used to prevent, inhibit, diagnose, and treat one or more disorders for which HTHBZ47 is useful. These disorders include cancer, inflammation, autoimmune disorders, allergic disorders, asthma, rheumatoid arthritis, inflammation of central nervous system tissues, cerebellar degeneration, Alzheimer's disease, Parkinson's disease, multiple sclerosis, amylotrophic lateral sclerosis, head injury damage and other neurological abnormalities, septic shock, sepsis, stroke, osteoporosis, osteoarthritis, ischemic reperfusion injury, cardiovascular disease, kidney disease, liver disease, ischemic injury, myocardial infarction, hypotension, hypertension, AIDS, myelodysplastic syndromes and other hematologic abnormalities, aplastic anemia, male pattern baldness, and bacterial, fungal, protozoan, and viral infections.

The presence of a CUB domain in both the murine and human TANGO 202 proteins indicates that this protein is involved in biological processes common to other CUB domain-containing proteins, such as developmental processes and binding to mediators. Therefore, TANGO 202 protein has a role in disorders which involve inappropriate developmental processes (e.g., abnormally high proliferation or un-differentiation of a differentiated tissue or abnormally low differentiation or proliferation of a non-developed or non-differentiated tissue) and modulation of cell growth, proliferation, survival, differentiation, and activity. Such disorders include, by way of example and not limitation, various cancers and birth and developmental defects.

Thus, proteins and nucleic acids of the invention which are identical to, similar to, or derived from human and murine TANGO 202 proteins and nucleic acids encoding them are useful for preventing, diagnosing, and treating, among others, vascular and cardiovascular disorders, hematological disorders, disorders related to mal-expression of growth factors, and cancer. Other uses for these proteins and nucleic acids of the invention relate to modulating cell growth (e.g., angiogenesis), proliferation (e.g., cancers), survival (e.g., apoptosis), differentiation (e.g., hematopoiesis), and activity (e.g., ligand-binding capacity). TANGO 202 proteins and nucleic acids encoding them are also useful for modulating cell dissociation and modulating migration of cells in extracellular matrices.

TANGO 234

A cDNA clone (designated jthsa104d11) encoding at least a portion of human TANGO 234 protein was isolated from a human fetal spleen cDNA library. The human TANGO 234 protein is predicted by structural analysis to be a transmembrane protein, although it can exist in a secreted form as well.

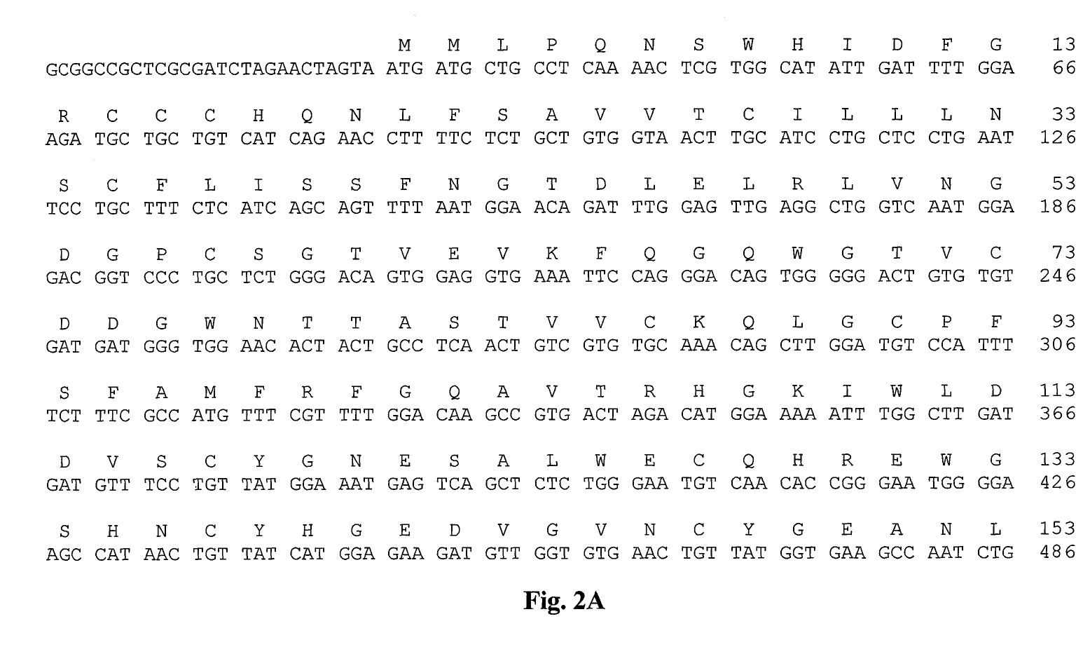

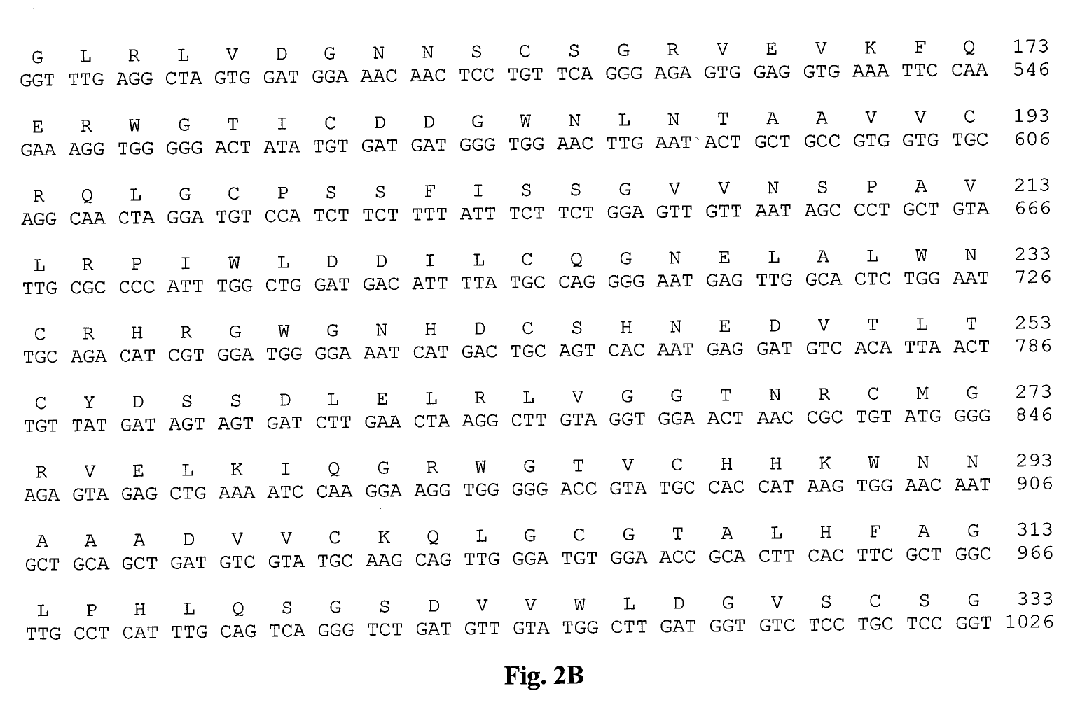

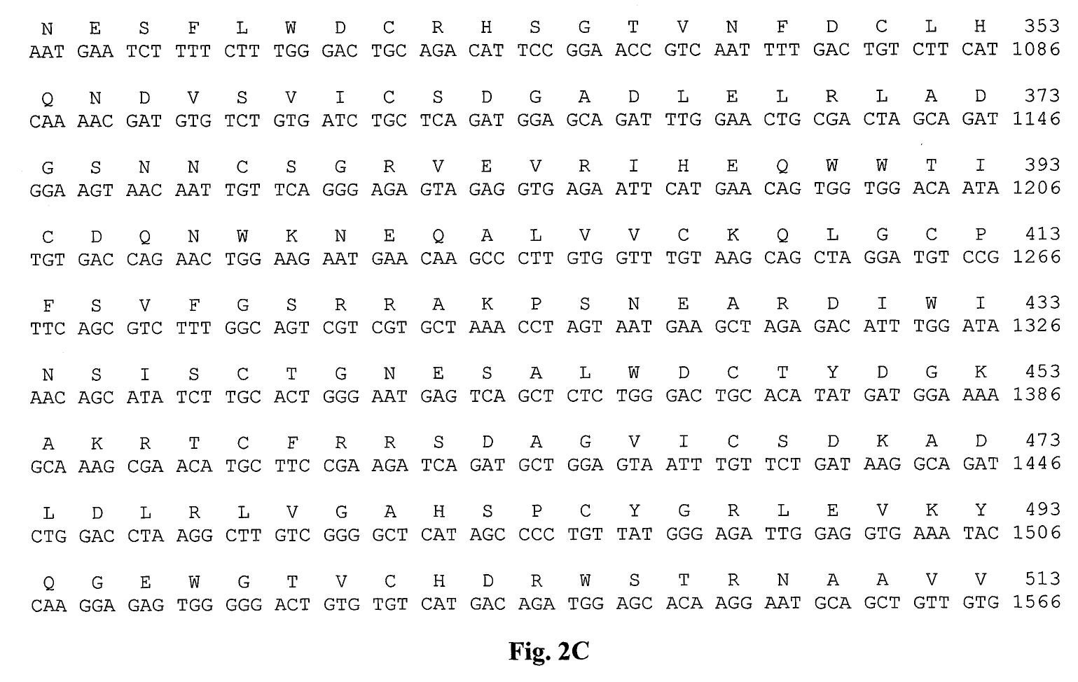

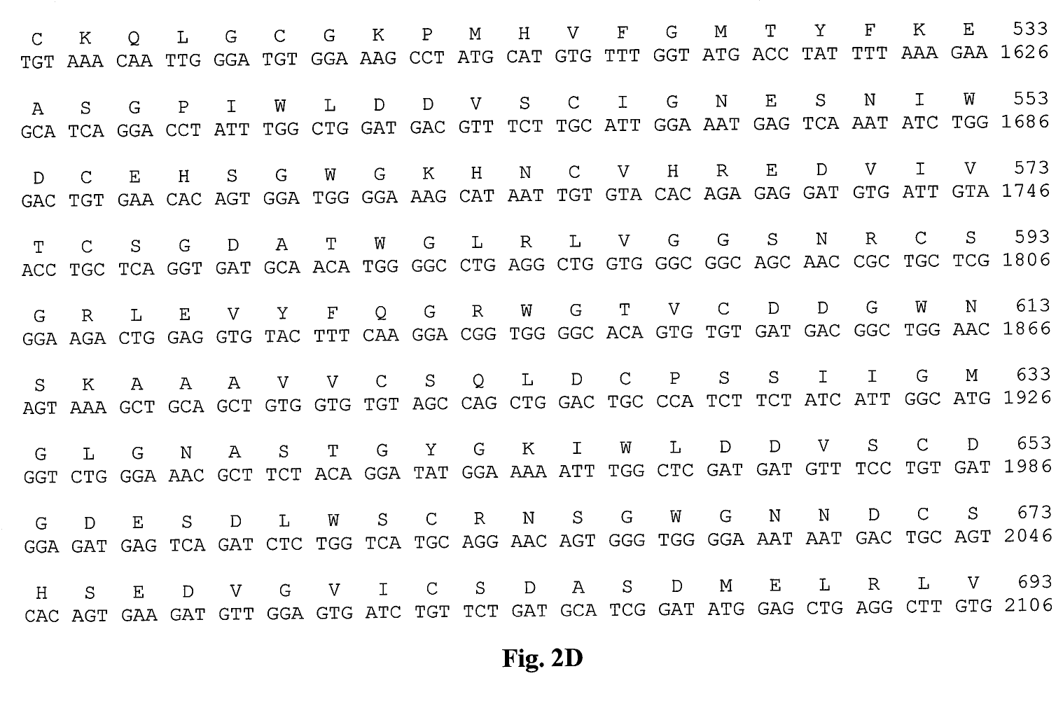

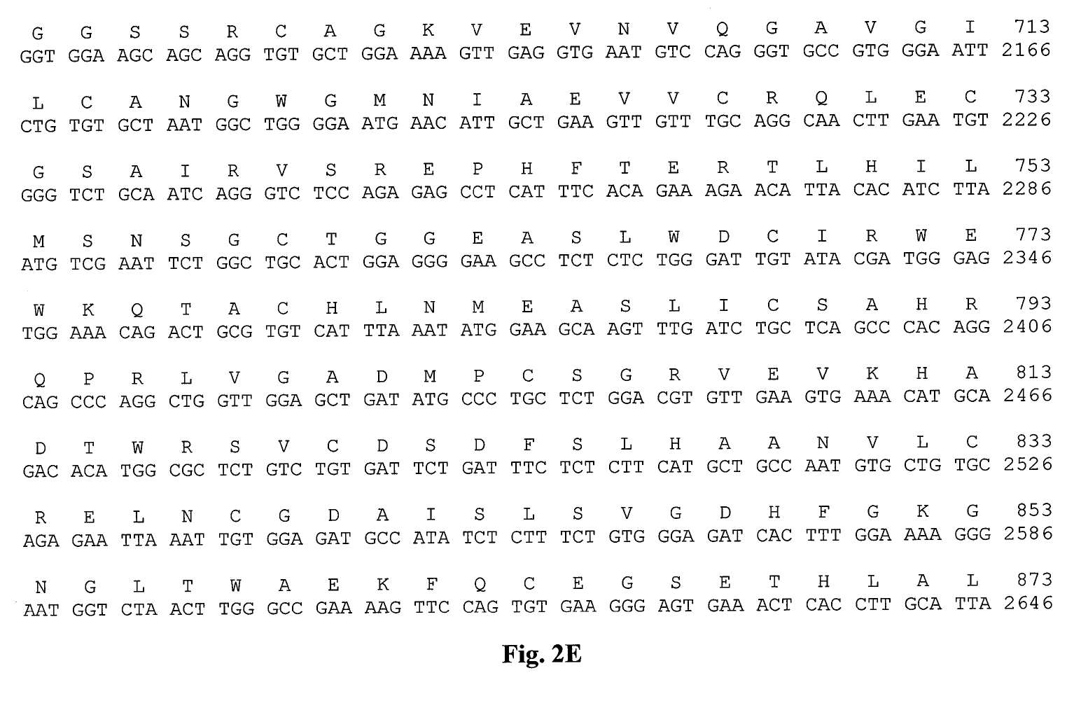

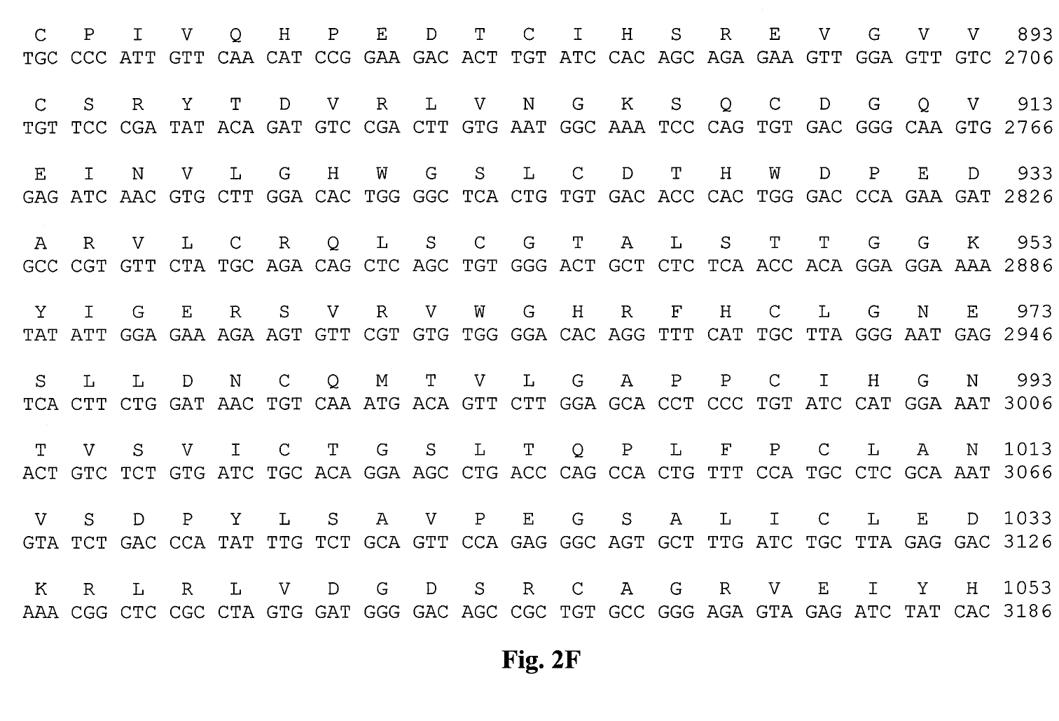

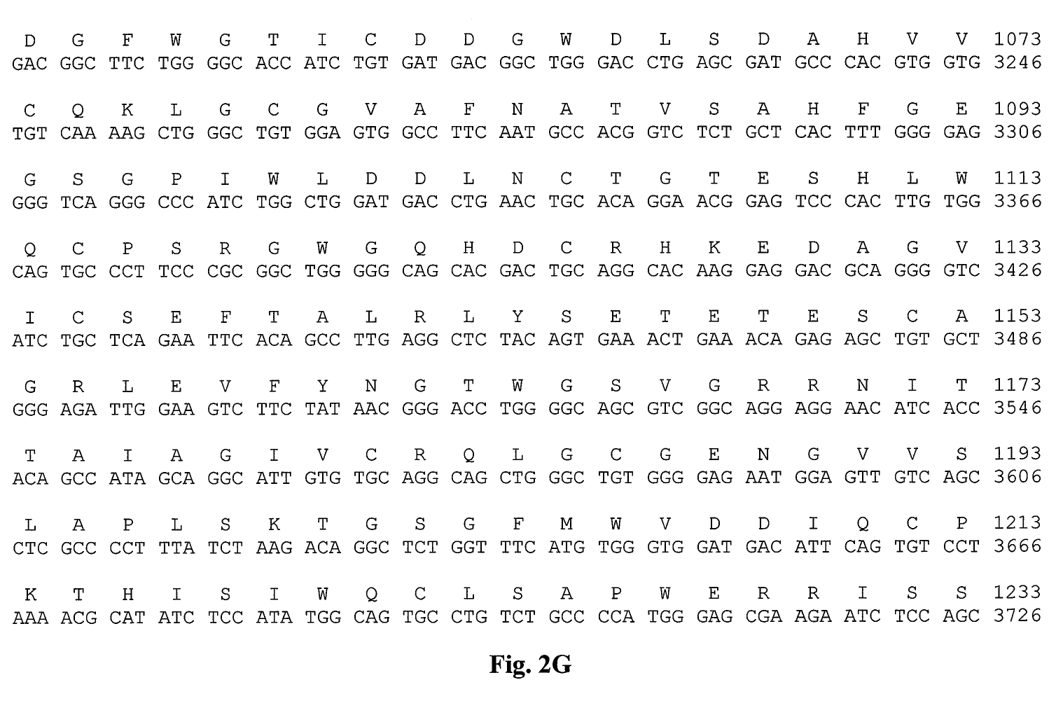

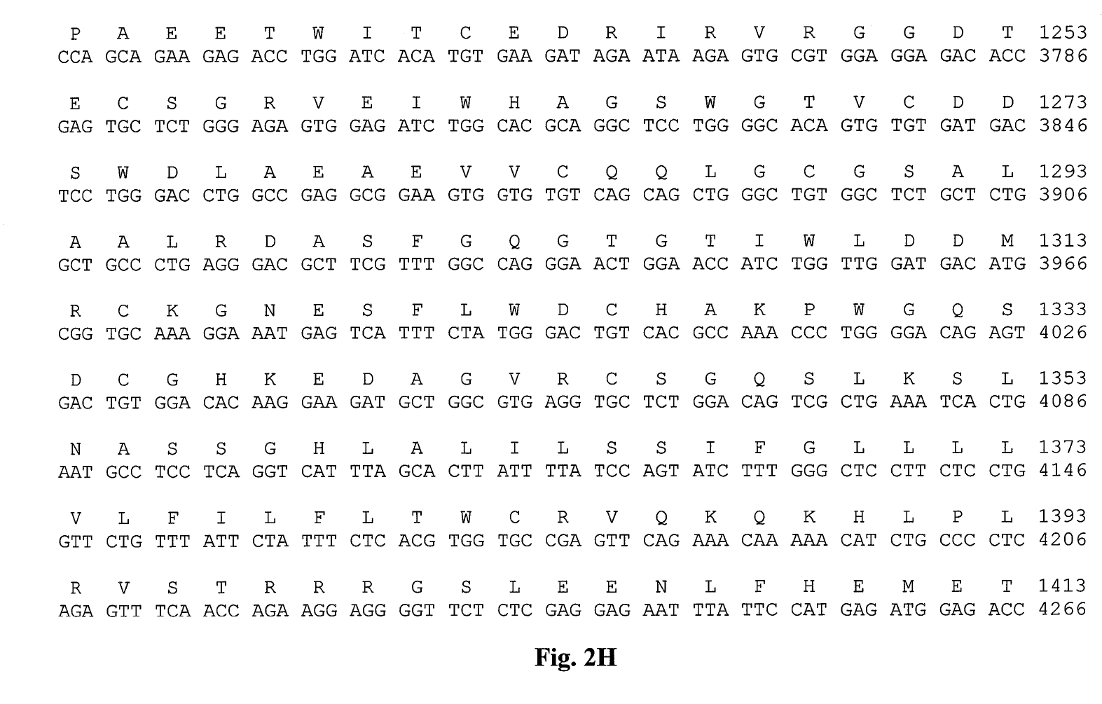

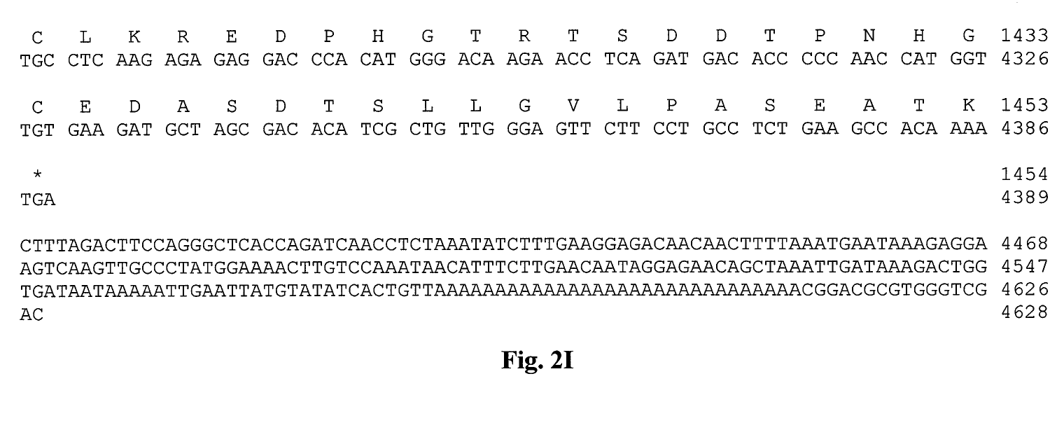

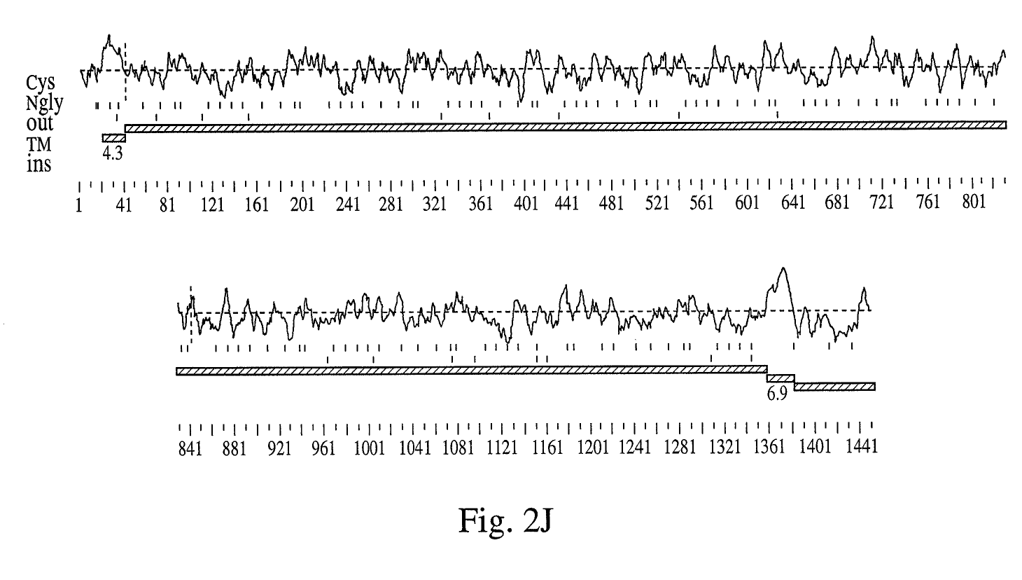

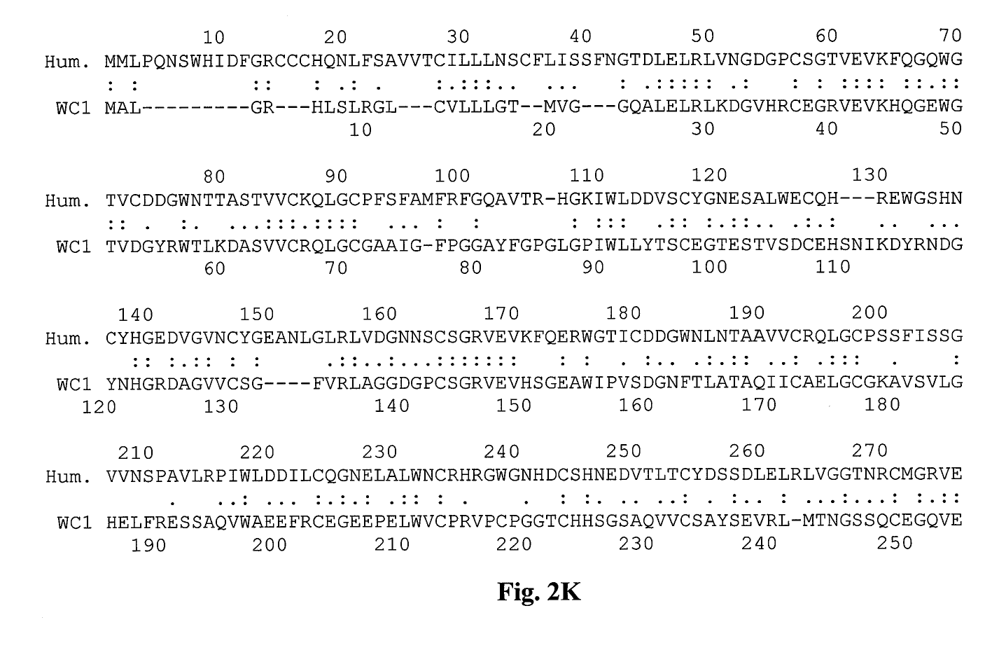

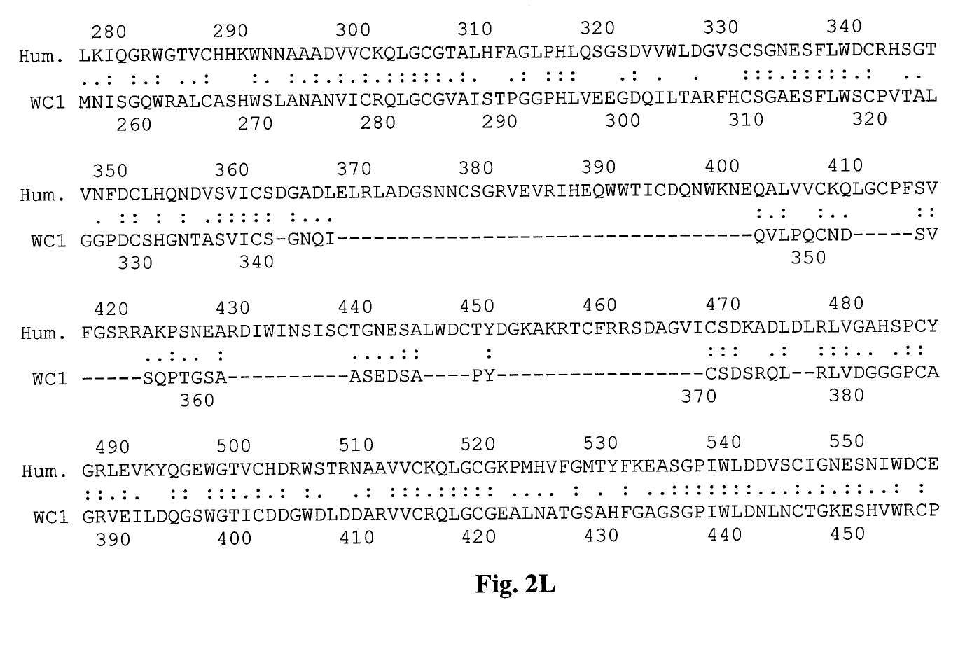

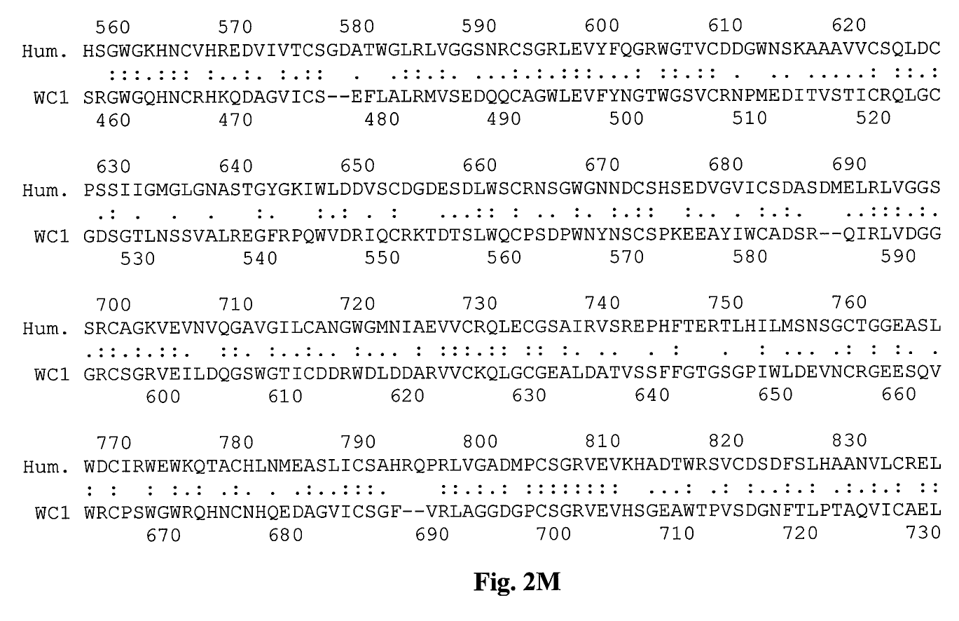

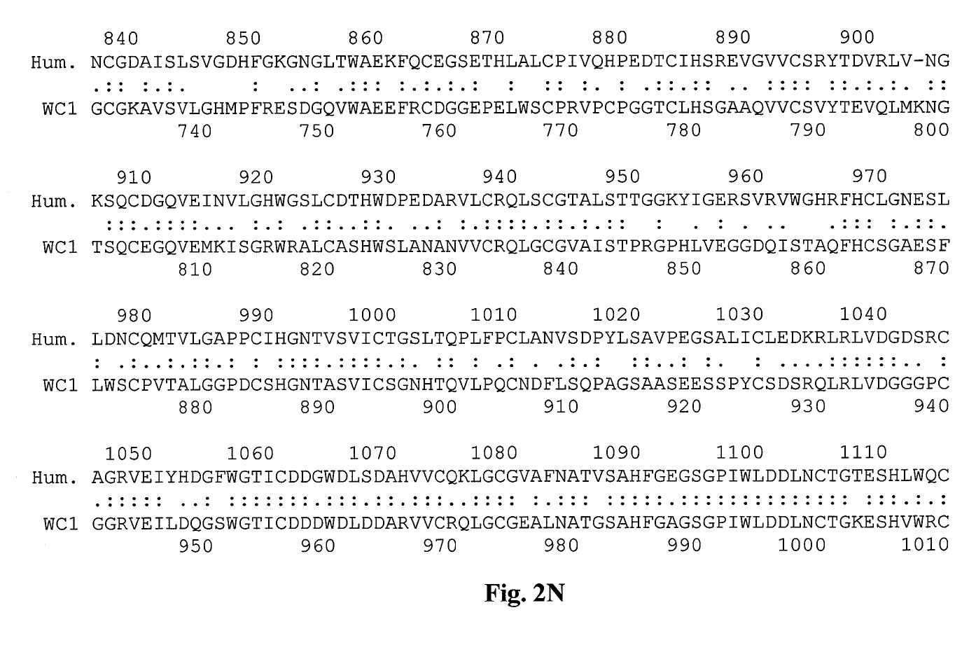

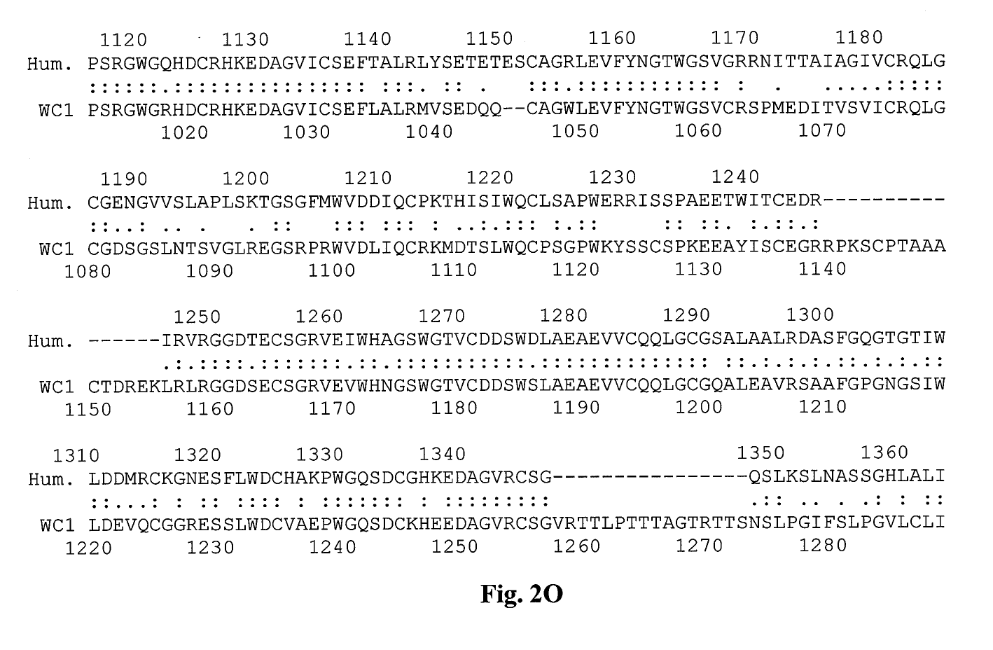

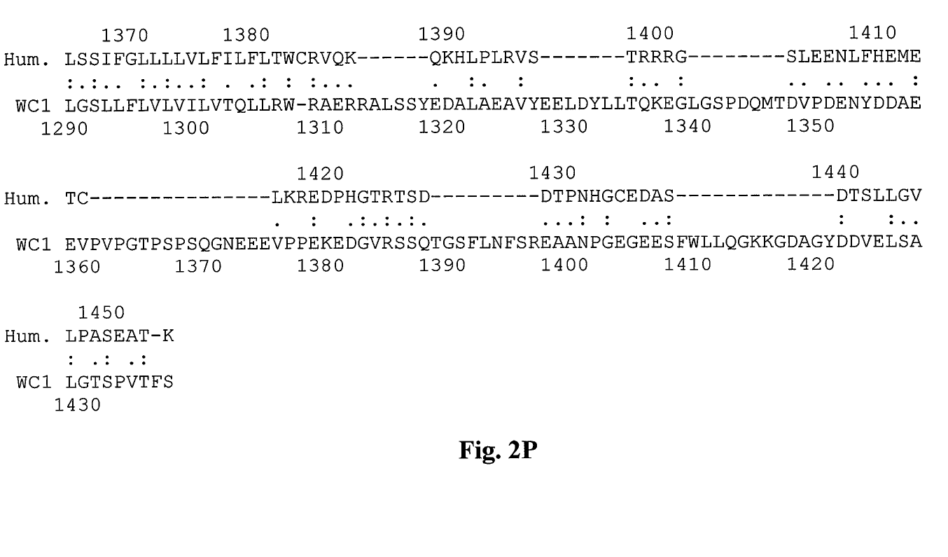

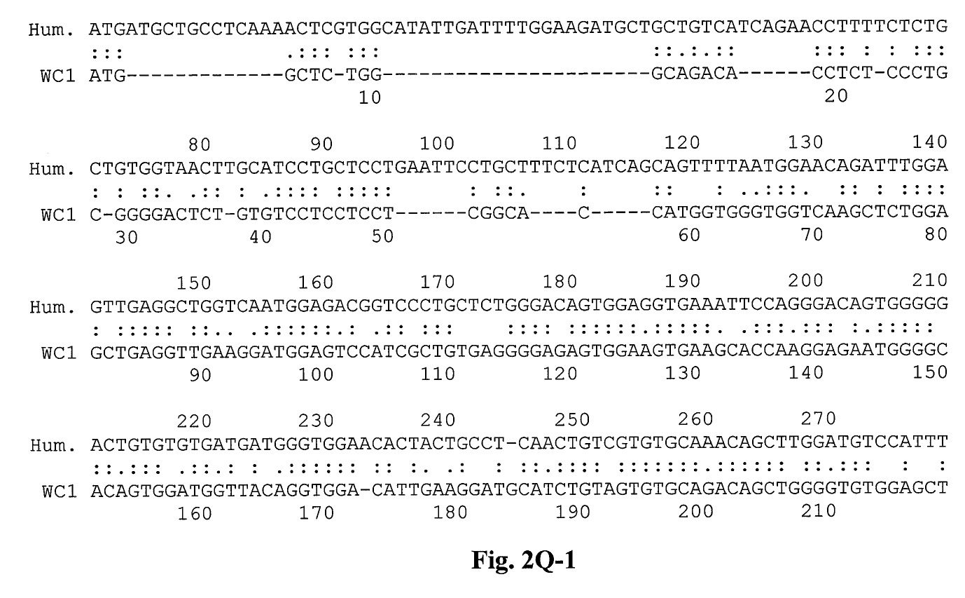

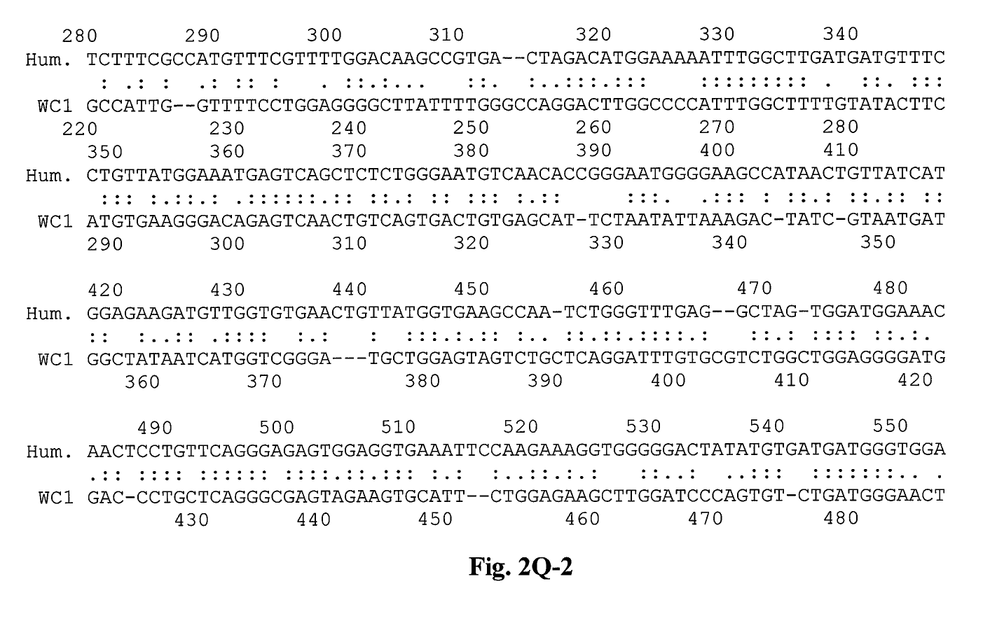

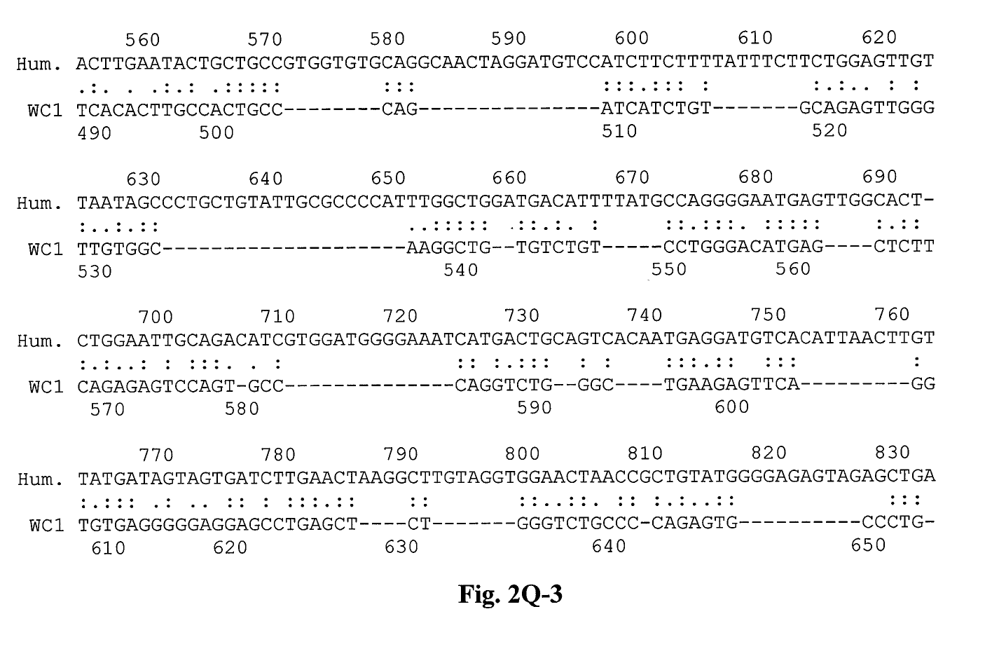

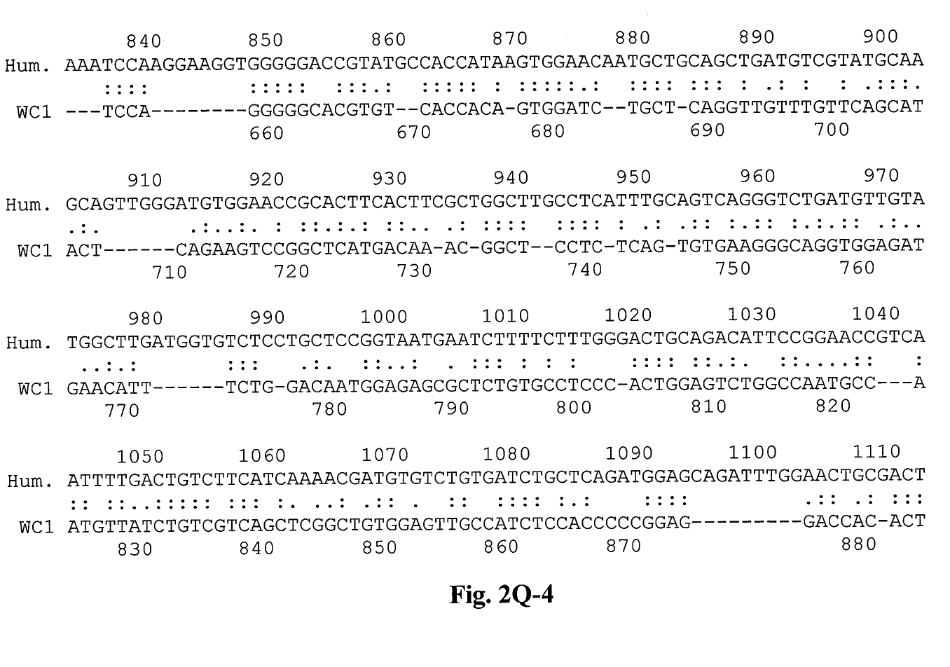

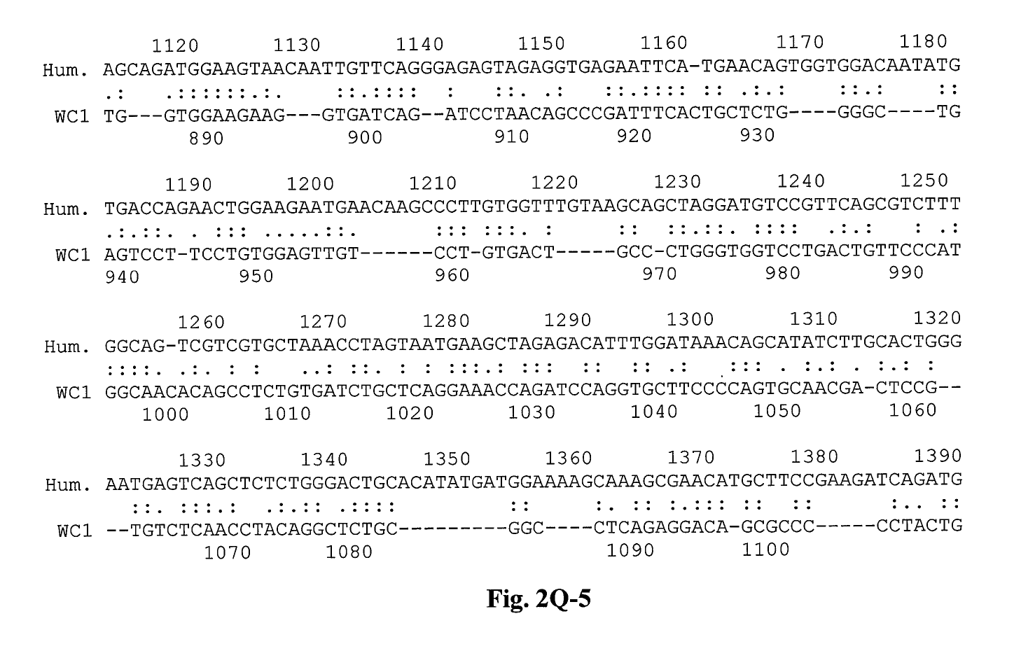

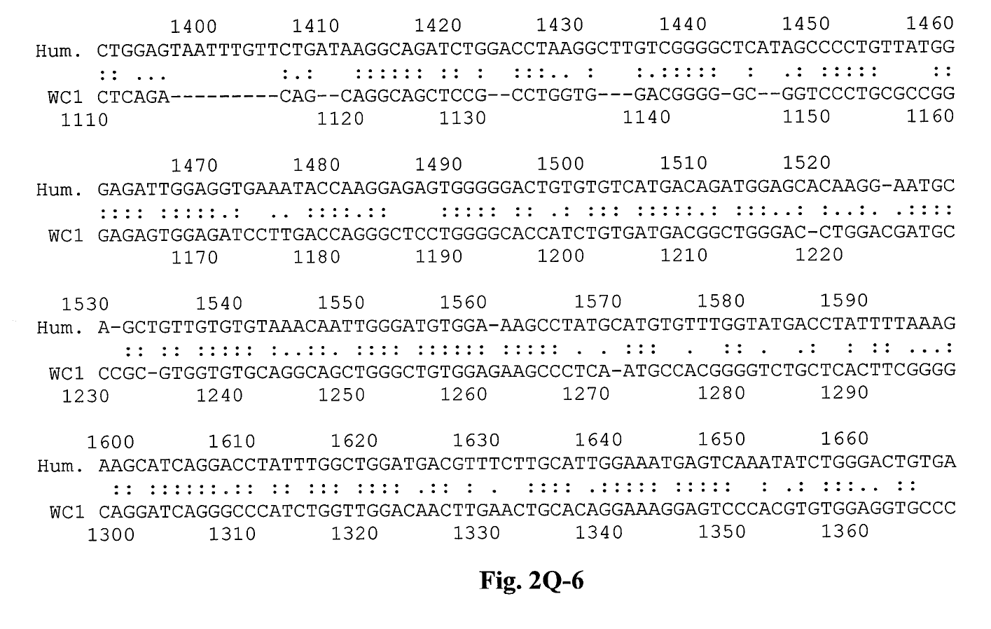

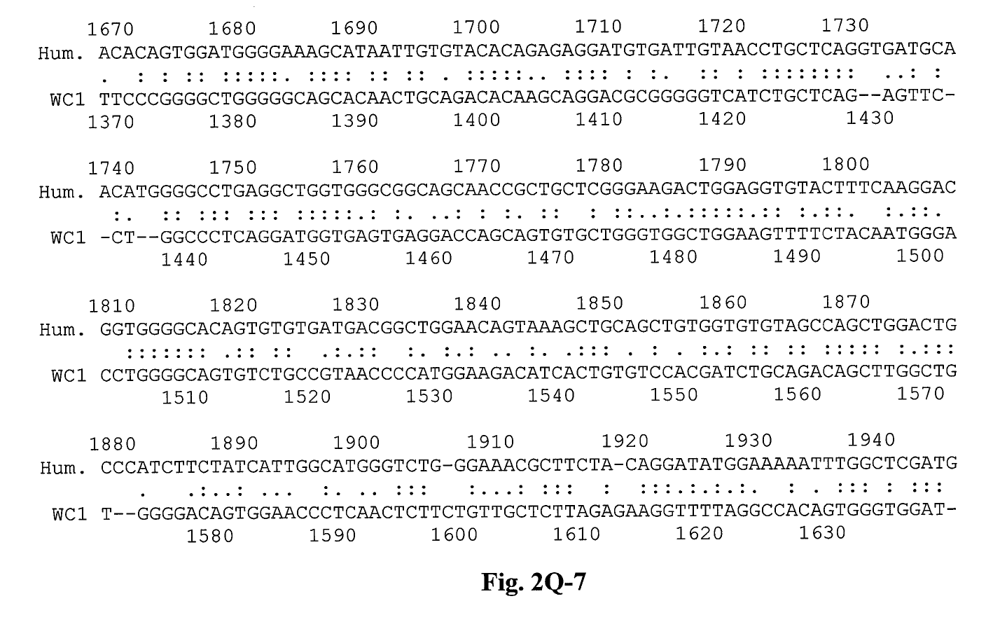

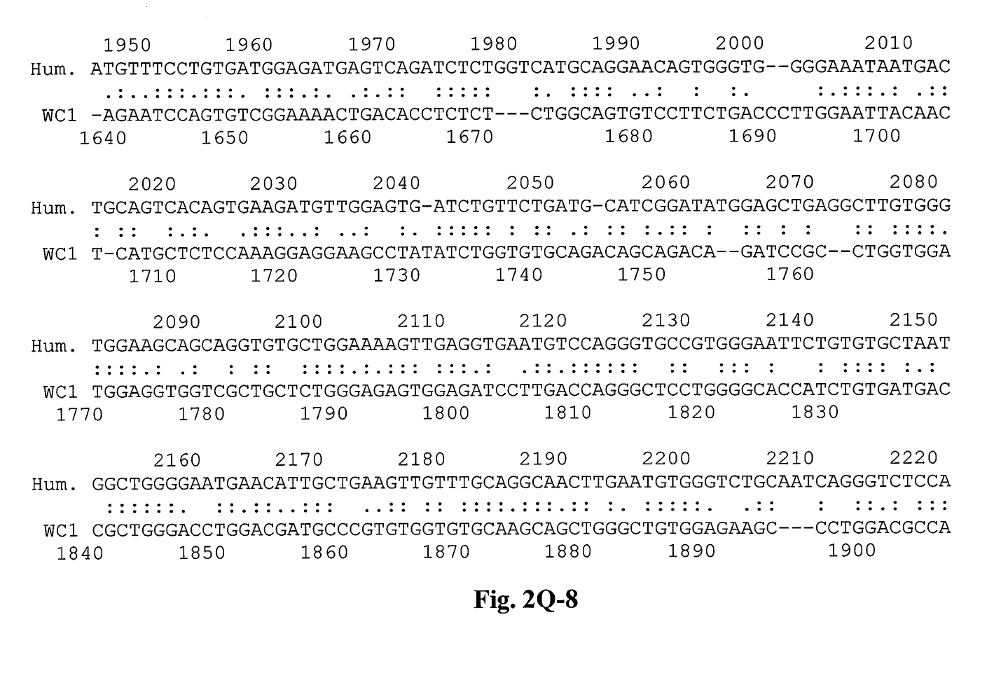

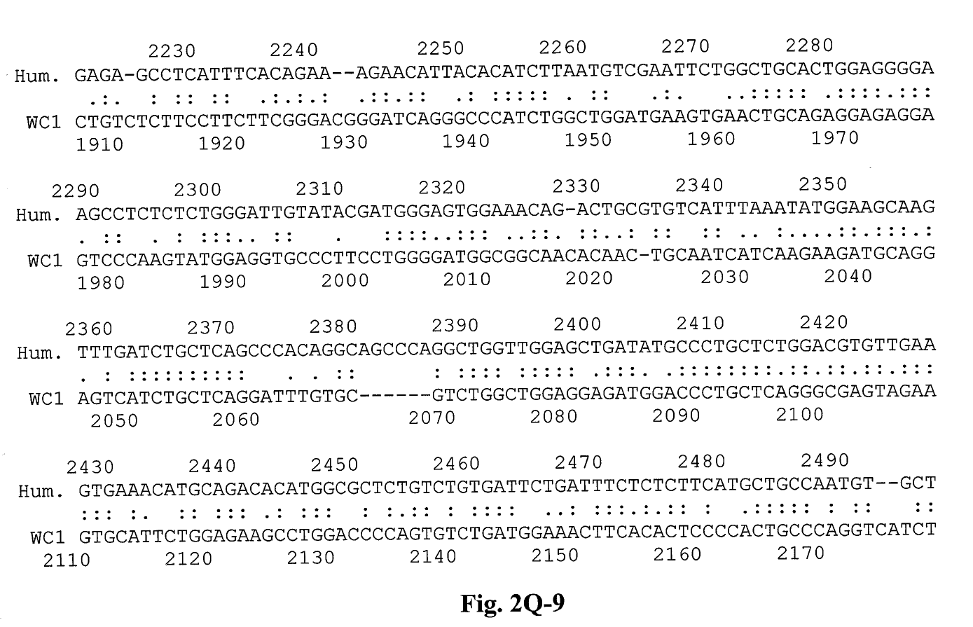

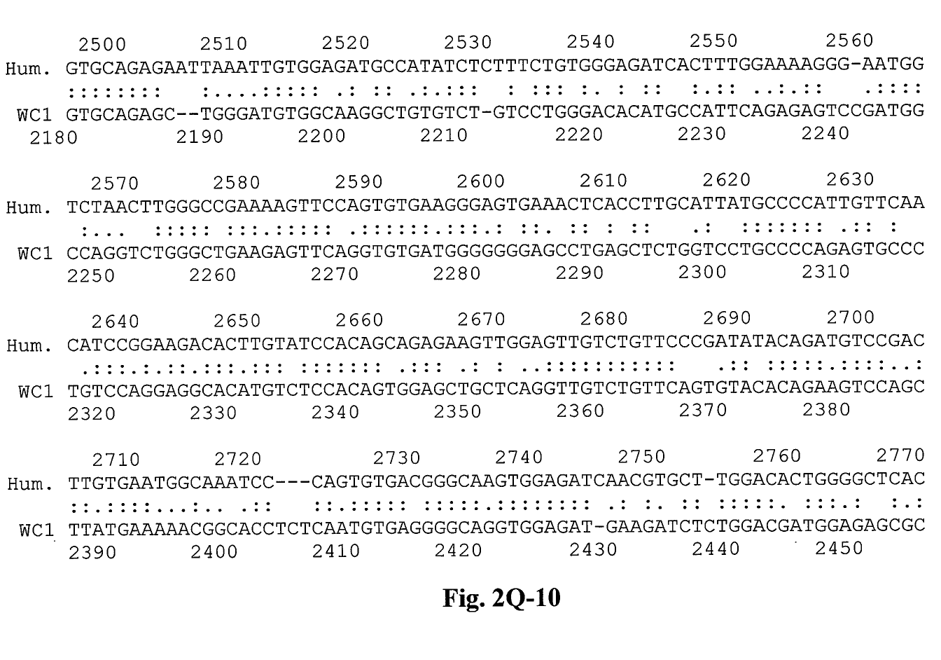

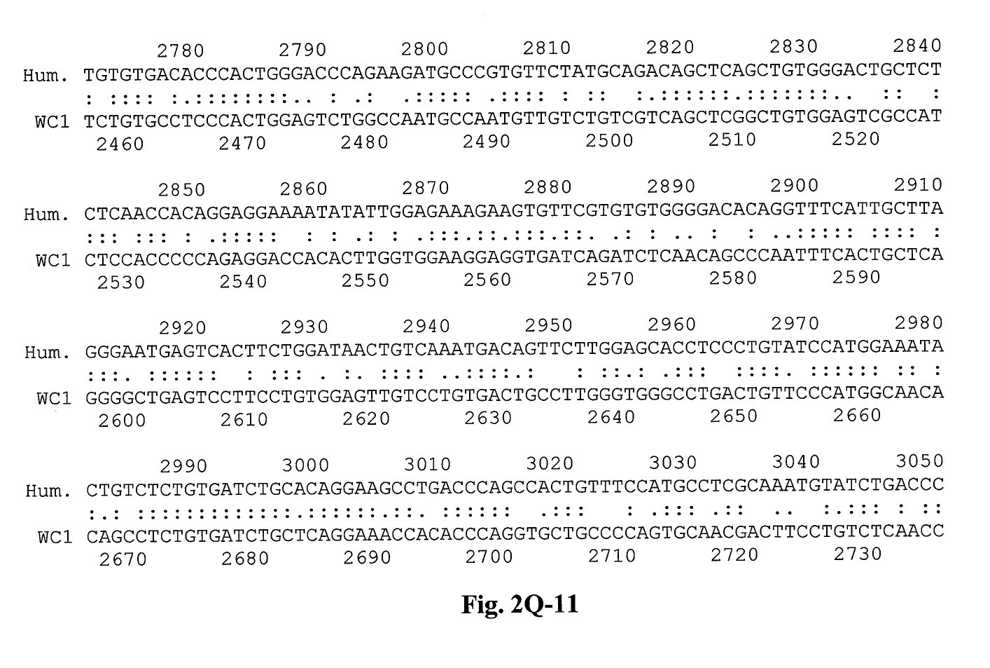

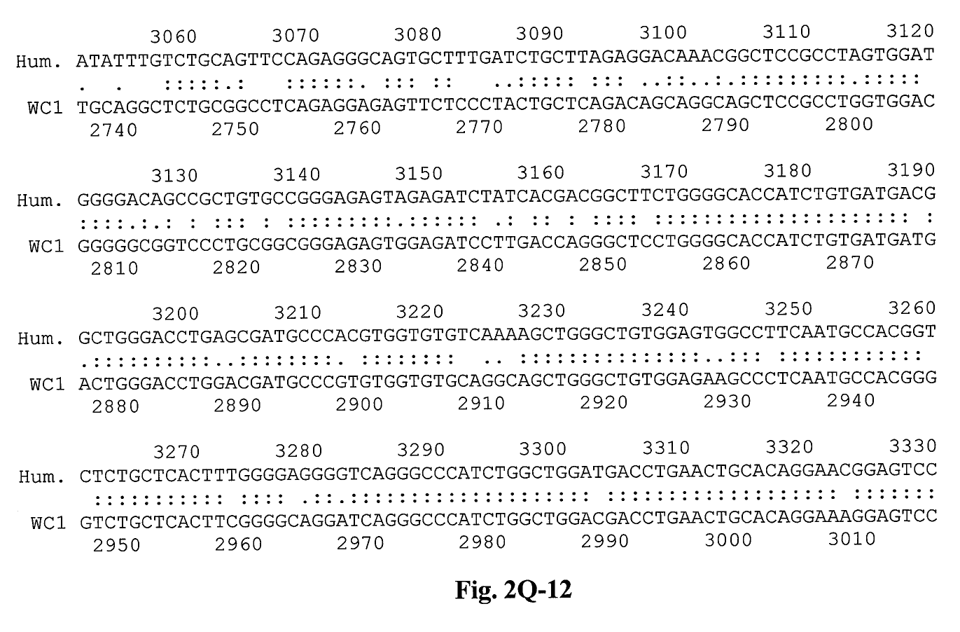

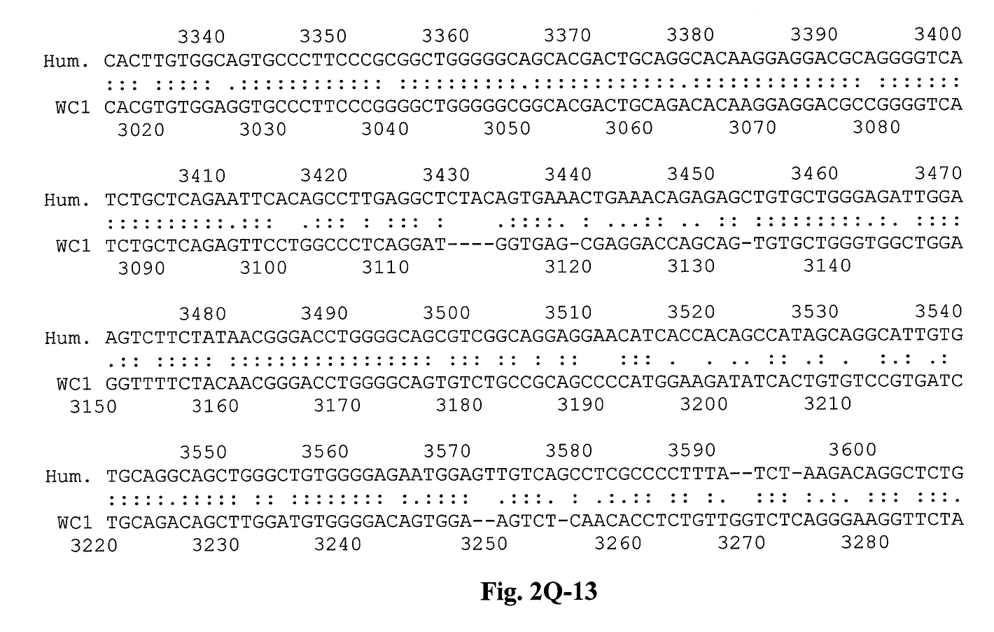

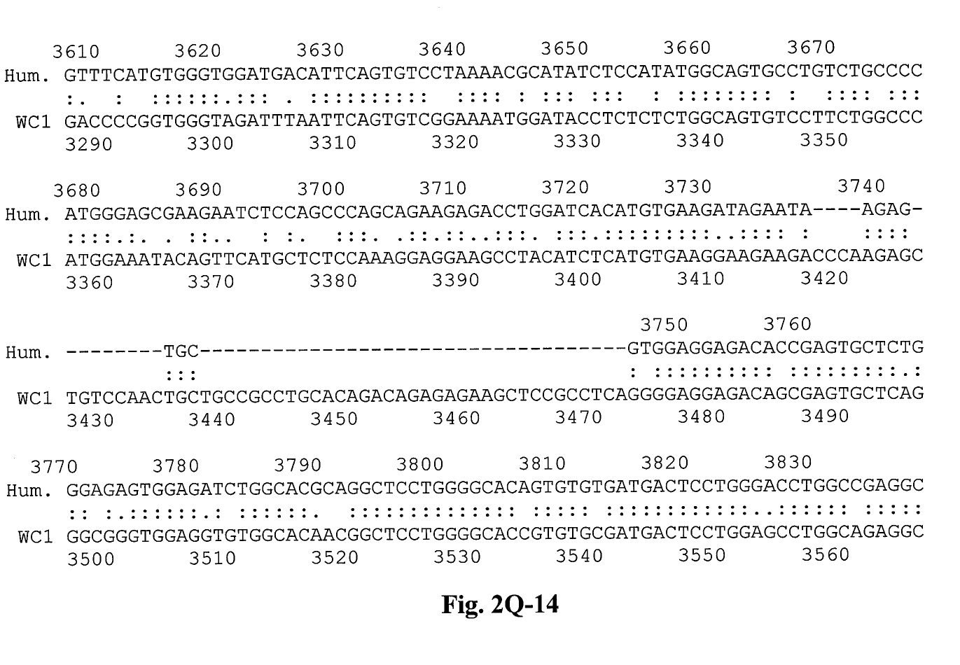

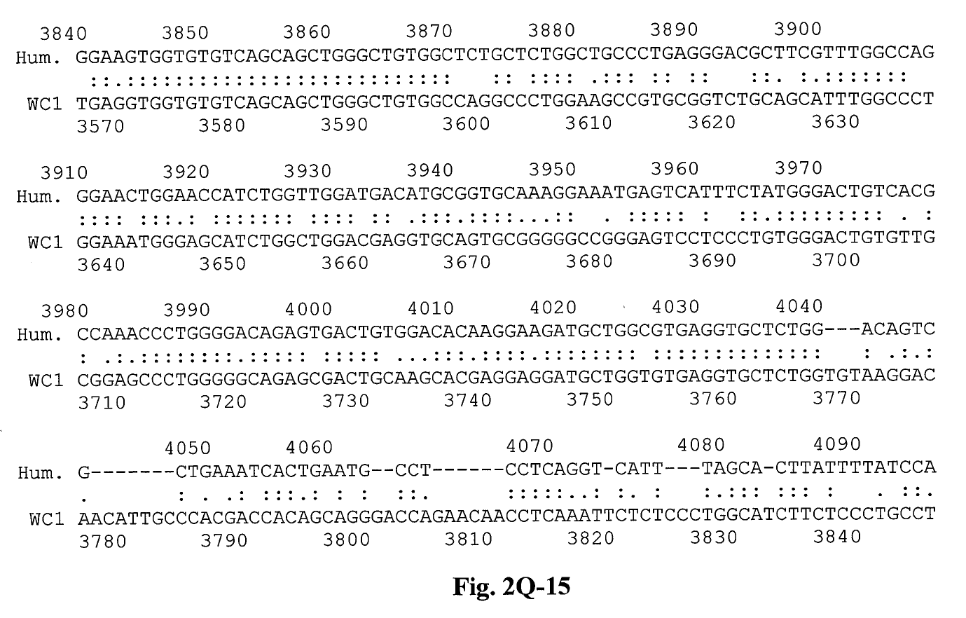

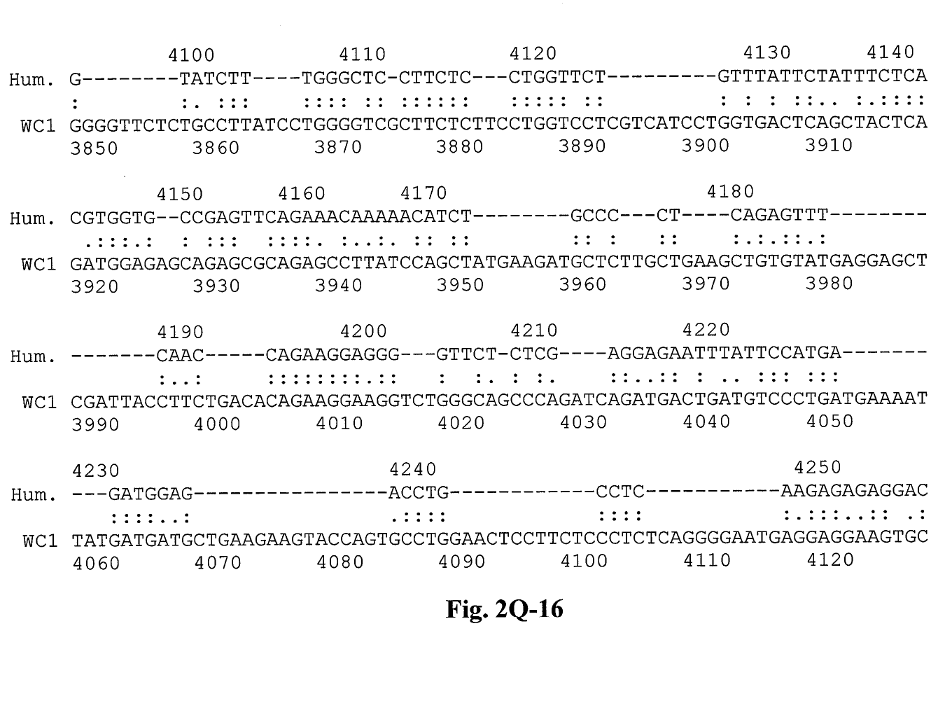

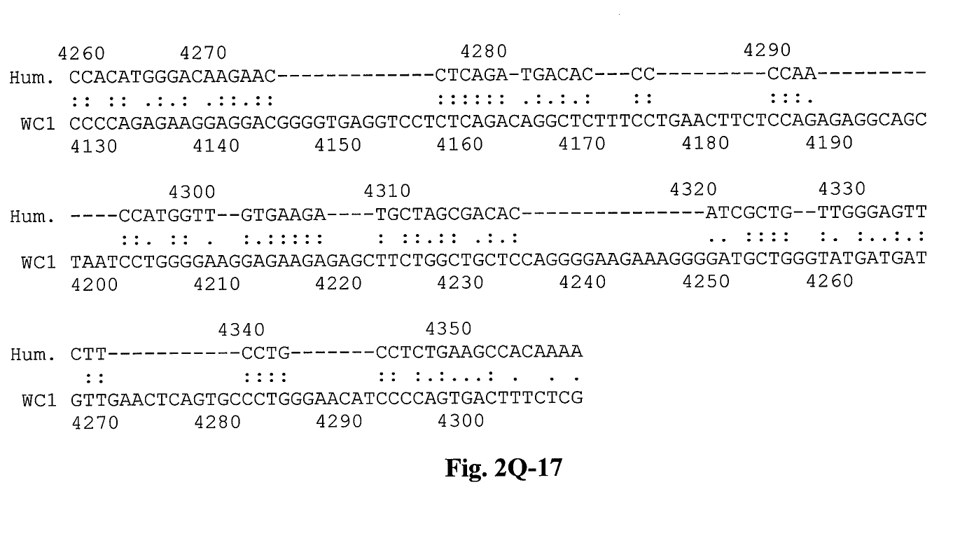

The full length of the cDNA encoding human TANGO 234 protein (FIG. 2; SEQ ID NO: 9) is 4628 nucleotide residues. The ORF of this cDNA, nucleotide residues 28 to 4386 of SEQ ID NO: 9 (i.e., SEQ ID NO: 10), encodes a 1453-amino acid transmembrane protein (FIG. 2; SEQ ID NO: 11).

The invention thus includes purified human TANGO 234 protein, both in the form of the immature 1453 amino acid residue protein (SEQ ID NO: 11) and in the form of the mature 1413 amino acid residue protein (SEQ ID NO: 13). Mature human TANGO 234 protein can be synthesized without the signal sequence polypeptide at the amino terminus thereof, or it can be synthesized by generating immature TANGO 234 protein and cleaving the signal sequence therefrom.

In addition to full length mature and immature human TANGO 234 proteins, the invention includes fragments, derivatives, and variants of these TANGO 234 proteins, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 9 or some portion thereof, such as the portion which encodes mature TANGO 234 protein, immature TANGO 234 protein, or a domain of TANGO 234 protein. These nucleic acids are collectively referred to as nucleic acids of the invention.

TANGO 234 proteins and nucleic acid molecules encoding them comprise a family of molecules having certain conserved structural and functional features, as indicated by the conservation of amino acid sequence between human TANGO 234 protein and bovine WC1 protein, as shown in FIGS. 2K through 2P, and the conservation of nucleotide sequence between the ORFs encoding human TANGO 234 protein and bovine WC1 protein, as shown in FIGS. 2Q-1 through 2Q-17.

A common domain present in TANGO 234 proteins is a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 234 protein contains a signal sequence corresponding to amino acid residues 1 to 40 of SEQ ID NO: 11 (SEQ ID NO: 12). The signal sequence is cleaved during processing of the mature protein.

TANGO 234 proteins can include an extracellular domain. The human TANGO 234 protein extracellular domain is located from about amino acid residue 41 to about amino acid residue 1359 of SEQ ID NO: 3. TANGO 234 can alternately exist in a secreted form, such as a mature protein having the amino acid sequence of amino acid residues 41 to 1453 or residues 41 to about 1359 of SEQ ID NO: 11.

In addition, TANGO 234 include a transmembrane domain. In one embodiment, a TANGO 234 protein of the invention contains a transmembrane domain corresponding to about amino acid residues 1360 to 1383 of SEQ ID NO: 11 (SEQ ID NO: 15).

The present invention includes TANGO 234 proteins having a cytoplasmic domain, particularly including proteins having a carboxyl-terminal cytoplasmic domain. The human TANGO 234 cytoplasmic domain is located from about amino acid residue 1384 to amino acid residue 1453 of SEQ ID NO: 11 (SEQ ID NO: 16).

TANGO 234 proteins typically comprise a variety of potential post-translational modification sites (often within an extracellular domain), such as those described herein in Table IV, as predicted by computerized sequence analysis of TANGO 234 proteins using amino acid sequence comparison software (comparing the amino acid sequence of TANGO 234 with the information in the PROSITE database {rel. 12.2; February, 1995} and the Hidden Markov Models database {Rel. PFAM 3.3 }). In certain embodiments, a protein of the invention has at least 1, 2, 4, 6, 10, 15, or 20 or more of the post-translational modification sites listed in Table IV.

TABLE IV

| | Amino Acid | |
|---|---|---|
| Type of Potential Modification Site or | Residues of | Amino Acid |
| Domain | SEQ ID NO: 11 | Sequence |
| N-glycosylation site | 42 to 45 | NGTD |
| | 78 to 81 | NTTA |
| | 120 to 123 | NESA |
| | 161 to 164 | NNSC |
| | 334 to 337 | NESF |
| | 377 to 380 | NCSG |
| | 441 to 444 | NESA |
| | 548 to 551 | NESN |
| | 637 to 640 | NAST |
| | 972 to 975 | NESL |
| | 1013 to 1016 | NVSD |
| | 1084 to 1087 | NATV |
| | 11O4 to 1107 | NCTG |
| | 1161 to 1164 | NGTW |
| | 1171 to 1174 | NITT |
| | 1318 to 1321 | NESF |
| | 1354 to 1357 | NASS |
| Glycosaminoglycan attachment site | 558 to 561 | SGWG |
| | 665 to 668 | SGWG |
| cAMP- or cGMP-dependent protein | 1229 to 1232 | RRIS |
| kinase phosphorylation site | 1399 to 1402 | RRGS |
| Protein kinase C phosphorylation site | 165 to 167 | SGR |
| | 268 to 270 | TNR |
| | 379 to 381 | SGR |
| | 419 to 421 | SRR |
| | 469 to 471 | SDK |
| | 506 to 508 | STR |
| | 589 to 591 | SNR |
| | 593 to 595 | SGR |
| | 661 to 663 | SCR |
| | 696 to 698 | SSR |
| | 746 to 748 | TER |
| | 805 to 807 | SGR |
| | 815 to 817 | TWR |
| | 959 to 961 | SVR |
| | 1256 to 1258 | SGR |
| | 1349 to 1351 | SLK |
| | 1396 to 1398 | STR |
| Casein kinase II phosphorylation site | 44 to 47 | TDLE |
| | 71 to 74 | TVCD |
| | 178 to 181 | TICD |
| | 245 to 248 | SHNE |
| | 253 to 256 | TCYD |
| | 258 to 261 | SDLE |
| | 319 to 322 | SGSD |
| | 332 to 335 | SGNE |
| | 392 to 395 | TICD |
| | 439 to 442 | TGNE |
| | 606 to 609 | TVCD |
| | 622 to 625 | SQLD |
| | 673 to 676 | SHSE |
| | 686 to 689 | SDME |
| | 760 to 763 | TGGE |
| | 765 to 768 | SLWD |
| | 818 to 821 | SVCD |
| | 845 to 848 | SVGD |
| | 857 to 860 | TWAE |
| | 907 to 910 | SQCD |
| | 923 to 926 | SLCD |
| | 927 to 930 | THWD |
| | 974 to 977 | SLLD |
| | 1059 to 1062 | TICD |
| | 1106 to 1109 | TGTE |
| | 1145 to 1148 | SETE |
| | 1233 to 1236 | SPAE |
| | 1241 to 1244 | TCED |
| | 1269 to 1272 | TVCD |
| | 1402 to 1405 | SLEE |
| | 1425 to 1428 | TSDD |
| N-myristoylation site | 67 to 72 | GQWGTV |
| | 90 to 95 | GCPFSF |
| | 101 to 106 | GQAVTR |
| | 119 to 124 | GNESAL |
| | 133 to 138 | GSHNCY |
| | 160 to 165 | GNNSCS |
| | 197 to 202 | GCPSSF |
| | 226 to 231 | GNELAL |
| | 240 to 245 | GNHDCS |
| | 267 to 272 | GTNRCM |
| | 304 to 309 | GCGTAL |
| | 328 to 333 | GVSCSG |
| | 374 to 379 | GSNNCS |
| | 411 to 416 | GCPFSV |
| | 418 to 423 | GSRRAK |
| | 440 to 445 | GNESAL |
| | 465 to 470 | GVICSD |
| | 547 to 552 | GNESNI |
| | 588 to 593 | GSNRCS |
| | 632 to 637 | GMGLGN |
| | 668 to 673 | GNNDCS |
| | 679 to 684 | GVICSD |
| | 695 to 700 | GSSRCA |
| | 712 to 717 | GILCAN |
| | 720 to 725 | GMNIAE |
| | 758 to 763 | GCTGGE |
| | 853 to 858 | GNGLTW |
| | 891 to 896 | GVVCSR |
| | 944 to 949 | GTALST |
| | 985 to 990 | GAPPCI |
| | 992 to 997 | GNTVSV |
| | 1078 to 1083 | GCGVAF |
| | 1121 to 1126 | GQHDCR |
| | 1132 to 1137 | GVICSE |
| | 1162 to 1167 | GTWGSV |
| | 1185 to 1190 | GCGENG |
| | 1265 to 1270 | GSWGTV |
| | 1288 to 1293 | GCGSAL |
| | 1302 to 1307 | GQGTGT |
| | 1331 to 1336 | GQSDCG |
| | 1342 to 1347 | GVRCSG |
| | 1422 to 1427 | GTRTSD |
| | 1443 to 1438 | GCEDAS |
| | 1444 to 1449 | GVLPAS |
| Amidation site | 1167 to 1170 | VGRR |
| Speract receptor repeated (SRR) | 53 to 90 | See FIG. 2 |
| domain signature | 160 to 197 | See FIG. 2 |
| | 267 to 304 | See FIG. 2 |
| | 1041 to 1078 | See FIG. 2 |
| | 1251 to 1288 | See FIG. 2 |
| Scavenger receptor cysteine-rich | 51 to 148 | See FIG. 2 |
| (SRCR) domain | 158 to 255 | See FIG. 2 |
| | 265 to 362 | See FIG. 2 |
| | 372 to 469 | See FIG. 2 |
| | 479 to 576 | See FIG. 2 |
| | 586 to 683 | See FIG. 2 |
| | 693 to 790 | See FIG. 2 |
| | 798 to 895 | See FIG. 2 |
| | 903 to 1000 | See FIG. 2 |
| | 1039 to 1136 | See FIG. 2 |
| | 1146 to 1243 | See FIG. 2 |
| | 1249 to 1346 | See FIG. 2 |

Among the domains that occur in TANGO 234 protein are SRR domains and SRCR domains. In one embodiment, the protein of the invention has at least one domain that is at least 55%, preferably at least about 65%, more preferably at least about 75%, yet more preferably at least about 85%, and most preferably at least about 95% identical to one of these domains. In other embodiments, the protein has at least two of the SRR and SRCR domains described herein in Table IV. In other embodiments, the protein has at least one SRR domain and at least one SRCR domain.

The SRR domain is named after a receptor domain identified in a sea urchin egg protein designated speract. The consensus sequence of this domain (using standard one-letter amino acid codes, wherein X is any amino acid residue) is as follows.

-G-X₅-G-X₂-E-X₆-W-G-X₂-C-X₃-(F or Y or W)-X₈-C-X₃-G-.

Speract is a transmembrane glycoprotein of 500 amino acid residues (Dangott et al. (1989) Proc. Natl. Acad. Sci. USA 86:2128-2132). Structurally, this receptor consists of a large extracellular domain of 450 residues, followed by a transmembrane region and a small cytoplasmic domain of 12 amino acid residues. The extracellular domain contains four repeats of an approximately 115 amino acid domain. There are 17 amino acid residues that are perfectly conserved in the four repeats in speract, including six cysteine residues, six glycine residues, and two glutamate residues. TANGO 234 has five SRR domains, in which 16 of the 17 conserved speract residues are present of four of the SRR domains and 15 are present in the remaining SRR domain. This domain is designated the speract receptor repeated domain. The amino acid sequence of mammalian macrophage scavenger receptor type I (MSRI) exhibits such a domain (Freeman et al. (1990) Proc. Natl. Acad. Sci. USA 87:8810-8814). MSRI proteins are membrane glycoproteins implicated in the pathologic deposition of cholesterol in arterial walls during atherogenesis. TANGO 234 is involved in one or more physiological processes related to cholesterol deposition and atherogenesis, as well as other vascular and cardiovascular disorders.

Scavenger receptor cysteine-rich (SRCR) domains are disulfide rich extracellular domains which are present in certain cell surface and secreted proteins. Proteins having SRCR domains exhibit diverse ligand binding specificity. For example, in addition to modified lipoproteins, some of these proteins bind a variety of surface components of pathogenic microorganisms, and some of the proteins bind apoptotic cells. SRCR domains are also involved in mediating immune development and response. Other SRCR-containing proteins are involved in binding of modified lipoproteins (e.g., oxidized low density lipoprotein {LDL}) by specialized macrophages, leading to the formation of macrophages filled with cholesteryl ester droplets (i.e., foam cells). TANGO 234 is involved in one or more physiological processes in which these other SRCR domain-containing proteins are involved, such as LDL uptake and metabolism, regulation of serum cholesterol level, atherogenesis, atherosclerosis, bacterial or viral infections, immune development, and generation and perseverance of immune responses.

WC1 is a ruminant protein having an SRCR domain. WC1 and gamma delta T-cell receptor are the only known gamma delta T-cell specific antigens. Antibodies which bind specifically with WC1 induce growth arrest in IL-2-dependent gamma delta T-cell and augment proliferation of gamma delta T-cells in an autologous mixed lymphocyte reaction or in the presence of anti-CD2 or anti-CD5 antibodies. Injection of antibodies which bind specifically with WC1 into calves results in long-lasting depletion of gamma delta T-cells. Furthermore, antibodies which bind specifically with WC1 can be used to purify gamma delta T-cells.

Gamma delta T-cells are involved in a variety of physiological processes. For example, these cells are potential mediators of allergic airway inflammation and lyme disease. Furthermore, these cells are involved in natural resistance to viral infections and can mediate autoimmune diseases. Elimination of gamma delta T-cells by injection of antibodies which bind specifically therewith can affect the outcomes of these disorders.

TANGO 234 is likely the human orthologue of ruminant protein WC1, and thus is involved with the physiological processes described above in humans. An alignment of the amino acid sequences of (human) TANGO 234 and bovine WC1 protein is shown in FIGS. 2K-2P. In this alignment (made using the ALIGN software {Myers and Miller (1989) CABIOS, ver. 2.0}; pam120.mat scoring matrix; gap penalties −12/−4), the proteins are 40.4% identical. An alignment of the nucleotide sequences of the ORFs encoding (human) TANGO 234 and bovine WC1 protein is shown in FIGS. 2Q-1 to 2Q-17. The two ORFs are 54.3% identical, as assessed using the same software and parameters.

The signal peptide prediction program SIGNALP (Nielsen et al. (1997) Protein Engineering 10:1-6) predicted that human TANGO 234 protein includes a 40 amino acid signal peptide (amino acid residues 1 to 40 of SEQ ID NO: 11; SEQ ID NO: 12) preceding the mature TANGO 234 protein (amino acid residues 41 to 4386 of SEQ ID NO: 11; SEQ ID NO: 13). Human TANGO 234 protein includes an extracellular domain (amino acid residues 41 to 1359 of SEQ ID NO: 11; SEQ ID NO: 14); a transmembrane domain (amino acid residues 1360 to 1383 of SEQ ID NO: 11; SEQ ID NO: 15); and a cytoplasmic domain (amino acid residues 1384 to 1453 of SEQ ID NO: 11; SEQ ID NO: 16).

FIG. 2J depicts a hydrophilicity plot of human TANGO 234 protein. Relatively hydrophobic regions are above the dashed horizontal line, and relatively hydrophilic regions are below the dashed horizontal line. The hydrophobic region which corresponds to amino acid residues 1 to 40 of SEQ ID NO: 11 is the signal sequence of human TANGO 234 (SEQ ID NO: 12). The hydrophobic region which corresponds to amino acid residues 1360 to 1383 of SEQ ID NO: 11 is the transmembrane domain of human TANGO 234 (SEQ ID NO: 15). As described elsewhere herein, relatively hydrophilic regions are generally located at or near the surface of a protein, and are more frequently effective immunogenic epitopes than are relatively hydrophobic regions. For example, the region of human TANGO 234 protein from about amino acid residue 225 to about amino acid residue 250 appears to be located at or near the surface of the protein, while the region from about amino acid residue 990 to about amino acid residue 1000 appears not to be located at or near the surface.

The predicted molecular weight of human TANGO 234 protein without modification and prior to cleavage of the signal sequence is about 159.3 kilodaltons. The predicted molecular weight of the mature human TANGO 234 protein without modification and after cleavage of the signal sequence is about 154.7 kilodaltons.

Chromosomal mapping to identify the location of the gene encoding human TANGO 234 protein indicated that the gene was located at chromosomal location h12p13 (with synteny to mo6). Flanking chromosomal markers include WI-6980 and GATA8A09.43. Nearby human loci include IBD2 (inflammatory bowel disease 2), FPF (familial periodic fever), and HPDR2 (hypophosphatemia vitamin D resistant rickets 2). Nearby genes are KLRC (killer cell receptor cluster), DRPLA (dentatorubro-pallidoluysian atrophy), GAPD (glyceraldehyde-3-phosphate) dehydrogenase, and PXR1 (peroxisome receptor 1). Murine chromosomal mapping indicated that the murine orthologue is located near the scr (scruffy) locus. Nearby mouse genes include drpla (dentatorubral phillidoluysian atrophy), prp (proline rich protein), and kap (kidney androgen regulated protein).

Northern analysis experiments indicated that mRNA corresponding to the cDNA encoding TANGO 234 is expressed in the tissues listed in Table V, wherein “++” indicates moderate expression, “+” indicates lower expression, and “−” indicates no detectable expression.

TABLE V

| Animal | Tissue | Relative Level of Expression |
|---|---|---|
| Human | spleen | ++ |
| | fetal lung | ++ |
| | lung | + |
| | thymus | + |
| | bone marrow | − |
| | peripheral blood leukocytes | − |

Biological Function of TANGO 234 Proteins, Nucleic Acids, and Modulators Thereof

TANGO 234 proteins are involved in disorders which affect both tissues in which they are normally expressed and tissues in which they are normally not expressed. Based on the observation that TANGO 234 is expressed in human fetal lung, spleen, and, to a lesser extent in adult lung and thymus tissue, TANGO 234 protein is involved in one or more biological processes which occur in these tissues. In particular, TANGO 234 is involved in modulating growth, proliferation, survival, differentiation, and activity of cells including, but not limited to, lung, spleen, thymus bone marrow, hematopoietic, peripheral blood leukocytes, and fetal cells of the animal in which it is normally expressed. Thus, TANGO 234 has a role in disorders which affect these cells and their growth, proliferation, survival, differentiation, and activity. Expression of TANGO 234 in an animal is also involved in modulating growth, proliferation, survival, differentiation, and activity of cells and viruses which are foreign to the host (i.e., bacterial, fungal, and viral infections).

Homology of human TANGO 234 with bovine WC1 protein indicates that TANGO 234 has physiological functions in humans analogous to the functions of WC1 in ruminants. Thus, TANGO 234 is involved in modulating growth, proliferation, survival, differentiation, and activity of gamma delta T cells. For example, TANGO 234 affects the ability of gamma delta T cells to interact with chemokines such as interleukin-2. TANGO 234 therefore is involved in the physiological processes associated with allergic airway inflammation, lyme arthritis, resistance to viral infection, auto-immune diseases, and the like.

In addition, presence in TANGO 234 of SRR and SRCR domains indicates that TANGO 234 is involved in physiological functions identical or analogous to the functions performed by other proteins having such domains. For example, like other SRR domain-containing proteins, TANGO 234 modulates cholesterol deposition in arterial walls, and is thus involved in development and persistence of atherogenesis and arteriosclerosis, as well as other vascular and cardiovascular disorders. Like other SRCR domain-containing proteins, TANGO 234 is involved in uptake and metabolism of LDL, regulation of serum cholesterol level, and can modulate these processes as well as the processes of atherogenesis, arteriosclerosis, immune development, and generation and perseverance of immune responses to bacterial, fungal, and viral infections.

TANGO 265

A cDNA clone (designated jthsa079g01) encoding at least a portion of human TANGO 265 protein was isolated from a human fetal spleen cDNA library. The human TANGO 265 protein is predicted by structural analysis to be a transmembrane membrane protein, although it can exist in a secreted form as well.

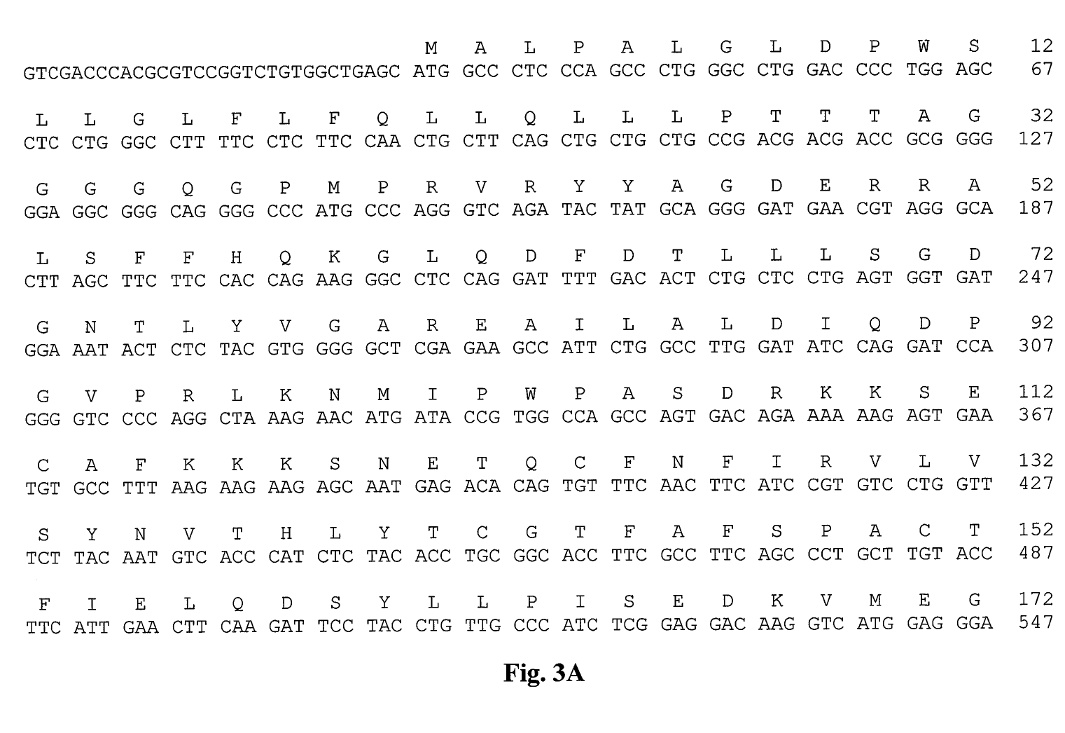

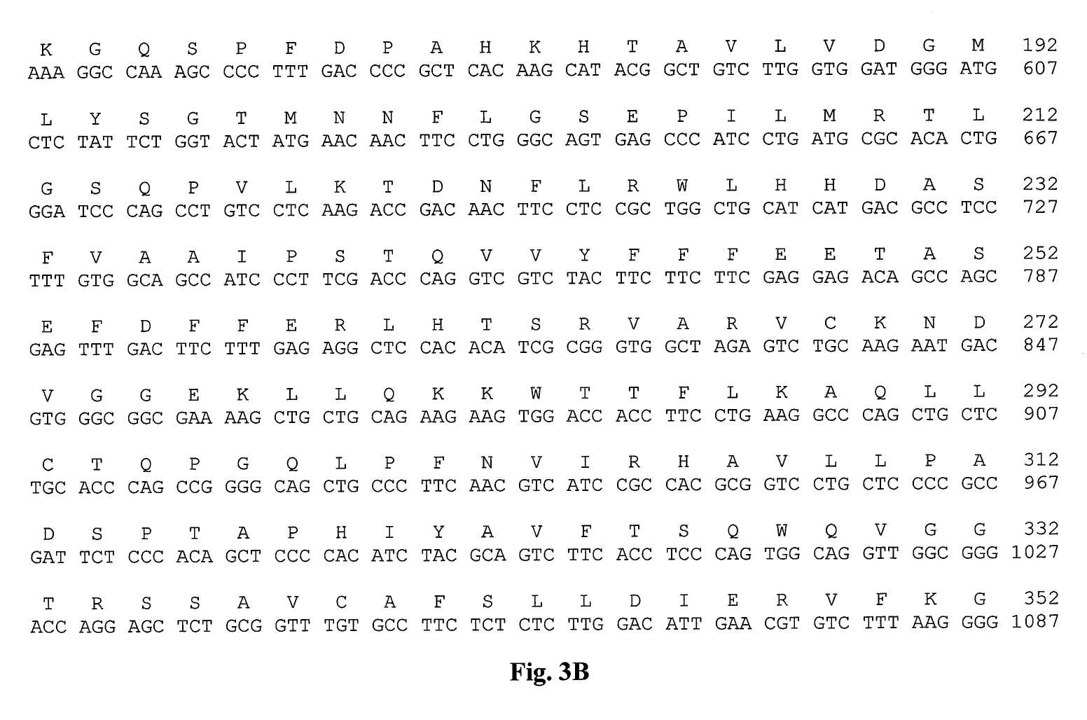

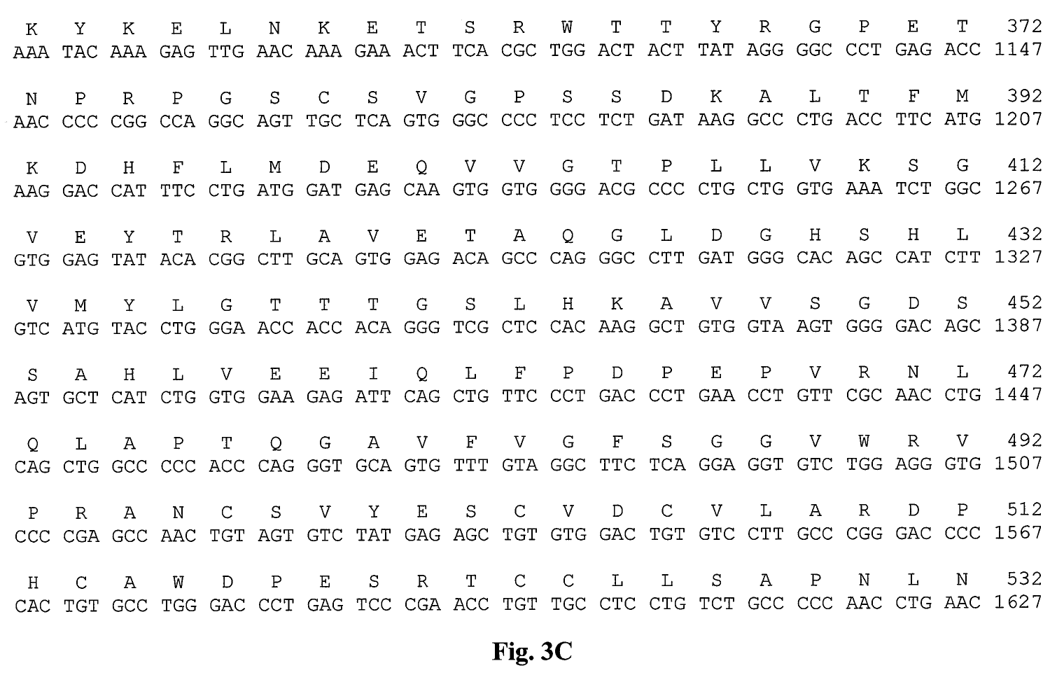

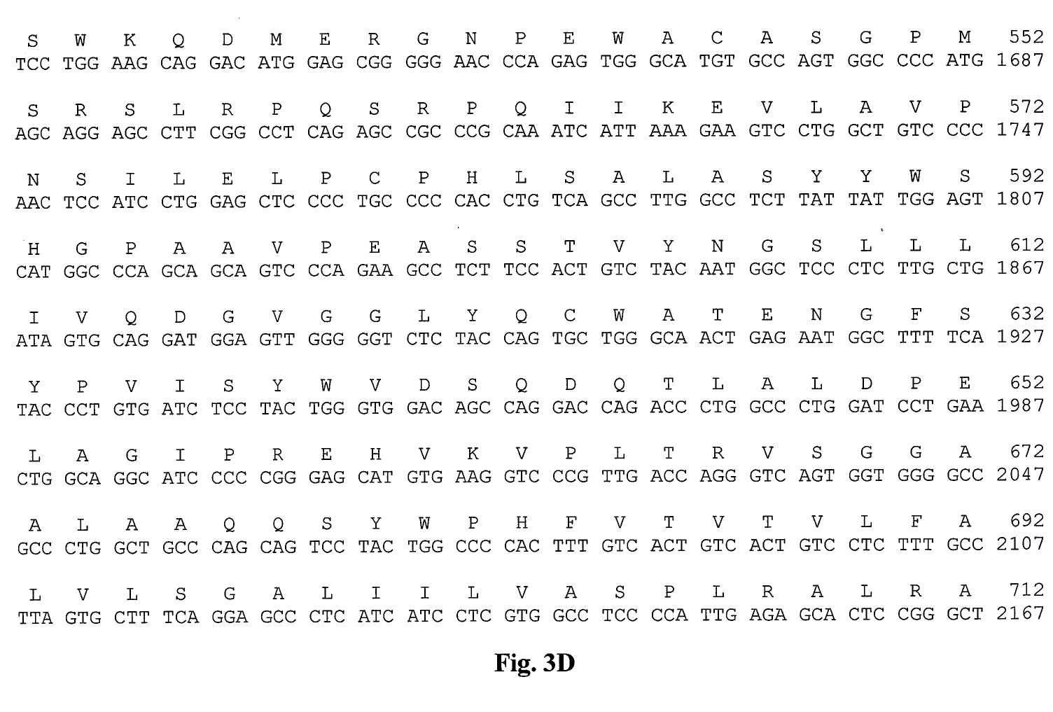

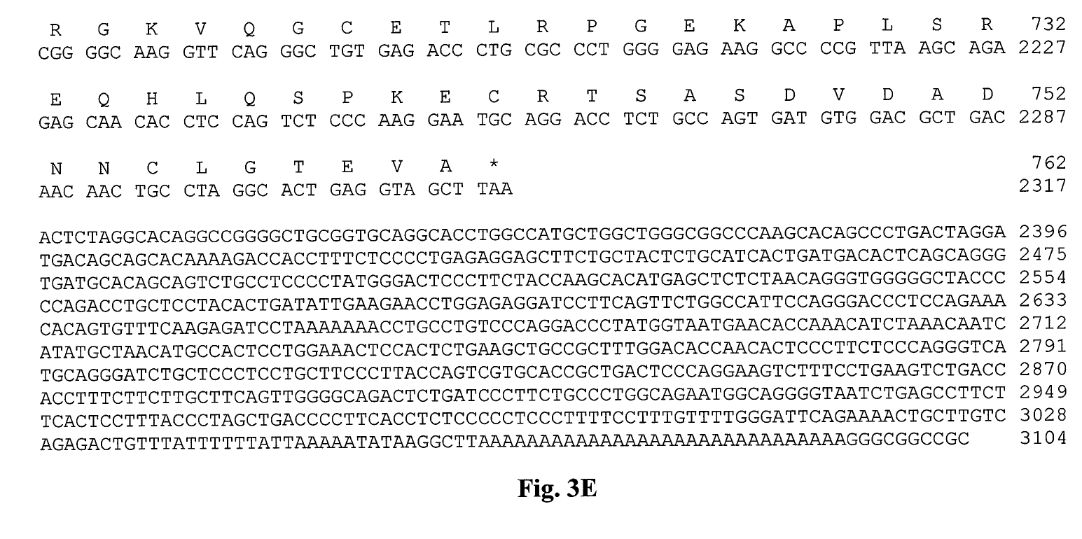

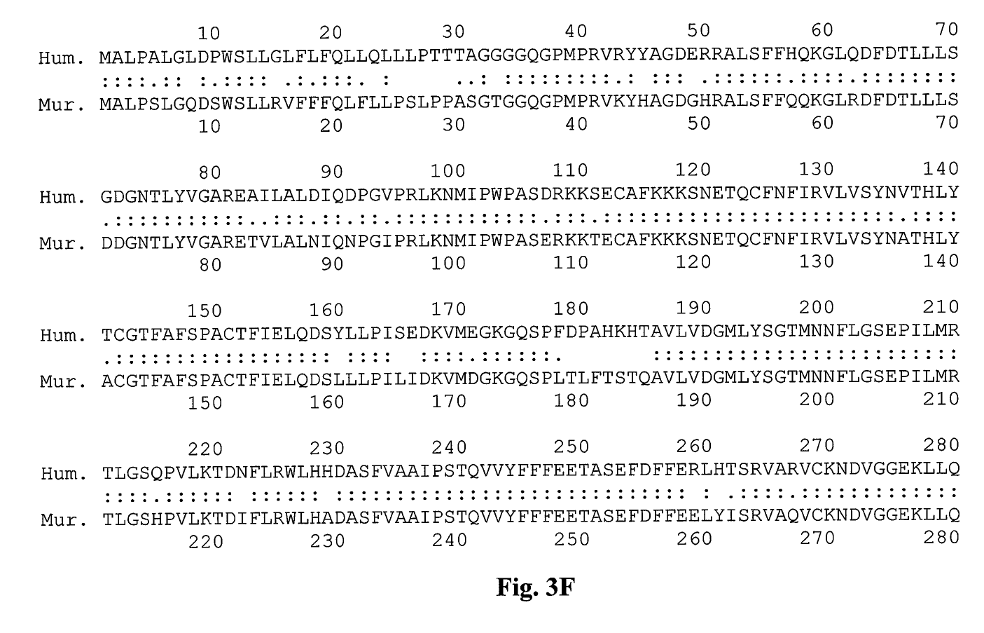

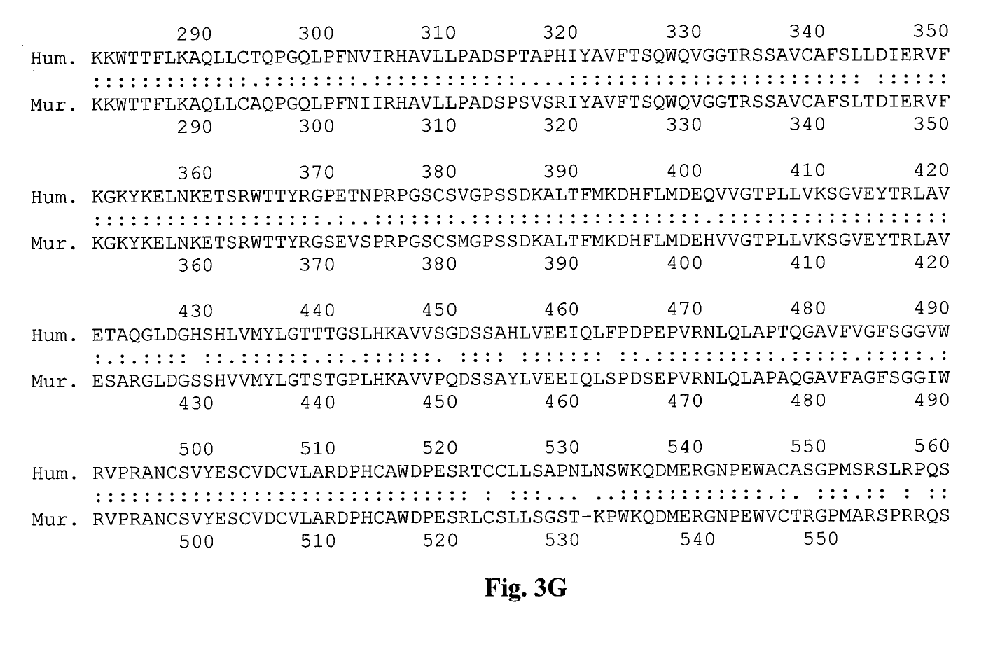

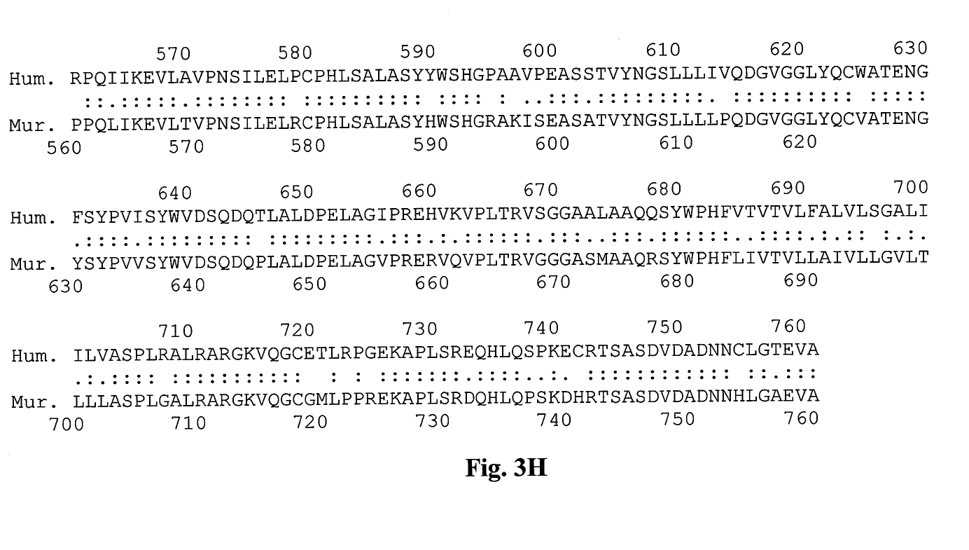

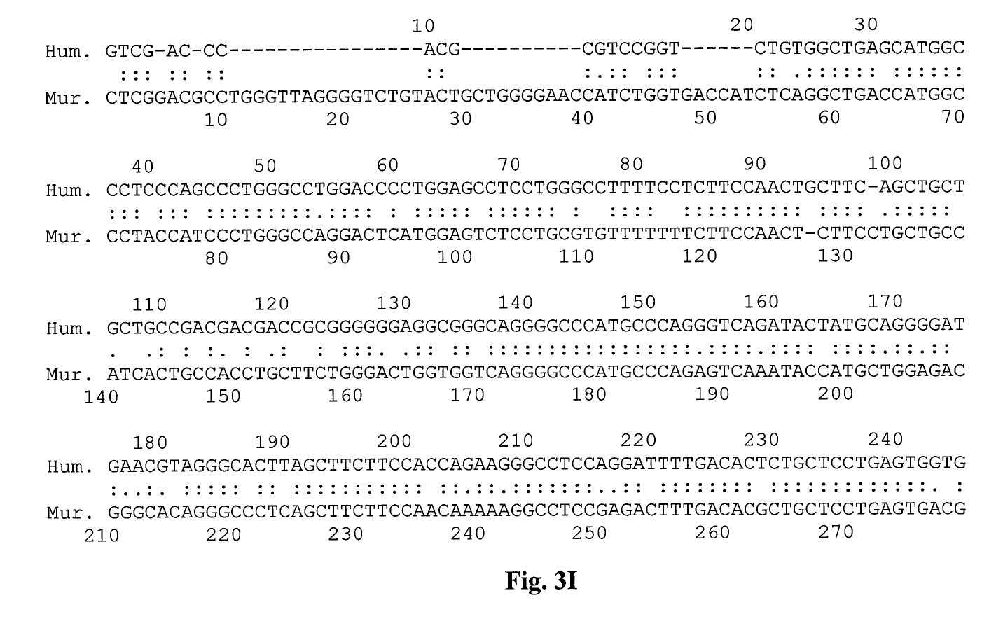

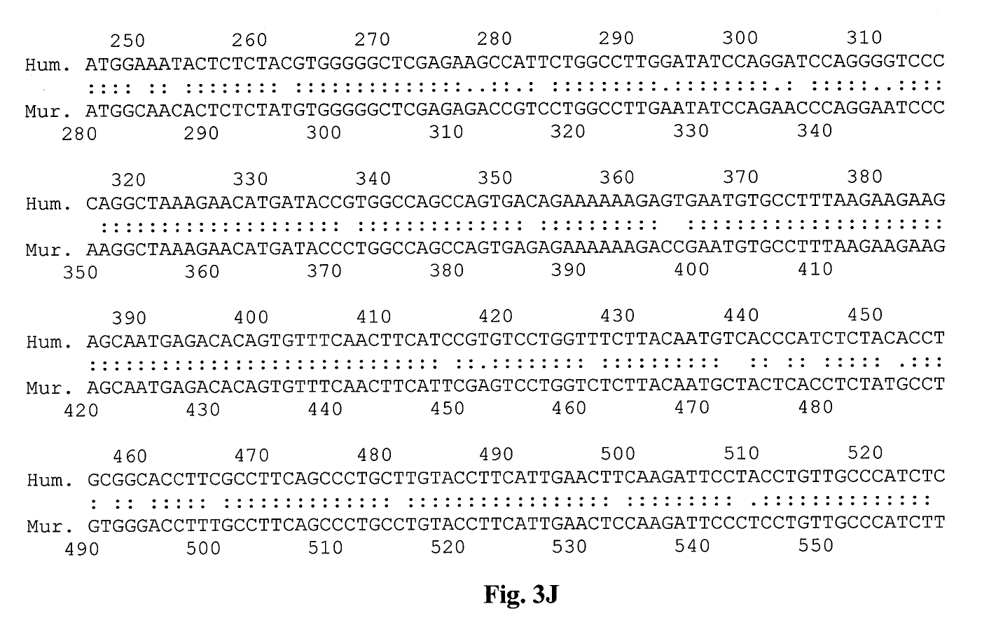

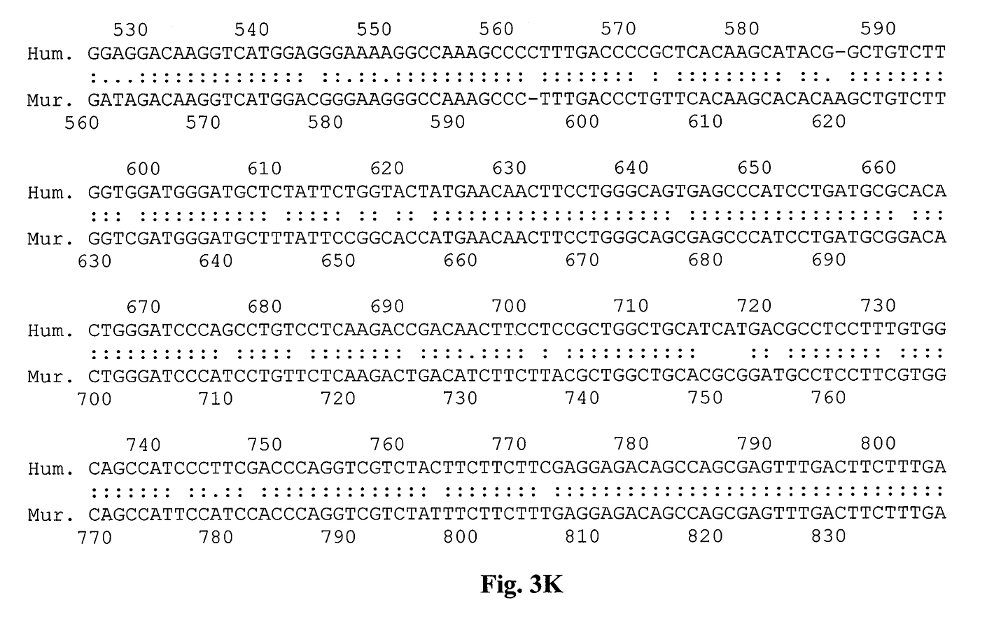

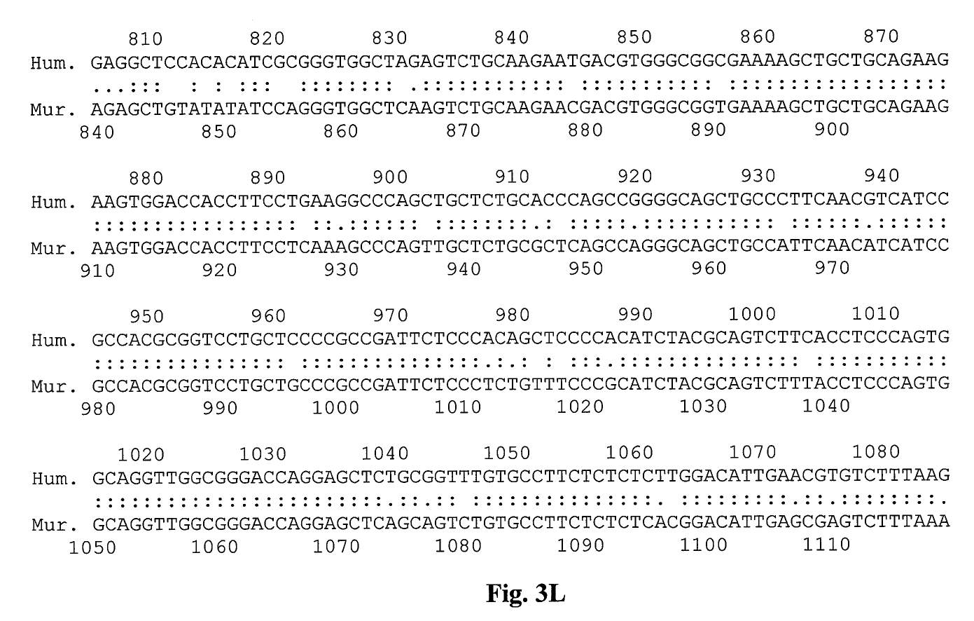

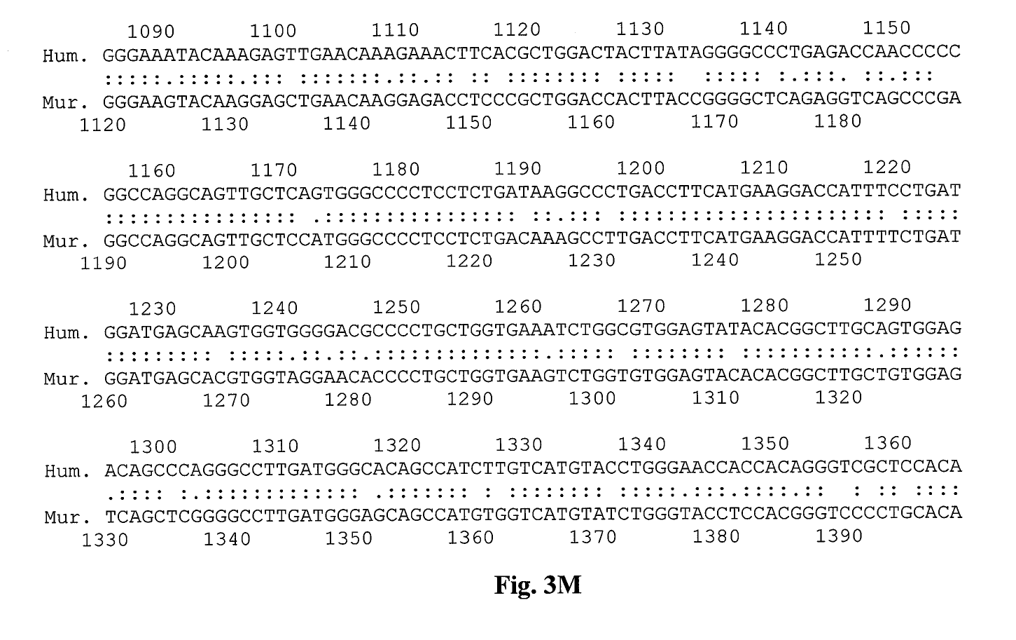

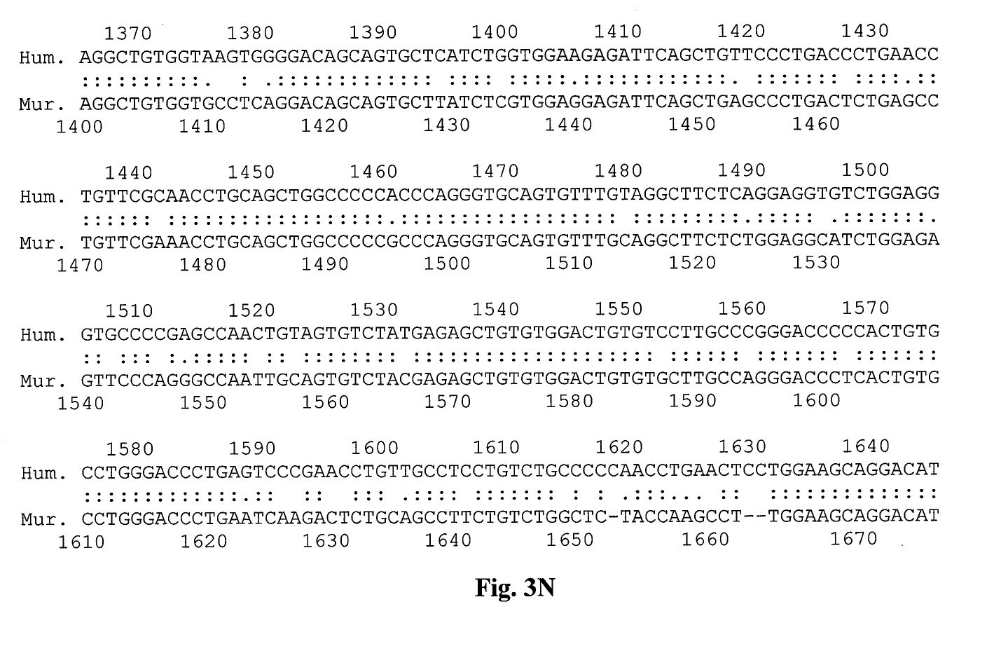

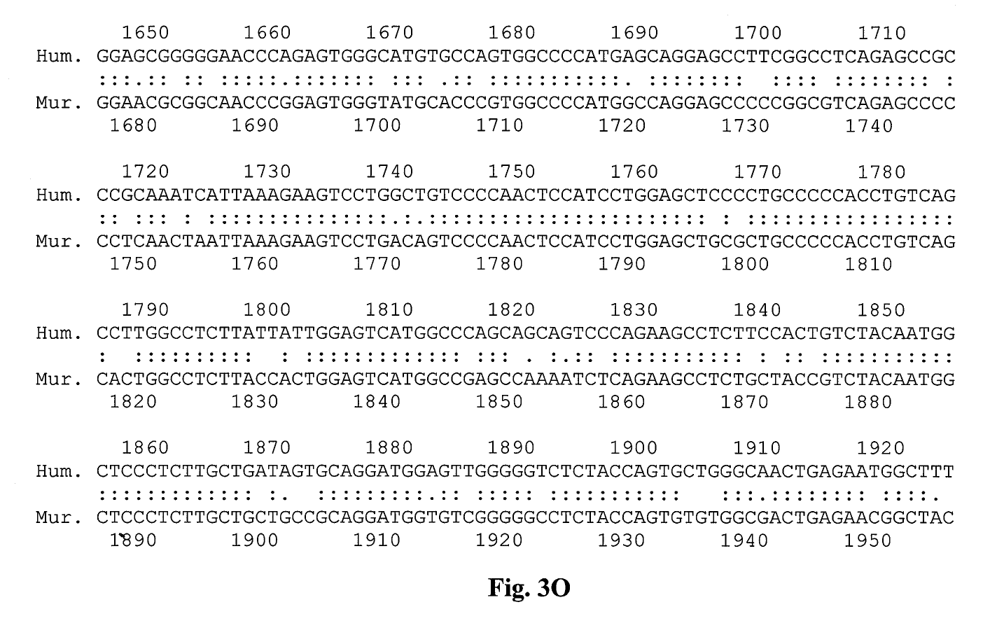

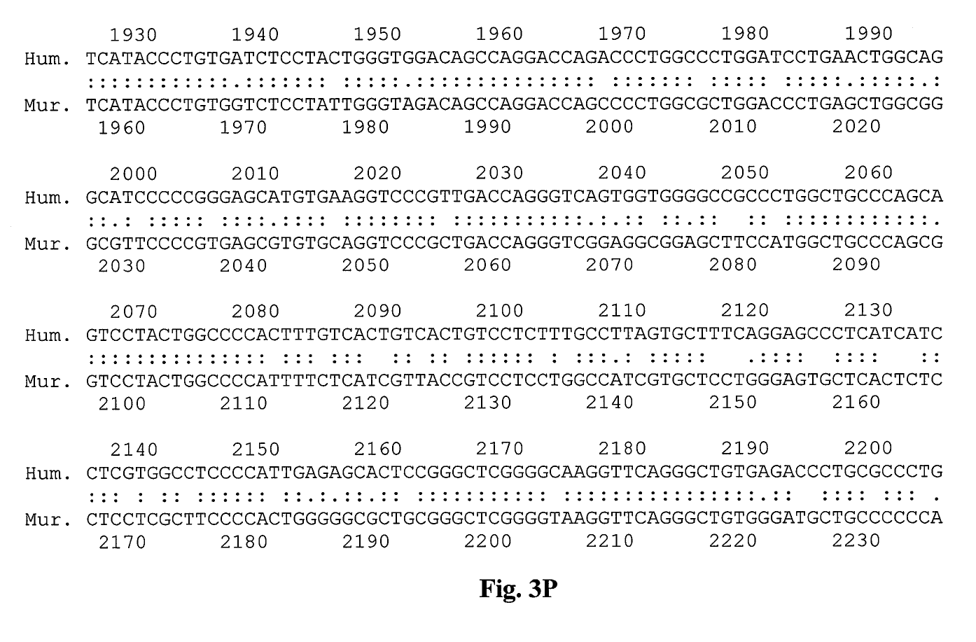

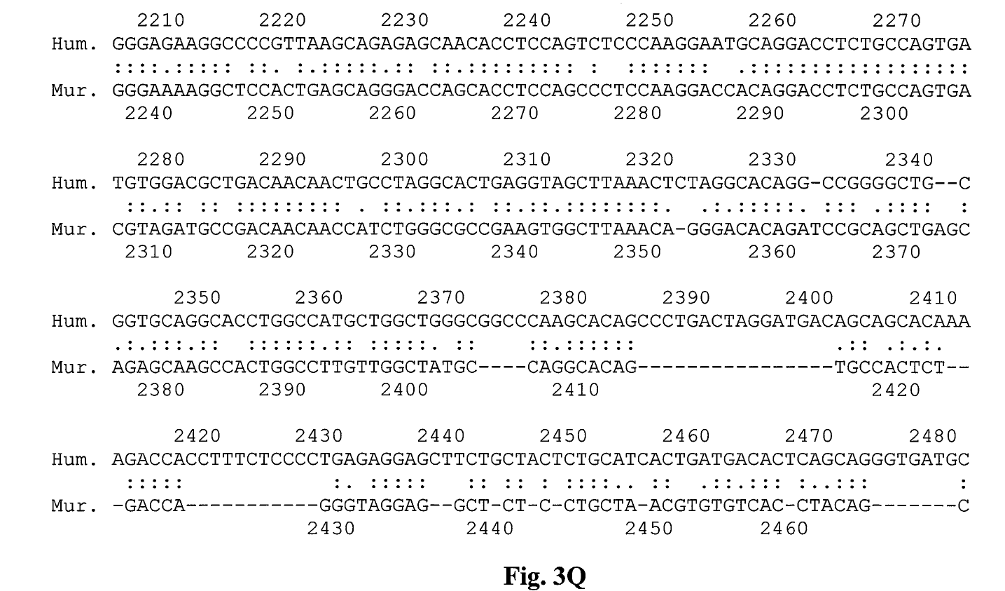

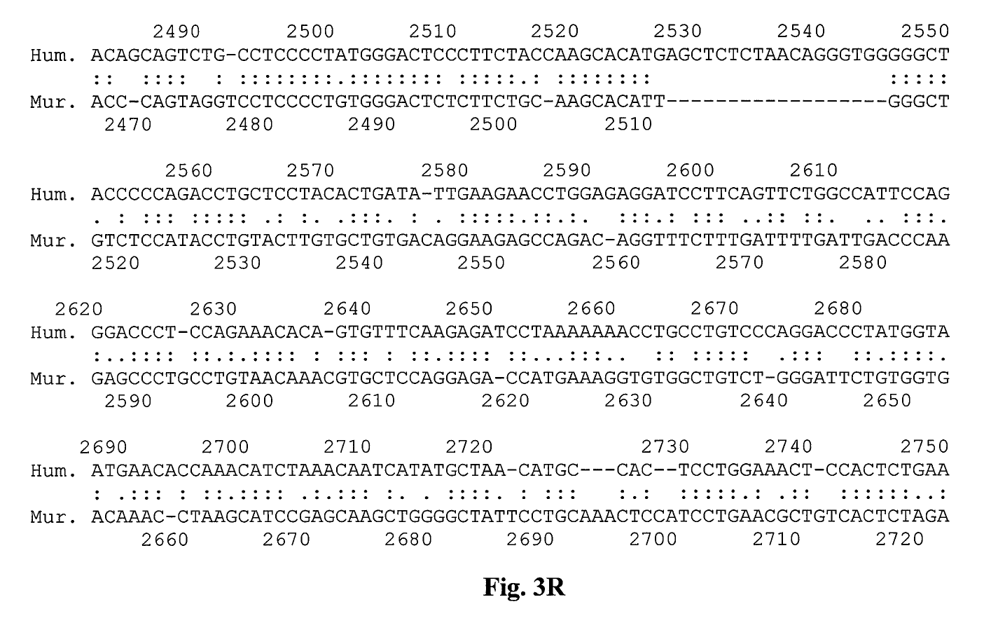

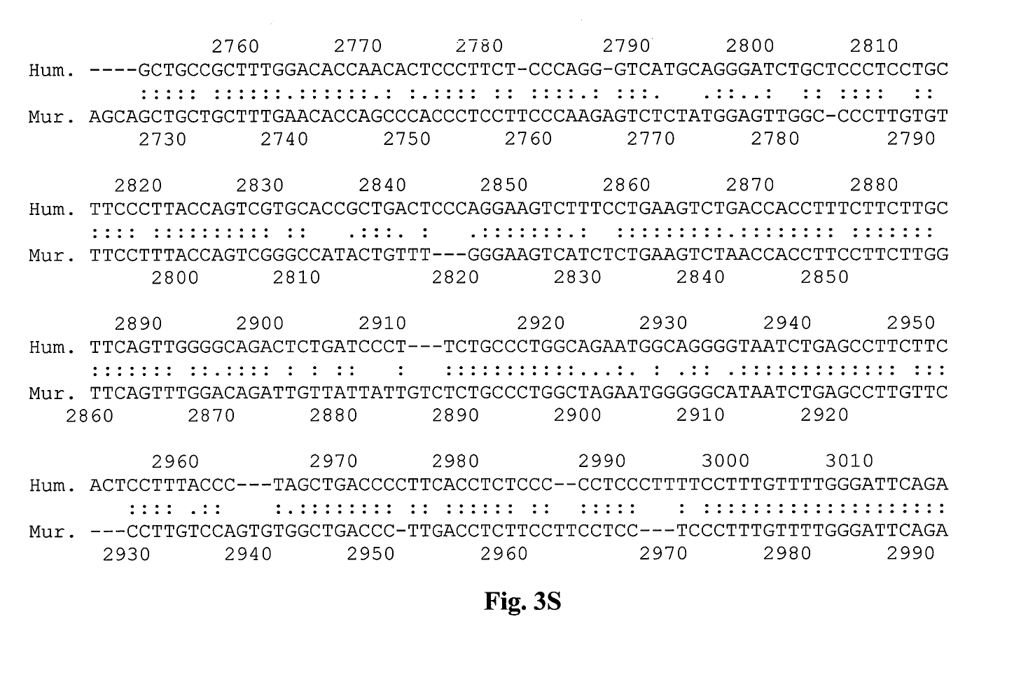

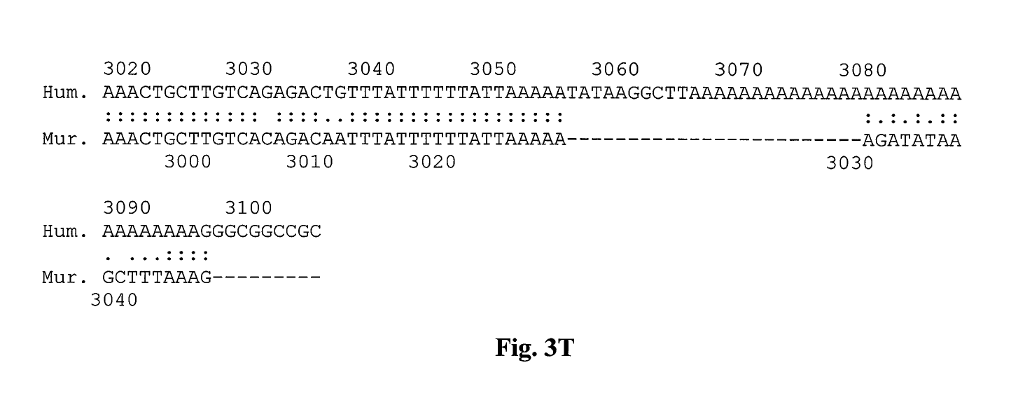

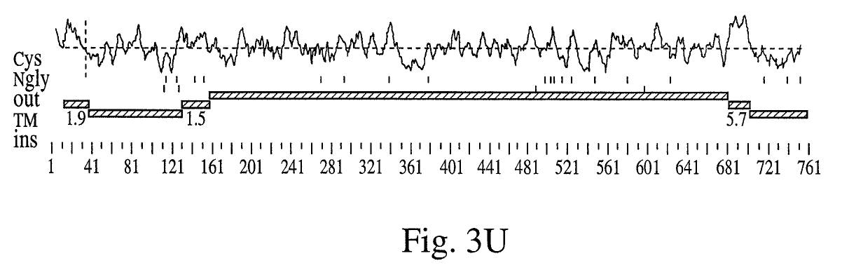

The full length of the cDNA encoding human TANGO 265 protein (FIG. 3; SEQ ID NO: 17) is 3104 nucleotide residues. The ORF of this cDNA, nucleotide residues 32 to 2314 of SEQ ID NO: 17 (i.e., SEQ ID NO: 18), encodes a 761-amino acid transmembrane protein (FIG. 3; SEQ ID NO: 19).

The invention thus includes purified TANGO 265 protein, both in the form of the immature 761 amino acid residue protein (SEQ ID NO: 19) and in the form of the mature 730 amino acid residue protein (SEQ ID NO: 21). Mature TANGO 265 protein can be synthesized without the signal sequence polypeptide at the amino terminus thereof, or it can be synthesized by generating immature TANGO 265 protein and cleaving the signal sequence therefrom.

In addition to full length mature and immature TANGO 265 proteins, the invention includes fragments, derivatives, and variants of TANGO 265 protein, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 17 or some portion thereof, such as the portion which encodes mature TANGO 265 protein, immature TANGO 265 protein, or a domain of TANGO 265 protein. These nucleic acids are collectively referred to as nucleic acids of the invention.

TANGO 265 proteins and nucleic acid molecules encoding them comprise a family of molecules having certain conserved structural and functional features.

A common domain present in TANGO 265 proteins is a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 265 protein contains a signal sequence corresponding to amino acid residues 1 to 31 of SEQ ID NO: 19 (SEQ ID NO: 20). The signal sequence is cleaved during processing of the mature protein

TANGO 265 proteins can also include an extracellular domain. The human TANGO 265 protein extracellular domain is located from about amino acid residue 32 to about amino acid residue 683 of SEQ ID NO: 17. TANGO 265 can alternately exist in a secreted form, such as a mature protein having the amino acid sequence of amino acid residues 32 to 761 or residues 32 to about 683 of SEQ ID NO: 19.

TANGO 265 proteins can also include a transmembrane domain. In one embodiment, a TANGO 265 protein of the invention contains a transmembrane domain corresponding to about amino acid residues 684 to 704 of SEQ ID NO: 19 (SEQ ID NO: 23).

In addition, TANGO 265 proteins include a cytoplasmic domain, particularly including proteins having a carboxyl-terminal cytoplasmic domain. The human TANGO 265 cytoplasmic domain is located from about amino acid residue 705 to amino acid residue 761 of SEQ ID NO: 19 (SEQ ID NO: 24).

TANGO 265 proteins typically comprise a variety of potential post-translational modification sites (often within an extracellular domain), such as those described herein in Table VI, as predicted by computerized sequence analysis of TANGO 265 proteins using amino acid sequence comparison software (comparing the amino acid sequence of TANGO 265 with the information in the PROSITE database {rel. 12.2; February, 1995} and the Hidden Markov Models database {Rel. PFAM 3.3 }). In certain embodiments, a protein of the invention has at least 1, 2, 4, 6, 10, 15, or 20 or more of the post-translational modification sites listed in Table VI.

TABLE VI

| | Amino Acid | |
|---|---|---|
| Type of Potential Modification Site or | Residues of | Amino Acid |
| Domain | SEQ ID NO: 19 | Sequence |
| N-glycosylation site | 120 to 123 | NETQ |
| | 135 to 138 | NVTH |
| | 496 to 499 | NCSV |
| | 607 to 610 | NGLS |
| Glycosaminoglycan attachment site | 70 to 73 | SGDG |
| cAMP- or cGMP-dependent protein | 108 to 111 | RKKS |
| kinase phosphorylation site | 116 to 119 | KKKS |
| | 281 to 284 | KKWT |
| Protein kinase C phosphorylation site | 106 to 108 | SDR |
| | 262 to 264 | TSR |
| | 361 to 363 | TSR |
| | 366 to 368 | TYR |
| | 385 to 387 | SDK |
| | 533 to 535 | SWK |
| | 555 to 557 | SLR |
| | 721 to 723 | TLR |
| | 738 to 740 | SPK |
| Casein kinase II phosphorylation site | 152 to 155 | TFIE |
| | 176 to 179 | SPFD |
| | 250 to 253 | TASE |
| | 342 to 345 | SLLD |
| | 411 to 414 | SGVE |
| | 498 to 501 | SVYE |
| | 502 to 505 | SCVD |
| | 574 to 577 | SILE |
| | 738 to 741 | SPKE |
| | 745 to 748 | SASD |
| N-myristoylation site | 79 to 84 | GAREAI |
| | 191 to 196 | GMLYSG |
| | 331 to 336 | GGTRSS |
| | 412 to 417 | GVEYTR |
| | 437 to 442 | GTTTGS |
| | 620 to 625 | GLYQCW |
| | 671 to 676 | GAALAA |
| Sema domain | 64 to 478 | See FIG. 3 |

An exemplary domains which occurs in TANGO 265 proteins is a sema domain. In one embodiment, the protein of the invention has at least one domain that is at least 55%, preferably at least about 65%, more preferably at least about 75%, yet more preferably at least about 85%, and most preferably at least about 95% identical to one of the sema domains described herein in Table VI.

Sema domains occur in semaphorin proteins. Semaphorins are a large family of secreted and transmembrane proteins, some of which function as repellent signals during neural axon guidance. The sema domain and a variety of semaphorin proteins in which it occurs are described, for example, in Winberg et al. (1998 Cell 95:903-916). Sema domains also occur in human hepatocyte growth factor receptor (Swissprot Accession no. P08581) and the similar neuronal and epithelial transmembrane receptor protein (Swissprot Accession no. P51805). The presence of an sema domain in human TANGO 265 protein indicates that TANGO 265 is involved in one or more physiological processes in which the semaphorins are involved, has biological activity in common with one or more of the semaphorins, or both.

Human TANGO 265 protein exhibits considerable sequence similarity to murine semaphorin B protein (GenBank Accession no. X85991), as indicated herein in FIGS. 3F-3H. FIGS. 3F-3H depict an alignment of the amino acid sequences of human TANGO 265 protein (SEQ ID NO: 19) and murine semaphorin B protein (SEQ ID NO: 76). In this alignment (pam120.mat scoring matrix, gap penalties −12/−4), the amino acid sequences of the proteins are 82.3% identical. FIGS. 31 through 3T depict an alignment of the nucleotide sequences of cDNA encoding human TANGO 265 protein (SEQ ID NO: 17) and murine cDNA encoding semaphorin B protein (SEQ ID NO: 77). In this alignment (Pam120.mat scoring matrix, gap penalties −12/−4), the nucleic acid sequences of the cDNAs are 76.2% identical. Thus, TANGO 265 is the human orthologue of murine semaphorin B and shares functional similarities to that protein.

It is known that semaphorins are bi-functional, capable of functioning either as attractive axonal guidance proteins or as repellent axonal guidance proteins (Wong et al. (1997) Development 124:3597-3607). Furthermore, semaphorins bind with neuronal cell surface proteins designated plexins, which are expressed on both neuronal cells and cells of the immune system (Comeau et al. (1998) Immunity 8:473-482; Jin and Strittmatter (1997) J. Neurosci. 17:6256-6263).

The signal peptide prediction program SIGNALP (Nielsen et al. (1997) Protein Engineering 10:1-6) predicted that human TANGO 265 protein includes a 31 amino acid signal peptide (amino acid residues 1 to 31 of SEQ ID NO: 19; SEQ ID NO: 20) preceding the mature TANGO 265 protein (amino acid residues 32 to 761 of SEQ ID NO: 19; SEQ ID NO: 21). Human TANGO 265 protein includes an extracellular domain (amino acid residues 32 to 683 of SEQ ID NO: 19; SEQ ID NO: 22); a transmembrane domain (amino acid residues 684 to 704 of SEQ ID NO: 19; SEQ ID NO: 23); and a cytoplasmic domain (amino acid residues 705 to 761 of SEQ ID NO: 19; SEQ ID NO: 24).

FIG. 3U depicts a hydrophilicity plot of human TANGO 265 protein. Relatively hydrophobic regions are above the dashed horizontal line, and relatively hydrophilic regions are below the dashed horizontal line. The hydrophobic region which corresponds to amino acid residues 1 to 31 of SEQ ID NO: 19 is the signal sequence of human TANGO 265 (SEQ ID NO: 20). The hydrophobic region which corresponds to amino acid residues 684 to 704 of SEQ ID NO: 19 is the transmembrane domain of human TANGO 265 (SEQ ID NO: 23). As described elsewhere herein, relatively hydrophilic regions are generally located at or near the surface of a protein, and are more frequently effective immunogenic epitopes than are relatively hydrophobic regions. For example, the region of human TANGO 265 protein from about amino acid residue 350 to about amino acid residue 375 appears to be located at or near the surface of the protein, while the region from about amino acid residue 230 to about amino acid residue 250 appears not to be located at or near the surface.

The predicted molecular weight of human TANGO 265 protein without modification and prior to cleavage of the signal sequence is about 83.6 kilodaltons. The predicted molecular weight of the mature human TANGO 265 protein without modification and after cleavage of the signal sequence is about 80.2 kilodaltons.

Chromosomal mapping was performed by computerized comparison of TANGO 265 cDNA sequences against a chromosomal mapping database in order to identify the approximate location of the gene encoding human TANGO 265 protein. This analysis indicated that the gene was located on chromosome 1 between markers D1S305 and D1S2635.

Biological Function of TANGO 265 Proteins, Nucleic Acids, and Modulators Thereof

TANGO 265 proteins are involved in disorders which affect both tissues in which they are normally expressed and tissues in which they are normally not expressed. Based on the observation that TANGO 265 is expressed in human fetal spleen, involvement of TANGO 202 protein in immune system development and modulation is indicated.

The presence of the sema domain in TANGO 265 indicates that this protein is involved in development of neuronal and epithelial tissues and also functions as a repellant protein which guides axonal development. TANGO 265 modulates nerve growth and regeneration and also modulates growth and regeneration of other epithelial tissues.

The observation that TANGO 265 shares significant identity with murine semaphorin B suggests that it has activity identical or analogous to the activity of this protein. These observations indicate that TANGO 265 modulates growth, proliferation, survival, differentiation, and activity of neuronal cells and immune system cells. Thus, TANGO 265 protein is useful, for example, for guiding neural axon development, for modulating differentiation of cells of the immune system, for modulating cytokine production by cells of the immune system, for modulating reactivity of cells of the immune system toward cytokines, for modulating initiation and persistence of an inflammatory response, and for modulating proliferation of epithelial cells.

TANGO 273

A cDNA clone (designated jthoc028g06) encoding at least a portion of human TANGO 273 protein was isolated from a lipopolysaccharide-(LPS-)stimulated human osteoblast cDNA library. The corresponding murine cDNA clone (designated jtmoa001c04) was isolated from an LPS-stimulated murine osteoblast cDNA library. The human and murine TANGO 273 proteins are predicted by structural analysis to be transmembrane proteins.

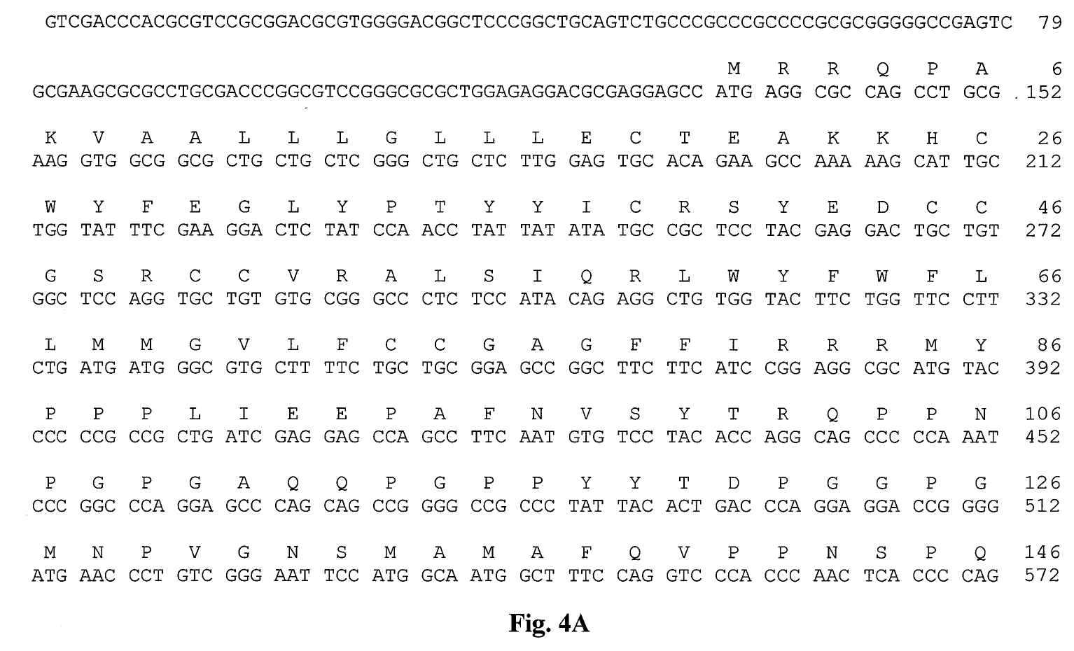

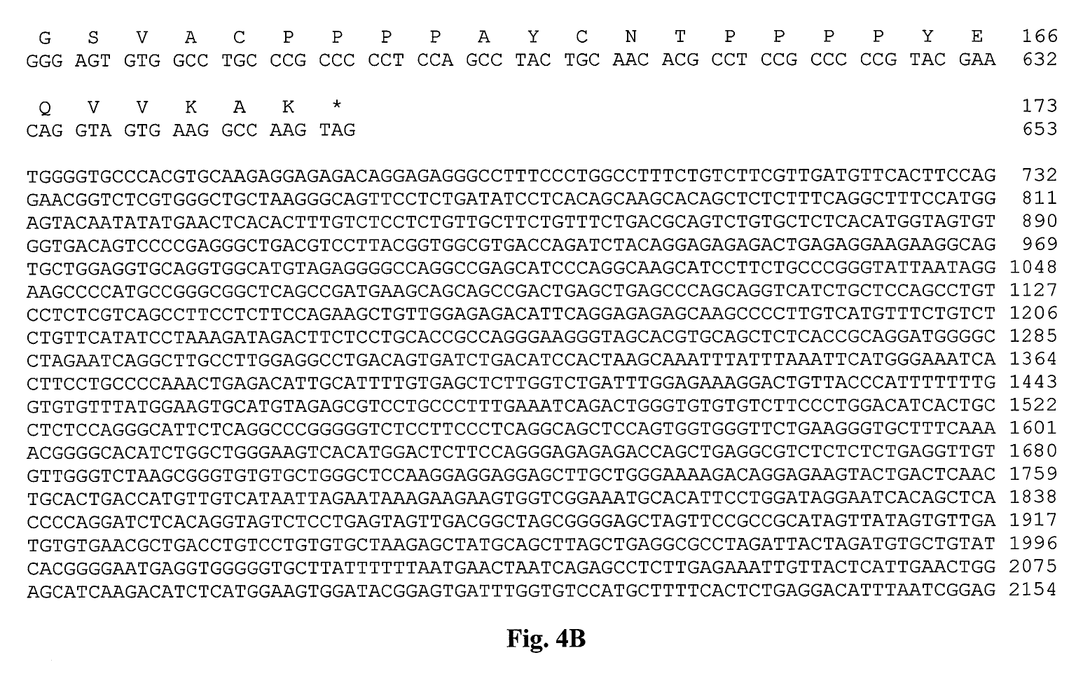

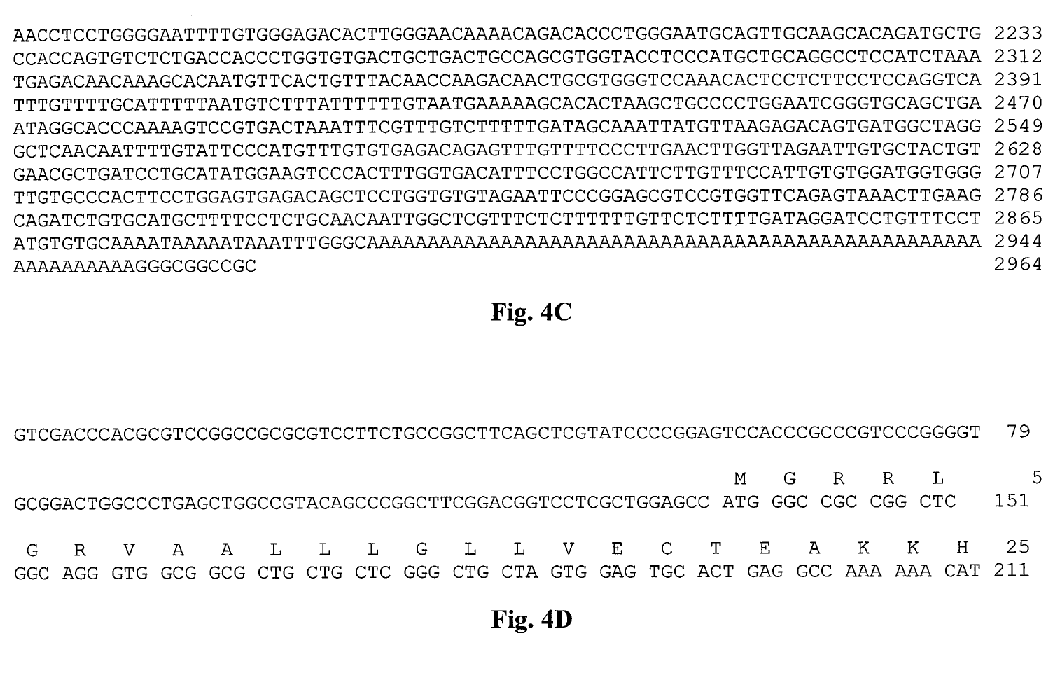

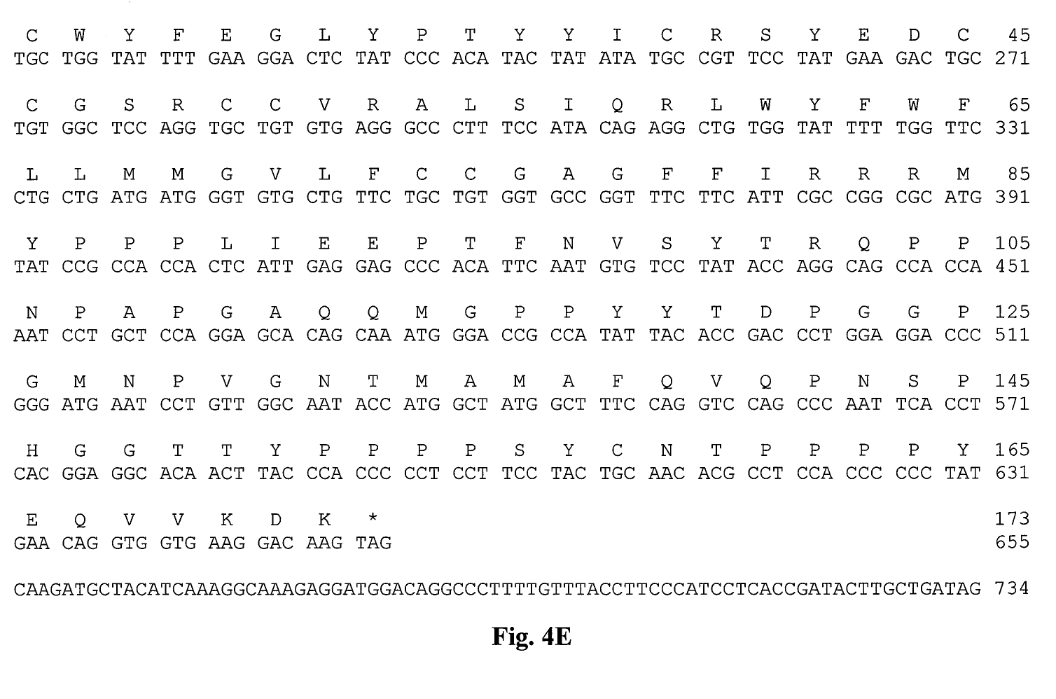

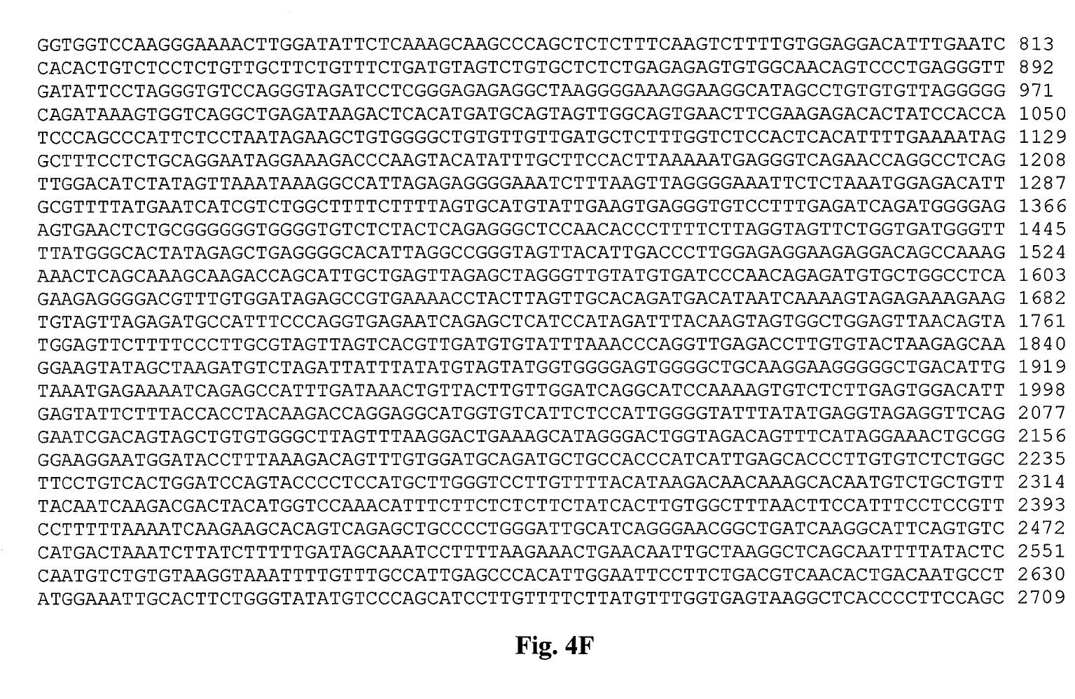

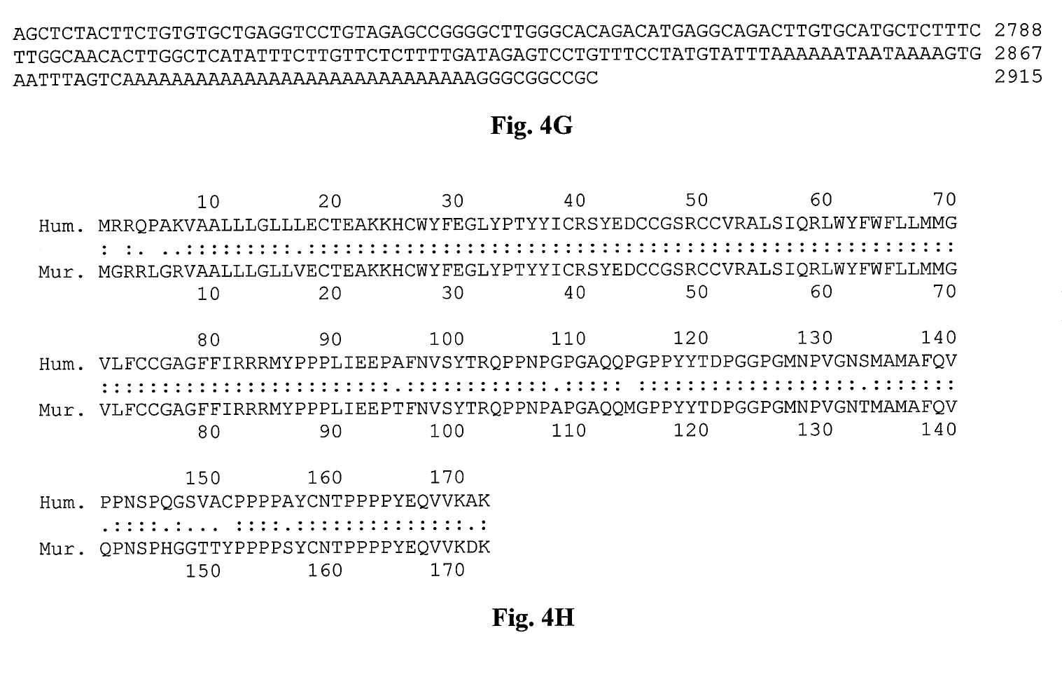

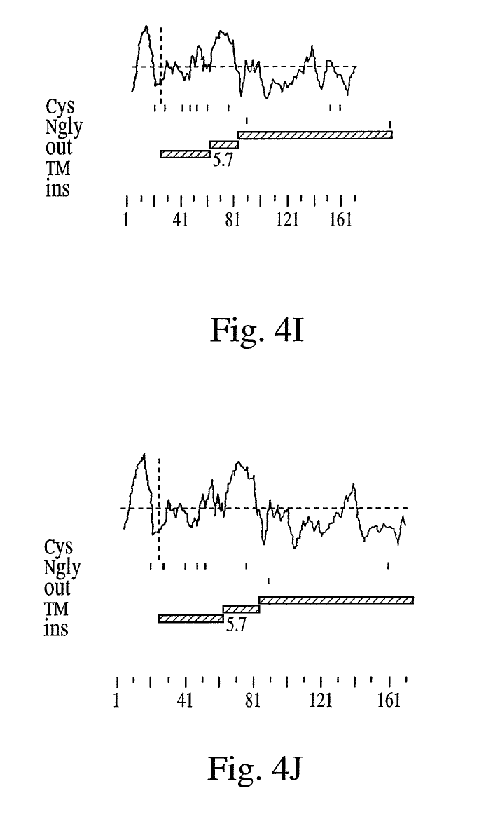

The full length of the cDNA encoding human TANGO 273 protein (FIG. 4; SEQ ID NO: 25) is 2964 nucleotide residues. The ORF of this cDNA, nucleotide residues 135 to 650 of SEQ ID NO: 25 (i.e., SEQ ID NO: 26), encodes a 172-amino acid transmembrane protein (FIG. 4; SEQ ID NO: 27).

The invention thus includes purified human TANGO 273 protein, both in the form of the immature 172 amino acid residue protein (SEQ ID NO: 27) and in the form of the mature 150 amino acid residue protein (SEQ ID NO: 29). The invention also includes purified murine TANGO 273 protein, both in the form of the immature 172 amino acid residue protein (SEQ ID NO: 74) and in the form of the mature 150 amino acid residue protein (SEQ ID NO: 44). Mature human or murine TANGO 273 proteins can be synthesized without the signal sequence polypeptide at the amino terminus thereof, or they can be synthesized by generating immature TANGO 273 protein and cleaving the signal sequence therefrom.

In addition to full length mature and immature human and murine TANGO 273 proteins, the invention includes fragments, derivatives, and variants of these TANGO 273 proteins, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 25 or some portion thereof or SEQ ID NO: 73 or some portion thereof, such as the portion which encodes mature TANGO 273 protein, immature TANGO 273 protein, or a domain of TANGO 273 protein. These nucleic acids are collectively referred to as nucleic acids of the invention.

TANGO 273 proteins and nucleic acid molecules encoding them comprise a family of molecules having certain conserved structural and functional features. This family includes, by way of example, the human and murine TANGO 273 proteins.

A common domain of TANGO 273 proteins is a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 273 protein contains a signal sequence corresponding to amino acid residues 1 to 22 of SEQ ID NO: 27 (SEQ ID NO: 28) or to amino acid residues 1 to 22 of SEQ ID NO: 74. The signal sequence is cleaved during processing of the mature protein.

TANGO 273 proteins can also include an extracellular domain. The human TANGO 273 protein extracellular domain is located from about amino acid residue 23 to about amino acid residue 60 of SEQ ID NO: 27, and the murine TANGO 273 protein extracellular domain is located from about amino acid residue 23 to about amino acid residue 60 of SEQ ID NO: 74.

The present invention also includes TANGO 273 proteins having a transmembrane domain. As used herein, a “transmembrane domain” refers to an amino acid sequence having at least about 15 to 30 amino acid residues in length and which contains at least about 65-70% hydrophobic amino acid residues such as alanine, leucine, phenylalanine, protein, tyrosine, tryptophan, or valine. In a preferred embodiment, a transmembrane domain contains at least about 15 to 20 amino acid residues, preferably about 20 to 25 amino acid residues, and has at least about 60-80%, more preferably 65-75%, and more preferably at least about 70% hydrophobic residues. Thus, in one embodiment, a human TANGO 273 protein of the invention contains a transmembrane domain corresponding to about amino acid residues 61 to 81 of SEQ ID NO: 27 (SEQ ID NO: 31). In another embodiment, a murine TANGO 273 protein of the invention contains a transmembrane domain corresponding to about amino acid residues 61 to 81 of SEQ ID NO: 74.

In addition, TANGO 273 proteins include a cytoplasmic domain. The human TANGO 273 cytoplasmic domain is located from about amino acid residue 82 to amino acid residue 172 of SEQ ID NO: 27 (SEQ ID NO: 32), and the murine TANGO 273 cytoplasmic domain is located from about amino acid residue 82 to amino acid residue 172 of SEQ ID NO: 74.

TANGO 273 proteins typically comprise a variety of potential post-translational modification sites (often within an extracellular domain), such as those described herein in Tables VII and VIII, as predicted by computerized sequence analysis of human and murine TANGO 273 proteins using amino acid sequence comparison software (comparing the amino acid sequence of TANGO 273 with the information in the PROSITE database {rel. 12.2; February, 1995} and the Hidden Markov Models database {Rel. PFAM 3.3}). In certain embodiments, a protein of the invention has at least 1, 2, 3, 4, 5, or all 6 of the post-translational modification sites listed in Table VII. In other embodiments, the protein of the invention has at least 1, 2, 3, 4, 5, 6, or all 7 of the post-translational modification sites listed in Table VIII.

TABLE VII

| | Amino Acid | |
|---|---|---|
| Type of Potential Modification Site or | Residues of | Amino Acid |
| Domain | SEQ ID NO: 27 | Sequence |
| N-glycosylation site | 97 to 100 | NVSY |
| Casein kinase II phosphorylation site | 41 to 44 | SYED |
| N-myristoylation site | 31 to 36 | GLYPTY |
| | 47 to 52 | GSRCCV |
| | 70 to 75 | GVLFCC |
| | 131 to 136 | GNSMAM |
| Src Homology 3 (SH3) domain binding | 86 to 90 | YPPPL |
| site | 103 to 107 | QPPNP |
| | 113 to 117 | QPGPP |
| | 121 to 125 | DPGGP |
| | 140 to 145 | VPPNSP |
| | 151 to 155 | CPPPP |
| | 160 to 164 | TPPPP |

TABLE VIII

| | Amino Acid | |
|---|---|---|
| Type of Potential Modification Site or | Residues of | Amino Acid |
| Domain | SEQ ID NO: 74 | Sequence |
| N-glycosylation site | 97 to 100 | NVSY |
| Casein kinase II phosphorylation site | 41 to 44 | SYED |
| N-myristoylation site | 31 to 36 | GLYPTY |
| | 47 to 52 | GSRCCV |
| | 70 to 75 | GVLFCC |
| | 131 to 136 | GNTMAM |
| Src Homology 3 (SH3) domain binding | 86 to 90 | YPPPL |
| site | 103 to 107 | QPPNP |
| | 115 to 119 | GPPYY |
| | 121 to 125 | DPGGP |
| | 141 to 145 | QPNSP |
| | 151 to 155 | YPPPP |
| | 160 to 164 | TPPPP |
| Amidation site | 1 to 4 | MGRR |

The amino acid sequence of TANGO 273 protein includes about seven potential proline-rich Src homology 3 (SH3) domain binding sites nearer the cytoplasmic portion of the protein. SH3 domains mediate specific assembly of protein complexes, presumably by interacting with proline-rich protein domains (Morton and Campbell (1994) Curr. Biol. 4:615-617). SH3 domains also mediate interactions between proteins involved in transmembrane signal transduction. Coupling of proteins mediated by SH3 domains has been implicated in a variety of physiological systems, including those involving regulation of cell growth and proliferation, endocytosis, and activation of respiratory burst.

SH3 domains have been described in the art (e.g., Mayer et al. (1988) Nature 332:272-275; Musacchio et al. (1992) FEBS Lett. 307:55-61; Pawson and Schlessinger (1993) Curr. Biol. 3:434-442; Mayer and Baltimore (1993) Trends Cell Biol. 3:8-13; Pawson (1993) Nature 373:573-580), and occur in a variety of cytoplasmic proteins, including several (e.g., protein tyrosine kinases) involved in transmembrane signal transduction. Among the proteins in which one or more SH3 domains occur are protein tyrosine kinases such as those of the Src, Abl, Bkt, Csk and ZAP70 families, mammalian phosphatidylinositol-specific phospholipases C-gamma-1 and -2, mammalian phosphatidylinositol 3-kinase regulatory p85 subunit, mammalian Ras GTPase-activating protein (GAP), proteins which mediate binding of guanine nucleotide exchange factors and growth factor receptors (e.g., vertebrate GRB2, Caenorhabditis elegans sem-5, and Drosophila DRK proteins), mammalian Vav oncoprotein, guanidine nucleotide releasing factors of the CDC 25 family (e.g., yeast CDC25, yeast SCD25, and fission yeast ste6 proteins), MAGUK proteins (e.g., mammalian tight junction protein ZO-1, vertebrate erythrocyte membrane protein p55, C. elegans protein lin-2, rat protein CASK, and mammalian synaptic proteins SAP90/PSD-95, CHAPSYN-110/PSD-93, SAP97/DLG1, and SAP102), proteins which interact with vertebrate receptor protein tyrosine kinases (e.g., mammalian cytoplasmic protein Nck and oncoprotein Crk), chicken Src substrate p80/85 protein (cortactin), human hemopoietic lineage cell specific protein Hs1, mammalian dihydrouridine-sensitive L-type calcium channel beta subunit, human myasthenic syndrome antigen B (MSYB), mammalian neutrophil cytosolic activators of NADPH oxidase (e.g., p47 {NCF-1}, p67 {NCF-2}, and C. elegans protein B0303.7) myosin heavy chains (MYO3) from amoebae, from slime molds, and from yeast, vertebrate and Drosophila spectrin and fodrin alpha chain proteins, human amphiphysin, yeast actin-binding proteins ABP1 and SLA3, yeast protein BEM 1, fission yeast protein scd2 (ra13), yeast BEM1-binding proteins BOI2 (BEB1) and BOB1 (BOI1), yeast fusion protein FUS1, yeast protein RSV167, yeast protein SSU81, yeast hypothetical proteins YAR014c, YFR024c, YHL002w, YHR016c, YJL020C, and YHR114w, hypothetical fission yeast protein SpAC12C2.05c, and C. elegans hypothetical protein F42H10.3. Of these proteins, multiple SH3 domains occur in vertebrate GRB2 protein, C. elegans sem-5 protein, Drosophila DRK protein, oncoprotein Crk, mammalian neutrophil cytosolic activators of NADPH oxidase p47 and p67, yeast protein BEM1, fission yeast protein scd2, yeast hypothetical protein YHR114w, mammalian cytoplasmic protein Nck, C. elegans neutrophil cytosolic activator of NADPH oxidase B0303.7, and yeast actin-binding protein SLA1. Of these proteins, three or more SH3 domains occur in mammalian cytoplasmic protein Nck, C. elegans neutrophil cytosolic activator of NADPH oxidase B0303.7, and yeast actin-binding protein SLA1. The presence of SH3 domain binding sites in TANGO 273 indicates that TANGO 273 interacts with one or more of these and other SH3 domain-containing proteins and is thus involved in physiological processes in which one or more of these or other SH3 domain-containing proteins are involved.

The signal peptide prediction program SIGNALP (Nielsen et al. (1997) Protein Engineering 10:1-6) predicted that human TANGO 273 protein includes a 22 amino acid signal peptide (amino acid residues 1 to 22 of SEQ ID NO: 27; SEQ ID NO: 28) preceding the mature TANGO 273 protein (amino acid residues 23 to 172 of SEQ ID NO: 27; SEQ ID NO: 29). Human TANGO 273 protein includes an extracellular domain (amino acid residues 23 to 60 of SEQ ID NO: 27; SEQ ID NO: 30); a transmembrane domain (amino acid residues 61 to 81 of SEQ ID NO: 27; SEQ ID NO: 31); and a cytoplasmic domain (amino acid residues 82 to 172 of SEQ ID NO: 27; SEQ ID NO: 32).

FIG. 4I depicts a hydrophilicity plot of human TANGO 273 protein. Relatively hydrophobic regions are above the dashed horizontal line, and relatively hydrophilic regions are below the dashed horizontal line. The hydrophobic region which corresponds to amino acid residues 1 to 22 of SEQ ID NO: 27 is the signal sequence of human TANGO 273 (SEQ ID NO: 28). The hydrophobic region which corresponds to amino acid residues 61 to 81 of SEQ ID NO: 27 is the transmembrane domain of human TANGO 273 (SEQ ID NO: 31). As described elsewhere herein, relatively hydrophilic regions are generally located at or near the surface of a protein, and are more frequently effective immunogenic epitopes than are relatively hydrophobic regions. For example, the region of human TANGO 273 protein from about amino acid residue 100 to about amino acid residue 120 appears to be located at or near the surface of the protein, while the region from about amino acid residue 130 to about amino acid residue 140 appears not to be located at or near the surface.

Chromosomal mapping was performed by computerized comparison of TANGO 273 cDNA sequences against a chromosomal mapping database in order to identify the approximate location of the gene encoding human TANGO 273 protein. This analysis indicated that the gene was located on chromosome 7 between markers D7S2467 and D7S2552.

The predicted molecular weight of human TANGO 273 protein without modification and prior to cleavage of the signal sequence is about 19.2 kilodaltons. The predicted molecular weight of the mature human TANGO 273 protein without modification and after cleavage of the signal sequence is about 16.8 kilodaltons.

Northern analysis experiments indicated that mRNA corresponding to the cDNA encoding TANGO 273 is expressed in the tissues listed in Table VIIa, wherein “++” indicates moderate expression and “+” indicates lower expression.

TABLE VIIa

| Animal | Tissue | Relative Level of Expression |
|---|---|---|
| Human | heart | ++ |
| | brain | ++ |
| | skeletal muscle | ++ |
| | pancreas | ++ |
| | placenta | + |
| | lung | + |
| | liver | + |
| | kidney | + |

The full length of the cDNA encoding murine TANGO 273 protein (FIG. 4; SEQ ID NO: 72) is 2915 nucleotide residues. The ORF of this cDNA, nucleotide residues 137 to 650 of SEQ ID NO: 72 (i.e., SEQ ID NO: 73), encodes a 172-amino acid transmembrane protein (FIG. 4; SEQ ID NO: 74).

The signal peptide prediction program SIGNALP (Nielsen et al. (1997) Protein Engineering 10:1-6) predicted that murine TANGO 273 protein includes a 22 amino acid signal peptide (amino acid residues 1 to 22 of SEQ ID NO: 74) preceding the mature TANGO 273 protein (amino acid residues 23 to 172 of SEQ ID NO: 74; SEQ ID NO: 44). Murine TANGO 273 protein includes an extracellular domain (amino acid residues 23 to 60 of SEQ ID NO: 74); a transmembrane domain (amino acid residues 61 to 81 of SEQ ID NO: 74); and a cytoplasmic domain (amino acid residues 82 to 172 of SEQ ID NO: 74).

FIG. 4J depicts a hydrophilicity plot of murine TANGO 273 protein. Relatively hydrophobic regions are above the dashed horizontal line, and relatively hydrophilic regions are below the dashed horizontal line. The hydrophobic region which corresponds to amino acid residues 1 to 22 of SEQ ID NO: 74 is the signal sequence of murine TANGO 273. As described elsewhere herein, relatively hydrophilic regions are generally located at or near the surface of a protein, and are more frequently effective immunogenic epitopes than are relatively hydrophobic regions. For example, the region of murine TANGO 273 protein from about amino acid residue 100 to about amino acid residue 120 appears to be located at or near the surface of the protein, while the region from about amino acid residue 130 to about amino acid residue 140 appears not to be located at or near the surface.

The predicted molecular weight of murine TANGO 273 protein without modification and prior to cleavage of the signal sequence is about 19.4 kilodaltons. The predicted molecular weight of the mature murine TANGO 273 protein without modification and after cleavage of the signal sequence is about 17.1 kilodaltons.

In situ analysis of murine TANGO 273 mRNA indicated that TANGO 273 is expressed with central nervous system (CNS) tissues during embryogenesis and into adulthood. Expression of TANGO 273 is widely observed in murine CNS tissues, including brain, spinal cord, eye, and olfactory epithelium at all embryonic ages examined (i.e., at embryonic days 13.5, 14.5, 15.5, 16.5, and 18.5 and at post-natal day 1.5).

Human and murine TANGO 273 cDNA sequences exhibit significant nucleotide sequence identity with an expressed sequence tag (EST) isolated from a library of ESTs corresponding to proteins secreted from prostate tissue, as described in PCT publication number WO 99/06550, published Feb. 11, 1999.

Human and murine TANGO 273 proteins exhibit considerable sequence similarity, as indicated herein in FIG. 4H. FIG. 4H depicts an alignment of human and murine TANGO 273 protein amino acid sequences (SEQ ID NOs: 27 and 74, respectively). In this alignment (pam120.mat scoring matrix, gap penalties −12/−4), the proteins are 89.5% identical. Alignment of the ORF encoding human TANGO 273 protein and the ORF encoding murine TANGO 273 protein using the same software and parameters indicated that the nucleotide sequences are 84.1% identical.

Biological Function of TANGO 273 Proteins, Nucleic Acids, and Modulators Thereof

cDNAs encoding the human and murine TANGO 273 proteins were each isolated from LPS-stimulated osteoblast cDNA libraries. These proteins are involved in bone-related metabolism, homeostasis, and development disorders. Thus, proteins and nucleic acids of the invention which are identical to, similar to, or derived from human and murine TANGO 273 proteins and nucleic acids encoding them are useful for preventing, diagnosing, and treating, among others, bone-related disorders such as osteoporosis, cancer, skeletal development disorders, bone fragility, and the like.

Expression of TANGO 273 in heart, brain, skeletal muscle, and pancreas, placenta, lung, liver, and kidney tissues is an indication that TANGO 273 proteins, nucleic acids encoding them, and agents that modulate activity or expression of either of these can be used to modulate growth, proliferation, survival, differentiation, adhesion, and activity of cells of these tissues, or to prognosticate, diagnose, and treat one or more disorders which affect these tissues.

The fact that TANGO 273 is expressed at high levels in neurological tissues is an indication that TANGO 273 proteins, nucleic acids, and modulators thereof can be used to modulate proliferation, differentiation, or function of neurological cells in these tissues (e.g., neuronal cells). Thus, TANGO 273 proteins, nucleic acids, and modulators thereof can be used to prognosticate, diagnose, and treat one or more neurological disorders. Examples of such disorders include CNS disorders, CNS-related disorders, focal brain disorders, global-diffuse cerebral disorders, and other neurological and cerebrovascular disorders.

CNS disorders include, but are not limited to cognitive and neurodegenerative disorders such as Alzheimer's disease, senile dementia, Huntington's disease, amyotrophic lateral sclerosis, and Parkinson's disease, as well as Gilles de la Tourette's syndrome, autonomic function disorders such as hypertension and sleep disorders (e.g., insomnia, hypersomnia, parasomnia, and sleep apnea); neuropsychiatric disorders (e.g., schizophrenia, schizoaffective disorder, attention deficit disorder, dysthymic disorder, major depressive disorder, mania, and obsessive-compulsive disorder); psychoactive substance use disorders; anxiety; panic disorder; and bipolar affective disorders (e.g., severe bipolar affective disorder and bipolar affective disorder with hypomania and major depression).

CNS-related disorders include disorders associated with developmental, cognitive, and autonomic neural and neurological processes, such as pain, appetite, long term memory, and short term memory.

Exemplary focal brain disorders include aphasia, apraxia, agnosia, and amnesias (e.g., posttraumatic amnesia, transient global amnesia, and psychogenic amnesia). Global-diffuse cerebral disorders with which TANGO 273 can be associated include coma, stupor, obtundation, and disorders of the reticular formation.

Other neurological disorders with which TANGO 273 can be associated include ischemic syndromes (e.g., stroke), hypertensive encephalopathy, hemorrhagic disorders, and disorders involving aberrant function of the blood-brain barrier (e.g., CNS infections such as meningitis and encephalitis, aseptic meningitis, metastasis of non-CNS tumor cells into the CNS, various pain disorders such as migraine, blindness and other vision problems, and CNS-related adverse drug reactions such as head pain, sleepiness, and confusion). TANGO 273 proteins, nucleic acids encoding them, and agents that modulate activity or expression of either of these can be used to prognosticate, diagnose, and treat one or more of these disorders.

Developmental regulation of TANGO 273 expression in fetal neurological tissues, as described herein, is an indication that TANGO 273 proteins, nucleic acids, and modulators thereof can be used to prognosticate, diagnose, and treat one or more disorders which involve aberrant fetal neurological development. Examples of such disorders include blindness, deafness, fetal death, mental retardation, dysraphia, anencephaly, malformation of cerebral hemispheres, encephalocele, porencephaly, hydranencephaly, hydrocephalus, and spina bifida.

The fact that TANGO 273 is expressed in tissues which were exposed to LPS indicates that TANGO 273 mediates one or more physiological responses of cells to bacterial infection. Thus, TANGO 273 is involved in one or more of detection of bacteria in a tissue in which it is expressed, movement of cells with relation to sites of bacterial infection, production of biological molecules which inhibit bacterial infection, and production of biological molecules which alleviate cellular or other physiological damage wrought by bacterial infection.

Presence in TANGO 273 protein of multiple SH3 domain binding sites indicates that TANGO 273 protein interacts with one or more SH3 domain- containing proteins. Thus, TANGO 273 protein mediates binding of proteins (i.e., binding of proteins to TANGO 273 and to one another to form protein complexes) in cells in which it is expressed. TANGO 273 is also involved in transduction of signals between the exterior environment of cells (i.e., including from other cells) and the interior of cells in which it is expressed. TANGO 273 mediates regulation of cell growth and proliferation, endocytosis, activation of respiratory burst, and other physiological processes triggered by transmission of a signal via a protein with which TANGO 273 interacts.

Sequence similarity of TANGO 273 cDNA with an EST expressed in prostate tissue indicates that TANGO 273 can be expressed in prostate tissue, and can thus be involved in disorders of the prostate. Thus, TANGO 273 proteins, nucleic acids encoding them, and agents that modulate activity or expression of either of these can be used to treat prostate disorders. Examples of prostate disorders which can be treated in this manner include inflammatory prostatic diseases (e.g., acute and chronic prostatitis and granulomatous prostatitis), prostatic hyperplasia (e.g., benign prostatic hypertrophy or hyperplasia), and prostate tumors (e.g., carcinomas).

In another example, TANGO 273 polypeptides, nucleic acids, or modulators thereof, can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cardiomyopathy).

In another example, TANGO 273 polypeptides, nucleic acids, or modulators thereof, can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, TANGO 273 polypeptides, nucleic acids, or modulators thereof, can be used to treat disorders of skeletal muscle, such as muscular dystrophy (e.g., Duchenne muscular dystrophy, Becker muscular dystrophy, Emery-Dreifuss muscular dystrophy, limb-girdle muscular dystrophy, facioscapulohumeral muscular dystrophy, myotonic dystrophy, oculopharyngeal muscular dystrophy, distal muscular dystrophy, and congenital muscular dystrophy), motor neuron diseases (e.g., amyotrophic lateral sclerosis, infantile progressive spinal muscular atrophy, intermediate spinal muscular atrophy, spinal bulbar muscular atrophy, and adult spinal muscular atrophy), myopathies (e.g., inflammatory myopathies such as dermatomyositis and polymyositis, myotonia congenita, paramyotonia congenita, central core disease, nemaline myopathy, myotubular myopathy, and periodic paralysis), and metabolic diseases of muscle (e.g., phosphorylase deficiency, acid maltase deficiency, phosphofructokinase deficiency, debrancher enzyme deficiency, mitochondrial myopathy, camitine deficiency, camitine palmityl transferase deficiency, phosphoglycerate kinase deficiency, phosphoglycerate mutase deficiency, lactate dehydrogenase deficiency, and myoadenylate deaminase deficiency).

In another example, TANGO 273 polypeptides, nucleic acids, or modulators thereof, can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

In another example, TANGO 273 polypeptides, nucleic acids, or modulators thereof, can be used to treat placental disorders, such as toxemia of pregnancy (e.g., preeclampsia and eclampsia), placentitis, or spontaneous abortion.

In another example, TANGO 273 polypeptides, nucleic acids, or modulators thereof, can be used to treat pulmonary disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchioalveolar carcinoma, bronchial carcinoid, hamartoma, and mesenchymal tumors).

In another example, TANGO 273 polypeptides, nucleic acids, or modulators thereof, can be used to treat hepatic (liver) disorders, such as jaundice, hepatic failure, hereditary hyperbilirubinemias (e.g., Gilbert's syndrome, Crigler-Naijar syndromes, and Dubin-Johnson and Rotor's syndromes), hepatic circulatory disorders (e.g., hepatic vein thrombosis and portal vein obstruction and thrombosis) hepatitis (e.g., chronic active hepatitis, acute viral hepatitis, and toxic and drug-induced hepatitis) cirrhosis (e.g., alcoholic cirrhosis, biliary cirrhosis, and hemochromatosis), or malignant tumors (e.g., primary carcinoma, hepatoblastoma, and angiosarcoma).

In another example, TANGO 273 polypeptides, nucleic acids, or modulators thereof, can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease such as systemic lupus erythematosus, Goodpasture's syndrome, multiple myeloma, diabetes, neoplasia, sickle cell disease, and chronic inflammatory diseases), tubular diseases (e.g., acute tubular necrosis and acute renal failure, polycystic renal disease, medullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

TANGO 286

A cDNA clone (designated jthkf042e03) encoding at least a portion of human TANGO 286 protein was isolated from a human keratinocyte cDNA library. The human TANGO 286 protein is predicted by structural analysis to be a secreted protein.

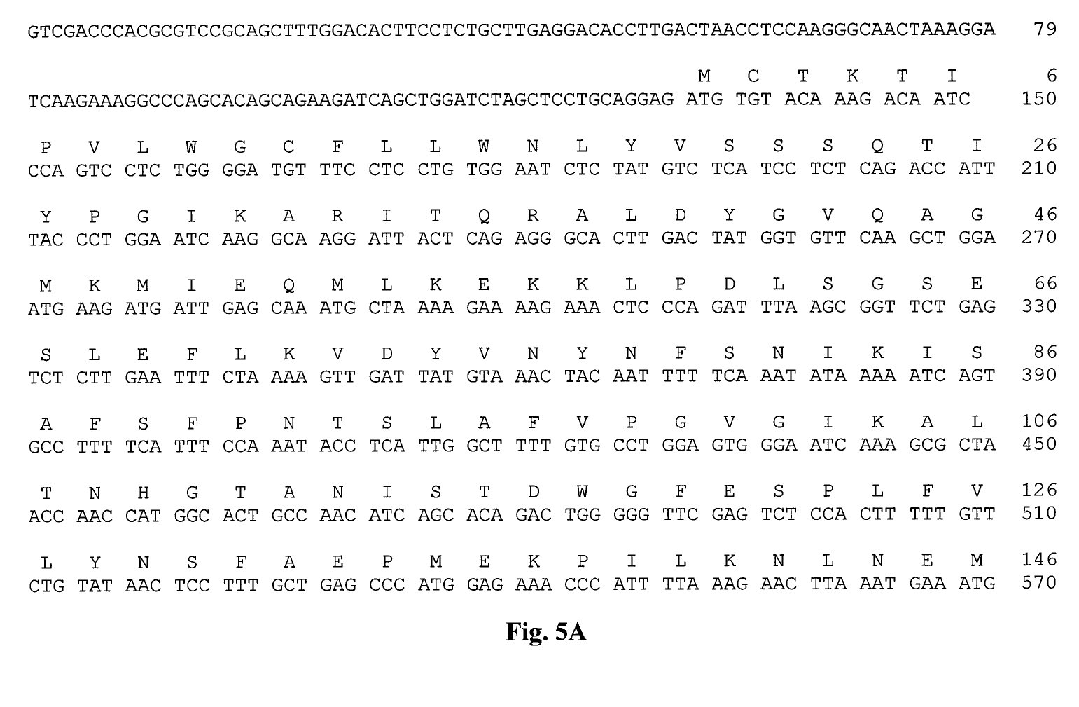

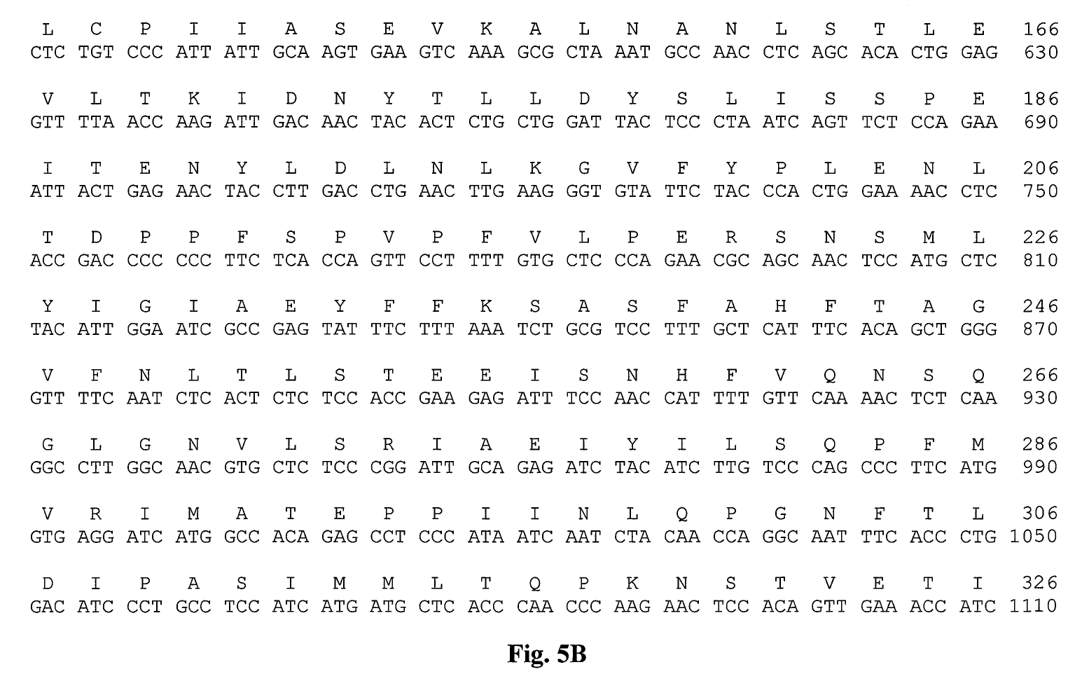

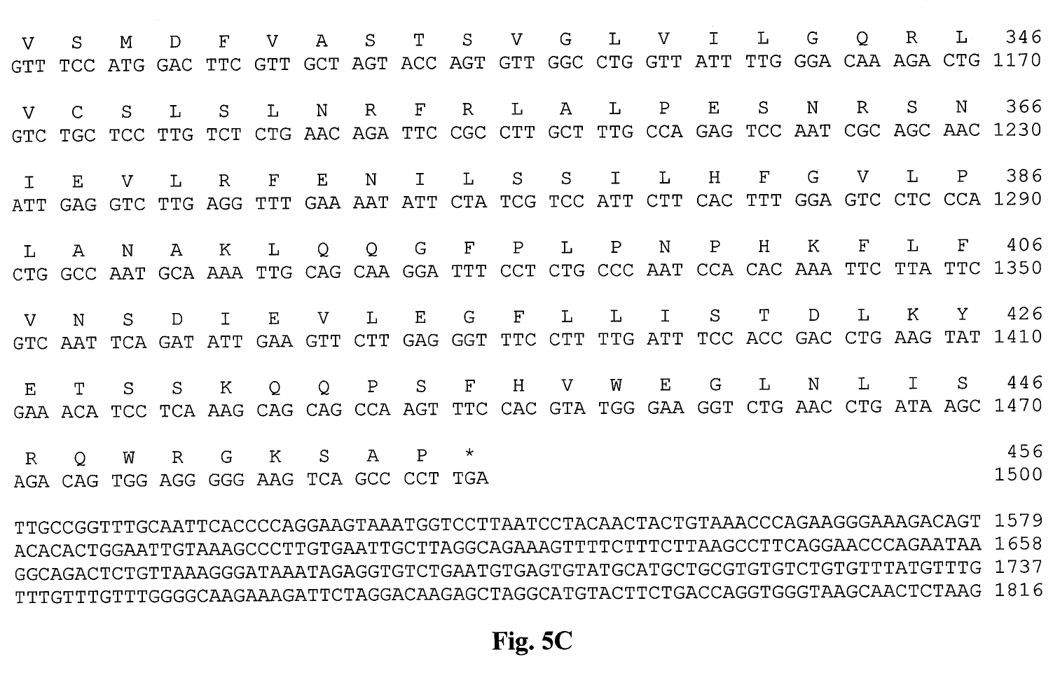

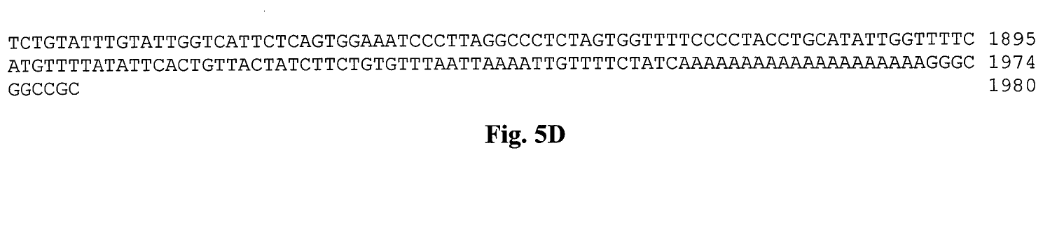

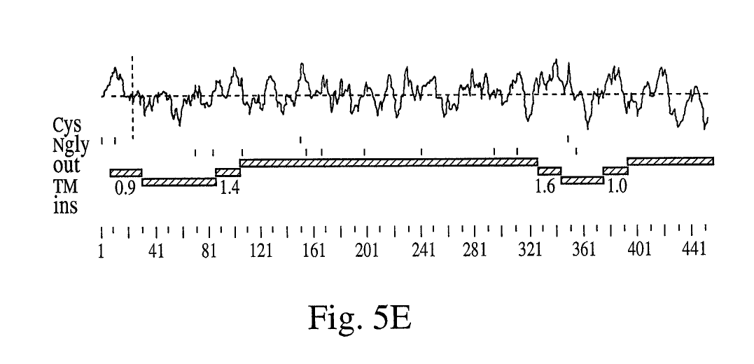

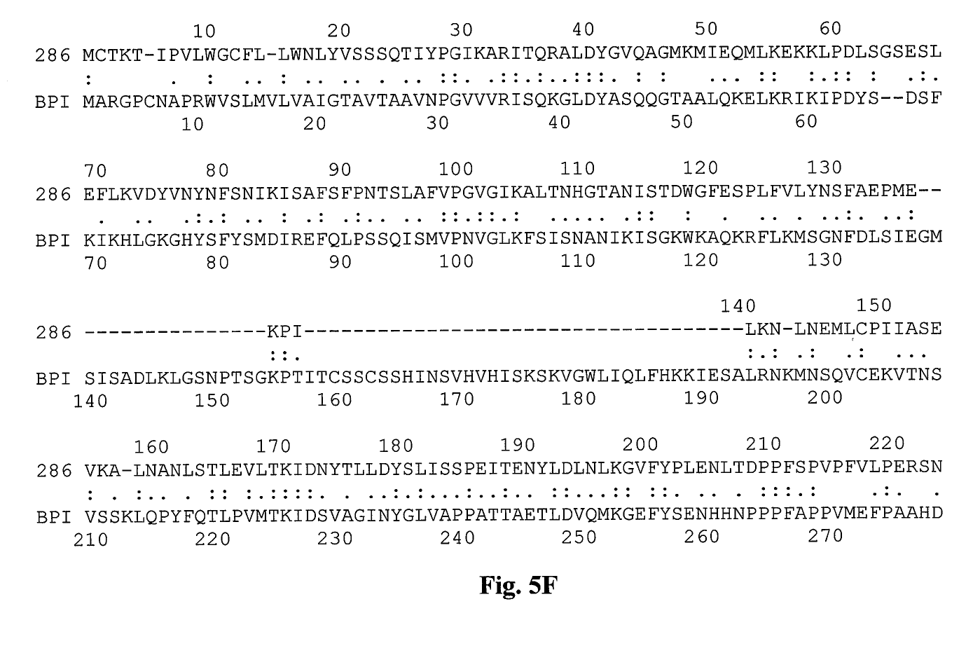

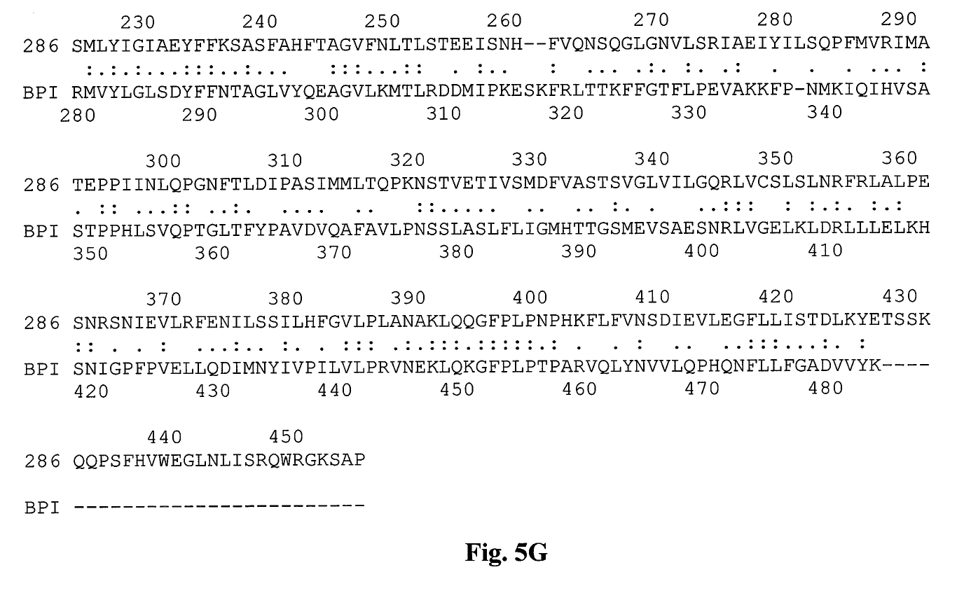

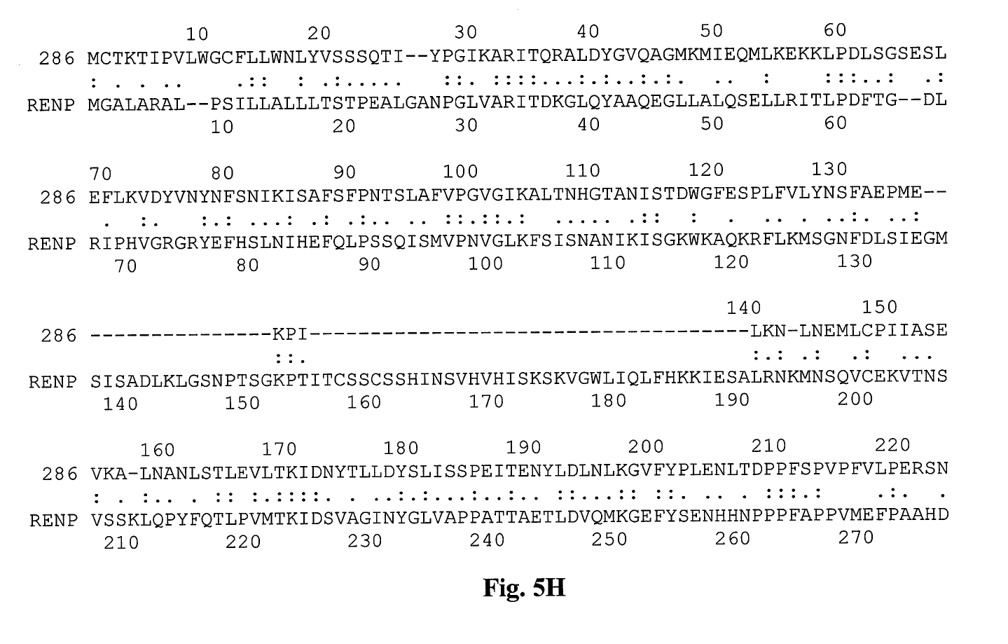

The full length of the cDNA encoding TANGO 286 protein (FIG. 5; SEQ ID NO: 33) is 1980 nucleotide residues. The ORF of this cDNA, nucleotide residues 133 to 1497 of SEQ ID NO: 33 (i.e., SEQ ID NO: 34), encodes a 455-amino acid secreted protein (FIG. 5; SEQ ID NO: 35).

The invention thus includes purified TANGO 286 protein, both in the form of the immature 455 amino acid residue protein (SEQ ID NO: 35) and in the form of the mature 432 amino acid residue protein (SEQ ID NO: 37). Mature TANGO 286 protein can be synthesized without the signal sequence polypeptide at the amino terminus thereof, or it can be synthesized by generating immature TANGO 286 protein and cleaving the signal sequence therefrom.

In addition to full length mature and immature TANGO 286 proteins, the invention includes fragments, derivatives, and variants of these TANGO 286 proteins, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 33 or some portion thereof, such as the portion which encodes mature TANGO 286 protein, immature TANGO 286 protein, or a domain of TANGO 286 protein. These nucleic acids are collectively referred to as nucleic acids of the invention.

TANGO 286 proteins and nucleic acid molecules encoding them comprise a family of molecules having certain conserved structural and functional features.

A common domain of TANGO 286 proteins is a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 286 protein contains a signal sequence corresponding to amino acid residues 1 to 23 of SEQ ID NO: 35 (SEQ ID NO: 36). The signal sequence is cleaved during processing of the mature protein.

TANGO 286 is a secreted soluble protein (i.e., a secreted protein having a single extracellular domain), as indicated by computerized sequence analysis and comparison of the amino acid sequence of TANGO 286 with related proteins, such as the soluble proteins designated bactericidal permeability increasing (BPI) protein and recombinant endotoxin neutralizing polypeptide (RENP).

TANGO 286 proteins typically comprise a variety of potential post-translational modification sites (often within an extracellular domain), such as those described herein in Table IX, as predicted by computerized sequence analysis of TANGO 286 proteins using amino acid sequence comparison software (comparing the amino acid sequence of TANGO 286 with the information in the PROSITE database {rel. 12.2; February, 1995} and the Hidden Markov Models database {Rel. PFAM 3.3 }). In certain embodiments, a protein of the invention has at least 1, 2, 4, 6, 10, 15, or 20 or more of the post-translational modification sites listed in Table IX.

TABLE IX

| | Amino Acid | |
|---|---|---|
| Type of Potential Modification Site or | Residues of | Amino Acid |
| Domain | SEQ ID NO: 35 | Sequence |
| N-glycosylation site | 79 to 82 | NFSN |
| | 92 to 95 | NTSL |
| | 113 to 116 | NIST |
| | 161 to 164 | NLST |
| | 173 to 176 | NYTL |
| | 205 to 208 | NLTD |
| | 249 to 252 | NLTL |
| | 303 to 306 | NFTL |
| | 320 to 323 | NSTV |
| | 363 to 366 | NRSN |
| Protein kinase C phosphorylation site | 35 to 37 | TQR |
| | 362 to 364 | SNR |
| | 429 to 431 | SSK |
| Casein kinase II phosphorylation site | 63 to 66 | SGSE |
| | 130 to 133 | SFAE |
| | 163 to 166 | STLE |
| | 169 to 172 | TKID |
| | 175 to 178 | TLLD |
| | 183 to 186 | SSPE |
| | 253 to 256 | STEE |
| | 321 to 324 | STVE |
| | 365 to 368 | SNIE |
| | 409 to 412 | SDIE |
| N-myristoylation site | 42 to 47 | GVQAGM |
| | 269 to 274 | GNVLSR |
| Lipid-binding serum glycoprotein | 12 to 427 | see FIG. 5 |
| domain | | |

Certain lipid-binding serum glycoproteins, such as LPS-binding protein (LBP), bactericidal permeability-increasing protein (BPI), cholesteryl ester transfer protein (CETP), and phospholipid transfer protein (PLTP), share regions of sequence similarity which are herein designated a lipid-binding serum glycoprotein domain (Schumann et al., (1990) Science 249:1429-1431; Gray et al., (1989) J. Biol. Chem. 264:9505-9509; Day et al., (1994) J. Biol. Chem. 269:9388-9391). The consensus pattern of lipid-binding serum glycoprotein domains is as follows (using standard single letter amino acid abbreviations wherein X is any amino acid residue).

-(P or A)-(G or A)-(L or I or V or M or C)-X₂-R-(I or V)-(S or T)-X₃-L-X_{(4 or 5)}-(E or Q)-X₄-(L or I or V or M)-X_{(0 or 1)}-(E or Q or K)-X₈-P-(e.g., amino acid residues 28-60 of SEQ ID NO: 35).

Proteins in which a lipid-binding serum glycoprotein domain occurs are often structurally related and exhibit related physiological activities. LBP binds to lipid A moieties of bacterial LPS and, once bound thereto, induces secretion of a-tumor necrosis factor, apparently by interacting with the CD14 receptor. BPI also binds LPS and exerts a cytotoxic effect on Gram-negative bacteria (Elsbach, (1998) J. Leukoc. Biol. 64:14-18). CETP is involved in transfer of insoluble cholesteryl esters during reverse cholesterol transport. PLTP appears to be involved in phospholipid transport and modulation of serum HDL particles.

The signal peptide prediction program SIGNALP (Nielsen et al. (1997) Protein Engineering 10:1-6) predicted that TANGO 286 protein includes a 23 amino acid signal peptide (amino acid residues 1 to 23 of SEQ ID NO: 35; SEQ ID NO: 36) preceding the mature TANGO 286 protein (amino acid residues 24 to 455 of SEQ ID NO: 35; SEQ ID NO: 37). Human TANGO 286 protein is a secreted soluble protein.

FIG. 5E depicts a hydrophilicity plot of TANGO 286 protein. Relatively hydrophobic regions are above the dashed horizontal line, and relatively hydrophilic regions are below the dashed horizontal line. As described elsewhere herein, relatively hydrophilic regions are generally located at or near the surface of a protein, and are more frequently effective immunogenic epitopes than are relatively hydrophobic regions For example, the region of human TANGO 286 protein from about amino acid residue 420 to about amino acid residue 435 appears to be located at or near the surface of the protein, while the region from about amino acid residue 325 to about amino acid residue 345 appears not to be located at or near the surface.

The predicted molecular weight of TANGO 286 protein without modification and prior to cleavage of the signal sequence is about 50.9 kilodaltons. The predicted molecular weight of the mature TANGO 286 protein without modification and after cleavage of the signal sequence is about 48.2 kilodaltons.

The gene encoding human TANGO 286 protein was determined to be located on chromosome 22 by comparison of matching genomic clones such as the clones assigned GenBank Accession numbers W16806 and AL021937.

A portion of TANGO 286 protein exhibits significant amino acid homology with a region of the human chromosome region 22q12-13 genomic nucleotide sequence having GenBank Accession number AL021937. Alignment of a 45 kilobase nucleotide sequence encoding TANGO 286 with AL021937, however, indicated the presence in TANGO 286 of exons which differ from those disclosed in L021937 (pam120.mat scoring matrix; gap penalties −12/−4). This region of chromosome 22 comprises an immunoglobulin lambda chain C (IGLC) pseudogene, the Ret finger protein-like 3 (RFPL3) and Ret finger protein-like 3 antisense (RFPL3S) genes, a gene encoding a novel immunoglobulin lambda chain V family protein, a novel gene encoding a protein similar both to mouse RGDS protein (RALGDS, RALGEF, guanine nucleotide dissociation stimulator A) and to rabbit oncogene RSC, a novel gene encoding the human orthologue of worm F16A11.2 protein, a novel gene encoding a protein similar both to BPI and to rabbit liposaccharide-binding protein, and a 5′-portion of a novel gene. This region also comprises various ESTs, STSs, GSSs, genomic marker D22S1175, a ca repeat polymorphism and putative CpG islands. TANGO 286 protein thus shares one or more structural or functional features of these molecules.

TANGO 286 protein exhibits considerable sequence similarity with BPI protein, having 23.9% amino acid sequence identity therewith, as assessed using the ALIGN v. 2.0 computer software using a pam120.mat scoring matrix and gap penalties of −12/−4. TANGO 286 protein also exhibits considerable sequence similarity with recombinant endotoxin neutralizing polypeptide (RENP), having 24.5% amino acid sequence identity therewith, as assessed using the ALIGN software. Physiological activities of BPI protein and RENP have been described (e.g., Gabay et al., (1989) Proc. Natl. Acad. Sci. USA 86:5610-5614; Elsbach, (1998) J. Leukoc. Biol. 64:14-18; Mahadeva et al., (1997) Chest 112:1699-1701; International patent application WO96/34873). RENP, for example, binds LPS and neutralizes bacterial endotoxins. BPI, RENP, and other proteins in which a lipid-binding serum glycoprotein domain occurs bind LPS and neutralize bacterial endotoxins, and are therefore useful for preventing, detecting, and treating LPS-related disorders such as shock, disseminated intravascular coagulation, anemia, thrombocytopenia, adult respiratory distress syndrome, renal failure, liver disease, and disorders associated with Gram negative bacterial infections. In addition to the physiological conditions described above, BPI protein is known to be involved in vasculitis and bronchiectasis, in that antibodies which bind specifically with BPI protein are present in at least some patients afflicted with these disorders (Mahadeva et al., supra).

Biological Function of TANGO 286 Proteins, Nucleic Acids, and Modulators Thereof

Expression of TANGO 286 in keratinocyte library indicates that this protein is involved in a disorders which involve keratinocytes. Such disorders include, for example, disorders involving extracellular matrix abnormalities, dermatological disorders, ocular disorders, inappropriate hair growth (e.g., baldness), infections of the nails of the fingers and toes, scalp disorders (e.g., dandruff), and the like.

The fact that TANGO 286 protein contains a lipid-binding serum glycoprotein domain indicates that TANGO 286 is involved in one or more physiological processes in which these other lipid-binding serum glycoprotein domain-containing proteins are involved. Thus, TANGO 286 is involved in one or more of lipid transport, metabolism, serum lipid particle regulation, host anti-microbial defensive mechanisms, and the like.

Human TANGO 286 shares physiological functionality with other proteins in which a lipid-binding serum glycoprotein domains occurs (e.g., LBP, BPI protein, CETP, and PLTP). Based on the amino acid sequence similarity of TANGO 286 with BPI protein and with RENP, TANGO 286 protein exhibits physiological activities exhibited by these proteins. Thus, TANGO 286 proteins are useful for preventing, diagnosing, and treating, among others, lipid transport disorders, lipid metabolism disorders, disorders of serum lipid particle regulation, obesity, disorders involving insufficient or inappropriate host anti-microbial defensive mechanisms, vasculitis, bronchiectasis, LPS-related disorders such as shock, disseminated intravascular coagulation, anemia, thrombocytopenia, adult respiratory distress syndrome, renal failure, liver disease, and disorders associated with Gram negative bacterial infections, such as bacteremia, endotoxemia, sepsis, and the like.

TANGO 294

A cDNA clone (designated jthrc145g07) encoding at least a portion of human TANGO 294 protein was isolated from a human pulmonary artery smooth muscle cell cDNA library. The human TANGO 294 protein is predicted by structural analysis to be a transmembrane membrane protein. Expression of DNA encoding TANGO 294 was observed, using a variety of methods, in pancreas, lung tumor, stomach, pulmonary artery smooth muscle cells, and colon tumor tissues and in activated peripheral blood mononuclear cells (PBMCs). More detailed expression data is described herein.

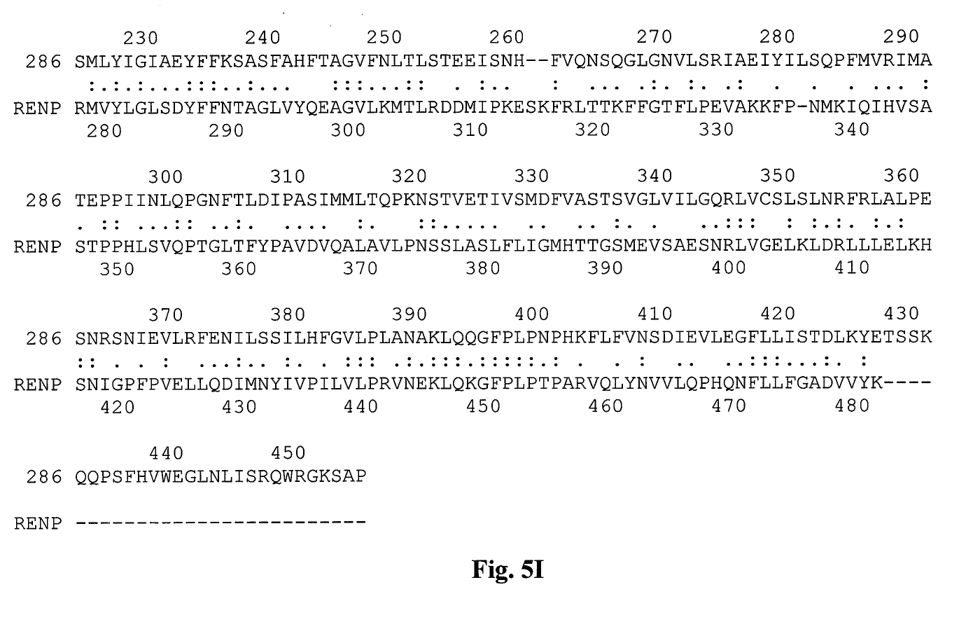

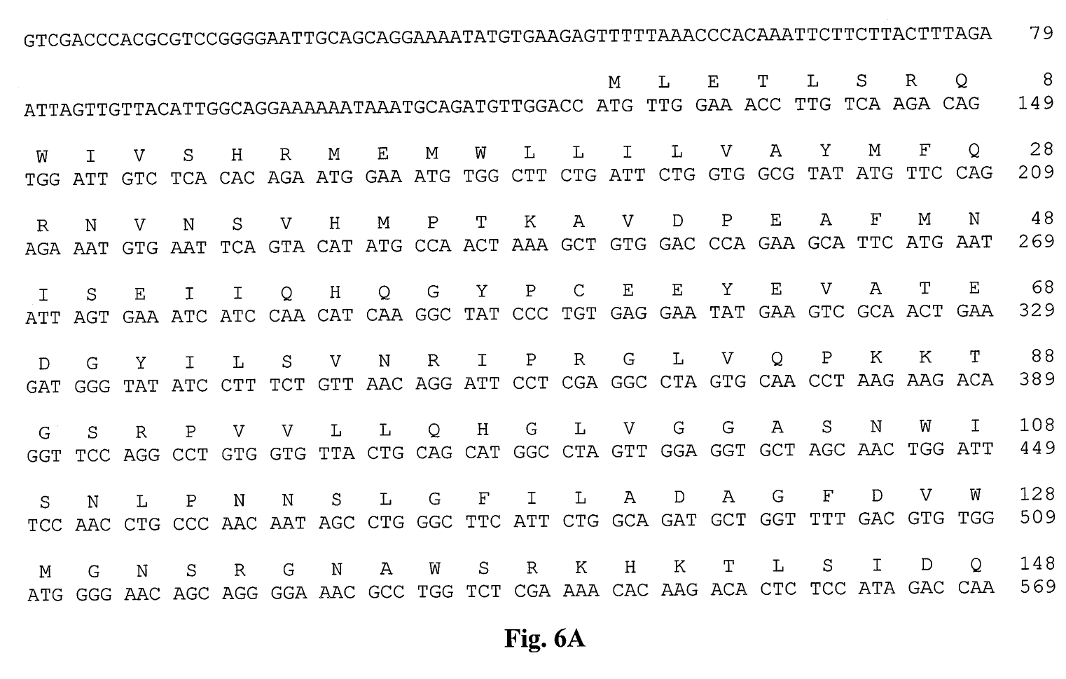

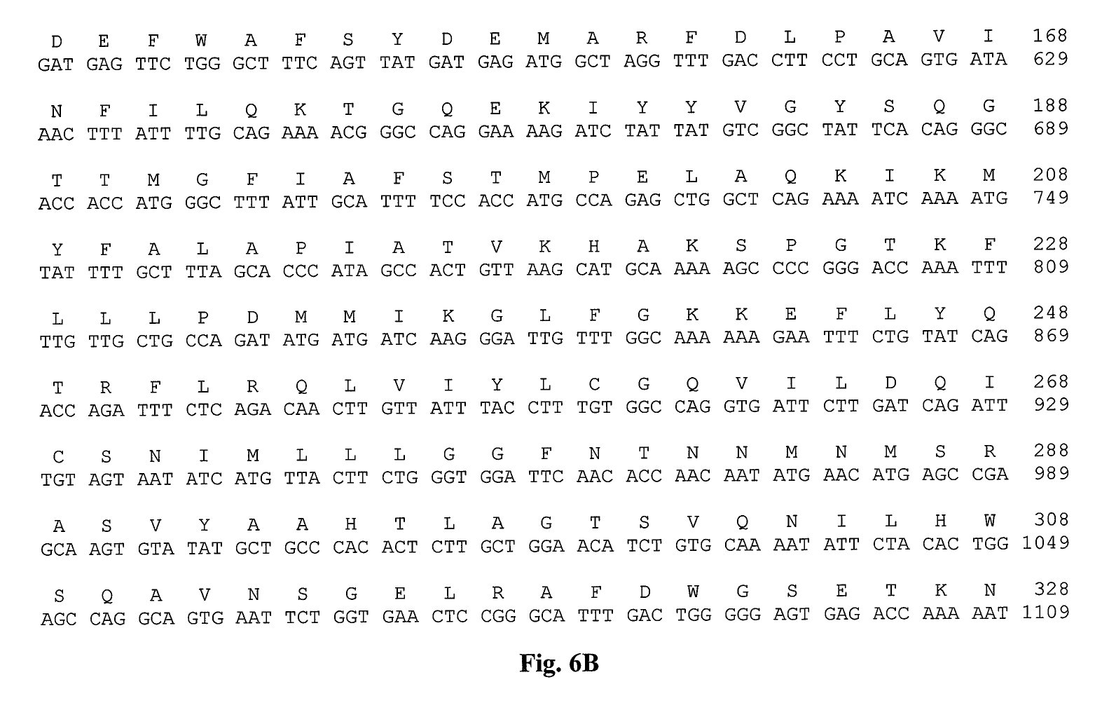

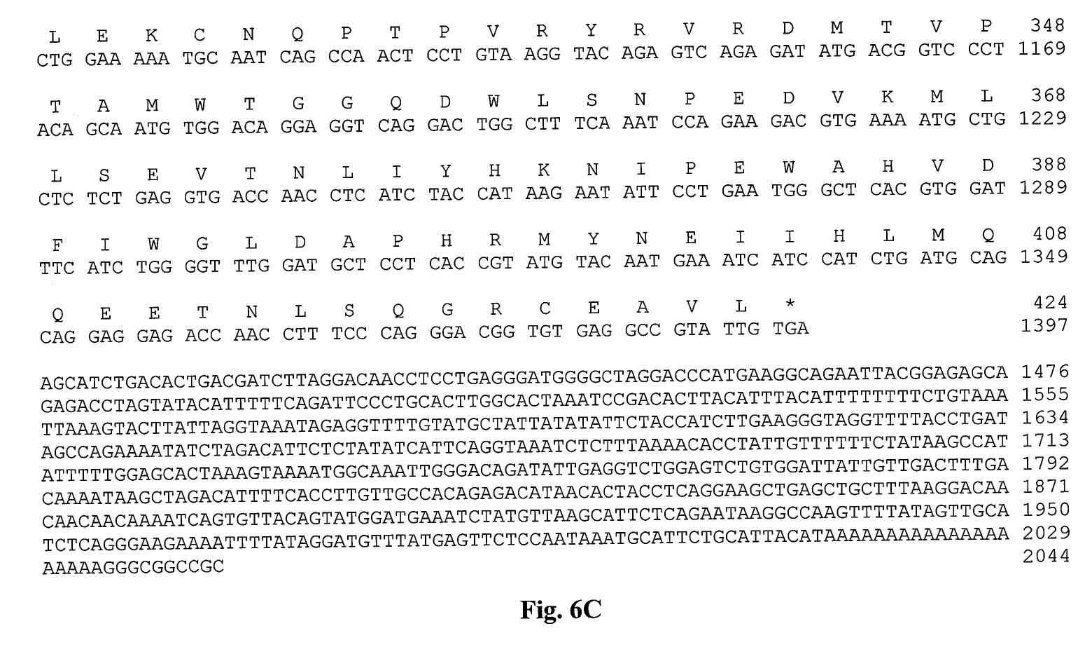

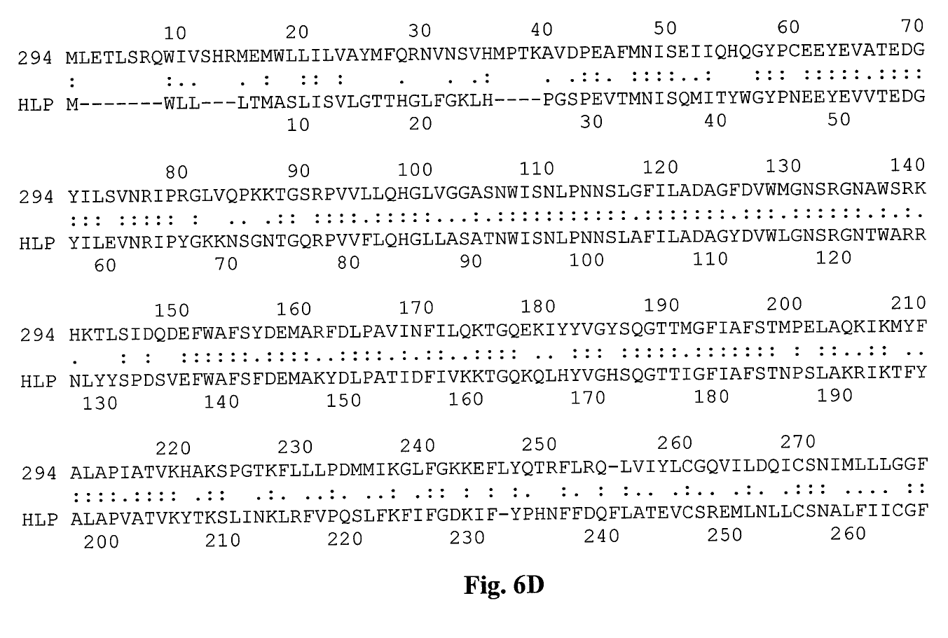

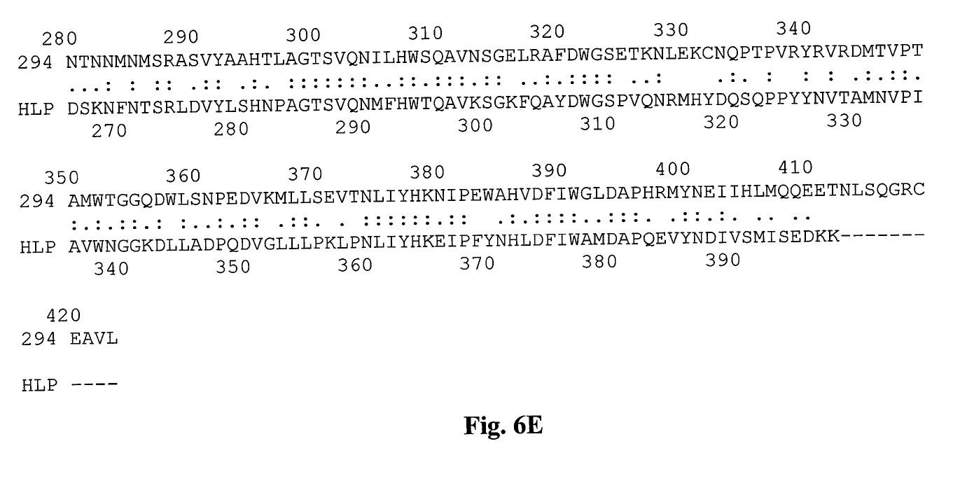

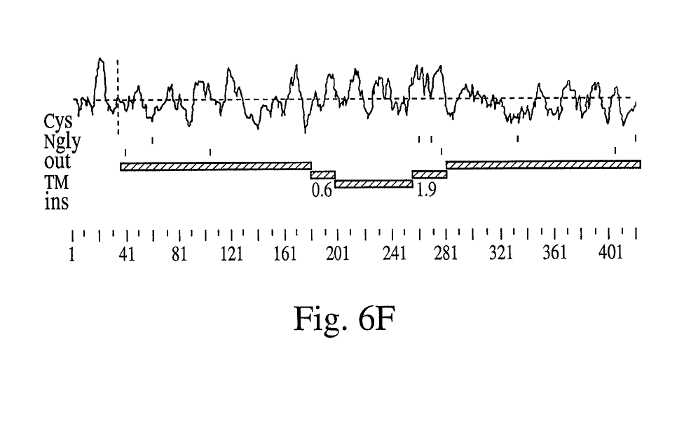

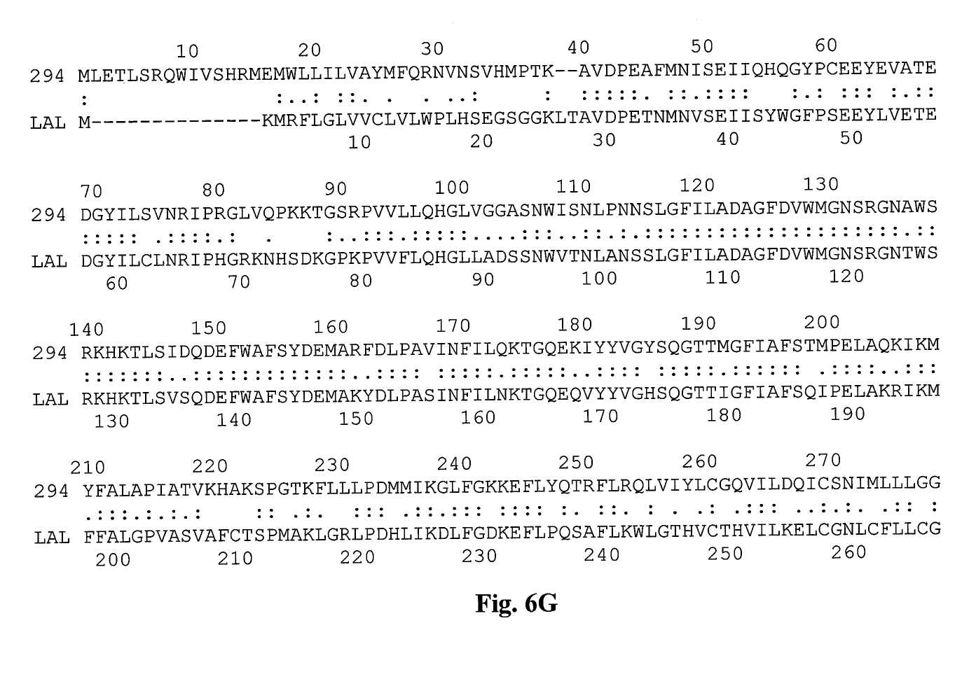

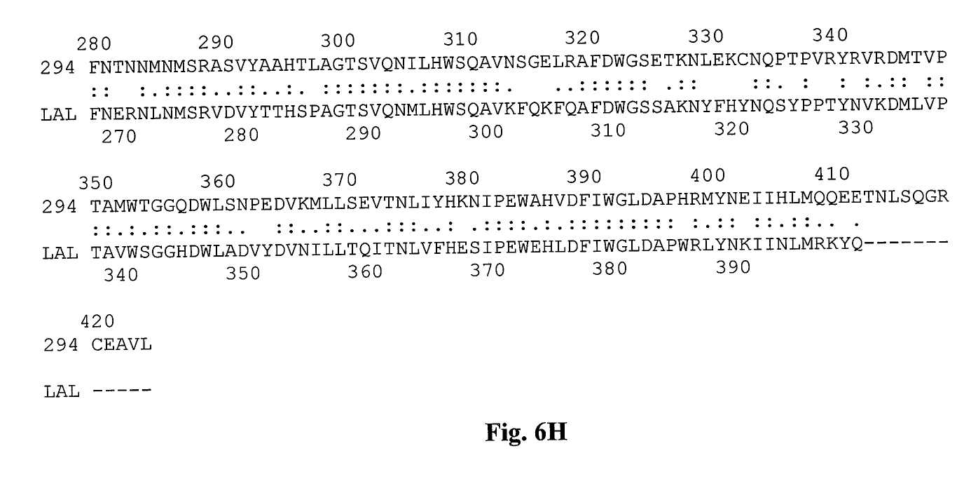

The full length of the cDNA encoding TANGO 294 protein (FIG. 6; SEQ ID NO: 45) is 2044 nucleotide residues. The ORF of this cDNA, nucleotide residues 126 to 1394 of SEQ ID NO: 45 (i.e., SEQ ID NO: 46), encodes a 423-amino acid transmembrane protein (FIG. 6; SEQ ID NO: 47).

The invention includes purified TANGO 294 protein, both in the form of the immature 423 amino acid residue protein (SEQ ID NO: 47) and in the form of the mature 390 amino acid residue protein (SEQ ID NO: 49). Mature TANGO 294 protein can be synthesized without the signal sequence polypeptide at the amino terminus thereof, or it can be synthesized by generating immature TANGO 294 protein and cleaving the signal sequence therefrom.

In addition to full length mature and immature TANGO 294 proteins, the invention includes fragments, derivatives, and variants of TANGO 294 protein, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence listed in SEQ ID NO: 45 or some portion thereof, such as the portion which encodes mature TANGO 294 protein, immature TANGO 294 protein, or a domain of TANGO 294 protein. These nucleic acids are collectively referred to as nucleic acids of the invention.

TANGO 294 proteins and nucleic acid molecules encoding them comprise a family of molecules having certain conserved structural and functional features.

Also included within the scope of the invention are TANGO 294 proteins having a signal sequence. As used herein, a signal sequence includes a peptide of at least about 10 amino acid residues in length which occurs at the amino terminus of membrane-bound proteins and which contains at least about 45% hydrophobic amino acid residues such as alanine, leucine, isoleucine, phenylalanine, proline, tyrosine, tryptophan, or valine. In a preferred embodiment, a signal sequence contains at least about 10 to 35 amino acid residues, preferably about 10 to 20 amino acid residues, and has at least about 35-60%, more preferably 40-50%, and more preferably at least about 45% hydrophobic residues. A signal sequence serves to direct a protein containing such a sequence to a lipid bilayer. Thus, in one embodiment, a TANGO 294 protein contains a signal sequence corresponding to amino acid residues 1 to 33 of SEQ ID NO: 47 (SEQ ID NO: 48). The signal sequence is cleaved during processing of the mature protein.

The naturally-occurring form of TANGO 294 protein is a secreted protein (i.e., not comprising the predicted signal sequence). However, in variant forms, TANGO 294 proteins can be transmembrane proteins which include an extracellular domain. In this transmembrane variant form, the predicted TANGO 294 protein extracellular domain is located from about amino acid residue 34 to about amino acid residue 254 of SEQ ID NO: 47, the predicted cytoplasmic domain is located from about amino acid residue 280 to amino acid residue 423 of SEQ ID NO: 47 (SEQ ID NO: 52), and the predicted transmembrane domain is located from about amino acid residues 255 to 279 of SEQ ID NO: 47 (SEQ ID NO: 51).

TANGO 294 proteins typically comprise a variety of potential post-translational modification sites (often within an extracellular domain), such as those described herein in Table X, as predicted by computerized sequence analysis of TANGO 294 proteins using amino acid sequence comparison software (comparing the amino acid sequence of TANGO 294 with the information in the PROSITE database {rel. 12.2; February, 1995} and the Hidden Markov Models database {Rel. PFAM 3.3}). In certain embodiments, a protein of the invention has at least 1, 2, 4, 6, 10, 15, or 20 or more of the post-translational modification sites listed in Table X.

TABLE X

| | Amino Acid | |
|---|---|---|
| Type of Potential Modification Site | Residues of | Amino Acid |
| or Domain | SEQ ID NO: 47 | Sequence |
| N-glycosylation site | 48 to 51 | NISE |
| | 113 to 116 | NNSL |
| | 285 to 288 | NMSR |
| | 413 to 416 | NLSQ |
| Protein kinase C phosphorylation | 12 to 14 | SHR |
| site | 138 to 140 | SRK |
| | 217 to 219 | TVK |
| Casein kinase II phosphorylation | 155 to 158 | SYDE |
| site | 175 to 178 | TGQE |
| | 198 to 201 | TMPE |
| | 360 to 363 | SNPE |
| Tyrosine kinase phosphorylation | 174 to 182 | KTGQEKIYY |
| site | | |
| N-myristoylation site | 99 to 104 | GLVGGA |
| | 130 to 135 | GNSRGN |
| | 188 to 193 | GTTMGF |
| | 277 to 282 | GGFNTN |
| Amidation site | 240 to 243 | FGKK |
| Lipase serine active site | 180 to 189 | IYYVGYSQGT |
| Lipase Conserved Active Site | 186 | 5 |
| Residues | 357 | D |
| | 386 | H |
| Lipase Conserved Cysteine | 260 | C |
| Residues | 269 | C |
| Lipase Conserved Oxyanion Hole | 100 | L |
| Residues | 187 | Q |
| Alpha/beta hydrolase fold domain | 125 to 404 | See FIG. 6 |

Alpha/beta hydrolase fold domains occur in a wide variety of enzymes (Ollis et al., (1992) Protein Eng. 5:197-211). The alpha/beta fold domain is a conserved topological domain in which sequence homology is not necessarily conserved. Conservation of topology in the alpha/beta fold domain preserves arrangement of catalytic residues, even though those residues, and the reactions they catalyze, can vary. In many enzymes, particularly including alpha/beta hydrolases, this domain encompasses the active site of the enzyme. In one embodiment, the protein of the invention has at least one domain that is at least 55%, preferably at least about 65%, more preferably at least about 75%, yet more preferably at least about 85%, and most preferably at least about 95% identical to the alpha/beta hydrolase fold domain described herein in Table X.

The signal peptide prediction program SIGNALP (Nielsen et al. (1997) Protein Engineering 10:1-6) predicted that human TANGO 294 protein includes a 33 amino acid signal peptide (amino acid residues 1 to 33 of SEQ ID NO: 47; SEQ ID NO: 48) preceding the mature TANGO 294 protein (amino acid residues 34 to 423 of SEQ ID NO: 47; SEQ ID NO: 49). Human TANGO 294 protein is a soluble secreted protein. However, in the transmembrane variant form, human TANGO 294 protein includes an extracellular domain (amino acid residues 34 to 254 of SEQ ID NO: 47; SEQ ID NO: 50); a transmembrane domain (amino acid residues 255 to 279 of SEQ ID NO: 47; SEQ ID NO: 51); and a cytoplasmic domain (amino acid residues 280 to 423 of SEQ ID NO: 47; SEQ ID NO: 52).

FIG. 6F depicts a hydrophilicity plot of human TANGO 294 protein. Relatively hydrophobic regions are above the dashed horizontal line, and relatively hydrophilic regions are below the dashed horizontal line. The hydrophobic region which corresponds to amino acid residues 1 to 33 of SEQ ID NO: 47 is the signal sequence of human TANGO 294 (SEQ ID NO: 49). The hydrophobic region which corresponds to amino acid residues 255 to 279 of SEQ ID NO: 47 is the predicted transmembrane domain of human TANGO 294 (SEQ ID NO: 51). As described elsewhere herein, relatively hydrophilic regions are generally located at or near the surface of a protein, and are more frequently effective immunogenic epitopes than are relatively hydrophobic regions. For example, the region of human TANGO 294 protein from about amino acid residue 130 to about amino acid residue 150 appears to be located at or near the surface of the protein, while the region from about amino acid residue 90 to about amino acid residue 100 appears not to be located at or near the surface.

The predicted molecular weight of human TANGO 294 protein without modification and prior to cleavage of the signal sequence is about 48.2 kilodaltons. The predicted molecular weight of the mature human TANGO 294 protein without modification and after cleavage of the signal sequence is about 44.2 kilodaltons.

It may be that amino acid residues 1 to 15 of SEQ ID NO: 47 do not occur in TANGO 294 protein. However, it is recognized that amino acid residues 16 to 33 of SEQ ID NO: 47 form a functional signal sequence even in the absence of residues 1 to 15. The amino acid sequence (and hence the properties) of mature TANGO 294 protein are unaffected by presence or absence of amino acid residues 1 to 15 of immature TANGO 294 protein.

Human TANGO 294 protein exhibits considerable sequence similarity (i.e., about 75% amino acid sequence identity) to lingual and gastric lipase proteins of rat (Swissprot Accession no. P04634; Docherty et al. (1985) Nucleic Acids Res. 13:1891-1903), dog (Swissprot Accession no. P80035; Carriere et al. (1991) Eur. J. Biochem. 202:75-83), and human (Swissprot Accession no. P07098; Bembaeck and Blaeckberg (1987) Biochim. Biophys. Acta 909:237-244), as assessed using the ALIGN v. 2.0 computer software using a pam12.mat scoring matrix and gap penalties of −12/−4. TANGO 294 is distinct from the known human lipase, as indicated in FIGS. 6D and 6E. FIGS. 6D and 6E depict an alignment of the amino acid sequences of human TANGO 294 protein (SEQ ID NO: 47) and the known human lipase protein (SEQ ID NO: 75), as assessed using the same software and parameters. In this alignment (pam120.mat scoring matrix, gap penalties −12/−4), the amino acid sequences of the proteins are 49.8% identical. TANGO 294 also is distinct from the known human lysosomal acid lipase, as indicated in FIGS. 6G and 6H. FIGS. 6G and 6H depicts an alignment of the amino acid sequences of human TANGO 294 protein (SEQ ID NO: 47) and the known human lysosomal acid lipase protein (SEQ ID NO: 41). In this alignment (pam120.mat scoring matrix, gap penalties −12/−4), the amino acid sequences of the proteins are 56.9% identical.

TANGO 294 is a human lipase distinct from the known human lipase and the known human lysosomal acid lipase. Furthermore, in view of the comparisons of the amino acid sequences of TANGO 294 and the two human lipases and the nature of transcriptional initiation sites, it is recognized that the transcriptional start site can correspond to either of the methionine residues located at residues 1 and 15 of SEQ ID NO: 47 The present invention thus includes proteins in which the initially transcribed amino acid residue is the methionine residue at position 1 of SEQ ID NO: 47 and proteins in which the initially transcribed amino acid residue is the methionine residue at position 15 of SEQ ID NO: 47 (i.e., proteins in which the amino acid sequence of TANGO 294 does not include residues 1 to 14 of SEQ ID NO: 47). Furthermore, because amino acid residues 1 to 14 of SEQ ID NO: 47 are predicted to be part of a signal sequence, it is recognized that the protein not comprising this portion of the amino acid sequence will nonetheless exhibit a functional signal sequence at its amino terminus.

TANGO 294 Expression Analysis

TaqMan™ Experiments

Total RNA was prepared from various human tissues by a single step extraction method using RNA STAT-60 according to the manufacturer's instructions (TelTest, Inc). Each RNA preparation was treated with DNase I (Ambion) at 37° C. for 1 hour. DNAse I treatment was determined to be complete if the sample required at least 38 PCR amplification cycles to reach a threshold level of fluorescence using beta-2 microglobulin as an internal amplicon reference. The integrity of the RNA samples following DNase I treatment was confirmed by agarose gel electrophoresis and ethidium bromide staining. After phenol extraction cDNA was prepared from the sample using the SUPERSCRIPT™ Choice System following the manufacturer's instructions (Gibco BRL). A negative control of RNA without reverse transcriptase was mock reverse transcribed for each RNA sample.

Novel TANGO 294 expression was measured by TaqMan™ quantitative PCR (Perkin Elmer Applied Biosystems) in cDNA prepared from the following human tissues:

Tissue Panel One (Phase I, General Expression in normal and tumorigenic tissues): Artery normal, Aorta diseased, Vein normal, Coronary SMC, HUVEC, Hemangioma, Heart (normal), Congestive Heart Failure, Kidney, Skeletal Muscle, Adipose (normal), Pancreas, Primary Osteoblasts, Osteoclasts (differentiated), Skin (normal), Spinal Cord (normal), Brain Cortex (normal), Brain Hypothalamus (normal), Nerve, Dorsal Root Ganglia, Breast (normal), Breast Tumor, Ovary (normal), Ovary Tumor, Prostate (normal), Prostate Tumor, Salivary Glands, Colon (pools of 3 normal colon tissues), Colon Tumor (3 colon adenocarcinomas), Lung (normal), Lung Tumor, Lung (COPD), Colon (3 colon IBD samples), Liver (normal), Liver Fibrosis, Spleen (normal), Tonsil (normal), Lymph Node (normal), Small Intestine (normal), Macrophages, Synovium, Bone Marrow-MNC, Activated Peripheral Blood Mononuclear Cells (PBMC), Neutrophils, Megakaryocytes, and Erthyroid.

Tissue Panel Two (Phase II, General Expression Pattern in Solid Tumors): Breast (normal, 3 separate samples), Breast (tumor, 5 separate samples), Lymph node (metastasized from breast), Lung (metastasized from breast), Ovary (normal, 2 separate samples), Ovary (tumor, 5 separate samples), Lung (normal, 3 separate samples), Lung (tumor, 6 separate samples), Colon (normal, 3 separate samples), Colon (Adenocarcinoma, 4 separate samples), Liver (metastasized from colon, 3 separate samples), Liver (normal, female), Cervix Squamous Carcinoma (2 separate samples), Human Microvascular Endothelial Cells- Arr, Human Microvascular Endothelial Cells- Prol, Pooled Hemangiomas, HCT116N22 Normoxic, and HCT116H22 Hypoxic.

Tissue Panel Three (Colon Cancer Expanded Panel: Specific expression pattern in various stages of colorectal cancer): Colon (normal, 6 separate samples), Colon (adenomas, 2 separate samples), Colon (stage B adenocarcinomas, 6 separate samples), Colon (stage C adenocarcinomas, 6 separate samples), Liver (normal, 6 separate samples), Liver (metastasized from colon, 6 separate samples), and 1 abdominal colon metastasis sample.

Tissue Panel Four (Xenograft Panel): MCF-7 Breast T, ZR75 Breast T, T47D Breast T, MDA 231 Breast T, MDA 435 Breast T, SKBr3 Breast, DLD 1 ColonT (stage C), SW480 Colon T (stage B), SW620 ColonT (stage C), HCT116, HT29, Colon 205, NCIH125, NCIH67, NCIH322, NCIH460, A549, NHBE, SKOV-3 ovary, OVCAR-3 ovary, 293 Baby Kidney, and 293T Baby Kidney.

Tissue Panel Five (Colon to Liver Metastases Panel: Specific expression patterns in late stage colon cancer (metastasis)): Colon (normal, 3 separate samples), Colonic ACA-C, Colonic ACA-C, Colonic ACA-B, Colon (adenocarcinoma), Liver (metastasized from colon, 17 separate samples), and Liver (normal, 3 separate samples).

Probes were designed by PrimerExpress software (PE Biosystems) based on the sequence of each gene. Each gene probe was labeled using FAM (6-carboxyfluorescein), and the beta-2 microglobulin reference probe was labeled with a different fluorescent dye, VIC. The differential labeling of the target gene and internal reference gene thus enabled measurement in same well. Forward and reverse primers and the probes for both beta-2 microglobulin and target gene were added to the TaqMan™ Universal PCR Master Mix (PE Applied Biosystems). Although the final concentration of primer and probe could vary, each was internally consistent within a given experiment. A typical experiment contained 200 nanomolar of forward and reverse primers plus 100 nanomolar probe for beta-2 microglobulin and 600 nanomolar forward and reverse primers plus 200 nanomolar probe for the target gene. TaqMan matrix experiments were carried out on an ABI PRISM 7700 Sequence Detection System (PE Applied Biosystems). The thermal cycler conditions were as follows: hold for 2 minutes at 50° C. and 10 minutes at 95° C., followed by two-step PCR for 40 cycles of 95° C. for 15 seconds followed by 60° C. for 1 minute.

The following method was used to quantitatively calculate TANGO 294 gene expression in the various tissues relative to beta-2 microglobulin expression in the same tissue. The threshold cycle (Ct) value is defined as the cycle at which a statistically significant increase in fluorescence is detected. A lower Ct value is indicative of a higher mRNA concentration. The Ct value of the TANGO 294 gene is normalized by subtracting the Ct value of the beta-2 microglobulin gene to obtain a _{Δ}Ct value using the following formula: _{Δ}Ct=Ct_{TANGO 294}-Ct_{beta-2 microglobulin}. Expression is then calibrated against a cDNA sample showing a comparatively low level of expression of the TANGO 294 gene. The _{Δ}Ct value for the calibrator sample is then subtracted from _{Δ}Ct for each tissue sample according to the following formula: _{ΔΔ}Ct=_{Δ}Ct-ₛₐₘₚₗₑ-_{Δ}Ct-_{calibrator}. Relative expression is then calculated using the arithmetic formula given by 2-^{ΔΔCt}. Expression of the target TANGO 294 gene in each of the tissues tested is discussed in more detail below.

The TaqMan expression from Panel One shows restricted TANGO 294 expression in pancreas, colon tumors, and activated peripheral blood mononuclear cells, with highest expression in colon tumors.

The TaqMan expression from Panel Two shows TANGO 294 expression restricted to colon tumors and lung tumors, with a 4-50 times increase in expression in colon tumor samples over normal colon samples. For example, the colon tumor sample NDR 210 shows about 6× expression over normal colon samples, the colon tumor sample CHT 382 shows about 9× expression over normal colon samples, and the colon tumor sample CHT 528 shows about 30× expression over normal colon samples.

The TaqMan expression from Panel Three shows TANGO 294 elevated expression in adenomas and stage A, B, and C, and metastatic tumors.

The TaqMan expression from Panel Four shows TANGO 294 expression in breast, colon, and lung cell lines (NCIH322 and NHBE).

The TaqMan expression from Panel Five shows upregulated TANGO 294 expression in 80% of colon to liver metastases. Both normal colon and normal liver show no expression.

Other TANGO 294 Expression

Transcriptional profiling data shows upregulation of TANGO 294 in early and late stage colon tumors, as compared to adenomas and normal colon tissue samples.

In situ hybridization experiments confirmed expression of TANGO 294 in various tumor samples (restricted to a certain subset of tumors and metastatic lesions), as well as in pancreas and inflammatory cells (e.g., PBMCs). Expression of TANGO 294 in colon tumor samples was also confirmed by quantitative PCR amplification. Expression of TANGO 294 was detected in portions of hyperplastic colonic epithelium associated with colonic polyps.

Biological Function of TANGO 294 proteins, nucleic acids, and modulators thereof

The sequence similarity of TANGO 294 and mammalian lingual, gastric, and lysosomal acid lipase proteins indicates that TANGO 294 is involved in physiological processes identical or analogous to those involving these lipases. Lipases such as TANGO 294 catalyze formation and breakage of ester bonds between a fatty acid and a lipid moiety such as an acylglycerol, a sterol (e.g., cholesterol), and a lipoprotein. The lipases with which TANGO 294 exhibits the greatest similarity have acylglycerols and sterols among their substrates, indicating that TANGO 294 can exhibit preference for these substrates. Thus, TANGO 294 is involved in facilitating absorption and metabolism of fat. TANGO 294 can thus be used, for example, to prevent, detect, and treat disorders relating to fat absorption and metabolism, such as inadequate expression of gastric/pancreatic lipase, cystic fibrosis, exocrine pancreatic insufficiency, obesity, medical treatments which alter fat absorption, and the like.

Lipases such as TANGO 294 are also involved in regulating transfer of fatty acid substrates such as linoleic acid and arachidonic acid among dietary lipids, intracellular lipid stores, and enzymes involved in biosynthesis of eicosanoids such as prostaglandins, thromboxanes, and leukotrienes. Eicosanoids are known to exert a variety of biological effects on cells, including modulating immune responses, growth of cells, and movement of cells. Prostaglandins produced by oxidation of arachidonic acid by cyclooxygenase-2 (Cox-2) enzymes have a role in formation, growth, and spread of colon tumors (Majerus, 1998, Curr. Biol. 8(3):R87-R89). It has been hypothesized that at least part of the preventive effect of aspirin and other non-steroidal anti-inflammatory drugs on colon tumor formation is attributable to the inhibitory action of these agents on Cox-2 enzymes (Gustafson-Svard, 1997, Ann. Med. 29(3):247-252). Furthermore, abnormal expression of hormone-sensitive lipases has been observed in certain colon cancer cell lines (Remaury et al., 1995, Biochem. Biophys. Res. Commun. 208(1):456), suggesting that these enzymes can have a role in colon cancer development. Taken together, these observations and the data disclosed herein indicate that TANGO 294 can modulate formation, growth, and metastasis of colon tumors.

While not being bound by any particular theory of operation, it is believed that TANGO 294 is able to modulate the supply of eicosanoid precursor fatty acids such as linoleic acid, gamma-linoleic acid, gamma-dihomolinoleic acid, and arachidonic acid by modulating the amounts of these acids available for eicosanoid synthesis in colon tissue. These acids can be obtained by action of TANGO 294 on dietary lipids (including lipids within the colon or serum) or by esterification or de-esterification of fatty acids and lipid stores within cells. By way of example, TANGO 294 can catalyze formation of triacylglycerols containing one or more of these fatty acids, whereupon the triacylglycerol is stored in a cellular membrane or within a lysosome. By modulating the cellular supply of these fatty acids, eicosanoid synthesis can be modulated, and cellular processes (e.g., cellular growth or proliferation or movement of cells) that are affected by the presence or absence of eicosanoids can be modulated. Thus, restriction of the availability of eicosanoid fatty acid precursors can inhibit eicosanoid synthesis and exert a colon cancer-preventive effect analogous to that observed by administration of a Cox-2 inhibitor such as aspirin or sulindac.

TANGO 294 protein is known to be expressed in human pulmonary artery smooth muscle tissue. This indicates that TANGO 294 protein is involved in transportation and metabolism of fats and lipids in the human vascular and cardiovascular systems. Thus, TANGO 294 proteins of the invention can be used to prevent, detect, and treat disorders involving these body systems. Various eicosanoids, including thromboxanes and prostaglandins, are known to modulate vascular smooth muscle contraction, affecting processes such as systemic blood pressure and local blood flow in tissues. The ability of TANGO 294 to modulate eicosanoid levels permits these processes to be affected. Thus, modulating expression or activity of TANGO 294 can be used to inhibit, prevent, or counteract hypo- or hypertension in a human or to modulate blood flow through a tissue in which the expression or activity is modulated. Similarly, assessment of expression or activity of TANGO 294 in a patient, or in a tissue of a patient, can predict or diagnose a blood flow or blood pressure disorder in a patient. Examples of such disorders include arterial hypertension, renovascular hypertension, syncope, orthostatic hypotension, and shock (including anaphylactic shock).

Certain eicosanoids are known to strongly influence epithelial and endothelial cell proliferation and the tightness of adhesion between epithelial or endothelial cells. By modulating eicosanoid production in a cell (e.g., a tumor cell or a PBMC), TANGO 294 can modulate the ability of the cell to pass through an epithelial or endothelial membrane. For example, many PBMCs exhibit the ability to travel within the bloodstream to a certain body location, at which point the cells adhere to the vessel wall and pass through the endothelial membrane of the vessel in order to move to a tissue adjoining the vessel (i.e., the PBMCs exhibit the ability to extravasate). Similarly, metastatic tumor cells often exhibit an enhanced ability to move through tissue membranes and colonize body sites at which they do not normally occur (e.g., colon tumor cells can metastasize to lung tissue and develop there into a tumor mass). The ability of TANGO 294 to modulate these processes indicates that TANGO 294 molecules can be used to prognosticate, diagnose, inhibit, prevent, or even reverse the ability of cells to exhibit these characteristics. Thus, TANGO 294 molecules are useful in prognosticating, diagnosing, inhibiting, preventing, or reversing conditions such as inappropriate inflammation, tumor growth, and tumor cell metastasis.

Expression of TANGO 294 in tissues having an epithelial or endothelial component (e.g., colon and colon tumor, lung tumor, stomach, and pancreas tissues) indicates that TANGO 294 can influence the cohesiveness of an endothelial or epithelial membrane in one of these tissues or the ability of cells to pass through that membrane. These observations indicate that TANGO 294 can have a role in inflammatory disorders of these and other endothelium- or epithelium-containing tissues and that TANGO 294 can affect the likelihood that a tumor cell metastasis will enter into or take up residence within one of these tissues. Examples of inflammatory disorders in which TANGO 294 can have a role include gastritis, ulcer, various types of colitis and other less definitively characterized lower gastrointestinal disorders (e.g., irritable and inflammatory bowel syndromes), and pancreatitis.

Expression of TANGO 294 was observed, at least at low levels, in a variety of tissues having an epithelial or endothelial portion, including colon, pancreas, small intestine, stomach, spleen, tonsil, and breast tissues. In many such tissues where corresponding tumor tissue samples were available (e.g., prostate, breast and lung tumor tissues), significantly greater expression of TANGO 294 was observed in the tumor tissue than in the non-tumor tissue. This observation indicates that TANGO 294 can have a role in a variety of tumors of epithelial and endothelial origin, including those in which TANGO 294 expression occurs. The TANGO 294 molecules described herein can therefore be used to modulate (e.g., inhibit, prevent, alleviate, reverse, or cure) tumorigenesis and growth, proliferation, invasion, and metastasis of these tumors.

Expression of DNA encoding TANGO 294 is between 4 and 50 times greater in colon tumor tissue samples than in corresponding normal colon tissue samples. Expression of TANGO 294 is elevated, relative to normal colon tissue in colon adenomas and in colon tumors at stages A, B, and C. Elevated TANGO 294 expression is also observed in metastases of apparent colon origin, including those which are found in liver tissue. In situ hybridization experiments confirmed expression of TANGO 294 in various colon tumor samples, as well as in pancreas and inflammatory cells (e.g., PBMCs). Expression of TANGO 294 in colon tumor samples and colon metastases was also confirmed by quantitative PCR amplification. Expression of TANGO 294 was detected in portions of hyperplastic colonic epithelium associated with colonic polyps.

The strong correlation described herein between expression of TANGO 294 and occurrence of colon tumor cells in a sample indicates that TANGO 294 is useful as a marker for detecting occurrence of colon cancer in an individual. Expression of TANGO 294 in a sample (e.g., a stool sample, a blood sample, or a polyp or other colon biopsy) obtained from a patient can indicate a predisposition for the patient to develop a colon cancer, that a growth observed in the patient's colon is a tumor, that a colon tumor in the patient is metastasizing or has significant metastatic potential, or that the patient has otherwise non-symptomatic colon cancer.

The strong correlation between TANGO 294 expression and colon tumor occurrence also indicates that expression of TANGO 294 can have a role in (e.g., can be used to inhibit, prevent, alleviate, reverse, or cure) one or more of tumorigenesis, colon tumor growth, or colon tumor metastasis. Compounds which modulate the activity or expression of TANGO 294 (e.g., antisense oligonucleotides, antibodies which specifically bind with TANGO 294, or relatively small molecules identified by screening) can affect the rate at which or the degree to which these processes occur. Because TANGO 294 expression appears to be up-regulated in colon tumor cells, inhibiting activity, expression, or both, of TANGO 294 can inhibit or stop colon cell tumorigenesis, colon tumor growth, or colon tumor metastasis. Expression or activity of TANGO 294 can therefore be used in a screening assay to identify compounds which can have one of these effects in colon cancer.

TANGO 294 is expressed in activated PBMCs. This expression is consistent with a role for TANGO 294 in eicosanoid (e.g., leukotriene) biosynthesis. Leukotrienes are known to strongly modulate inflammation and other immune processes mediated by PBMCs. Aberrant expression of TANGO 294 can induce PBMCs to proliferate or activate in an inappropriate manner, resulting in any of a variety of disorders. Inappropriate proliferation of PBMCs can lead to cancers such as leukemias, lymphomas, and plasma cell dyscrasias, and these cancers can be inhibited, prevented, alleviated, reversed, or cured using modulators of TANGO 294 expression or activity. Inappropriate activation of PBMCs can result in a variety of inflammatory and immune disorders. By way of example, many autoimmune disorders involve activation of PBMCs in the absence of a pathogen, and others involve activation of PBMCs in the presence of a pathogen, but to an extent that immune-mediated cytotoxic activities seriously damage one or more tissues of the patient.

Pancreatic cancer cell proliferation and survival requires a sufficient level of eicosanoid synthesis (see, e.g., Ding et al., 2000, Anticancer Res. 20(4):2625-2631). Involvement of TANGO 294 in eicosanoid synthesis and metabolism indicates that TANGO 294 can contribute to proliferation and survival of pancreatic cancer cells. TANGO 294 molecules described herein can be used to inhibit, prevent, alleviate, reverse, or cure one or more of tumorigenesis, tumor cell growth, tumor cell proliferation, tumor cell invasion, and metastasis in pancreatic tissue.

It has been observed that circulating fatty acids can affect the inflammatory activity of leukocytes and ameliorate inflammatory disorders such as rheumatoid arthritis (Crocker et al., 2001, Q.J.M. 94(9):475-484). Synthesis and release of eicosanoids by leukocytes can affect the reactivity of those leukocytes and can also affect the properties of other leukocytes and eicosanoid-sensitive tissues in the physiological neighborhood of those leukocytes. The ability of TANGO 294 to modulate synthesis and release of fatty acids in PBMCs which express it indicates that modulation of TANGO 294 expression, activity, or both, can predict, diagnose, inhibit, or prevent inappropriate immune and inflammatory responses in patients. Thus, modulation of TANGO 294 expression or activity can be used to treat patients afflicted with disorders such as arthritis (e.g., rheumatoid arthritis), psoriasis, myasthenia gravis, dermatitis (e.g., contact dermatitis), allergies, insulin resistance, systemic lupus erythematosus, scleroderma, and autoimmune diabetes mellitus.

Involvement of TANGO 294 in activation of PBMCs indicates that inappropriately low activation of PBMCs can be predicted, diagnosed, inhibited, prevented, or reversed by increasing expression or activity of TANGO 294 in PBMCs. Certain infectious agents (e.g., the human immunodeficiency virus) can deplete certain types of cells within the immune system. Other infectious agents provoke an immune response, but the provoked response is not sufficient to remove the infectious agent from the patient's system. Enhancing expression, activation, or both of TANGO 294 in PBMCs in a patient can enhance the ability of the patient's immune system to react to the presence of an immunogen, thereby assisting the patient in clearing the pathogen from the patient or minimizing the pathogen's impact on the patient's health.

Various eicosanoid compounds, including several thromboxanes, influence the ability of blood platelets to aggregate with one another or to adhere to cells of another type. The ability of TANGO 294 to modulate production of eicosanoids, including thromboxanes, indicates that modulation of TANGO 294 expression, activity, or both, can influence the course of thrombotic disorders and disorders involving inappropriate platelet adherence. By way of example, some thrombotic disorders (e.g., hemophilia) are characterized by insufficient thrombosis. Expression or activity of TANGO 294 can be modulated to enhance cellular production of thrombosis-enhancing eicosanoids, and assessing TANGO 294 expression or activity in a tissue can predict or diagnose whether the patient is afflicted with a disorder characterized by insufficient thrombosis. Conversely, assessment of TANGO 294 expression or activity can be used to predict or diagnose if a patient is afflicted with (or predisposed to develop) a disorder characterized by inappropriate thrombus formation (e.g., stroke, myocardial infarction, or other abnormal blood coagulation) by inappropriate adherence of platelets to other tissues (e.g., coronary artery disease or atherosclerosis). Modulating TANGO 294 expression or activity can inhibit or prevent one of these disorders, or alleviate the disorder if it is already occurring in a patient.

Other platelet associated disorders that TANGO 294 (or modulators thereof) can be used to treat, for at least the above-mentioned reasons, include, but are not limited to, thrombocytopenia due to a reduced number of megakaryocytes in the bone marrow, for example, as a result of chemotherapy; invasive disorders, such as leukemia, idiopathic or drug- or toxin-induced aplasia of the marrow, or rare hereditary amegakaryocytic thrombocytopenias; ineffective thrombopoiesis, for example, as a result of megaloblastic anemia, alcohol toxicity, vitamin B12 or folate deficiency, myelodysplastic disorders, or rare hereditary disorders (e.g., Wiskott-Aldrich syndrome and May-hegglin anomaly); a reduction in platelet distribution, for example, as a result of cirrhosis, a splenic invasive disease (e.g., Gaucher's disease), or myelofibrosis with extramedullary myeloid metaplasia; increased platelet destruction, for example, as a result of removal of IgG-coated platelets by the mononuclear phagocytic system (e.g., idiopathic thrombocytopenic purpura (ITP), secondary immune thrombocytopenia (e.g., systemic lupus erythematosus, lymphoma, or chronic lymphocytic leukemia), drug-related immune thrombocytopenias (e.g., as with quinidine, aspirin, and heparin), post-transfusion purpura, and neonatal thrombocytopenia as a result of maternal platelet autoantibodies or maternal platelet alloantibodies). Also included are thrombocytopenia secondary to intravascular clotting and thrombin induced damage to platelets as a result of, for example, obstetric complications, metastatic tumors, severe gram-negative bacteremia, thrombotic thrombocytopenic purpura, or severe illness. Also included is dilutional thrombocytopenia, for example, due to massive hemorrhage.

Platelet associated disorders also include, but are not limited to, essential thrombocytosis and thrombocytosis associated with, for example, splenectomy, acute or chronic inflammatory diseases, hemolytic anemia, carcinoma, Hodgkin's disease, lymphoproliferative disorders, and malignant lymphomas.

The disorders mentioned in this section in connection with TANGO 294 are collectively referred to as “TANGO 294-related disorders.”

INTERCEPT 296

A cDNA clone (designated jthEa030h09) encoding at least a portion of human INTERCEPT 296 protein was isolated from a human esophagus cDNA library. The human INTERCEPT 296 protein is predicted by structural analysis to be a transmembrane protein having three or more transmembrane domains. Expression of DNA encoding INTERCEPT 296 tissue has been detected by northern analysis of human lung tissue. In human lung tissue, two moieties corresponding to INTERCEPT 296 have been identified in Northern blots. It is recognized that these two moieties may represent alternatively polyadenylated INTERCEPT 296 mRNAs or alternatively spliced INTERCEPT 296 mRNAs. It has furthermore been observed that INTERCEPT 296 does not appear to be expressed in any of heart, brain, placenta, skeletal muscle, kidney, and pancreas tissues.

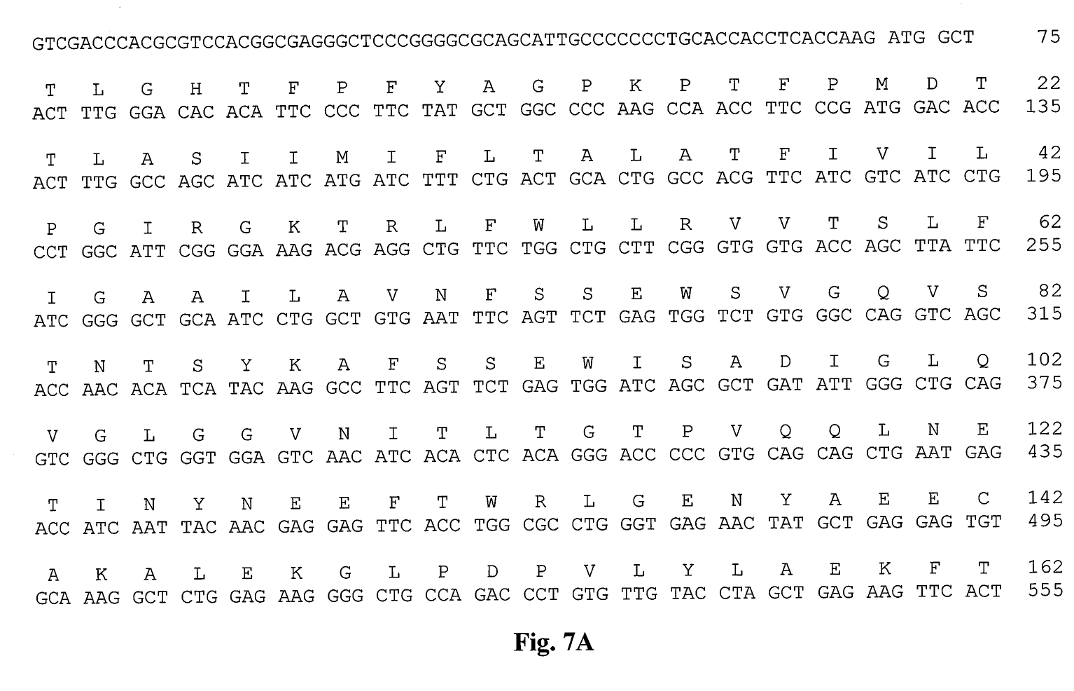

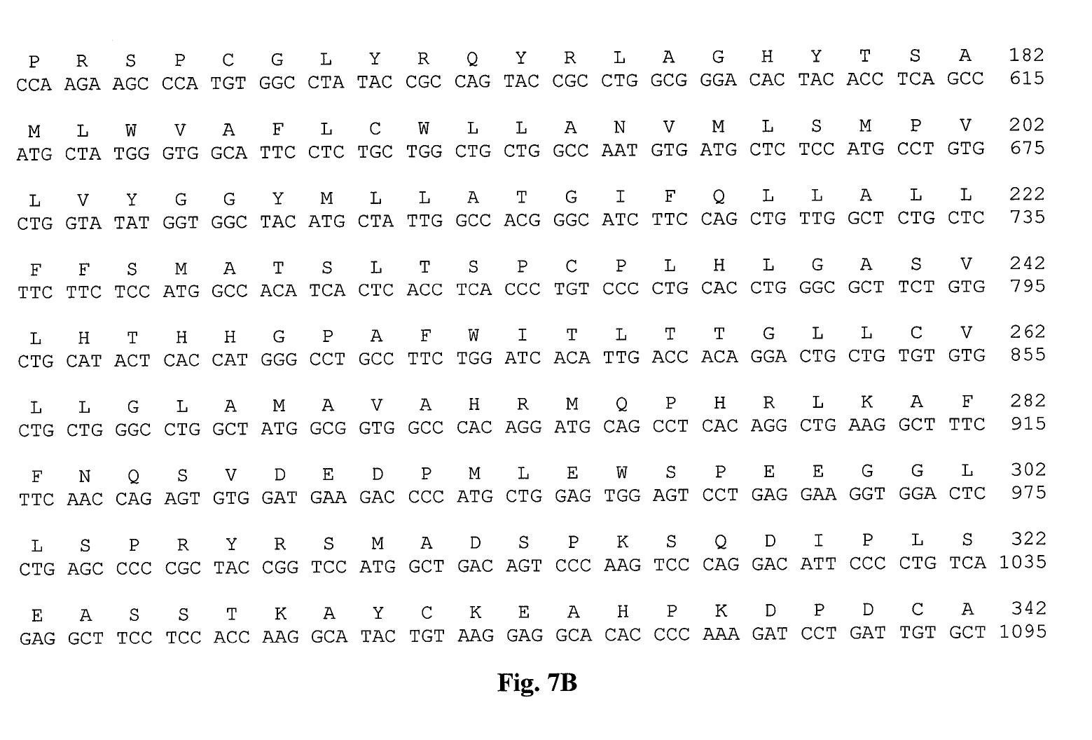

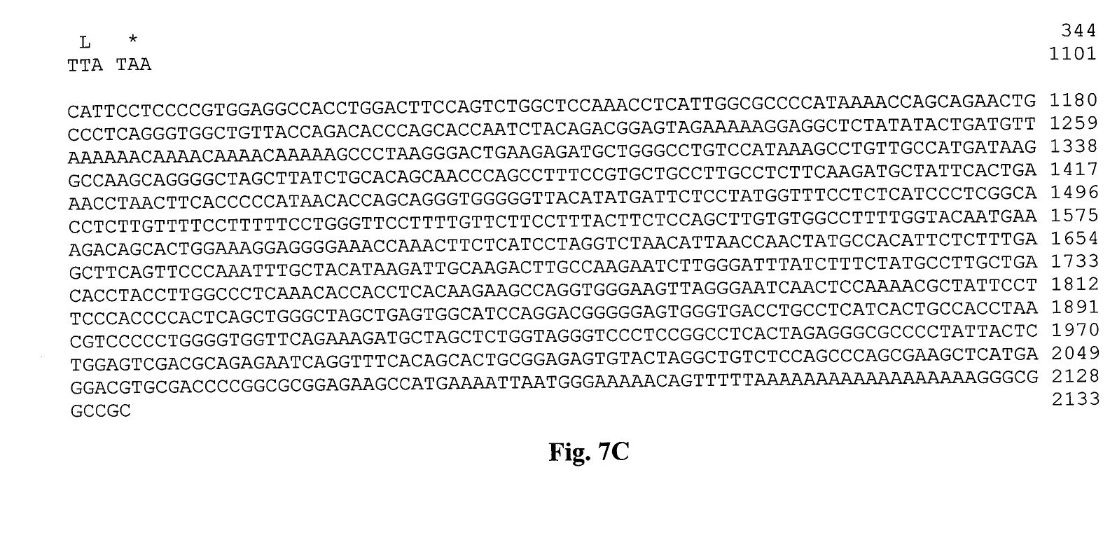

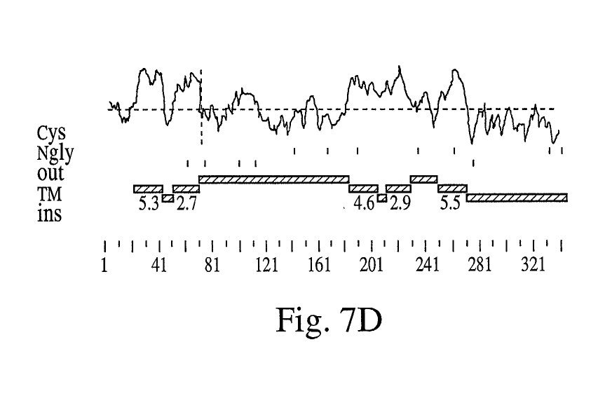

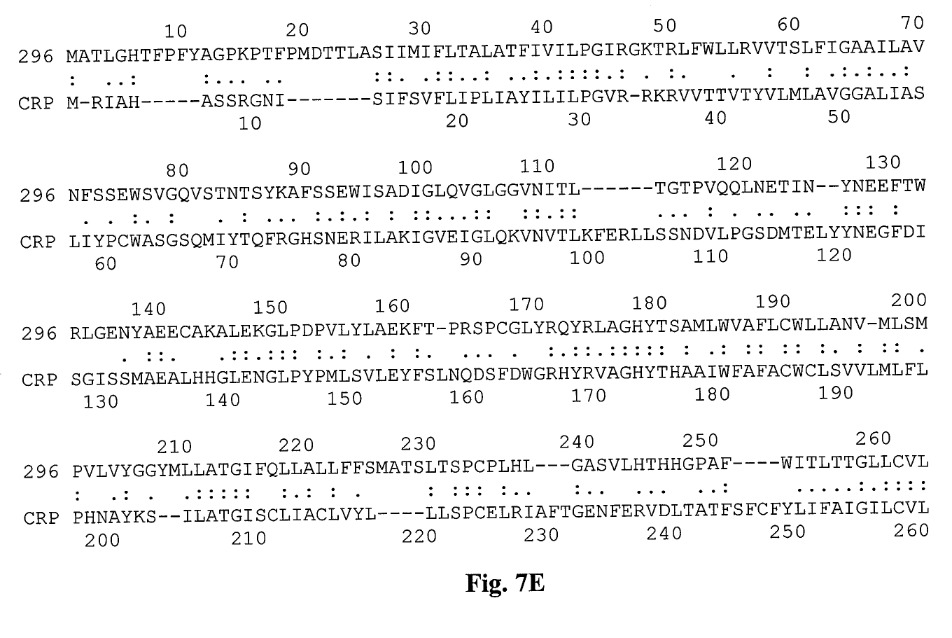

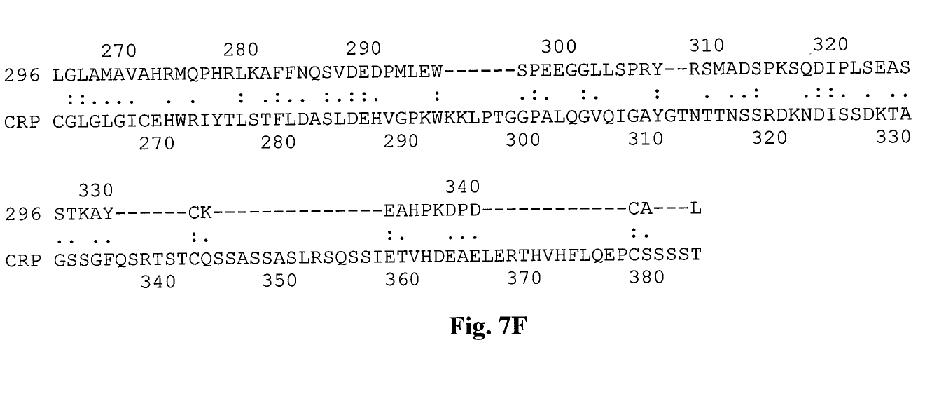

The full length of the cDNA encoding INTERCEPT 296 protein (FIG. 7; SEQ ID NO: 53) is 2133 nucleotide residues. The ORF of this cDNA, nucleotide residues 70 to 1098 of SEQ ID NO: 53 (i.e., SEQ ID NO: 54), encodes a 343-amino acid transmembrane protein (FIG. 7; SEQ ID NO: 55).

The invention includes purified INTERCEPT 296 protein, which has the amino acid sequence listed in SEQ ID NO: 55. In addition to full length INTERCEPT 296 proteins, the invention includes fragments, derivatives, and variants of these INTERCEPT 296 proteins, as described herein. These proteins, fragments, derivatives, and variants are collectively referred to herein as polypeptides of the invention or proteins of the invention.

The invention also includes nucleic acid molecules which encode a polypeptide of the invention. Such nucleic acids include, for example, a DNA molecule having the nucleotide sequence SEQ ID NO: 53 or some portion thereof, such as the portion which encodes INTERCEPT 296 protein or a domain thereof. These nucleic acids are collectively referred to as nucleic acids of the invention.

INTERCEPT 296 proteins and nucleic acid molecules encoding them comprise a family of molecules having certain conserved structural and functional features, such as the five transmembrane domains which occur in the protein.

INTERCEPT 296 comprises at least five transmembrane domains, at least three cytoplasmic domains, and at least two extracellular domains. INTERCEPT 296 does not appear to comprise a cleavable signal sequence. Amino acid residues 1 to 70 of SEQ ID NO: 55 likely directs insertion of the protein into the cytoplasmic membrane. There are at least two mechanisms by which this can occur. Sequence analysis of residues 1 to 70 of SEQ ID NO: 55 indicates that this entire region may represent a signal sequence or that residues 1 to 47 represent a signal sequence, with residues 48-70 representing a transmembrane region. Human INTERCEPT 296 protein extracellular domains are located from about amino acid residue 70 to about amino acid residue 182 (SEQ ID NO: 57) and from about amino acid residue 228 to about amino acid residue 249 (SEQ ID NO: 58) of SEQ ID NO: 55. Human INTERCEPT 296 cytoplasmic domains are located from about amino acid residue 43 to amino acid residue 50 (SEQ ID NO: 64), from about amino acid residue 205 to amino acid residue 210 (SEQ ID NO: 65), and from amino acid residue 272 to amino acid residue 343 (SEQ ID NO: 66) of SEQ ID NO: 55. The five transmembrane domains of INTERCEPT 296 are located from about amino acid residues 24 to 42 (SEQ ID NO: 59), 51 to 70 (SEQ ID NO: 60), 183 to 204 (SEQ ID NO: 61),211 to 227 (SEQ ID NO: 62), and 250 to 271 (SEQ ID NO: 63) of SEQ ID NO: 55.

INTERCEPT 296 proteins typically comprise a variety of potential post-translational modification sites (often within an extracellular domain), such as those described herein in Table XI, as predicted by computerized sequence analysis of INTERCEPT 296 proteins using amino acid sequence comparison software (comparing the amino acid sequence of INTERCEPT 296 with the information in the PROSITE database {rel. 12.2; February, 1995} and the Hidden Markov Models database {Rel. PFAM 3.3}). In certain embodiments, a protein of the invention has at least 1, 2, 4, 6, 10, 15, or 20 or more of the post-translational modification sites listed in Table XI.

TABLE XI

| | Amino Acid | |
|---|---|---|
| Type of Potential Modification Site | Residues of | Amino Acid |
| or Domain | SEQ ID NO: 55 | Sequence |
| N-glycosylation site | 71 to 74 | NFSS |
| | 84 to 87 | NTSY |
| | 109 to 112 | NITL |
| | 121 to 124 | NETI |
| | 284 to 287 | NQSV |
| Protein kinase C phosphorylation | 86 to 88 | SYK |
| site | 131 to 133 | TWR |
| | 162 to 164 | TPR |
| | 304 to 306 | SPR |
| | 313 to 315 | SPK |
| | 326 to 328 | STK |
| Casein kinase II phosphorylation | 286 to 289 | SVDE |
| site | 296 to 299 | SPEE |
| | 309 to 312 | SMAD |
| Tyrosine kinase phosphorylation | 148 to 156 | KGLPDPVLY |
| site | | |
| N-myristoylation site | 79 to 84 | GQVSTN |
| | 100 to 105 | GLQVGL |
| | 107 to 112 | GVNITL |
| | 265 to 270 | GLAMAV |

FIG. 7D depicts a hydrophilicity plot of INTERCEPT 296 protein. Relatively hydrophobic regions are above the dashed horizontal line, and relatively hydrophilic regions are below the dashed horizontal line. The hydrophobic regions which corresponds to amino acid residues 24 to 42, 51 to 70, 183 to 204, 211 to 227, and 250 to 271 of SEQ ID NO: 55 are the transmembrane domains of human INTERCEPT 296 (SEQ ID NOs: 59 through 63, respectively). As described elsewhere herein, relatively hydrophilic regions are generally located at or near the surface of a protein, and are more frequently effective immunogenic epitopes than are relatively hydrophobic regions. For example, the region of human INTERCEPT 296 protein from about amino acid residue 120 to about amino acid residue 140 appears to be located at or near the surface of the protein, while the region from about amino acid residue 95 to about amino acid residue 110 appears not to be located at or near the surface.

The predicted molecular weight of INTERCEPT 296 protein without modification and prior to cleavage of the signal sequence is about 37.8 kilodaltons. The predicted molecular weight of the mature INTERCEPT 296 protein without modification and after cleavage of the signal sequence is about 30.2 kilodaltons.

FIGS. 7E and 7F depicts an alignment of the amino acid sequences of human INTERCEPT 296 protein (SEQ ID NO: 55) and Caenorhabditis elegans C06E1.3 related protein (SEQ ID NO: 399). In this alignment (pam120.mat scoring matrix, gap penalties −12/−4), the amino acid sequences of the proteins are 26.8% identical. The C. elegans protein has five predicted transmembrane domains.

Biological Function of INTERCEPT 296 Proteins, Nucleic Acids, and Modulators Thereof

The cDNA encoding INTERCEPT 296 protein was obtained from a human esophagus cDNA library, and INTERCEPT 296 is expressed in lung tissue. The INTERCEPT 296-related proteins and nucleic acids of the invention are therefore useful for prevention, detection, and treatment of disorders of the human lung and esophagus. Such disorders include, for example, various cancers, bronchitis, cystic fibrosis, respiratory infections (e.g., influenza, bronchiolitis, pneumonia, and tuberculosis), asthma, emphysema, chronic bronchitis, bronchiectasis, pulmonary edema, pleural effusion, pulmonary embolus, adult and infant respiratory distress syndromes, heartburn, and gastric reflux esophageal disease.

Tables A and B summarize sequence data corresponding to the human proteins herein designated TANGO 202, TANGO 234, TANGO 265, TANGO 273, TANGO 286, TANGO 294, and INTERCEPT 296.

TABLE A

| Protein | SEQ ID NOs | | | Depicted in | ATCC ® |
|---|---|---|---|---|---|
| Designation | cDNA | ORF | Protein | Figure # | Accession # |
| TANGO 202 | 1 | 2 | 3 | 1 | 207219 |
| TANGO 234 | 9 | 10 | 11 | 2 | 207184 |
| TANGO 265 | 17 | 18 | 19 | 3 | 207228 |
| TANGO 273 | 25 | 26 | 27 | 4 | 207185 |
| TANGO 286 | 33 | 34 | 35 | 5 | 207220 |
| TANGO 294 | 45 | 46 | 47 | 6 | 207220 |
| INTERCEPT | 53 | 54 | 55 | 7 | 207220 |
| 296 | | | | | |

TABLE B
Amino Acid Residues

| | | | | | SEQ ID NOs | | | | | |
|---|---|---|---|---|---|---|---|---|---|---|
| | | | | | Extracellular | | Transmembrane | | Cytoplasmic | |
| Protein Desig. | Signal Sequence | | Mature Protein | | Domain(s) | | Domain(s) | | Domain(s) | |
| TANGO 202 | 1 to 19 | 4 | 20 to 475 | 5 | 20 to 392 | 6 | 393 to 415 | 7 | 416 to 475 | 8 |
| (variant) | (1 to 19) | (4) | (20 to 475) | (5) | (20 to 475) | (5) | (N/A) | | (N/A) | |
| TANGO 234 | 1 to 40 | 12 | 41 to 1453 | 13 | 41 to 1359 | 14 | 1360 to 1383 | 15 | 1384 to 1453 | 16 |
| TANGO 265 | 1 to 31 | 20 | 32 to 761 | 21 | 32 to 683 | 22 | 684 to 704 | 23 | 705 to 761 | 24 |
| TANGO 273 | 1 to 22 | 28 | 23 to 172 | 29 | 23 to 60 | 30 | 61 to 81 | 31 | 82 to 172 | 32 |
| TANGO 286 | 1 to 23 | 36 | 24 to 455 | 37 | 24 to 455 | 37 | N/A | | N/A | |
| TANGO 294 | 1 to 33 | 48 | 34 to 423 | 49 | 34 to 254 | 50 | 255 to 279 | 51 | 280 to 423 | 52 |
| (variant 1) | (15 to 33) | (40) | (34 to 423) | (49) | (34 to 254) | (50) | (255 to 279) | (51) | (280 to 423) | (52) |
| <variant 2> | <1 to 33> | <48> | <34 to 423> | <49> | <34 to 423> | <49> | <N/A> | | <N/A> | |
| {variant 3} | {15 to 33} | {40} | {34 to 423} | {49} | {34 to 423} | {49} | { N/A } | | { N/A } | |
| INTERCEPT | N/A | | 1 to 343 | 55 | 1 to 23 | 56 | 24 to 42 | 59 | 43 to 50 | 64 |
| 296 | | | | | 71 to 182 | 57 | 51 to 70 | 60 | 205 to 210 | 65 |
| | | | | | 228 to 249 | 58 | 183 to 204 | 61 | 272 to 343 | 66 |
| | | | | | | | 211 to 227 | 62 | | |
| | | | | | | | 250 to 271 | 63 | | |

Various aspects of the invention are described in further detail in the following subsections.

Isolated Nucleic Acid Molecules

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of all or a portion of any of SEQ ID NOs: 1, 2, 9, 10, 17, 18, 25, 26, 33, 34, 45, 46, 53, 54, 67, 68, 72, and 73, or a complement thereof, or which has a nucleotide sequence comprising one of these sequences, can be isolated using standard molecular biology techniques and the sequence information provided herein. Using a nucleic acid comprising at least one of the sequences of SEQ ID NOs: 1, 2, 9, 10, 17, 18, 25, 26, 33, 34, 45, 46, 53, 54, 67, 68, 72, and 73 as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of any of SEQ ID NOs: 1, 2, 9, 10, 17, 18, 25, 26, 33, 34, 45, 46, 53, 54, 67, 68, 72, and 73, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 15, preferably about 25, more preferably about 50, 75, 100, 125, 150, 175, 200, 250, 300, 350, or 400 or more consecutive nucleotides of the sense or anti-sense sequence of any of SEQ ID NOs: 1, 2, 9, 10, 17, 18, 25, 26, 33, 34, 45, 46, 53, 54, 67, 68, 72, and 73, or of a naturally occurring mutant of any of SEQ ID NOs: 1, 2, 9, 10, 17, 18, 25, 26, 33, 34, 45, 46, 53, 54, 67, 68, 72, and 73.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of any of SEQ ID NOs: 2, 10, 18, 26, 34, 46, 54, 68, and 73, expressing the encoded portion of the polypeptide protein (e.g., by recombinant expression in vitro), and assessing the activity of the encoded portion of the polypeptide.

The invention further encompasses nucleic acid molecules that differ from the nucleotide sequence of any of SEQ ID NOs: 1, 2, 9, 10, 17, 18, 25, 26, 33, 34, 45, 46, 53, 54, 67, 68, 72, and 73 due to degeneracy of the genetic code and thus encode the same protein as that encoded by the nucleotide sequence of any of SEQ ID NOs: 2, 10, 18, 26, 34, 46, 54, 68,and 73.

In addition to the nucleotide sequences of SEQ ID NOs: 2, 10, 18, 26, 34, 46, 54, 68, and 73, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus.

As used herein, the phrase “allelic variant” refers to a nucleotide sequence which occurs at a given locus or to a polypeptide encoded by the nucleotide sequence. For example, chromosomal mapping has been used to locate the gene encoding human TANGO 234 at chromosomal location h12p13 (with synteny to mo6), between chromosomal markers WI-6980 and GATA8A09.43. Thus, human TANGO 234 allelic variants can include TANGO 234 nucleotide sequence polymorphisms (e.g., nucleotide sequences that vary from SEQ ID NO: 9) that map to this chromosomal region. Similarly, chromosomal mapping has been used to locate the gene encoding human TANGO 265 protein on chromosome 1, between markers D1S305 and D1S2635. Allelic variants of TANGO 265 occur at this chromosomal location. Further by way of example, the gene encoding human TANGO 273 protein has been located by chromosomal mapping on chromosome 7, between markers D7S2467 and D7S2552. Allelic variants of TANGO 273 occur at this chromosomal location.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologs), which have a nucleotide sequence which differs from that of the specific proteins described herein are intended to be within the scope of the invention. Nucleic acid molecules corresponding to natural allelic variants and homologs of a cDNA of the invention can be isolated based on their homology with nucleic acid molecules described herein, using the specific cDNAs described herein, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 15 (25, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or 4928) nucleotides in length and hybridizes under stringent conditions to the nucleic acid molecule comprising the nucleotide sequence, preferably the coding sequence, of any of SEQ ID NOs: 1, 2, 9, 10, 17, 18, 25, 26, 33, 34, 45, 46, 53, 54, 67, 68, 72, and 73, or a complement thereof. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions with the sequence of any of SEQ ID NOs: 1, 2, 9, 10, 17, 18, 25, 26, 33, 34, 45, 46, 53, 54, 67, 68, 72, and 73, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that can exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the sequence of any of SEQ ID NOs: 3-8, 11-16, 19-24, 27-32, 35-44, 47-52, 55-66, 69, and 74, yet retain biological activity. In one embodiment, the isolated nucleic acid molecule includes a nucleotide sequence encoding a protein that includes an amino acid sequence that is at least about 40% identical, 50%, 60%, 70%, 80%, 90%, 95%, or 98% identical to the amino acid sequence of any of SEQ ID NOs: 3-8, 11-16, 19-24, 27-32, 35-44, 47-52, 55-66, 69, and 74.

An isolated nucleic acid molecule encoding a variant protein can be created by introducing one or more nucleotide substitutions, additions or deletions into the nucleotide sequence of any of SEQ ID NOs: 1, 2, 9, 10, 17, 18, 25, 26, 33, 34, 45, 46, 53, 54, 67, 68, 72, and 73, such that one or more amino acid residue substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with one or more polypeptides of the invention (e.g., in a signaling pathway); (2) the ability to bind a ligand of a polypeptide of the invention (e.g., another protein identified herein); (3) the ability to bind to an intracellular target protein of a polypeptide of the invention (e.g., a modulator or substrate of the polypeptide); or (4) the ability to modulate a physiological activity of the protein, such as one of those disclosed herein (e.g., ability to modulate cell proliferation, cell migration, chemotaxis, or cellular differentiation).

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N₆-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an alpha-anomeric nucleic acid molecule. An a-anomeric nucleic acid molecule forms specific double-stranded hybrids with complementary RNA in which, contrary to the usual beta-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach (1988) Nature 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or anti-gene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a step-wise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide can be conjugated to another molecule, e.g., a peptide, hybridization triggered cross-linking agent, transport agent, hybridization-triggered cleavage agent, etc.

Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of SEQ ID NOs: 3-8, 11-16, 19-24, 27-32, 35-44, 47-52, 55-66, 69, and 74), which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of any of SEQ ID NOs: 3-8, 11-16, 19-24, 27-32, 35-44, 47-52, 55-66, 69, and 74. Other useful proteins are substantially identical (e.g., at least about 40%, preferably 50%, 60%, 70%, 80%, 90%, 95%, or 99%) to any of SEQ ID NOs: 3-8, 11-16, 19-24, 27-32, 35-44, 47-52, 55-66, 69, and 74 and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which the polypeptide of the invention is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction can be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention (e.g., the signal sequence in one of SEQ ID NOs: 3, 4, 11, 12, 19, 20, 27, 28, 35, 36, 47, 48, 69, and 74) can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, re-naturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of any of SEQ ID NOs: 3-8, 11-16, 19-24, 27-32, 35-44, 47-52, 55-66, 69, and 74, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions. FIGS. 1L, 1M, 2J, 3U, 4I, 4J, 5E, 6F, and 7D are hydrophobicity plots of the proteins of the invention. These plots or similar analyses can be used to identify hydrophilic regions.

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptide. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a polypeptide of the invention (e.g., an epitope of a polypeptide of the invention). A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against (i.e., which bind specifically with) one or more polypeptides of the invention. Particularly preferred polyclonal antibody preparations are ones that contain only antibodies directed against one or more polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be harvested or isolated from the subject (e.g., from the blood or serum of the subject) and further purified by well-known techniques, such as protein A chromatography to obtain the IgG fraction. Alternatively, antibodies which bind specifically with a protein or polypeptide of the invention can be selected or purified (e.g., partially purified) using chromatographic methods, such as affinity chromatography. For example, a recombinantly expressed and purified (or partially purified) protein of the invention can be produced as described herein, and covalently or non-covalently coupled with a solid support such as, for example, a chromatography column. The column thus exhibits specific affinity for antibody substances which bind specifically with the protein of the invention, and these antibody substances can be purified from a sample containing antibody substances directed against a large number of different epitopes, thereby generating a substantially purified antibody substance composition, i.e., one that is substantially free of antibody substances which do not bind specifically with the protein. A substantially purified antibody composition, in this context, means an antibody sample that contains at most only 30% (by dry weight) of contaminating antibodies directed against epitopes other than those on the desired protein or polypeptide of the invention, preferably at most 20%, more preferably at most 10%, most preferably at most 5% (by dry weight of the sample is contaminating antibodies). A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SURFZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions of the antibody amino acid sequence are derived from different animal species, such as those having a variable region derived from a murine monoclonal antibody and a constant region derived from a human immunoglobulin. (See, e.g., Cabilly et al., U.S. Pat. No. 4,816,567; and Boss et al., U.S. Pat. No. 4,816,397). Humanized antibodies are antibody molecules which are obtained from non-human species, which have one or more complementarity-determining regions (CDRs) derived from the non-human species, and which have a framework region derived from a human immunoglobulin molecule. (See, e.g., Queen, U.S. Pat. No. 5,585,089). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody substance can be conjugated with a therapeutic moiety such as a cytotoxin, a therapeutic agent, or a radioactive metal ion. Cytotoxins and cytotoxic agents include any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs or homologs of these compounds. Therapeutic agents include, antimetabolites (e.g., methotrexate, 6-mercaptopurine, 6-thioguanine, cytarabine, 5-fluorouracil, and decarbazine), alkylating agents (e.g., mechlorethamine, thioepa chlorambucil, melphalan, carmustine {BSNU}, lomustine {CCNU}, cyclothosphamide, busulfan, dibromomannitol, streptozotocin, mitomycin C, and cis-dichlorodiamine platinum (II) (DDP) cisplatin), anthracyclines (e.g., daunorubicin {formerly daunomycin} and doxorubicin), antibiotics (e.g., dactinomycin {formerly actinomycin}, bleomycin, mithramycin, and anthramycin {AMC}), and anti-mitotic agents (e.g., vincristine and vinblastine).

The conjugates of the invention can be used to modify a biological response; the drug moiety is not to be construed as limited to classical chemical therapeutic agents. For example, the drug moiety can be a protein or polypeptide which exhibits a desired biological activity. Such proteins include, for example, toxins such as abrin, ricin A, pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), and other growth factors.

Techniques for conjugating a therapeutic moiety with an antibody substance are well known (see, e.g., Arnon et al., “Monoclonal Antibodies For Immunotargeting Of Drugs In Cancer Therapy”, in Monoclonal Antibodies and Cancer Therapy, Reisfeld et al., eds., pp. 243-256, Alan R. Liss, Inc., 1985; Hellstrom et al., “Antibodies For Drug Delivery”, in Controlled Drug Delivery, 2nd Ed., Robinson et al., eds., pp. 623-653, Marcel Dekker, Inc., 1987; Thorpe, “Antibody Carriers Of Cytotoxic Agents In Cancer Therapy: A Review”, in Monoclonal Antibodies '84: Biological and Clinical Applications, Pinchera et al., eds., pp. 475-506, 1985; “Analysis, Results, And Future Prospective Of The Therapeutic Use Of Radiolabeled Antibody In Cancer Therapy”, in Monoclonal Antibodies for Cancer Detection and Therapy, Baldwin et al., eds., pp. 303-316, Academic Press, 1985; and Thorpe et al., “The Preparation And Cytotoxic Properties Of Antibody-Toxin Conjugates”, Immunol. Rev. 62:119-58, 1982). Alternatively, an antibody can be conjugated with a second antibody to form an antibody heteroconjugate as described by Segal in U.S. Pat. No. 4,676,980.

Accordingly, in one aspect, the invention provides substantially purified antibodies or fragment thereof, and non-human antibodies or fragments thereof, which antibodies or fragments specifically bind with a polypeptide having an amino acid sequence which comprises a sequence selected from the group consisting of:

(i) SEQ ID NOs: 3-8, 11-16, 19-24, 27-32, 35-44, 47-52, 55-66, 69, and 74;

(ii) the amino acid sequence encoded by a cDNA of a clone deposited as one of ATCC® 207219, 207184, 207228, 207185, 207220, and 207221;

(iii) at least 15 amino acid residues of the amino acid sequence of any of SEQ ID NOs: 3-8, 11-16, 19-24, 27-32, 35-44, 47-52, 55-66, 69, and 74;

(iv) an amino acid sequence which is at least 95% identical to the amino acid sequence of any of SEQ ID NOs: 3-8, 11-16, 19-24, 27-32, 35-44, 47-52, 55-66, 69, and 74, wherein the percent identity is determined using the ALIGN program of the GCG software package with a PAM120 weight residue table, a gap length penalty of 12, and a gap penalty of 4; and

(v) an amino acid sequence which is encoded by a nucleic acid molecule which hybridizes with a nucleic acid having a sequence selected from the group consisting of SEQ ID NOs: 1, 2, 9, 10, 17, 18, 25, 26, 33, 34, 45, 46, 53, 54, 67, 68, 72, and 73 under conditions of hybridization of 6× SSC (standard saline citrate) at 45° C. and washing in 0.2× SSC, 0.1% SDS at 65° C.

In another aspect, the invention provides non-human antibodies or fragments thereof, which antibodies or fragments specifically bind with a polypeptide having an amino acid sequence which comprises a sequence selected from the group consisting of:

(i) SEQ ID NOs: 3-8, 11-16, 19-24, 27-32, 35-44, 47-52, 55-66, 69, and 74;

(ii) the amino acid sequence encoded by a cDNA of a clone deposited as one of ATCC® 207219, 207184, 207228, 207185, 207220, and 207221;

(iii) at least 15 amino acid residues of the amino acid sequence of any of SEQ ID NOs: 3-8, 11-16, 19-24, 27-32, 35-44, 47-52, 55-66, 69, and 74;

(iv) an amino acid sequence which is at least 95% identical to the amino acid sequence of any of SEQ ID NOs: 3-8, 11-16, 19-24, 27-32, 35-44, 47-52, 55-66, 69, and 74, wherein the percent identity is determined using the ALIGN program of the GCG software package with a PAM120 weight residue table, a gap length penalty of 12, and a gap penalty of 4; and

(v) an amino acid sequence which is encoded by a nucleic acid molecule which hybridizes with a nucleic acid having a sequence selected from the group consisting of SEQ ID NOs: 1, 2, 9, 10, 17, 18, 25, 26, 33, 34, 45, 46, 53, 54, 67, 68, 72, and 73 under conditions of hybridization of 6× SSC (standard saline citrate) at 45° C. and washing in 0.2× SSC, 0.1% SDS at 65° C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind with a polypeptide having an amino acid sequence which comprises a sequence selected from the group consisting of:

(i) SEQ ID NOs: 3-8, 11-16, 19-24, 27-32, 35-44, 47-52, 55-66, 69, and 74;

(ii) the amino acid sequence encoded by a cDNA of a clone deposited as one of ATCC® 207219, 207184, 207228, 207185, 207220, and 207221;

(iii) at least 15 amino acid residues of the amino acid sequence of any of SEQ ID NOs: 3-8, 11-16, 19-24, 27-32, 35-44, 47-52, 55-66, 69, and 74;

(iv) an amino acid sequence which is at least 95% identical to the amino acid sequence of any of SEQ ID NOs: 3-8, 11-16, 19-24, 27-32, 35-44, 47-52, 55-66, 69, and 74, wherein the percent identity is determined using the ALIGN program of the GCG software package with a PAM120 weight residue table, a gap length penalty of 12, and a gap penalty of 4; and

(v) an amino acid sequence which is encoded by a nucleic acid molecule which hybridizes with a nucleic acid having a sequence selected from the group consisting of SEQ ID NOs: 1, 2, 9, 10, 17, 18, 25, 26, 33, 34, 45, 46, 53, 54, 67, 68, 72, and 73 under conditions of hybridization of 6× SSC (standard saline citrate) at 45° C. and washing in 0.2× SSC, 0.1% SDS at 65° C. The monoclonal antibodies can be human, humanized, chimeric and/or non-human antibodies.

The substantially purified antibodies or fragments thereof can specifically bind with a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the non-human antibodies or fragments thereof and/or the monoclonal antibodies or fragments thereof, of the invention specifically bind with a secreted sequence or with an extracellular domain of one of TANGO 202, TANGO 234, TANGO 265, TANGO 273, TANGO 286, TANGO 294, and INTERCEPT 296. Preferably, the extracellular domain with which the antibody substance binds has an amino acid sequence selected from the group consisting of SEQ ID NOs: 5, 6, 14, 22, 30, 37, 49, 50, and 56-58.

Any of the antibody substances of the invention can be conjugated with a therapeutic moiety or to a detectable substance. Non-limiting examples of detectable substances that can be conjugated with the antibody substances of the invention include an enzyme, a prosthetic group, a fluorescent material (i.e., a fluorophore), a luminescent material, a bioluminescent material, and a radioactive material (e.g., a radionuclide or a substituent comprising a radionuclide).

The invention also provides a kit containing an antibody substance of the invention conjugated with a detectable substance, and instructions for use. Still another aspect of the invention is a pharmaceutical composition comprising an antibody substance of the invention and a pharmaceutically acceptable carrier. In preferred embodiments, the pharmaceutical composition contains an antibody substance of the invention, a therapeutic moiety (preferably conjugated with the antibody substance), and a pharmaceutically acceptable carrier.

Still another aspect of the invention is a method of making an antibody that specifically recognizes one of TANGO 202, TANGO 234, TANGO 265, TANGO 273, TANGO 286, TANGO 294, and INTERCEPT 296. This method comprises immunizing a vertebrate (e.g., a mammal such as a rabbit, goat, or pig) with a polypeptide. The polypeptide used as an immunogen has an amino acid sequence that comprises a sequence selected from the group consisting of:

(i) SEQ ID NOs: 3-8, 11-16, 19-24, 27-32, 35-44, 47-52, 55-66, 69, and 74;

(ii) the amino acid sequence encoded by a cDNA of a clone deposited as one of ATCC® 207219, 207184, 207228, 207185, 207220, and 207221;

(iii) at least 15 amino acid residues of the amino acid sequence of any of SEQ ID NOs: 3-8, 11-16, 19-24, 27-32, 35-44, 47-52, 55-66, 69, and 74;

(iv) an amino acid sequence which is at least 95% identical to the amino acid sequence of any of SEQ ID NOs: 3-8, 11-16, 19-24, 27-32, 35-44, 47-52, 55-66, 69, and 74, wherein the percent identity is determined using the ALIGN program of the GCG software package with a PAM120 weight residue table, a gap length penalty of 12, and a gap penalty of 4; and

(v) an amino acid sequence which is encoded by a nucleic acid molecule which hybridizes with a nucleic acid having a sequence selected from the group consisting of SEQ ID NOs: 1, 2, 9, 10, 17, 18, 25, 26, 33, 34, 45, 46, 53, 54, 67, 68, 72, and 73 under conditions of hybridization of 6× SSC (standard saline citrate) at 45° C. and washing in 0.2× SSC, 0.1% SDS at 65° C.

After immunization, a sample is collected from the vertebrate that contains an antibody that specifically recognizes the polypeptide with which the vertebrate was immunized. Preferably, the polypeptide is recombinantly produced using a non-human host cell. Optionally, an antibody substance can be further purified from the sample using techniques well known to those of skill in the art. The method can further comprise making a monoclonal antibody-producing cell from a cell of the vertebrate. Optionally, antibodies can be collected from the antibody-producing cell.

Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Phannacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 1 Id (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident lambda prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In another embodiment, the expression characteristics of an endogenous nucleic acid within a cell, cell line, or microorganism (e.g., a TANGO 202, TANGO 234, TANGO 265, TANGO 273, TANGO 286, TANGO 294, or INTERCEPT 296 nucleic acid, as described herein) can be modified by inserting a heterologous DNA regulatory element (i.e., one that is heterologous with respect to the endogenous gene) into the genome of the cell, stable cell line, or cloned microorganism. The inserted regulatory element can be operatively linked with the endogenous gene (e.g., TANGO 202, TANGO 234, TANGO 265, TANGO 273, TANGO 286, TANGO 294, or INTERCEPT 296) and thereby control, modulate, or activate the endogenous gene. For example, an endogenous TANGO 202, TANGO 234, TANGO 265, TANGO 273, TANGO 286, TANGO 294, or INTERCEPT 296 gene which is normally “transcriptionally silent” (i.e., a TANGO 202, TANGO 234, TANGO 265, TANGO 273, TANGO 286, TANGO 294, or INTERCEPT 296 gene which is normally not expressed, or is normally expressed only at only a very low level) can be activated by inserting a regulatory element which is capable of promoting expression of the gene in the cell, cell line, or microorganism. Alternatively, a transcriptionally silent, endogenous TANGO 202, TANGO 234, TANGO 265, TANGO 273, TANGO 286, TANGO 294, or INTERCEPT 296 gene can be activated by inserting a promiscuous regulatory element that works across cell types.

A heterologous regulatory element can be inserted into a stable cell line or cloned microorganism such that it is operatively linked with and activates expression of an endogenous TANGO 202, TANGO 234, TANGO 265, TANGO 273, TANGO 286, TANGO 294, or INTERCEPT 296 gene, using techniques, such as targeted homologous recombination, which are well known to those of skill in the art (described e.g., in Chappel, U.S. Pat. No. 5,272,071; PCT publication No. WO 91/06667, published May 16, 1991).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce non-human transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191, in Hogan, Manipulating the Mouse Embryo, Cold Spring Harbor Laboratory Press, Cold Spring Harbor, N.Y., 1986, and in Wakayama et al., 1999, Proc. Natl. Acad. Sci. USA 96:14984-14989. Similar methods can be used to produce other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create a homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 and WO 97/07669.

Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

The agent which modulates expression or activity can, for example, be a small molecule other than a nucleic acid, polypeptide, or antibody of the invention. For example, such small molecules include peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes which can be targeted to bind with virus-infected cells using a monoclonal antibody which binds specifically with a viral antigen) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Uses and Methods of the Invention

The nucleic acid molecules, proteins, protein homologs, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used for all of the purposes identified herein in portions of the disclosure relating to individual types of protein of the invention (e.g., TANGO 202 proteins, TANGO 234 proteins, TANGO 265 proteins, TANGO 273 proteins, TANGO 286 proteins, TANGO 294 proteins, and INTERCEPT 296 proteins). Polypeptides of the invention can also be used to modulate cellular proliferation, cellular differentiation, cellular adhesion, or some combination of these. The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the and modulate activity of a protein of the invention.

This invention further pertains to novel agents identified by the above-described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radio-emission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, the assay involves assessment of an activity characteristic of the polypeptide, wherein binding of the test compound with the polypeptide or a biologically active portion thereof alters (i.e., increases or decreases) the activity of the polypeptide.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide to bind to or interact with a target molecule or to transport molecules across the cytoplasmic membrane.

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., an mRNA, intracellular Ca²⁺, diacylglycerol, IP3, and the like), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule For example, the catalytic activity, the enzymatic activity, or both, of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it can be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAP S O), or N-dodecyl-N,N-dimethyl-3-ammonio-1-propane sulfonate.

In one or more embodiments of the above assay methods of the present invention, it can be desirable to immobilize either the polypeptide of the invention or its target molecule to facilitate separation of complexed from non-complexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione SEPHAROSE™ beads (Sigma Chemical; St. Louis, Mo.) or glutathione derivatized microtiter plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtiter plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS (N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (i.e., statistically significantly greater) in the presence of the candidate compound than in its absence, the candidate compound is identified as a stimulator of the selected mRNA or protein expression. Alternatively, when expression of the selected mRNA or protein is less (i.e., statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 base pairs in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sub-localization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al. (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to non-coding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

In the instant case, the human gene for TANGO 265 is located on chromosome 1 between markers D1 S305 and D1S2635, and the human gene for TANGO 273 is located on chromosome 7 between markers D7S2467 and D7S2552.

In the instant case, the human gene for TANGO 286 exhibits significant amino acid homology with a region of the human chromosome region 22q12-13 genomic nucleotide sequence having GenBank Accession number AL021937. Alignment of a 45 kilobase nucleotide sequence encoding TANGO 286 with AL021937, however, indicated the presence in TANGO 286 of exons which differ from those disclosed in L021937 (pam120.mat scoring matrix; gap penalties −12/−4). This region of chromosome 22 comprises an immunoglobulin lambda chain C (IGLC) pseudogene, the Ret finger protein-like 3 (RFPL3) and Ret finger protein-like 3 antisense (RFPL3S) genes, a gene encoding a novel immunoglobulin lambda chain V family protein, a novel gene encoding a protein similar both to mouse RGDS protein (RALGDS, RALGEF, guanine nucleotide dissociation stimulator A) and to rabbit oncogene RSC, a novel gene encoding the human orthologue of worm F16A11.2 protein, a novel gene encoding a protein similar both to BPI and to rabbit liposaccharide-binding protein, and a 5′-portion of a novel gene. This region also comprises various ESTs, STSs, GSSs, genomic marker D22S1175, a ca repeat polymorphism and putative CpG islands.

A polypeptide and fragments and sequences thereof and antibodies which bind specifically with such polypeptides/fragments can be used to map the location of the gene encoding the polypeptide on a chromosome. This mapping can be performed by specifically detecting the presence of the polypeptide/fragments in members of a panel of somatic cell hybrids between cells obtained from a first species of animal from which the protein originates and cells obtained from a second species of animal, determining which somatic cell hybrid(s) expresses the polypeptide, and noting the chromosome(s) of the first species of animal that it contains. For examples of this technique (see Pajunen et al., 1988, Cytogenet. Cell Genet. 47:37-41 and Van Keuren et al., 1986, Hum. Genet. 74:34-40). Alternatively, the presence of the polypeptide in the somatic cell hybrids can be determined by assaying an activity or property of the polypeptide (e.g., enzymatic activity, as described in Bordelon-Riser et al., 1979, Som. Cell Genet. 5:597-613 and Owerbach et al., 1978, Proc. Natl. Acad. Sci. USA 75:5640-5644).

In the instant case, the human gene for TANGO 234 protein indicated that the gene is located at chromosomal location h12p13. Flanking chromosomal markers include WI-6980 and GATA8A09.43. Nearby human loci include IBD2 (inflammatory bowel disease 2), FPF (familial periodic fever), and HPDR2 (hypophosphatemia vitamin D resistant rickets 2). Nearby genes are KLRC (killer cell receptor cluster), DRPLA (dentatorubro-pallidoluysian atrophy), GAPD (glyceraldehyde-3-phosphate) dehydrogenase, and PXR1 peroxisome receptor 1). This region is syntenic to mouse chromosome mo6. Murine chromosomal mapping indicated that the murine orthologue is located near the scr (scruffy) locus. Nearby mouse genes include drpla (dentatorubral phillidoluysian atrophy), prp (proline rich protein), and kap (kidney androgen regulated protein).

Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the non-coding regions, fewer sequences are necessary to differentiate individuals. The non-coding sequences of any of SEQ ID NOs: 1, 9, 17, 25, 33, 45, and 53 can comfortably provide positive individual identification with a panel of perhaps 10 to 1,000 primers which each yield a non-coding amplified sequence of 100 bases. If predicted coding sequences, such as those in any of SEQ ID NOs: 2, 10, 18, 26, 34, 46, and 54 are used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to non-coding regions are particularly appropriate for this use as greater numbers of polymorphisms occur in the non-coding regions, making it easier to differentiate individuals using this technique. Examples of polynucleotide reagents include the nucleic acid sequences of the invention or portions thereof, e.g., fragments derived from non-coding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention (e.g., expression or activity of one of TANGO 202, TANGO 234, TANGO 265, TANGO 273, TANGO 286, TANGO 294, or INTERCEPT 296 genes or proteins), in the context of a biological sample (e.g., blood, serum, cells, tissue) to thereby determine whether an individual is afflicted with a disease or disorder, or is at risk of developing a disorder, associated with aberrant expression or activity of a polypeptide of the invention. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

As an alternative to making determinations based on the absolute expression level of a selected gene, determinations can be based on normalized expression levels of the gene. A gene expression level is normalized by correcting the absolute expression level of the gene (e.g., a TANGO 202, TANGO 234, TANGO 265, TANGO 273, TANGO 286, TANGO 294, or INTERCEPT 296 gene as described herein) by comparing its expression to expression of a gene for which expression is not believed to be co-regulated with the gene of interest, e.g., a housekeeping gene that is constitutively expressed. Suitable genes for normalization include housekeeping genes such as the actin gene. Such normalization allows comparison of the expression level in one sample, e.g., a patient sample, with the expression level in another sample, e.g., a sample obtained from a patient known not to be afflicted with a disease or condition, or between samples obtained from different sources.

Alternatively, the expression level can be assessed as a relative expression level. To assess a relative expression level for a gene (e.g., a TANGO 202, TANGO 234, TANGO 265, TANGO 273, TANGO 286, TANGO 294, or INTERCEPT 296 gene, as described herein), the level of expression of the gene is determined for 10 or more samples (preferably 50 or more samples) of different isolates of cells in which the gene is believed to be expressed, prior to assessing the level of expression of the gene in the sample of interest. The mean expression level of the gene detected in the large number of samples is determined, and this value is used as a baseline expression level for the gene. The expression level of the gene assessed in the test sample (i.e., its absolute level of expression) is divided by the mean expression value to yield a relative expression level. Such a method can identify tissues or individuals which are afflicted with a disorder associated with aberrant expression of a gene of the invention.

Preferably, the samples used in the baseline determination are generated either using cells obtained from a tissue or individual known to be afflicted with a disorder (e.g., a disorder associated with aberrant expression of one of the TANGO 202, TANGO 234, TANGO 265, TANGO 273, TANGO 286, TANGO 294, or INTERCEPT 296 genes) or using cells obtained from a tissue or individual known not to be afflicted with the disorder. Alternatively, levels of expression of these genes in tissues or individuals known to be or not to be afflicted with the disorder can be used to assess whether the aberrant expression of the gene is associated with the disorder (e.g., with onset of the disorder, or as a symptom of the disorder over time).

Another aspect of the invention pertains to monitoring the influence of agents (e.g., drugs or other compounds) on the expression or activity of one or more of TANGO 202, TANGO 234, TANGO 265, TANGO 273, TANGO 286, TANGO 294, and INTERCEPT 296 in clinical trials. These and other agents are described in further detail in the following sections.

Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of any of SEQ ID NOs: 1, 9, 17, 25, 33, 45, 53, 67, and 72, or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, preferably an antibody with a detectable label Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (e.g., one of the disorders described in the section of this disclosure wherein the individual polypeptide of the invention is discussed). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention (e.g., one of the disorders described in the section of this disclosure wherein the individual polypeptide of the invention is discussed). Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. PCR and/or LCR can be desirable to use as a preliminary amplification step in conjunction with any of the techniques used for detecting mutations described herein.

Alternative amplification methods include: self-sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, (optionally) amplified, digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to base pair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called DNA mismatch repair enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) can be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to re-nature. The secondary structure of single-stranded nucleic acids varies according to sequence, and the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp’ of approximately 40 base pairs of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers can be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification can be used in conjunction with the instant invention. Oligonucleotides used as primers for specific amplification can carry the mutation of interest in the center of the molecule (so that amplification depends on differential hybridization; Gibbs et al. (1989) Nucleic Acids Res. 17:2437-2448) or at the extreme 3′ end of one primer where, under appropriate conditions, mismatching can prevent or reduce polymerase extension (Prossner (1993) Tibtech 11:238). In addition, it can be desirable to introduce a novel restriction site in the region of the mutation to create cleavage-based detection (Gasparini et al. (1992) Mol. Cell Probes 6:1). Amplification can also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein can be performed, for example, using pre-packaged diagnostic kits comprising at least one probe nucleic acid or antibody reagent described herein, which can be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the polypeptide of the invention is expressed can be utilized in the prognostic assays described herein.

Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

Monitoring of Effects During Clinical Trials

Monitoring the influence of agents (e.g., drug compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels, or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state can be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent can be desirable to increase the expression or activity of the polypeptide to higher levels than detected, i.e., to increase the effectiveness of the agent. Alternatively, decreased administration of the agent can be desirable to decrease expression or activity of the polypeptide to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant expression or activity of a polypeptide of the invention and/or in which the polypeptide of the invention is involved. Disorders characterized by aberrant expression or activity of the polypeptides of the invention are described elsewhere in this disclosure.

Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrance, such that a disease or disorder is prevented or, alternatively, delayed in its progression. Depending on the type of aberrance, for example, an agonist or antagonist agent can be used for treating the subject. The appropriate agent can be determined based on screening assays described herein.

Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., up-regulates or down-regulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or down-regulated and/or in which increased activity is likely to have a beneficial effect. Conversely, inhibition of activity is desirable in situations in which activity or expression is abnormally high or up-regulated and/or in which decreased activity is likely to have a beneficial effect.

The contents of all references, patents, and published patent applications cited throughout this application are hereby incorporated by reference.

Deposit of Clones

Each of these deposits was made merely as a convenience to those of skill in the art. These deposits are not an admission that a deposit is required under 35 U.S.C. §112.

Clone EpT202, encoding human TANGO 202 was deposited with the American Type Culture Collection (ATCC®, 10801 University Boulevard, Manassas, V. 20110-2209) on Apr. 21, 1999 and was assigned Accession Number 207219. This deposit will be maintained under the terms of the Budapest Treaty on the International Recognition of the Deposit of Microorganisms for the Purposes of Patent Procedure. Clone EpTm202, encoding murine TANGO 202 was deposited with ATCC® on Apr. 21, 1999 and was assigned (composite) Accession Number 207221. This deposit will be maintained under the terms of the Budapest Treaty on the International Recognition of the Deposit of Microorganisms for the Purposes of Patent Procedure.

Clone EpT234, encoding human TANGO 234 was deposited with ATCC® on Apr. 2, 1999 and was assigned Accession Number 207184. This deposit will be maintained under the terms of the Budapest Treaty on the International Recognition of the Deposit of Microorganisms for the Purposes of Patent Procedure.

Clone EpT265, encoding human TANGO 265 was deposited with ATCC® on Apr. 28, 1999 and was assigned Accession Number 207228. This deposit will be maintained under the terms of the Budapest Treaty on the International Recognition of the Deposit of Microorganisms for the Purposes of Patent Procedure.

Clone EpT273, encoding human TANGO 273 was deposited with ATCC® on Apr. 2, 1999 and was assigned Accession Number 207185. This deposit will be maintained under the terms of the Budapest Treaty on the International Recognition of the Deposit of Microorganisms for the Purposes of Patent Procedure.

Clone EpTm273, encoding murine TANGO 273 was deposited with ATCC® on Apr. 2, 1999 and was assigned (composite) Accession Number 207221. This deposit will be maintained under the terms of the Budapest Treaty on the International Recognition of the Deposit of Microorganisms for the Purposes of Patent Procedure.

Clone EpT286, encoding human TANGO 286 was deposited with ATCC® on Apr. 20, 1999 and was assigned (composite) Accession Number 207220. This deposit will be maintained under the terms of the Budapest Treaty on the International Recognition of the Deposit of Microorganisms for the Purposes of Patent Procedure.

Clone EpT294, encoding human TANGO 294 was deposited with ATCC® on Apr. 20, 1999 and was assigned (composite) Accession Number 207220. This deposit will be maintained under the terms of the Budapest Treaty on the International Recognition of the Deposit of Microorganisms for the Purposes of Patent Procedure.

Clone EpT296, encoding human INTERCEPT 296 was deposited with ATCC® on Apr. 20, 1999 and was assigned (composite) Accession Number 207220. This deposit will be maintained under the terms of the Budapest Treaty on the International Recognition of the Deposit of Microorganisms for the Purposes of Patent Procedure.

Clones containing cDNA molecules encoding human TANGO 286, human TANGO 294, and INTERCEPT 296 were deposited with ATCC® on Apr. 21, 1999 as Accession Number 207220, as part of a composite deposit representing a mixture of five strains, each carrying one recombinant plasmid harboring a particular cDNA clone.

To distinguish the strains and isolate a strain harboring a particular cDNA clone, an aliquot of the mixture is streaked out to single colonies on nutrient medium (e.g., LB plates) supplemented with 100 mg/ml ampicillin, single colonies are grown, and then plasmid DNA is extracted using a standard mini-preparation procedure. Next, a sample of the DNA mini-preparation is digested with a combination of the restriction enzymes SalI, NotI, and DraII and the resulting products are resolved on a 0.8% agarose gel using standard DNA electrophoresis conditions. This digestion procedure liberates fragments as follows:

1. human TANGO 286 (clone EpT286): 1.85 kB and 0.1 kB (human TANGO 286 has a DraII cut site at about base pair 1856).

2. human TANGO 294 (clone EpT294): 1.4 kB and 0.6 kB (human TANGO 294 has a DraII cut site at about base pair 1447).

3. human INTERCEPT 296 (clone EpT296): 0.4 kB, 1.6 kB, and 0.1 kB (human INTERCEPT 296 has DraII cut sites at about base pair 410 and at about base pair 1933). The identity of the strains can be inferred from the fragments liberated.

Clones containing cDNA molecules encoding mouse TANGO 202 and mouse TANGO 273 were deposited with ATCC® on Apr. 21, 1999 and were assigned Accession Number 207221, as part of a composite deposit representing a mixture of five strains, each carrying one recombinant plasmid harboring a particular cDNA clone. To distinguish the strains and isolate a strain harboring a particular cDNA clone, an aliquot of the mixture is streaked out to single colonies on nutrient medium (e.g., LB plates) supplemented with 100 mg/ml ampicillin, single colonies are grown, and then plasmid DNA is extracted using a standard mini-preparation procedure. Next, a sample of the DNA mini-preparation is digested with a combination of the restriction enzymes Sal I, Not I, and Apa I, and the resultant products are resolved on a 0.8% agarose gel using standard DNA electrophoresis conditions. This digestion procedure liberates fragments as follows:

1. mouse TANGO 202 (clone EpTm202): 3.5 kB and 1.4 kB (mouse TANGO 202 has a Apa I cut site at about base pair 3519).

2. mouse TANGO 273 (clone EpTm273): 0.3 kB and 2.6 kB (mouse TANGO 273 has a Apa I cut site at about base pair 298).

The identity of the strains can be inferred from the fragments liberated.

Human TANGO 202, human TANGO 234, human TANGO 265, and human TANGO 273 were each deposited as single deposits. Their clone names, deposit dates, and accession numbers are as follows:

1. human TANGO 202: clone EpT202 was deposited with ATCC® on Apr. 21, 1999, and was assigned Accession Number 207219.

2. human TANGO 234: clone EpT234 was deposited with ATCC® on Apr. 2, 1999, and was assigned Accession Number 207184.

3. human TANGO 265: clone EpT265 was deposited with ATCC® on Apr. 28, 1999, and was assigned Accession Number 207228.

4. human TANGO 273: clone EpT273 was deposited with ATCC® on Apr. 2, 1999, and was assigned Accession Number 207185.

All publications, patents, and patent applications referenced in this specification are incorporated by reference into the specification to the same extent as if each individual publication, patent, or patent application had been specifically and individually indicated to be incorporated herein by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 90% identical to the nucleotide sequence of any of SEQ ID NOs: 45, 46, the nucleotide sequence of a cDNA of a clone deposited as ATCC® 207220, or a complement thereof;
b) a nucleic acid molecule comprising at least 100 nucleotide residues and having a nucleotide sequence identical to at least 100 consecutive nucleotide residues of any of SEQ ID NOs: 45, 46, and the nucleotide sequence of a cDNA of a clone deposited as ATCC® 207220, or a complement thereof;
c) a nucleic acid molecule which encodes a polypeptide comprising the amino acid sequence of any of SEQ ID NOs: 47-52, and the amino acid sequence encoded by a cDNA of a clone deposited as ATCC® 207220, or a complement thereof;
d) a nucleic acid molecule which encodes a fragment of a polypeptide comprising the amino acid sequence of any of SEQ ID NOs: 47-52 or the amino acid sequence encoded by a cDNA of a clone deposited as ATCC® 207220, wherein the fragment comprises at least 25 consecutive amino acid residues of any of SEQ ID NOs: 47-52 or the amino acid sequence encoded by a cDNA of a clone deposited as ATCC® 207220; and
e) a nucleic acid molecule which encodes a naturally occurring allelic variant of a polypeptide comprising the amino acid sequence of any of SEQ ID NOs: 47-52, wherein the nucleic acid molecule hybridizes with a nucleic acid molecule consisting of the nucleotide sequence of any of SEQ ID NOs: 45, 46, and the nucleotide sequence of a cDNA of a clone deposited as ATCC® 207220, or a complement thereof under stringent conditions.

2. The isolated nucleic acid molecule of claim 1, which is selected from the group consisting of:
a) a nucleic acid having the nucleotide sequence of any of SEQ ID NOs: 45, 46, and the nucleotide sequence of a cDNA of a clone deposited as ATCC® 207220, or a complement thereof; and
b) a nucleic acid molecule which encodes a polypeptide having the amino acid sequence of any of SEQ ID NOs: 47-52 or the amino acid sequence encoded by a cDNA of a clone deposited as ATCC® 207220, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NOs: 47-52 or the amino acid sequence encoded by a cDNA of a clone deposited as ATCC® 207220, wherein the fragment comprises at least 25 contiguous amino acids of any of SEQ ID NOs: 47-52 or the amino acid sequence encoded by a cDNA of a clone deposited as ATCC® 207220;
b) a naturally occurring allelic variant of a polypeptide comprising the amino acid sequence of any of SEQ ID NOs: 47-52 or the amino acid sequence encoded by a cDNA of a clone deposited as ATCC® 207220, wherein the polypeptide is encoded by a nucleic acid molecule which hybridizes to a nucleic acid molecule consisting of the nucleotide sequence of any of SEQ ID NOs: 45, 46, and the nucleotide sequence of a cDNA of a clone deposited as ATCC® 207220, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NOs: 45, 46, and the nucleotide sequence of a cDNA of a clone deposited as ATCC® 207220, or a complement thereof.

9. The isolated polypeptide of claim 8 having the amino acid sequence of any of SEQ ID NOs: 47-52 or the amino acid sequence encoded by a cDNA of a clone deposited as ATCC® 207220, or a complement thereof.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide having an amino acid sequence comprising any of SEQ ID NOs: 47-52 or the amino acid sequence encoded by a cDNA of a clone deposited as ATCC® 207220, or a complement thereof;
b) a polypeptide comprising a fragment of a protein having the amino acid sequence of any of SEQ ID NOs: 47-52 or the amino acid sequence encoded by a cDNA of a clone deposited as ATCC® 207220, or a complement thereof, wherein the fragment comprises at least 25 contiguous amino acid residues of any of SEQ ID NOs: 47-52 or the amino acid sequence encoded by a cDNA of a clone deposited as ATCC® 207220, or a complement thereof; and
c) a naturally occurring allelic variant of a polypeptide having an amino acid sequence comprising the sequence of any of SEQ ID NOs: 47-52 or the amino acid sequence encoded by a cDNA of a clone deposited as ATCC® 207220, or a complement thereof, wherein the polypeptide is encoded by a nucleic acid molecule which hybridizes with a nucleic acid molecule consisting of the nucleotide sequence of any of SEQ ID NOs: 45, 46, and the nucleotide sequence of a cDNA of a clone deposited as ATCC® 207220, or a complement thereof under stringent conditions,
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8, the method comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound with the polypeptide is detected by a method selected from the group consisting of:
a) detection of binding by direct detecting of test compound/polypeptide binding;
b) detection of binding using a competition binding assay; and
c) detection of binding using an assay for an activity characteristic of the polypeptide.

21. A method for modulating the activity of a polypeptide of claim 8 comprising contacting the polypeptide or a cell expressing the polypeptide with a compound which binds with the polypeptide in a sufficient concentration to modulate the activity of the polypeptide.

22. A method for identifying a compound which modulates the activity of a polypeptide of claim 8, comprising:
a) contacting the polypeptide with a test compound; and
b) determining the effect of the test compound on the activity of the polypeptide to thereby identify a compound which modulates the activity of the polypeptide.

23. An antibody substance which selectively binds to the polypeptide of claim 8, wherein the antibody substance is made by providing the polypeptide to an immunocompetent vertebrate and thereafter harvesting blood or serum from the vertebrate.

24. A method of assessing whether a first human patient is afflicted with an epithelial or endothelial tumor, the method comprising comparing:
a) occurrence of a nucleic acid molecule of claim 1 in a sample obtained from the first patient and
b) occurrence of the nucleic acid molecule in a control sample selected from the group consisting of
i) a control sample obtained from a tissue that is obtained from the first patient and that is known not to comprise the tumor; and
ii) a control sample obtained from a second patient who is known not to be afflicted with the tumor,
whereby a difference between the first sample and the control sample is an indication that the patient is afflicted with the tumor.

25. The method of claim 24, wherein the tumor is selected from the group consisting of a colon tumor, a prostate tumor, a lung tumor, a pancreatic tumor, and a breast tumor.

26. The method of claim 25, wherein the tumor is a colon tumor.

27. A method of assessing whether a first human patient is afflicted with an epithelial or endothelial tumor, the method comprising comparing
a) occurrence of a nucleic acid molecule of claim 1 in a sample obtained from the first patient and
b) occurrence of the nucleic acid molecule in a control sample selected from the group consisting of
i) a control sample obtained from a tissue that is obtained from the first patient and that is known to comprise the tumor; and
ii) a control sample obtained from a second patient who is known to be afflicted with the tumor,
whereby a difference between the first sample and the control sample is an indication that the patient is not afflicted with the tumor.

28. A method of screening for agents which decrease the activity of a TANGO-294-like lipase protein, the method comprising:
contacting a test compound with a TANGO 294-like lipase polypeptide encoded by an isolated nucleic acid molecule of claim 1 and
detecting binding between the test compound and the TANGO 294-like lipase polypeptide,
wherein binding between the test compound and the TANGO 294-like lipase polypeptide is an indication that the test compound is an agent which decreases the activity of the TANGO 294-like lipase protein.

29. A method of screening for agents which modulate the activity of a TANGO 294-like lipase protein, the method comprising:
contacting a test compound with a TANGO 294-like lipase polypeptide encoded by an isolated nucleic acid molecule of claim 1 and
detecting a TANGO 294-like lipase activity of the polypeptide,
wherein increased TANGO 294-like lipase activity in the presence of the test compound is an indication that the test compound is an agent useful for increasing the activity of the TANGO 294-like lipase protein, and decreased TANGO 294-like lipase activity in the presence of the test compound is an indication that the test compound is an agent useful for decreasing the activity of the TANGO 294-like lipase protein.

30. A method of screening for agents which decrease the activity of a TANGO 294-like lipase protein, the method comprising:
contacting a test compound with an isolated nucleic acid molecule of claim 1 and
detecting binding of the test compound with the isolated nucleic acid molecule,
wherein binding between the test compound and the isolated nucleic acid molecule is an indication that the test compound is a agent useful for decreasing the activity of the TANGO 294-like lipase protein.

31. A method of reducing the activity of a TANGO 294-like lipase protein of a cell, the method comprising contacting the cell with a reagent which specifically binds with an isolated nucleic acid molecule of claim 1, whereby the activity of the TANGO 294-like lipase protein is reduced.

32. A method of reducing the activity of a TANGO 294-like lipase protein of a cell, the method comprising contacting the cell with a reagent which specifically binds with an isolated polypeptide of claim 8, whereby the activity of the TANGO 294-like lipase protein is reduced.

33. A method of making a pharmaceutical composition, the method comprising identifying an agent according to the method of claim 28 and combining the agent and a pharmaceutically acceptable carrier to form the pharmaceutical composition.

34. A method of modulating the activity of a TANGO 294-like lipase protein in a TANGO 294-related disorder, the method comprising making the pharmaceutical composition according to claim 33 and administering the pharmaceutical composition to a human afflicted with the disorder.

35. The method of claim 34, wherein the disorder is selected from the group consisting of a tumor, a disorder of fat absorption, a disorder of fat metabolism, a blood flow disorder, a blood pressure disorder, an inflammatory disorder, an immune disorder, a thrombotic disorder, and a disorder involving inappropriate platelet adherence.

36. The method of claim 35, wherein the disorder is a tumor of endothelial or epithelial origin.

37. The method of claim 35, wherein the disorder is a colon tumor.

38. The method of claim 35, wherein the disorder is a pancreatic tumor.

39. The method of claim 35, wherein the disorder is selected from the group consisting of inadequate expression of gastric lipase, inadequate expression of pancreatic lipase, cystic fibrosis, exocrine pancreatic insufficiency, and obesity.

40. The method of claim 35, wherein the disorder is selected from the group consisting of arterial hypertension, renovascular hypertension, syncope, orthostatic hypotension, and shock.

41. The method of claim 35, wherein the disorder is an inflammatory disorder selected from the group consisting of gastritis, gastric ulcer, colitis, irritable bowel syndrome, inflammatory bowel syndrome, dermatitis, and pancreatitis.

42. The method of claim 35, wherein the disorder is an autoimmune disorder selected from the group consisting of rheumatoid arthritis, psoriasis, myasthenia gravis, an allergy, insulin resistance, systemic lupus erythematosus, scleroderma, and autoimmune diabetes mellitus.

43. The method of claim 35, wherein the disorder is an infection of a human by an infectious agent.

44. The method of claim 43, wherein the infectious agent is human immunodeficiency virus.

45. The method of claim 35, wherein the disorder is selected from the group consisting of hemophilia, stroke, myocardial infarction, coronary artery disease, and atherosclerosis.

46. A method of making a pharmaceutical composition, the method comprising identifying an agent according to the method of claim 29 and combining the agent and a pharmaceutically acceptable carrier to form the pharmaceutical composition.

47. A method of modulating the activity of a TANGO 294-like lipase protein in a TANGO 294-related disorder, the method comprising making the pharmaceutical composition according to claim 46 and administering the pharmaceutical composition to a human afflicted with the disorder.

48. The method of claim 47, wherein the disorder is selected from the group consisting of a tumor, a disorder of fat absorption, a disorder of fat metabolism, a blood flow disorder, a blood pressure disorder, an inflammatory disorder, an immune disorder, a thrombotic disorder, and a disorder involving inappropriate platelet adherence.

49. A method of making a pharmaceutical composition, the method comprising identifying an agent according to the method of claim 30 and combining the agent and a pharmaceutically acceptable carrier to form the pharmaceutical composition.

50. A method of modulating the activity of a TANGO 294-like lipase protein in a TANGO 294-related disorder, the method comprising making the pharmaceutical composition according to claim 49 and administering the pharmaceutical composition to a human afflicted with the disorder.

51. The method of claim 50, wherein the disorder is selected from the group consisting of a tumor, a disorder of fat absorption, a disorder of fat metabolism, a blood flow disorder, a blood pressure disorder, an inflammatory disorder, an immune disorder, a thrombotic disorder, and a disorder involving inappropriate platelet adherence.


SEQUENCE LISTING (79 sequences)

SEQ ID NO: 1 - DNA, 1656 bp, Homo sapiens
gtcgacccacgcgtccgcccacgcgtccggcccatggcgccgcccgccgcccgcctcgccctgctctccgccgcggcgctcacgctggcggcccggcccgcgcctagccccggcctcggccccggacccgagtgtttcacagccaatggtgcggattataggggaacacagaactggacagcactacaaggcgggaagccatgtctgttttggaacgagactttccagcatccatacaacactctgaaataccccaacggggaggggggcctgggtgagcacaactattgcagaaatccagatggagacgtgagcccctggtgctatgtggcagagcacgaggatggtgtctactggaagtactgtgagatacctgcttgccagatgcctggaaaccttggctgctacaaggatcatggaaacccacctcctctaactggcaccagtaaaacgtccaacaaactcaccatacaaacttgcatcagtttttgtcggagtcagaggttcaagtttgctgggatggagtcaggctatgcttgcttctgtggaaacaatcctgattactggaagtacggggaggcagccagtaccgaatgcaacagcgtctgcttcggggatcacacccaaccctgtggtggcgatggcaggatcatcctctttgatactctcgtgggcgcctgcggtgggaactactcagccatgtcttctgtggtctattcccctgacttccccgacacctatgccacggggagggtctgctactggaccatccgggttccgggggcctcccacatccacttcagcttccccctatttgacatcagggactcggcggacatggtggagcttctggatggctacacccaccgtgtcctagcccgcttccacgggaggagccgcccacctctgtccttcaacgtctctctggacttcgtcatcttgtatttcttctctgatcgcatcaatcaggcccagggatttgctgttttataccaagccgtcaaggaagaactgccacaggagaggcccgctgtcaaccagacggtggccgaggtgatcacggagcaggccaacctcagtgtcagcgctgcccggtcctccaaagtcctctatgtcatcaccaccagccccagccacccacctcagactgtcccaggtagcaattcctgggcgccacccatgggggctggaagccacagagttgaaggatggacagtctatggtctggcaactctcctcatcctcacagtcacagccattgtagcaaagatacttctgcacgtcacattcaaatcccatcgtgttcctgcttcaggggaccttagggattgtcatcaaccagggacttcgggggaaatctggagcattttttacaagccttccacttcaatttccatctttaagaagaaactcaagggtcagagtcaacaagatgaccgcaatccccttgtgagtgactaaaaaccccactgtgcctaggacttgaggtccctctttgagctcaaggctgccgtggtcaacctctcctgtggttcttctctgacagactcttccctcctctccctctgcctcggcctcttcggggaaaccctcctcctacagactaggaagaggcacctgctgccagggcaggcagagcctggattcctcctgctt

SEQ ID NO: 2 - DNA, 1425 bp, Homo sapiens
atggcgccgcccgccgcccgcctcgccctgctctccgccgcggcgctcacgctggcggcccggcccgcgcctagccccggcctcggccccggacccgagtgtttcacagccaatggtgcggattataggggaacacagaactggacagcactacaaggcgggaagccatgtctgttttggaacgagactttccagcatccatacaacactctgaaataccccaacggggaggggggcctgggtgagcacaactattgcagaaatccagatggagacgtgagcccctggtgctatgtggcagagcacgaggatggtgtctactggaagtactgtgagatacctgcttgccagatgcctggaaaccttggctgctacaaggatcatggaaacccacctcctctaactggcaccagtaaaacgtccaacaaactcaccatacaaacttgcatcagtttttgtcggagtcagaggttcaagtttgctgggatggagtcaggctatgcttgcttctgtggaaacaatcctgattactggaagtacggggaggcagccagtaccgaatgcaacagcgtctgcttcggggatcacacccaaccctgtggtggcgatggcaggatcatcctctttgatactctcgtgggcgcctgcggtgggaactactcagccatgtcttctgtggtctattcccctgacttccccgacacctatgccacggggagggtctgctactggaccatccgggttccgggggcctcccacatccacttcagcttccccctatttgacatcagggactcggcggacatggtggagcttctggatggctacacccaccgtgtcctagcccgcttccacgggaggagccgcccacctctgtccttcaacgtctctctggacttcgtcatcttgtatttcttctctgatcgcatcaatcaggcccagggatttgctgttttataccaagccgtcaaggaagaactgccacaggagaggcccgctgtcaaccagacggtggccgaggtgatcacggagcaggccaacctcagtgtcagcgctgcccggtcctccaaagtcctctatgtcatcaccaccagccccagccacccacctcagactgtcccaggtagcaattcctgggcgccacccatgggggctggaagccacagagttgaaggatggacagtctatggtctggcaactctcctcatcctcacagtcacagccattgtagcaaagatacttctgcacgtcacattcaaatcccatcgtgttcctgcttcaggggaccttagggattgtcatcaaccagggacttcgggggaaatctggagcattttttacaagccttccacttcaatttccatctttaagaagaaactcaagggtcagagtcaacaagatgaccgcaatccccttgtgagtgac

SEQ ID NO: 3 - protein, 475 aa, Homo sapiens
MAPPAARLALLSAAALTLAARPAPSPGLGPGPECFTANGADYRGTQNWTALQGGKPCLFWNETFQHPYNTLKYPNGEGGLGEHNYCRNPDGDVSPWCYVAEHEDGVYWKYCEIPACQMPGNLGCYKDHGNPPPLTGTSKTSNKLTIQTCISFCRSQRFKFAGMESGYACFCGNNPDYWKYGEAASTECNSVCFGDHTQPCGGDGRIILFDTLVGACGGNYSAMSSVVYSPDFPDTYATGRVCYWTIRVPGASHIHFSFPLFDIRDSADMVELLDGYTHRVLARFHGRSRPPLSFNVSLDFVILYFFSDRINQAQGFAVLYQAVKEELPQERPAVNQTVAEVITEQANLSVSAARSSKVLYVITTSPSHPPQTVPGSNSWAPPMGAGSHRVEGWTVYGLATLLILTVTAIVAKILLHVTFKSHRVPASGDLRDCHQPGTSGEIWSIFYKPSTSISIFKKKLKGQSQQDDRNPLVSD

SEQ ID NO: 4 - protein, 19 aa, Homo sapiens
MAPPAARLALLSAAALTLA

SEQ ID NO: 5 - protein, 456 aa, Homo sapiens
ARPAPSPGLGPGPECFTANGADYRGTQNWTALQGGKPCLFWNETFQHPYNTLKYPNGEGGLGEHNYCRNPDGDVSPWCYVAEHEDGVYWKYCEIPACQMPGNLGCYKDHGNPPPLTGTSKTSNKLTIQTCISFCRSQRFKFAGMESGYACFCGNNPDYWKYGEAASTECNSVCFGDHTQPCGGDGRIILFDTLVGACGGNYSAMSSVVYSPDFPDTYATGRVCYWTIRVPGASHIHFSFPLFDIRDSADMVELLDGYTHRVLARFHGRSRPPLSFNVSLDFVILYFFSDRINQAQGFAVLYQAVKEELPQERPAVNQTVAEVITEQANLSVSAARSSKVLYVITTSPSHPPQTVPGSNSWAPPMGAGSHRVEGWTVYGLATLLILTVTAIVAKILLHVTFKSHRVPASGDLRDCHQPGTSGEIWSIFYKPSTSISIFKKKLKGQSQQDDRNPLVSD

SEQ ID NO: 6 - protein, 373 aa, Homo sapiens
ARPAPSPGLGPGPECFTANGADYRGTQNWTALQGGKPCLFWNETFQHPYNTLKYPNGEGGLGEHNYCRNPDGDVSPWCYVAEHEDGVYWKYCEIPACQMPGNLGCYKDHGNPPPLTGTSKTSNKLTIQTCISFCRSQRFKFAGMESGYACFCGNNPDYWKYGEAASTECNSVCFGDHTQPCGGDGRIILFDTLVGACGGNYSAMSSVVYSPDFPDTYATGRVCYWTIRVPGASHIHFSFPLFDIRDSADMVELLDGYTHRVLARFHGRSRPPLSFNVSLDFVILYFFSDRINQAQGFAVLYQAVKEELPQERPAVNQTVAEVITEQANLSVSAARSSKVLYVITTSPSHPPQTVPGSNSWAPPMGAGSHRVEG

SEQ ID NO: 7 - protein, 23 aa, Homo sapiens
WTVYGLATLLILTVTAIVAKILL

SEQ ID NO: 8 - protein, 60 aa, Homo sapiens
HVTFKSHRVPASGDLRDCHQPGTSGEIWSIFYKPSTSISIFKKKLKGQSQQDDRNPLVSD

SEQ ID NO: 9 - DNA, 4628 bp, Homo sapiens
gcggccgctcgcgatctagaactagtaatgatgctgcctcaaaactcgtggcatattgattttggaagatgctgctgtcatcagaaccttttctctgctgtggtaacttgcatcctgctcctgaattcctgctttctcatcagcagttttaatggaacagatttggagttgaggctggtcaatggagacggtccctgctctgggacagtggaggtgaaattccagggacagtgggggactgtgtgtgatgatgggtggaacactactgcctcaactgtcgtgtgcaaacagcttggatgtccattttctttcgccatgtttcgttttggacaagccgtgactagacatggaaaaatttggcttgatgatgtttcctgttatggaaatgagtcagctctctgggaatgtcaacaccgggaatggggaagccataactgttatcatggagaagatgttggtgtgaactgttatggtgaagccaatctgggtttgaggctagtggatggaaacaactcctgttcagggagagtggaggtgaaattccaagaaaggtgggggactatatgtgatgatgggtggaacttgaatactgctgccgtggtgtgcaggcaactaggatgtccatcttcttttatttcttctggagttgttaatagccctgctgtattgcgccccatttggctggatgacattttatgccaggggaatgagttggcactctggaattgcagacatcgtggatggggaaatcatgactgcagtcacaatgaggatgtcacattaacttgttatgatagtagtgatcttgaactaaggcttgtaggtggaactaaccgctgtatggggagagtagagctgaaaatccaaggaaggtgggggaccgtatgccaccataagtggaacaatgctgcagctgatgtcgtatgcaagcagttgggatgtggaaccgcacttcacttcgctggcttgcctcatttgcagtcagggtctgatgttgtatggcttgatggtgtctcctgctccggtaatgaatcttttctttgggactgcagacattccggaaccgtcaattttgactgtcttcatcaaaacgatgtgtctgtgatctgctcagatggagcagatttggaactgcgactagcagatggaagtaacaattgttcagggagagtagaggtgagaattcatgaacagtggtggacaatatgtgaccagaactggaagaatgaacaagcccttgtggtttgtaagcagctaggatgtccgttcagcgtctttggcagtcgtcgtgctaaacctagtaatgaagctagagacatttggataaacagcatatcttgcactgggaatgagtcagctctctgggactgcacatatgatggaaaagcaaagcgaacatgcttccgaagatcagatgctggagtaatttgttctgataaggcagatctggacctaaggcttgtcggggctcatagcccctgttatgggagattggaggtgaaataccaaggagagtgggggactgtgtgtcatgacagatggagcacaaggaatgcagctgttgtgtgtaaacaattgggatgtggaaagcctatgcatgtgtttggtatgacctattttaaagaagcatcaggacctatttggctggatgacgtttcttgcattggaaatgagtcaaatatctgggactgtgaacacagtggatggggaaagcataattgtgtacacagagaggatgtgattgtaacctgctcaggtgatgcaacatggggcctgaggctggtgggcggcagcaaccgctgctcgggaagactggaggtgtactttcaaggacggtggggcacagtgtgtgatgacggctggaacagtaaagctgcagctgtggtgtgtagccagctggactgcccatcttctatcattggcatgggtctgggaaacgcttctacaggatatggaaaaatttggctcgatgatgtttcctgtgatggagatgagtcagatctctggtcatgcaggaacagtgggtggggaaataatgactgcagtcacagtgaagatgttggagtgatctgttctgatgcatcggatatggagctgaggcttgtgggtggaagcagcaggtgtgctggaaaagttgaggtgaatgtccagggtgccgtgggaattctgtgtgctaatggctggggaatgaacattgctgaagttgtttgcaggcaacttgaatgtgggtctgcaatcagggtctccagagagcctcatttcacagaaagaacattacacatcttaatgtcgaattctggctgcactggaggggaagcctctctctgggattgtatacgatgggagtggaaacagactgcgtgtcatttaaatatggaagcaagtttgatctgctcagcccacaggcagcccaggctggttggagctgatatgccctgctctggacgtgttgaagtgaaacatgcagacacatggcgctctgtctgtgattctgatttctctcttcatgctgccaatgtgctgtgcagagaattaaattgtggagatgccatatctctttctgtgggagatcactttggaaaagggaatggtctaacttgggccgaaaagttccagtgtgaagggagtgaaactcaccttgcattatgccccattgttcaacatccggaagacacttgtatccacagcagagaagttggagttgtctgttcccgatatacagatgtccgacttgtgaatggcaaatcccagtgtgacgggcaagtggagatcaacgtgcttggacactggggctcactgtgtgacacccactgggacccagaagatgcccgtgttctatgcagacagctcagctgtgggactgctctctcaaccacaggaggaaaatatattggagaaagaagtgttcgtgtgtggggacacaggtttcattgcttagggaatgagtcacttctggataactgtcaaatgacagttcttggagcacctccctgtatccatggaaatactgtctctgtgatctgcacaggaagcctgacccagccactgtttccatgcctcgcaaatgtatctgacccatatttgtctgcagttccagagggcagtgctttgatctgcttagaggacaaacggctccgcctagtggatggggacagccgctgtgccgggagagtagagatctatcacgacggcttctggggcaccatctgtgatgacggctgggacctgagcgatgcccacgtggtgtgtcaaaagctgggctgtggagtggccttcaatgccacggtctctgctcactttggggaggggtcagggcccatctggctggatgacctgaactgcacaggaacggagtcccacttgtggcagtgcccttcccgcggctgggggcagcacgactgcaggcacaaggaggacgcaggggtcatctgctcagaattcacagccttgaggctctacagtgaaactgaaacagagagctgtgctgggagattggaagtcttctataacgggacctggggcagcgtcggcaggaggaacatcaccacagccatagcaggcattgtgtgcaggcagctgggctgtggggagaatggagttgtcagcctcgcccctttatctaagacaggctctggtttcatgtgggtggatgacattcagtgtcctaaaacgcatatctccatatggcagtgcctgtctgccccatgggagcgaagaatctccagcccagcagaagagacctggatcacatgtgaagatagaataagagtgcgtggaggagacaccgagtgctctgggagagtggagatctggcacgcaggctcctggggcacagtgtgtgatgactcctgggacctggccgaggcggaagtggtgtgtcagcagctgggctgtggctctgctctggctgccctgagggacgcttcgtttggccagggaactggaaccatctggttggatgacatgcggtgcaaaggaaatgagtcatttctatgggactgtcacgccaaaccctggggacagagtgactgtggacacaaggaagatgctggcgtgaggtgctctggacagtcgctgaaatcactgaatgcctcctcaggtcatttagcacttattttatccagtatctttgggctccttctcctggttctgtttattctatttctcacgtggtgccgagttcagaaacaaaaacatctgcccctcagagtttcaaccagaaggaggggttctctcgaggagaatttattccatgagatggagacctgcctcaagagagaggacccacatgggacaagaacctcagatgacacccccaaccatggttgtgaagatgctagcgacacatcgctgttgggagttcttcctgcctctgaagccacaaaatgactttagacttccagggctcaccagatcaacctctaaatatctttgaaggagacaacaacttttaaatgaataaagaggaagtcaagttgccctatggaaaacttgtccaaataacatttcttgaacaataggagaacagctaaattgataaagactggtgataataaaaattgaattatgtatatcactgttaaaaaaaaaaaaaaaaaaaaaaaaaaaaaacggacgcgtgggtcgac

SEQ ID NO: 10 - DNA, 4359 bp, Homo sapiens
atgatgctgcctcaaaactcgtggcatattgattttggaagatgctgctgtcatcagaaccttttctctgctgtggtaacttgcatcctgctcctgaattcctgctttctcatcagcagttttaatggaacagatttggagttgaggctggtcaatggagacggtccctgctctgggacagtggaggtgaaattccagggacagtgggggactgtgtgtgatgatgggtggaacactactgcctcaactgtcgtgtgcaaacagcttggatgtccattttctttcgccatgtttcgttttggacaagccgtgactagacatggaaaaatttggcttgatgatgtttcctgttatggaaatgagtcagctctctgggaatgtcaacaccgggaatggggaagccataactgttatcatggagaagatgttggtgtgaactgttatggtgaagccaatctgggtttgaggctagtggatggaaacaactcctgttcagggagagtggaggtgaaattccaagaaaggtgggggactatatgtgatgatgggtggaacttgaatactgctgccgtggtgtgcaggcaactaggatgtccatcttcttttatttcttctggagttgttaatagccctgctgtattgcgccccatttggctggatgacattttatgccaggggaatgagttggcactctggaattgcagacatcgtggatggggaaatcatgactgcagtcacaatgaggatgtcacattaacttgttatgatagtagtgatcttgaactaaggcttgtaggtggaactaaccgctgtatggggagagtagagctgaaaatccaaggaaggtgggggaccgtatgccaccataagtggaacaatgctgcagctgatgtcgtatgcaagcagttgggatgtggaaccgcacttcacttcgctggcttgcctcatttgcagtcagggtctgatgttgtatggcttgatggtgtctcctgctccggtaatgaatcttttctttgggactgcagacattccggaaccgtcaattttgactgtcttcatcaaaacgatgtgtctgtgatctgctcagatggagcagatttggaactgcgactagcagatggaagtaacaattgttcagggagagtagaggtgagaattcatgaacagtggtggacaatatgtgaccagaactggaagaatgaacaagcccttgtggtttgtaagcagctaggatgtccgttcagcgtctttggcagtcgtcgtgctaaacctagtaatgaagctagagacatttggataaacagcatatcttgcactgggaatgagtcagctctctgggactgcacatatgatggaaaagcaaagcgaacatgcttccgaagatcagatgctggagtaatttgttctgataaggcagatctggacctaaggcttgtcggggctcatagcccctgttatgggagattggaggtgaaataccaaggagagtgggggactgtgtgtcatgacagatggagcacaaggaatgcagctgttgtgtgtaaacaattgggatgtggaaagcctatgcatgtgtttggtatgacctattttaaagaagcatcaggacctatttggctggatgacgtttcttgcattggaaatgagtcaaatatctgggactgtgaacacagtggatggggaaagcataattgtgtacacagagaggatgtgattgtaacctgctcaggtgatgcaacatggggcctgaggctggtgggcggcagcaaccgctgctcgggaagactggaggtgtactttcaaggacggtggggcacagtgtgtgatgacggctggaacagtaaagctgcagctgtggtgtgtagccagctggactgcccatcttctatcattggcatgggtctgggaaacgcttctacaggatatggaaaaatttggctcgatgatgtttcctgtgatggagatgagtcagatctctggtcatgcaggaacagtgggtggggaaataatgactgcagtcacagtgaagatgttggagtgatctgttctgatgcatcggatatggagctgaggcttgtgggtggaagcagcaggtgtgctggaaaagttgaggtgaatgtccagggtgccgtgggaattctgtgtgctaatggctggggaatgaacattgctgaagttgtttgcaggcaacttgaatgtgggtctgcaatcagggtctccagagagcctcatttcacagaaagaacattacacatcttaatgtcgaattctggctgcactggaggggaagcctctctctgggattgtatacgatgggagtggaaacagactgcgtgtcatttaaatatggaagcaagtttgatctgctcagcccacaggcagcccaggctggttggagctgatatgccctgctctggacgtgttgaagtgaaacatgcagacacatggcgctctgtctgtgattctgatttctctcttcatgctgccaatgtgctgtgcagagaattaaattgtggagatgccatatctctttctgtgggagatcactttggaaaagggaatggtctaacttgggccgaaaagttccagtgtgaagggagtgaaactcaccttgcattatgccccattgttcaacatccggaagacacttgtatccacagcagagaagttggagttgtctgttcccgatatacagatgtccgacttgtgaatggcaaatcccagtgtgacgggcaagtggagatcaacgtgcttggacactggggctcactgtgtgacacccactgggacccagaagatgcccgtgttctatgcagacagctcagctgtgggactgctctctcaaccacaggaggaaaatatattggagaaagaagtgttcgtgtgtggggacacaggtttcattgcttagggaatgagtcacttctggataactgtcaaatgacagttcttggagcacctccctgtatccatggaaatactgtctctgtgatctgcacaggaagcctgacccagccactgtttccatgcctcgcaaatgtatctgacccatatttgtctgcagttccagagggcagtgctttgatctgcttagaggacaaacggctccgcctagtggatggggacagccgctgtgccgggagagtagagatctatcacgacggcttctggggcaccatctgtgatgacggctgggacctgagcgatgcccacgtggtgtgtcaaaagctgggctgtggagtggccttcaatgccacggtctctgctcactttggggaggggtcagggcccatctggctggatgacctgaactgcacaggaacggagtcccacttgtggcagtgcccttcccgcggctgggggcagcacgactgcaggcacaaggaggacgcaggggtcatctgctcagaattcacagccttgaggctctacagtgaaactgaaacagagagctgtgctgggagattggaagtcttctataacgggacctggggcagcgtcggcaggaggaacatcaccacagccatagcaggcattgtgtgcaggcagctgggctgtggggagaatggagttgtcagcctcgcccctttatctaagacaggctctggtttcatgtgggtggatgacattcagtgtcctaaaacgcatatctccatatggcagtgcctgtctgccccatgggagcgaagaatctccagcccagcagaagagacctggatcacatgtgaagatagaataagagtgcgtggaggagacaccgagtgctctgggagagtggagatctggcacgcaggctcctggggcacagtgtgtgatgactcctgggacctggccgaggcggaagtggtgtgtcagcagctgggctgtggctctgctctggctgccctgagggacgcttcgtttggccagggaactggaaccatctggttggatgacatgcggtgcaaaggaaatgagtcatttctatgggactgtcacgccaaaccctggggacagagtgactgtggacacaaggaagatgctggcgtgaggtgctctggacagtcgctgaaatcactgaatgcctcctcaggtcatttagcacttattttatccagtatctttgggctccttctcctggttctgtttattctatttctcacgtggtgccgagttcagaaacaaaaacatctgcccctcagagtttcaaccagaaggaggggttctctcgaggagaatttattccatgagatggagacctgcctcaagagagaggacccacatgggacaagaacctcagatgacacccccaaccatggttgtgaagatgctagcgacacatcgctgttgggagttcttcctgcctctgaagccacaaaa

SEQ ID NO: 11 - protein, 1453 aa, Homo sapiens
MMLPQNSWHIDFGRCCCHQNLFSAVVTCILLLNSCFLISSFNGTDLELRLVNGDGPCSGTVEVKFQGQWGTVCDDGWNTTASTVVCKQLGCPFSFAMFRFGQAVTRHGKIWLDDVSCYGNESALWECQHREWGSHNCYHGEDVGVNCYGEANLGLRLVDGNNSCSGRVEVKFQERWGTICDDGWNLNTAAVVCRQLGCPSSFISSGVVNSPAVLRPIWLDDILCQGNELALWNCRHRGWGNHDCSHNEDVTLTCYDSSDLELRLVGGTNRCMGRVELKIQGRWGTVCHHKWNNAAADVVCKQLGCGTALHFAGLPHLQSGSDVVWLDGVSCSGNESFLWDCRHSGTVNFDCLHQNDVSVICSDGADLELRLADGSNNCSGRVEVRIHEQWWTICDQNWKNEQALVVCKQLGCPFSVFGSRRAKPSNEARDIWINSISCTGNESALWDCTYDGKAKRTCFRRSDAGVICSDKADLDLRLVGAHSPCYGRLEVKYQGEWGTVCHDRWSTRNAAVVCKQLGCGKPMHVFGMTYFKEASGPIWLDDVSCIGNESNIWDCEHSGWGKHNCVHREDVIVTCSGDATWGLRLVGGSNRCSGRLEVYFQGRWGTVCDDGWNSKAAAVVCSQLDCPSSIIGMGLGNASTGYGKIWLDDVSCDGDESDLWSCRNSGWGNNDCSHSEDVGVICSDASDMELRLVGGSSRCAGKVEVNVQGAVGILCANGWGMNIAEVVCRQLECGSAIRVSREPHFTERTLHILMSNSGCTGGEASLWDCIRWEWKQTACHLNMEASLICSAHRQPRLVGADMPCSGRVEVKHADTWRSVCDSDFSLHAANVLCRELNCGDAISLSVGDHFGKGNGLTWAEKFQCEGSETHLALCPIVQHPEDTCIHSREVGVVCSRYTDVRLVNGKSQCDGQVEINVLGHWGSLCDTHWDPEDARVLCRQLSCGTALSTTGGKYIGERSVRVWGHRFHCLGNESLLDNCQMTVLGAPPCIHGNTVSVICTGSLTQPLFPCLANVSDPYLSAVPEGSALICLEDKRLRLVDGDSRCAGRVEIYHDGFWGTICDDGWDLSDAHVVCQKLGCGVAFNATVSAHFGEGSGPIWLDDLNCTGTESHLWQCPSRGWGQHDCRHKEDAGVICSEFTALRLYSETETESCAGRLEVFYNGTWGSVGRRNITTAIAGIVCRQLGCGENGVVSLAPLSKTGSGFMWVDDIQCPKTHISIWQCLSAPWERRISSPAEETWITCEDRIRVRGGDTECSGRVEIWHAGSWGTVCDDSWDLAEAEVVCQQLGCGSALAALRDASFGQGTGTIWLDDMRCKGNESFLWDCHAKPWGQSDCGHKEDAGVRCSGQSLKSLNASSGHLALILSSIFGLLLLVLFILFLTWCRVQKQKHLPLRVSTRRRGSLEENLFHEMETCLKREDPHGTRTSDDTPNHGCEDASDTSLLGVLPASEATK

SEQ ID NO: 12 - protein, 40 aa, Homo sapiens
MMLPQNSWHIDFGRCCCHQNLFSAVVTCILLLNSCFLISS

SEQ ID NO: 13 - protein, 1413 aa, Homo sapiens
FNGTDLELRLVNGDGPCSGTVEVKFQGQWGTVCDDGWNTTASTVVCKQLGCPFSFAMFRFGQAVTRHGKIWLDDVSCYGNESALWECQHREWGSHNCYHGEDVGVNCYGEANLGLRLVDGNNSCSGRVEVKFQERWGTICDDGWNLNTAAVVCRQLGCPSSFISSGVVNSPAVLRPIWLDDILCQGNELALWNCRHRGWGNHDCSHNEDVTLTCYDSSDLELRLVGGTNRCMGRVELKIQGRWGTVCHHKWNNAAADVVCKQLGCGTALHFAGLPHLQSGSDVVWLDGVSCSGNESFLWDCRHSGTVNFDCLHQNDVSVICSDGADLELRLADGSNNCSGRVEVRIHEQWWTICDQNWKNEQALVVCKQLGCPFSVFGSRRAKPSNEARDIWINSISCTGNESALWDCTYDGKAKRTCFRRSDAGVICSDKADLDLRLVGAHSPCYGRLEVKYQGEWGTVCHDRWSTRNAAVVCKQLGCGKPMHVFGMTYFKEASGPIWLDDVSCIGNESNIWDCEHSGWGKHNCVHREDVIVTCSGDATWGLRLVGGSNRCSGRLEVYFQGRWGTVCDDGWNSKAAAVVCSQLDCPSSIIGMGLGNASTGYGKIWLDDVSCDGDESDLWSCRNSGWGNNDCSHSEDVGVICSDASDMELRLVGGSSRCAGKVEVNVQGAVGILCANGWGMNIAEVVCRQLECGSAIRVSREPHFTERTLHILMSNSGCTGGEASLWDCIRWEWKQTACHLNMEASLICSAHRQPRLVGADMPCSGRVEVKHADTWRSVCDSDFSLHAANVLCRELNCGDAISLSVGDHFGKGNGLTWAEKFQCEGSETHLALCPIVQHPEDTCIHSREVGVVCSRYTDVRLVNGKSQCDGQVEINVLGHWGSLCDTHWDPEDARVLCRQLSCGTALSTTGGKYIGERSVRVWGHRFHCLGNESLLDNCQMTVLGAPPCIHGNTVSVICTGSLTQPLFPCLANVSDPYLSAVPEGSALICLEDKRLRLVDGDSRCAGRVEIYHDGFWGTICDDGWDLSDAHVVCQKLGCGVAFNATVSAHFGEGSGPIWLDDLNCTGTESHLWQCPSRGWGQHDCRHKEDAGVICSEFTALRLYSETETESCAGRLEVFYNGTWGSVGRRNITTAIAGIVCRQLGCGENGVVSLAPLSKTGSGFMWVDDIQCPKTHISIWQCLSAPWERRISSPAEETWITCEDRIRVRGGDTECSGRVEIWHAGSWGTVCDDSWDLAEAEVVCQQLGCGSALAALRDASFGQGTGTIWLDDMRCKGNESFLWDCHAKPWGQSDCGHKEDAGVRCSGQSLKSLNASSGHLALILSSIFGLLLLVLFILFLTWCRVQKQKHLPLRVSTRRRGSLEENLFHEMETCLKREDPHGTRTSDDTPNHGCEDASDTSLLGVLPASEATK

SEQ ID NO: 14 - protein, 1319 aa, Homo sapiens
FNGTDLELRLVNGDGPCSGTVEVKFQGQWGTVCDDGWNTTASTVVCKQLGCPFSFAMFRFGQAVTRHGKIWLDDVSCYGNESALWECQHREWGSHNCYHGEDVGVNCYGEANLGLRLVDGNNSCSGRVEVKFQERWGTICDDGWNLNTAAVVCRQLGCPSSFISSGVVNSPAVLRPIWLDDILCQGNELALWNCRHRGWGNHDCSHNEDVTLTCYDSSDLELRLVGGTNRCMGRVELKIQGRWGTVCHHKWNNAAADVVCKQLGCGTALHFAGLPHLQSGSDVVWLDGVSCSGNESFLWDCRHSGTVNFDCLHQNDVSVICSDGADLELRLADGSNNCSGRVEVRIHEQWWTICDQNWKNEQALVVCKQLGCPFSVFGSRRAKPSNEARDIWINSISCTGNESALWDCTYDGKAKRTCFRRSDAGVICSDKADLDLRLVGAHSPCYGRLEVKYQGEWGTVCHDRWSTRNAAVVCKQLGCGKPMHVFGMTYFKEASGPIWLDDVSCIGNESNIWDCEHSGWGKHNCVHREDVIVTCSGDATWGLRLVGGSNRCSGRLEVYFQGRWGTVCDDGWNSKAAAVVCSQLDCPSSIIGMGLGNASTGYGKIWLDDVSCDGDESDLWSCRNSGWGNNDCSHSEDVGVICSDASDMELRLVGGSSRCAGKVEVNVQGAVGILCANGWGMNIAEVVCRQLECGSAIRVSREPHFTERTLHILMSNSGCTGGEASLWDCIRWEWKQTACHLNMEASLICSAHRQPRLVGADMPCSGRVEVKHADTWRSVCDSDFSLHAANVLCRELNCGDAISLSVGDHFGKGNGLTWAEKFQCEGSETHLALCPIVQHPEDTCIHSREVGVVCSRYTDVRLVNGKSQCDGQVEINVLGHWGSLCDTHWDPEDARVLCRQLSCGTALSTTGGKYIGERSVRVWGHRFHCLGNESLLDNCQMTVLGAPPCIHGNTVSVICTGSLTQPLFPCLANVSDPYLSAVPEGSALICLEDKRLRLVDGDSRCAGRVEIYHDGFWGTICDDGWDLSDAHVVCQKLGCGVAFNATVSAHFGEGSGPIWLDDLNCTGTESHLWQCPSRGWGQHDCRHKEDAGVICSEFTALRLYSETETESCAGRLEVFYNGTWGSVGRRNITTAIAGIVCRQLGCGENGVVSLAPLSKTGSGFMWVDDIQCPKTHISIWQCLSAPWERRISSPAEETWITCEDRIRVRGGDTECSGRVEIWHAGSWGTVCDDSWDLAEAEVVCQQLGCGSALAALRDASFGQGTGTIWLDDMRCKGNESFLWDCHAKPWGQSDCGHKEDAGVRCSGQSLKSLNASSGH

SEQ ID NO: 15 - protein, 24 aa, Homo sapiens
LALILSSIFGLLLLVLFILFLTWC

SEQ ID NO: 16 - protein, 70 aa, Homo sapiens
RVQKQKHLPLRVSTRRRGSLEENLFHEMETCLKREDPHGTRTSDDTPNHGCEDASDTSLLGVLPASEATK

SEQ ID NO: 17 - DNA, 3104 bp, Homo sapiens
gtcgacccacgcgtccggtctgtggctgagcatggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtggtaagtggggacagcagtgctcatctggtggaagagattcagctgttccctgaccctgaacctgttcgcaacctgcagctggcccccacccagggtgcagtgtttgtaggcttctcaggaggtgtctggagggtgccccgagccaactgtagtgtctatgagagctgtgtggactgtgtccttgcccgggacccccactgtgcctgggaccctgagtcccgaacctgttgcctcctgtctgcccccaacctgaactcctggaagcaggacatggagcgggggaacccagagtgggcatgtgccagtggccccatgagcaggagccttcggcctcagagccgcccgcaaatcattaaagaagtcctggctgtccccaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagcttaaactctaggcacaggccggggctgcggtgcaggcacctggccatgctggctgggcggcccaagcacagccctgactaggatgacagcagcacaaaagaccacctttctcccctgagaggagcttctgctactctgcatcactgatgacactcagcagggtgatgcacagcagtctgcctcccctatgggactcccttctaccaagcacatgagctctctaacagggtgggggctacccccagacctgctcctacactgatattgaagaacctggagaggatccttcagttctggccattccagggaccctccagaaacacagtgtttcaagagatcctaaaaaaacctgcctgtcccaggaccctatggtaatgaacaccaaacatctaaacaatcatatgctaacatgccactcctggaaactccactctgaagctgccgctttggacaccaacactcccttctcccagggtcatgcagggatctgctccctcctgcttcccttaccagtcgtgcaccgctgactcccaggaagtctttcctgaagtctgaccacctttcttcttgcttcagttggggcagactctgatcccttctgccctggcagaatggcaggggtaatctgagccttcttcactcctttaccctagctgaccccttcacctctccccctcccttttcctttgttttgggattcagaaaactgcttgtcagagactgtttattttttattaaaaatataaggcttaaaaaaaaaaaaaaaaaaaaaaaaaaaaaagggcggccgc

SEQ ID NO: 18 - DNA, 2283 bp, Homo sapiens
atggccctcccagccctgggcctggacccctggagcctcctgggccttttcctcttccaactgcttcagctgctgctgccgacgacgaccgcggggggaggcgggcaggggcccatgcccagggtcagatactatgcaggggatgaacgtagggcacttagcttcttccaccagaagggcctccaggattttgacactctgctcctgagtggtgatggaaatactctctacgtgggggctcgagaagccattctggccttggatatccaggatccaggggtccccaggctaaagaacatgataccgtggccagccagtgacagaaaaaagagtgaatgtgcctttaagaagaagagcaatgagacacagtgtttcaacttcatccgtgtcctggtttcttacaatgtcacccatctctacacctgcggcaccttcgccttcagccctgcttgtaccttcattgaacttcaagattcctacctgttgcccatctcggaggacaaggtcatggagggaaaaggccaaagcccctttgaccccgctcacaagcatacggctgtcttggtggatgggatgctctattctggtactatgaacaacttcctgggcagtgagcccatcctgatgcgcacactgggatcccagcctgtcctcaagaccgacaacttcctccgctggctgcatcatgacgcctcctttgtggcagccatcccttcgacccaggtcgtctacttcttcttcgaggagacagccagcgagtttgacttctttgagaggctccacacatcgcgggtggctagagtctgcaagaatgacgtgggcggcgaaaagctgctgcagaagaagtggaccaccttcctgaaggcccagctgctctgcacccagccggggcagctgcccttcaacgtcatccgccacgcggtcctgctccccgccgattctcccacagctccccacatctacgcagtcttcacctcccagtggcaggttggcgggaccaggagctctgcggtttgtgccttctctctcttggacattgaacgtgtctttaaggggaaatacaaagagttgaacaaagaaacttcacgctggactacttataggggccctgagaccaacccccggccaggcagttgctcagtgggcccctcctctgataaggccctgaccttcatgaaggaccatttcctgatggatgagcaagtggtggggacgcccctgctggtgaaatctggcgtggagtatacacggcttgcagtggagacagcccagggccttgatgggcacagccatcttgtcatgtacctgggaaccaccacagggtcgctccacaaggctgtggtaagtggggacagcagtgctcatctggtggaagagattcagctgttccctgaccctgaacctgttcgcaacctgcagctggcccccacccagggtgcagtgtttgtaggcttctcaggaggtgtctggagggtgccccgagccaactgtagtgtctatgagagctgtgtggactgtgtccttgcccgggacccccactgtgcctgggaccctgagtcccgaacctgttgcctcctgtctgcccccaacctgaactcctggaagcaggacatggagcgggggaacccagagtgggcatgtgccagtggccccatgagcaggagccttcggcctcagagccgcccgcaaatcattaaagaagtcctggctgtccccaactccatcctggagctcccctgcccccacctgtcagccttggcctcttattattggagtcatggcccagcagcagtcccagaagcctcttccactgtctacaatggctccctcttgctgatagtgcaggatggagttgggggtctctaccagtgctgggcaactgagaatggcttttcataccctgtgatctcctactgggtggacagccaggaccagaccctggccctggatcctgaactggcaggcatcccccgggagcatgtgaaggtcccgttgaccagggtcagtggtggggccgccctggctgcccagcagtcctactggccccactttgtcactgtcactgtcctctttgccttagtgctttcaggagccctcatcatcctcgtggcctccccattgagagcactccgggctcggggcaaggttcagggctgtgagaccctgcgccctggggagaaggccccgttaagcagagagcaacacctccagtctcccaaggaatgcaggacctctgccagtgatgtggacgctgacaacaactgcctaggcactgaggtagct

SEQ ID NO: 19 - protein, 761 aa, Homo sapiens
MALPALGLDPWSLLGLFLFQLLQLLLPTTTAGGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 20 - protein, 31 aa, Homo sapiens
MALPALGLDPWSLLGLFLFQLLQLLLPTTTA

SEQ ID NO: 21 - protein, 730 aa, Homo sapiens
GGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 22 - protein, 652 aa, Homo sapiens
GGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPH

SEQ ID NO: 23 - protein, 21 aa, Homo sapiens
FVTVTVLFALVLSGALIILVA

SEQ ID NO: 24 - protein, 57 aa, Homo sapiens
SPLRALRARGKVQGCETLRPGEKAPLSREQHLQSPKECRTSASDVDADNNCLGTEVA

SEQ ID NO: 25 - DNA, 2964 bp, Homo sapiens
gtcgacccacgcgtccgcggacgcgtggggacggctcccggctgcagtctgcccgcccgccccgcgcgggggccgagtcgcgaagcgcgcctgcgacccggcgtccgggcgcgctggagaggacgcgaggagccatgaggcgccagcctgcgaaggtggcggcgctgctgctcgggctgctcttggagtgcacagaagccaaaaagcattgctggtatttcgaaggactctatccaacctattatatatgccgctcctacgaggactgctgtggctccaggtgctgtgtgcgggccctctccatacagaggctgtggtacttctggttccttctgatgatgggcgtgcttttctgctgcggagccggcttcttcatccggaggcgcatgtaccccccgccgctgatcgaggagccagccttcaatgtgtcctacaccaggcagcccccaaatcccggcccaggagcccagcagccggggccgccctattacactgacccaggaggaccggggatgaaccctgtcgggaattccatggcaatggctttccaggtcccacccaactcaccccaggggagtgtggcctgcccgccccctccagcctactgcaacacgcctccgcccccgtacgaacaggtagtgaaggccaagtagtggggtgcccacgtgcaagaggagagacaggagagggcctttccctggcctttctgtcttcgttgatgttcacttccaggaacggtctcgtgggctgctaagggcagttcctctgatatcctcacagcaagcacagctctctttcaggctttccatggagtacaatatatgaactcacactttgtctcctctgttgcttctgtttctgacgcagtctgtgctctcacatggtagtgtggtgacagtccccgagggctgacgtccttacggtggcgtgaccagatctacaggagagagactgagaggaagaaggcagtgctggaggtgcaggtggcatgtagaggggccaggccgagcatcccaggcaagcatccttctgcccgggtattaataggaagccccatgccgggcggctcagccgatgaagcagcagccgactgagctgagcccagcaggtcatctgctccagcctgtcctctcgtcagccttcctcttccagaagctgttggagagacattcaggagagagcaagccccttgtcatgtttctgtctctgttcatatcctaaagatagacttctcctgcaccgccagggaagggtagcacgtgcagctctcaccgcaggatggggcctagaatcaggcttgccttggaggcctgacagtgatctgacatccactaagcaaatttatttaaattcatgggaaatcacttcctgccccaaactgagacattgcattttgtgagctcttggtctgatttggagaaaggactgttacccatttttttggtgtgtttatggaagtgcatgtagagcgtcctgccctttgaaatcagactgggtgtgtgtcttccctggacatcactgcctctccagggcattctcaggcccgggggtctccttccctcaggcagctccagtggtgggttctgaagggtgctttcaaaacggggcacatctggctgggaagtcacatggactcttccagggagagagaccagctgaggcgtctctctctgaggttgtgttgggtctaagcgggtgtgtgctgggctccaaggaggaggagcttgctgggaaaagacaggagaagtactgactcaactgcactgaccatgttgtcataattagaataaagaagaagtggtcggaaatgcacattcctggataggaatcacagctcaccccaggatctcacaggtagtctcctgagtagttgacggctagcggggagctagttccgccgcatagttatagtgttgatgtgtgaacgctgacctgtcctgtgtgctaagagctatgcagcttagctgaggcgcctagattactagatgtgctgtatcacggggaatgaggtgggggtgcttattttttaatgaactaatcagagcctcttgagaaattgttactcattgaactggagcatcaagacatctcatggaagtggatacggagtgatttggtgtccatgcttttcactctgaggacatttaatcggagaacctcctggggaattttgtgggagacacttgggaacaaaacagacaccctgggaatgcagttgcaagcacagatgctgccaccagtgtctctgaccaccctggtgtgactgctgactgccagcgtggtacctcccatgctgcaggcctccatctaaatgagacaacaaagcacaatgttcactgtttacaaccaagacaactgcgtgggtccaaacactcctcttcctccaggtcatttgttttgcatttttaatgtctttattttttgtaatgaaaaagcacactaagctgcccctggaatcgggtgcagctgaataggcacccaaaagtccgtgactaaatttcgtttgtctttttgatagcaaattatgttaagagacagtgatggctagggctcaacaattttgtattcccatgtttgtgtgagacagagtttgttttcccttgaacttggttagaattgtgctactgtgaacgctgatcctgcatatggaagtcccactttggtgacatttcctggccattcttgtttccattgtgtggatggtgggttgtgcccacttcctggagtgagacagctcctggtgtgtagaattcccggagcgtccgtggttcagagtaaacttgaagcagatctgtgcatgcttttcctctgcaacaattggctcgtttctcttttttgttctcttttgataggatcctgtttcctatgtgtgcaaaataaaaataaatttgggcaaaaaaaaaaaaaaaaaaaaaaaaaaaaaaaaaaaaaaaaaaaaaaaaaaaaaaaaaaaagggcggccgc

SEQ ID NO: 26 - DNA, 516 bp, Homo sapiens
atgaggcgccagcctgcgaaggtggcggcgctgctgctcgggctgctcttggagtgcacagaagccaaaaagcattgctggtatttcgaaggactctatccaacctattatatatgccgctcctacgaggactgctgtggctccaggtgctgtgtgcgggccctctccatacagaggctgtggtacttctggttccttctgatgatgggcgtgcttttctgctgcggagccggcttcttcatccggaggcgcatgtaccccccgccgctgatcgaggagccagccttcaatgtgtcctacaccaggcagcccccaaatcccggcccaggagcccagcagccggggccgccctattacactgacccaggaggaccggggatgaaccctgtcgggaattccatggcaatggctttccaggtcccacccaactcaccccaggggagtgtggcctgcccgccccctccagcctactgcaacacgcctccgcccccgtacgaacaggtagtgaaggccaag

SEQ ID NO: 27 - protein, 172 aa, Homo sapiens
MRRQPAKVAALLLGLLLECTEAKKHCWYFEGLYPTYYICRSYEDCCGSRCCVRALSIQRLWYFWFLLMMGVLFCCGAGFFIRRRMYPPPLIEEPAFNVSYTRQPPNPGPGAQQPGPPYYTDPGGPGMNPVGNSMAMAFQVPPNSPQGSVACPPPPAYCNTPPPPYEQVVKAK

SEQ ID NO: 28 - protein, 22 aa, Homo sapiens
MRRQPAKVAALLLGLLLECTEA

SEQ ID NO: 29 - protein, 150 aa, Homo sapiens
KKHCWYFEGLYPTYYICRSYEDCCGSRCCVRALSIQRLWYFWFLLMMGVLFCCGAGFFIRRRMYPPPLIEEPAFNVSYTRQPPNPGPGAQQPGPPYYTDPGGPGMNPVGNSMAMAFQVPPNSPQGSVACPPPPAYCNTPPPPYEQVVKAK

SEQ ID NO: 30 - protein, 38 aa, Homo sapiens
KKHCWYFEGLYPTYYICRSYEDCCGSRCCVRALSIQRL

SEQ ID NO: 31 - protein, 21 aa, Homo sapiens
WYFWFLLMMGVLFCCGAGFFI

SEQ ID NO: 32 - protein, 91 aa, Homo sapiens
RRRMYPPPLIEEPAFNVSYTRQPPNPGPGAQQPGPPYYTDPGGPGMNPVGNSMAMAFQVPPNSPQGSVACPPPPAYCNTPPPPYEQVVKAK

SEQ ID NO: 33 - DNA, 1980 bp, Homo sapiens
gtcgacccacgcgtccgcagctttggacacttcctctgcttgaggacaccttgactaacctccaagggcaactaaaggatcaagaaaggcccagcacagcagaagatcagctggatctagctcctgcaggagatgtgtacaaagacaatcccagtcctctggggatgtttcctcctgtggaatctctatgtctcatcctctcagaccatttaccctggaatcaaggcaaggattactcagagggcacttgactatggtgttcaagctggaatgaagatgattgagcaaatgctaaaagaaaagaaactcccagatttaagcggttctgagtctcttgaatttctaaaagttgattatgtaaactacaatttttcaaatataaaaatcagtgccttttcatttccaaatacctcattggcttttgtgcctggagtgggaatcaaagcgctaaccaaccatggcactgccaacatcagcacagactgggggttcgagtctccactttttgttctgtataactcctttgctgagcccatggagaaacccattttaaagaacttaaatgaaatgctctgtcccattattgcaagtgaagtcaaagcgctaaatgccaacctcagcacactggaggttttaaccaagattgacaactacactctgctggattactccctaatcagttctccagaaattactgagaactaccttgacctgaacttgaagggtgtattctacccactggaaaacctcaccgacccccccttctcaccagttccttttgtgctcccagaacgcagcaactccatgctctacattggaatcgccgagtatttctttaaatctgcgtcctttgctcatttcacagctggggttttcaatctcactctctccaccgaagagatttccaaccattttgttcaaaactctcaaggccttggcaacgtgctctcccggattgcagagatctacatcttgtcccagcccttcatggtgaggatcatggccacagagcctcccataatcaatctacaaccaggcaatttcaccctggacatccctgcctccatcatgatgctcacccaacccaagaactccacagttgaaaccatcgtttccatggacttcgttgctagtaccagtgttggcctggttattttgggacaaagactggtctgctccttgtctctgaacagattccgccttgctttgccagagtccaatcgcagcaacattgaggtcttgaggtttgaaaatattctatcgtccattcttcactttggagtcctcccactggccaatgcaaaattgcagcaaggatttcctctgcccaatccacacaaattcttattcgtcaattcagatattgaagttcttgagggtttccttttgatttccaccgacctgaagtatgaaacatcctcaaagcagcagccaagtttccacgtatgggaaggtctgaacctgataagcagacagtggagggggaagtcagccccttgattgccggtttgcaattcaccccaggaagtaaatggtccttaatcctacaactactgtaaacccagaagggaaagacagtacacactggaattgtaaagcccttgtgaattgcttaggcagaaagttttctttcttaagccttcaggaacccagaataaggcagactctgttaaagggataaatagaggtgtctgaatgtgagtgtatgcatgctgcgtgtgtctgtgtttatgtttgtttgtttgtttggggcaagaaagattctaggacaagagctaggcatgtacttctgaccaggtgggtaagcaactctaagtctgtatttgtattggtcattctcagtggaaatcccttaggccctctagtggttttcccctacctgcatattggttttcatgttttatattcactgttactatcttctgtgtttaattaaaattgttttctatcaaaaaaaaaaaaaaaaaaaagggcggccgc

SEQ ID NO: 34 - DNA, 1365 bp, Homo sapiens
atgtgtacaaagacaatcccagtcctctggggatgtttcctcctgtggaatctctatgtctcatcctctcagaccatttaccctggaatcaaggcaaggattactcagagggcacttgactatggtgttcaagctggaatgaagatgattgagcaaatgctaaaagaaaagaaactcccagatttaagcggttctgagtctcttgaatttctaaaagttgattatgtaaactacaatttttcaaatataaaaatcagtgccttttcatttccaaatacctcattggcttttgtgcctggagtgggaatcaaagcgctaaccaaccatggcactgccaacatcagcacagactgggggttcgagtctccactttttgttctgtataactcctttgctgagcccatggagaaacccattttaaagaacttaaatgaaatgctctgtcccattattgcaagtgaagtcaaagcgctaaatgccaacctcagcacactggaggttttaaccaagattgacaactacactctgctggattactccctaatcagttctccagaaattactgagaactaccttgacctgaacttgaagggtgtattctacccactggaaaacctcaccgacccccccttctcaccagttccttttgtgctcccagaacgcagcaactccatgctctacattggaatcgccgagtatttctttaaatctgcgtcctttgctcatttcacagctggggttttcaatctcactctctccaccgaagagatttccaaccattttgttcaaaactctcaaggccttggcaacgtgctctcccggattgcagagatctacatcttgtcccagcccttcatggtgaggatcatggccacagagcctcccataatcaatctacaaccaggcaatttcaccctggacatccctgcctccatcatgatgctcacccaacccaagaactccacagttgaaaccatcgtttccatggacttcgttgctagtaccagtgttggcctggttattttgggacaaagactggtctgctccttgtctctgaacagattccgccttgctttgccagagtccaatcgcagcaacattgaggtcttgaggtttgaaaatattctatcgtccattcttcactttggagtcctcccactggccaatgcaaaattgcagcaaggatttcctctgcccaatccacacaaattcttattcgtcaattcagatattgaagttcttgagggtttccttttgatttccaccgacctgaagtatgaaacatcctcaaagcagcagccaagtttccacgtatgggaaggtctgaacctgataagcagacagtggagggggaagtcagcccct

SEQ ID NO: 35 - protein, 455 aa, Homo sapiens
MCTKTIPVLWGCFLLWNLYVSSSQTIYPGIKARITQRALDYGVQAGMKMIEQMLKEKKLPDLSGSESLEFLKVDYVNYNFSNIKISAFSFPNTSLAFVPGVGIKALTNHGTANISTDWGFESPLFVLYNSFAEPMEKPILKNLNEMLCPIIASEVKALNANLSTLEVLTKIDNYTLLDYSLISSPEITENYLDLNLKGVFYPLENLTDPPFSPVPFVLPERSNSMLYIGIAEYFFKSASFAHFTAGVFNLTLSTEEISNHFVQNSQGLGNVLSRIAEIYILSQPFMVRIMATEPPIINLQPGNFTLDIPASIMMLTQPKNSTVETIVSMDFVASTSVGLVILGQRLVCSLSLNRFRLALPESNRSNIEVLRFENILSSILHFGVLPLANAKLQQGFPLPNPHKFLFVNSDIEVLEGFLLISTDLKYETSSKQQPSFHVWEGLNLISRQWRGKSAP

SEQ ID NO: 36 - protein, 23 aa, Homo sapiens
MCTKTIPVLWGCFLLWNLYVSSS

SEQ ID NO: 37 - protein, 432 aa, Homo sapiens
QTIYPGIKARITQRALDYGVQAGMKMIEQMLKEKKLPDLSGSESLEFLKVDYVNYNFSNIKISAFSFPNTSLAFVPGVGIKALTNHGTANISTDWGFESPLFVLYNSFAEPMEKPILKNLNEMLCPIIASEVKALNANLSTLEVLTKIDNYTLLDYSLISSPEITENYLDLNLKGVFYPLENLTDPPFSPVPFVLPERSNSMLYIGIAEYFFKSASFAHFTAGVFNLTLSTEEISNHFVQNSQGLGNVLSRIAEIYILSQPFMVRIMATEPPIINLQPGNFTLDIPASIMMLTQPKNSTVETIVSMDFVASTSVGLVILGQRLVCSLSLNRFRLALPESNRSNIEVLRFENILSSILHFGVLPLANAKLQQGFPLPNPHKFLFVNSDIEVLEGFLLISTDLKYETSSKQQPSFHVWEGLNLISRQWRGKSAP

SEQ ID NO: 38 - protein, 483 aa, Homo sapiens
MARGPCNAPRWVSLMVLVAIGTAVTAAVNPGVVVRISQKGLDYASQQGTAALQKELKRIKIPDYSDSFKIKHLGKGHYSFYSMDIREFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSKLQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQAFAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK

SEQ ID NO: 39 - protein, 481 aa, Homo sapiens
MGALARALPSILLALLLTSTPEALGANPGLVARITDKGLQYAAQEGLLALQSELLRITLPDFTGDLRIPHVGRGRYEFHSLNIHEFQLPSSQISMVPNVGLKFSISNANIKISGKWKAQKRFLKMSGNFDLSIEGMSISADLKLGSNPTSGKPTITCSSCSSHINSVHVHISKSKVGWLIQLFHKKIESALRNKMNSQVCEKVTNSVSSKLQPYFQTLPVMTKIDSVAGINYGLVAPPATTAETLDVQMKGEFYSENHHNPPPFAPPVMEFPAAHDRMVYLGLSDYFFNTAGLVYQEAGVLKMTLRDDMIPKESKFRLTTKFFGTFLPEVAKKFPNMKIQIHVSASTPPHLSVQPTGLTFYPAVDVQALAVLPNSSLASLFLIGMHTTGSMEVSAESNRLVGELKLDRLLLELKHSNIGPFPVELLQDIMNYIVPILVLPRVNEKLQKGFPLPTPARVQLYNVVLQPHQNFLLFGADVVYK

SEQ ID NO: 40 - protein, 383 aa, Caenorhabditis elegans
MRIAHASSRGNISIFSVFLIPLIAYILILPGVRRKRVVTTVTYVLMLAVGGALIASLIYPCWASGSQMIYTQFRGHSNERILAKIGVEIGLQKVNVTLKFERLLSSNDVLPGSDMTELYYNEGFDISGISSMAEALHHGLENGLPYPMLSVLEYFSLNQDSFDWGRHYRVAGHYTHAAIWFAFACWCLSVVLMLFLPHNAYKSILATGISCLIACLVYLLLSPCELRIAFTGENFERVDLTATFSFCFYLIFAIGILCVLCGLGLGICEHWRIYTLSTFLDASLDEHVGPKWKKLPTGGPALQGVQIGAYGTNTTNSSRDKNDISSDKTAGSSGFQSRTSTCQSSASSASLRSQSSIETVHDEAELERTHVHFLQEPCSSSST

SEQ ID NO: 41 - protein, 399 aa, Homo sapiens
MKMRFLGLVVCLVLWPLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 42 - protein, 19 aa, Mus sp.
MAPPAARLALLSAAALTLA

SEQ ID NO: 43 - protein, 451 aa, Mus sp.
ARPAPGPRSGPECFTANGADYRGTQSWTALQGGKPCLFWNETFQHPYNTLKYPNGEGGLGEHNYCRNPDGDVSPWCYVAEHEDGVYWKYCEIPACQMPGNLGCYKDHGNPPPLTGTSKTSNKLTIQTCISFCRSQRFKFAGMESGYACFCGNNPDYWKHGEAASTECNSVCFGDHTQPCGGDGRIILFDTLVGACGGNYSAMAAVVYSPDFPDTYATGRVCYWTIRVPGASRIHFNFTLFDIRDSADMVELLDGYTHRVLVRLSGRSRPPLSFNVSLDFVILYFFSDRINQAQGFAVLYQATKEEPPQERPAVNQTLAEVITEQANLSVSAAHSSKVLYVITPSPSHPPQTAQVAIPGHRQLGPTATEWKDGLCTAWRPSSSSQSQQLSQRFFCMSHLNLIESLHQETLGTVVSLGLLEISGPFSMNLPLQSPSLRRSSRVRVNKMTAIPS

SEQ ID NO: 44 - protein, 150 aa, Mus sp.
KKHCWYFEGLYPTYYICRSYEDCCGSRCCVRALSIQRLWYFWFLLMMGVLFCCGAGFFIRRRMYPPPLIEEPTFNVSYTRQPPNPAPGAQQMGPPYYTDPGGPGMNPVGNTMAMAFQVQPNSPHGGTTYPPPPSYCNTPPPPYEQVVKDK

SEQ ID NO: 45 - DNA, 2044 bp, Homo sapiens
gtcgacccacgcgtccggggaattgcagcaggaaaatatgtgaagagtttttaaacccacaaattcttcttactttagaattagttgttacattggcaggaaaaaataaatgcagatgttggaccatgttggaaaccttgtcaagacagtggattgtctcacacagaatggaaatgtggcttctgattctggtggcgtatatgttccagagaaatgtgaattcagtacatatgccaactaaagctgtggacccagaagcattcatgaatattagtgaaatcatccaacatcaaggctatccctgtgaggaatatgaagtcgcaactgaagatgggtatatcctttctgttaacaggattcctcgaggcctagtgcaacctaagaagacaggttccaggcctgtggtgttactgcagcatggcctagttggaggtgctagcaactggatttccaacctgcccaacaatagcctgggcttcattctggcagatgctggttttgacgtgtggatggggaacagcaggggaaacgcctggtctcgaaaacacaagacactctccatagaccaagatgagttctgggctttcagttatgatgagatggctaggtttgaccttcctgcagtgataaactttattttgcagaaaacgggccaggaaaagatctattatgtcggctattcacagggcaccaccatgggctttattgcattttccaccatgccagagctggctcagaaaatcaaaatgtattttgctttagcacccatagccactgttaagcatgcaaaaagccccgggaccaaatttttgttgctgccagatatgatgatcaagggattgtttggcaaaaaagaatttctgtatcagaccagatttctcagacaacttgttatttacctttgtggccaggtgattcttgatcagatttgtagtaatatcatgttacttctgggtggattcaacaccaacaatatgaacatgagccgagcaagtgtatatgctgcccacactcttgctggaacatctgtgcaaaatattctacactggagccaggcagtgaattctggtgaactccgggcatttgactgggggagtgagaccaaaaatctggaaaaatgcaatcagccaactcctgtaaggtacagagtcagagatatgacggtccctacagcaatgtggacaggaggtcaggactggctttcaaatccagaagacgtgaaaatgctgctctctgaggtgaccaacctcatctaccataagaatattcctgaatgggctcacgtggatttcatctggggtttggatgctcctcaccgtatgtacaatgaaatcatccatctgatgcagcaggaggagaccaacctttcccagggacggtgtgaggccgtattgtgaagcatctgacactgacgatcttaggacaacctcctgagggatggggctaggacccatgaaggcagaattacggagagcagagacctagtatacatttttcagattccctgcacttggcactaaatccgacacttacatttacattttttttctgtaaattaaagtacttattaggtaaatagaggttttgtatgctattatatattctaccatcttgaagggtaggttttacctgatagccagaaaatatctagacattctctatatcattcaggtaaatctctttaaaacacctattgttttttctataagccatatttttggagcactaaagtaaaatggcaaattgggacagatattgaggtctggagtctgtggattattgttgactttgacaaaataagctagacattttcaccttgttgccacagagacataacactacctcaggaagctgagctgctttaaggacaacaacaacaaaatcagtgttacagtatggatgaaatctatgttaagcattctcagaataaggccaagttttatagttgcatctcagggaagaaaattttataggatgtttatgagttctccaataaatgcattctgcattacataaaaaaaaaaaaaaaaaaaagggcggccgc

SEQ ID NO: 46 - DNA, 1269 bp, Homo sapiens
atgttggaaaccttgtcaagacagtggattgtctcacacagaatggaaatgtggcttctgattctggtggcgtatatgttccagagaaatgtgaattcagtacatatgccaactaaagctgtggacccagaagcattcatgaatattagtgaaatcatccaacatcaaggctatccctgtgaggaatatgaagtcgcaactgaagatgggtatatcctttctgttaacaggattcctcgaggcctagtgcaacctaagaagacaggttccaggcctgtggtgttactgcagcatggcctagttggaggtgctagcaactggatttccaacctgcccaacaatagcctgggcttcattctggcagatgctggttttgacgtgtggatggggaacagcaggggaaacgcctggtctcgaaaacacaagacactctccatagaccaagatgagttctgggctttcagttatgatgagatggctaggtttgaccttcctgcagtgataaactttattttgcagaaaacgggccaggaaaagatctattatgtcggctattcacagggcaccaccatgggctttattgcattttccaccatgccagagctggctcagaaaatcaaaatgtattttgctttagcacccatagccactgttaagcatgcaaaaagccccgggaccaaatttttgttgctgccagatatgatgatcaagggattgtttggcaaaaaagaatttctgtatcagaccagatttctcagacaacttgttatttacctttgtggccaggtgattcttgatcagatttgtagtaatatcatgttacttctgggtggattcaacaccaacaatatgaacatgagccgagcaagtgtatatgctgcccacactcttgctggaacatctgtgcaaaatattctacactggagccaggcagtgaattctggtgaactccgggcatttgactgggggagtgagaccaaaaatctggaaaaatgcaatcagccaactcctgtaaggtacagagtcagagatatgacggtccctacagcaatgtggacaggaggtcaggactggctttcaaatccagaagacgtgaaaatgctgctctctgaggtgaccaacctcatctaccataagaatattcctgaatgggctcacgtggatttcatctggggtttggatgctcctcaccgtatgtacaatgaaatcatccatctgatgcagcaggaggagaccaacctttcccagggacggtgtgaggccgtattg

SEQ ID NO: 47 - protein, 423 aa, Homo sapiens
MLETLSRQWIVSHRMEMWLLILVAYMFQRNVNSVHMPTKAVDPEAFMNISEIIQHQGYPCEEYEVATEDGYILSVNRIPRGLVQPKKTGSRPVVLLQHGLVGGASNWISNLPNNSLGFILADAGFDVWMGNSRGNAWSRKHKTLSIDQDEFWAFSYDEMARFDLPAVINFILQKTGQEKIYYVGYSQGTTMGFIAFSTMPELAQKIKMYFALAPIATVKHAKSPGTKFLLLPDMMIKGLFGKKEFLYQTRFLRQLVIYLCGQVILDQICSNIMLLLGGFNTNNMNMSRASVYAAHTLAGTSVQNILHWSQAVNSGELRAFDWGSETKNLEKCNQPTPVRYRVRDMTVPTAMWTGGQDWLSNPEDVKMLLSEVTNLIYHKNIPEWAHVDFIWGLDAPHRMYNEIIHLMQQEETNLSQGRCEAVL

SEQ ID NO: 48 - protein, 33 aa, Homo sapiens
MLETLSRQWIVSHRMEMWLLILVAYMFQRNVNS

SEQ ID NO: 49 - protein, 390 aa, Homo sapiens
VHMPTKAVDPEAFMNISEIIQHQGYPCEEYEVATEDGYILSVNRIPRGLVQPKKTGSRPVVLLQHGLVGGASNWISNLPNNSLGFILADAGFDVWMGNSRGNAWSRKHKTLSIDQDEFWAFSYDEMARFDLPAVINFILQKTGQEKIYYVGYSQGTTMGFIAFSTMPELAQKIKMYFALAPIATVKHAKSPGTKFLLLPDMMIKGLFGKKEFLYQTRFLRQLVIYLCGQVILDQICSNIMLLLGGFNTNNMNMSRASVYAAHTLAGTSVQNILHWSQAVNSGELRAFDWGSETKNLEKCNQPTPVRYRVRDMTVPTAMWTGGQDWLSNPEDVKMLLSEVTNLIYHKNIPEWAHVDFIWGLDAPHRMYNEIIHLMQQEETNLSQGRCEAVL

SEQ ID NO: 50 - protein, 221 aa, Homo sapiens
VHMPTKAVDPEAFMNISEIIQHQGYPCEEYEVATEDGYILSVNRIPRGLVQPKKTGSRPVVLLQHGLVGGASNWISNLPNNSLGFILADAGFDVWMGNSRGNAWSRKHKTLSIDQDEFWAFSYDEMARFDLPAVINFILQKTGQEKIYYVGYSQGTTMGFIAFSTMPELAQKIKMYFALAPIATVKHAKSPGTKFLLLPDMMIKGLFGKKEFLYQTRFLRQ

SEQ ID NO: 51 - protein, 25 aa, Homo sapiens
LVIYLCGQVILDQICSNIMLLLGGF

SEQ ID NO: 52 - protein, 144 aa, Homo sapiens
NTNNMNMSRASVYAAHTLAGTSVQNILHWSQAVNSGELRAFDWGSETKNLEKCNQPTPVRYRVRDMTVPTAMWTGGQDWLSNPEDVKMLLSEVTNLIYHKNIPEWAHVDFIWGLDAPHRMYNEIIHLMQQEETNLSQGRCEAVL

SEQ ID NO: 53 - DNA, 2133 bp, Homo sapiens
gtcgacccacgcgtccacggcgagggctcccggggcgcagcattgccccccctgcaccacctcaccaagatggctactttgggacacacattccccttctatgctggccccaagccaaccttcccgatggacaccactttggccagcatcatcatgatctttctgactgcactggccacgttcatcgtcatcctgcctggcattcggggaaagacgaggctgttctggctgcttcgggtggtgaccagcttattcatcggggctgcaatcctggctgtgaatttcagttctgagtggtctgtgggccaggtcagcaccaacacatcatacaaggccttcagttctgagtggatcagcgctgatattgggctgcaggtcgggctgggtggagtcaacatcacactcacagggacccccgtgcagcagctgaatgagaccatcaattacaacgaggagttcacctggcgcctgggtgagaactatgctgaggagtgtgcaaaggctctggagaaggggctgccagaccctgtgttgtacctagctgagaagttcactccaagaagcccatgtggcctataccgccagtaccgcctggcgggacactacacctcagccatgctatgggtggcattcctctgctggctgctggccaatgtgatgctctccatgcctgtgctggtatatggtggctacatgctattggccacgggcatcttccagctgttggctctgctcttcttctccatggccacatcactcacctcaccctgtcccctgcacctgggcgcttctgtgctgcatactcaccatgggcctgccttctggatcacattgaccacaggactgctgtgtgtgctgctgggcctggctatggcggtggcccacaggatgcagcctcacaggctgaaggctttcttcaaccagagtgtggatgaagaccccatgctggagtggagtcctgaggaaggtggactcctgagcccccgctaccggtccatggctgacagtcccaagtcccaggacattcccctgtcagaggcttcctccaccaaggcatactgtaaggaggcacaccccaaagatcctgattgtgctttataacattcctccccgtggaggccacctggacttccagtctggctccaaacctcattggcgccccataaaaccagcagaactgccctcagggtggctgttaccagacacccagcaccaatctacagacggagtagaaaaaggaggctctatatactgatgttaaaaaacaaaacaaaacaaaaagccctaagggactgaagagatgctgggcctgtccataaagcctgttgccatgataaggccaagcaggggctagcttatctgcacagcaacccagcctttccgtgctgccttgcctcttcaagatgctattcactgaaacctaacttcacccccataacaccagcagggtgggggttacatatgattctcctatggtttcctctcatccctcggcacctcttgttttcctttttcctgggttccttttgttcttcctttacttctccagcttgtgtggccttttggtacaatgaaagacagcactggaaaggaggggaaaccaaacttctcatcctaggtctaacattaaccaactatgccacattctctttgagcttcagttcccaaatttgctacataagattgcaagacttgccaagaatcttgggatttatctttctatgccttgctgacacctaccttggccctcaaacaccacctcacaagaagccaggtgggaagttagggaatcaactccaaaacgctattccttcccaccccactcagctgggctagctgagtggcatccaggacgggggagtgggtgacctgcctcatcactgccacctaacgtccccctggggtggttcagaaagatgctagctctggtagggtccctccggcctcactagagggcgcccctattactctggagtcgacgcagagaatcaggtttcacagcactgcggagagtgtactaggctgtctccagcccagcgaagctcatgaggacgtgcgaccccggcgcggagaagccatgaaaattaatgggaaaaacagtttttaaaaaaaaaaaaaaaaaagggcggccgc

SEQ ID NO: 54 - DNA, 1029 bp, Homo sapiens
atggctactttgggacacacattccccttctatgctggccccaagccaaccttcccgatggacaccactttggccagcatcatcatgatctttctgactgcactggccacgttcatcgtcatcctgcctggcattcggggaaagacgaggctgttctggctgcttcgggtggtgaccagcttattcatcggggctgcaatcctggctgtgaatttcagttctgagtggtctgtgggccaggtcagcaccaacacatcatacaaggccttcagttctgagtggatcagcgctgatattgggctgcaggtcgggctgggtggagtcaacatcacactcacagggacccccgtgcagcagctgaatgagaccatcaattacaacgaggagttcacctggcgcctgggtgagaactatgctgaggagtgtgcaaaggctctggagaaggggctgccagaccctgtgttgtacctagctgagaagttcactccaagaagcccatgtggcctataccgccagtaccgcctggcgggacactacacctcagccatgctatgggtggcattcctctgctggctgctggccaatgtgatgctctccatgcctgtgctggtatatggtggctacatgctattggccacgggcatcttccagctgttggctctgctcttcttctccatggccacatcactcacctcaccctgtcccctgcacctgggcgcttctgtgctgcatactcaccatgggcctgccttctggatcacattgaccacaggactgctgtgtgtgctgctgggcctggctatggcggtggcccacaggatgcagcctcacaggctgaaggctttcttcaaccagagtgtggatgaagaccccatgctggagtggagtcctgaggaaggtggactcctgagcccccgctaccggtccatggctgacagtcccaagtcccaggacattcccctgtcagaggcttcctccaccaaggcatactgtaaggaggcacaccccaaagatcctgattgtgcttta

SEQ ID NO: 55 - protein, 343 aa, Homo sapiens
MATLGHTFPFYAGPKPTFPMDTTLASIIMIFLTALATFIVILPGIRGKTRLFWLLRVVTSLFIGAAILAVNFSSEWSVGQVSTNTSYKAFSSEWISADIGLQVGLGGVNITLTGTPVQQLNETINYNEEFTWRLGENYAEECAKALEKGLPDPVLYLAEKFTPRSPCGLYRQYRLAGHYTSAMLWVAFLCWLLANVMLSMPVLVYGGYMLLATGIFQLLALLFFSMATSLTSPCPLHLGASVLHTHHGPAFWITLTTGLLCVLLGLAMAVAHRMQPHRLKAFFNQSVDEDPMLEWSPEEGGLLSPRYRSMADSPKSQDIPLSEASSTKAYCKEAHPKDPDCAL

SEQ ID NO: 56 - protein, 23 aa, Homo sapiens
MATLGHTFPFYAGPKPTFPMDTT

SEQ ID NO: 57 - protein, 112 aa, Homo sapiens
NFSSEWSVGQVSTNTSYKAFSSEWISADIGLQVGLGGVNITLTGTPVQQLNETINYNEEFTWRLGENYAEECAKALEKGLPDPVLYLAEKFTPRSPCGLYRQYRLAGHYTSA

SEQ ID NO: 58 - protein, 22 aa, Homo sapiens
TSLTSPCPLHLGASVLHTHHGP

SEQ ID NO: 59 - protein, 19 aa, Homo sapiens
LASIIMIFLTALATFIVIL

SEQ ID NO: 60 - protein, 20 aa, Homo sapiens
LFWLLRVVTSLFIGAAILAV

SEQ ID NO: 61 - protein, 22 aa, Homo sapiens
MLWVAFLCWLLANVMLSMPVLV

SEQ ID NO: 62 - protein, 17 aa, Homo sapiens
LATGIFQLLALLFFSMA

SEQ ID NO: 63 - protein, 22 aa, Homo sapiens
AFWITLTTGLLCVLLGLAMAVA

SEQ ID NO: 64 - protein, 8 aa, Homo sapiens
PGIRGKTR

SEQ ID NO: 65 - protein, 6 aa, Homo sapiens
YGGYML

SEQ ID NO: 66 - protein, 72 aa, Homo sapiens
HRMQPHRLKAFFNQSVDEDPMLEWSPEEGGLLSPRYRSMADSPKSQDIPLSEASSTKAYCKEAHPKDPDCAL

SEQ ID NO: 67 - DNA, 4928 bp, Mus sp.
gtcgacccacgcgtccgcccggctcccggtgctgccccctctgccccgggccgcgcccgggggtcccgcactgacggcccatggcgccgcccgccgcccgtctcgcgctgctctccgccgctgcgctcactctggcggcccggcccgcgcccggtccccgctccggccccgagtgcttcacagccaacggtgcagattacaggggaacacagagctggacagcgctgcaaggtgggaagccatgtctgttctggaacgagactttccagcatccgtacaacacgctgaagtaccccaacggggaaggaggactgggcgagcacaattattgcagaaatccagatggagacgtgagcccttggtgctacgtggccgagcatgaggacggagtctactggaagtactgtgaaattcctgcctgccagatgcctggaaaccttggctgctacaaggatcatggaaacccacctcctctcacgggcaccagtaaaacctctaacaagctcaccatacaaacctgtatcagcttctgtcggagtcagagattcaagtttgctgggatggagtcaggctatgcctgcttctgtgggaacaatcctgactactggaagcacggggaggcggccagcaccgagtgcaatagtgtctgcttcggggaccacacgcagccctgcggtggggacggcaggattatcctctttgacactctcgtgggcgcctgcggtgggaactactcagccatggcagccgtggtgtactcccctgacttccctgacacctacgccactggcagagtctgctactggaccatccgggttccaggagcctctcgcatccatttcaacttcaccctgtttgatatcagggactctgcagacatggtggagctgctggacggctacacccaccgcgtcctggtccggctcagtgggaggagccgcccgcctctgtctttcaatgtctctctggattttgtcattttgtatttcttctctgatcgcatcaatcaggcccagggatttgctgtgttgtaccaagccaccaaggaggaaccgccacaggagagacctgctgtcaaccagaccctggcagaggtgatcaccgagcaagccaacctcagtgtcagcgctgcccactcctccaaagtcctctatgtcatcacccccagccccagccaccctccgcagactgcccaggtagccattcctgggcaccgtcagttggggccaacagccacagagtggaaggatggactgtgtacggcctggcgaccctcctcatcctcacagtcacagcagttgtcgcaaagattcttctgcatgtcacatttaaatctcatcgagtccctgcatcaggagaccttagggactgtcgtcagcctggggcttctggagatatctggaccattttctatgaaccttccactacaatctccatctttaagaagaagctcaagggtcagagtcaacaagatgaccgcaatcccctcgtgagtgactgaagcccacgcctgcatgagaggctccgctccaagctcgagtttgctcccctgagttctcctctgatgagttccctgccttcccattcaccaccatctcttttgggagcaccctgctttagaggcagcccagcctgggatcctccatcacatgtaccagcctggctgctctgctggggatggtaagacaggcccaggctgacaggacacagctggacctgactccagaagactcttgggtggtggggaggtatagtgtaggatgagttttcttgcttcttctctgttttgtccacatacagatcggtttcccctgtctttacagtttgcaatagagccagactgaaagaactgtcaggttttctaggctggcctggttccccactaagagtggcattggcgccctagaggcccagaggcccagtgtaggcttggagctttctctgctgccaactaccatgtgtcatctagtccgaggggactgagagcagggccacaccagatgtcatctttctagagggttctttttagtacccactgaccaatggggcaagcctgaggattggtccatctgtttgtccatggaacagacacagtgaacttcctggatactagacttaactagcctagccctcaagtagttgccaatcctgtggaatcagaattcagcctgtcttcctgtcctcagcccaagcctgtagcctagagctggggctgtagcctagagctggggctgtagcctagagctggggctgtagcacagagctggggctgtagcctagagctggggctgtagcacagagctggggctgtagcctagagctggggctgtagcacagagctggggctgtagcacagagctggggctgtagcctagagctggggctgtagcacagagctggggctgtaactcagcgatcaagagcttgctttgtatacatcggaccctaggttctatcccagcactatcagaaggtgggagagaaaaagactgcaccatagcatgcgggcagcatctgtggttcctacgtgaggtgtcatcattttaaaagcagatcaaaactaccgcgagttttgtcctttgtcccttatcatgggagcagagtaggagtaagggctctggtcttgctcattgtcccccagacagggaggcaggaaaaggtcaggcttgggaactggagatcctcccaggaaaagctgcaagattgagagacccagctgcagttgggagaggaagggccatccccgactgagaagtcctgcagtctggaagtggcctttgtcagcagcagctgtgccctgaaggtagaccttggtcactctcctgccagcccttgagcctctgctctcctgggtaccctcctggaacaccatgctaaccttccccgagtctctcagtcactgccattgaggcctctcctctagctgctgctccccaggactgtctggggccatctggggatcagggagaggcagcaggagtactgacgaggcagtgacctgagctgatgagtcaaccagaggacaccagagtctacagtgggctggctgctggctcagctcctatgggaggcctacaggggtactaagctagggggtcatcatctcatttgatctgggaaaggctacaggctcctggatgtgaagacaggcccactacataagaagaccactggaaatagactgacaggagcaggttccactctaggctgtccatagcgtttgcaggactcccctgagaccaagtgttgagtcacagagtgccatgtgcgtagtgcataaaggatatgggttcttaaccagggaaggctcatagcaggccaggacattttttcagctcagagcactggccccaggcttcctctaagccaccactcacctgtctcttcctatctcggacacaggaagcaagccccagtgtggtggcagctgcggctcagcattggtgtccccaggaagggcggtggatgtgcccacgctccttttgctgtgggcctggcacagcccaacactgcagggcccaccttctctcttggggggtagggacacataaggaaaactaacccacctccaacaacagcagaggacagtgggaaggaagggctgtaaatcacccaggccagacctccagaaatgacaggcacagtctgttagaacctgtaggcagccagtcacagagggcctttgtgctggtaacaccctgcctggagcataggggtaagccgagggagaagagcagccctcagagacatcagctaaaaacataggtgccctatgtccctcccttcctgtcacactgcttacaaagcagagacagagtaggaaagaggtcttcatcctctcccacatcagcaaggatagggctgcggctgcctaaagtgagcaaggagaacagagctctggacttctctaaatgtgggctctggcttcagactcctcagccaaaagctcttgaagatcaaagctctggcgggtacagctgtcctggcctgtgggccagcccatgggatgtgcctgggccaggtgccaccccacggctcactgtcatcccaggagggaccccacctgatgctcctcatcatccgctggcctgacactatcagagctcgcgccggctgttgccagggacagactgactacacttgaccttcaagagcacttagaagtggatggcctccagactctgtcagcctctgcaggggccacacaagtctcccgagccaagtccacaagcctccatggttccctggctcctctcctgtggagtgtcctgtttgatgtctgaggtctgctttgggtaccgccctgggaactgctaacctccgattggtccctttgtgtctctgtttactgtcctcttctacctccaggtcacttagctctggctgctctggctgggagtggggggtggggatgctggctgcacccccaccctggtctgccaacagaacctgggggcctcacacgggctcctgtcttgccaagctggagctgagcacactggcccaggctgagtggggcagagcaaacaagtggaaggggatctctctccttagagggaggtggccgaaggtgtagatccagcgagggagctgccatccccgccaccttcatagcagcaagaccttcccatttccaatctcaccctccagcagggatatgactttggacaacaaggctttatttgtaaatatgctcttaatatgcaactttgagaataagatagaaacatcatgtattttaaaatataaaatgaagtgtgacacactgtatacaatttaatatatatttttaggattttgttatttaagaaaatggaatgtgatggtacttaacttttacaaaagagagaaaatgttatttttactgtttgaagaaaataaatattctcattgttgtagaaaaaaaaaaaaaaaaaaagggcggccgc

SEQ ID NO: 68 - DNA, 1410 bp, Mus sp.
atggcgccgcccgccgcccgtctcgcgctgctctccgccgctgcgctcactctggcggcccggcccgcgcccggtccccgctccggccccgagtgcttcacagccaacggtgcagattacaggggaacacagagctggacagcgctgcaaggtgggaagccatgtctgttctggaacgagactttccagcatccgtacaacacgctgaagtaccccaacggggaaggaggactgggcgagcacaattattgcagaaatccagatggagacgtgagcccttggtgctacgtggccgagcatgaggacggagtctactggaagtactgtgaaattcctgcctgccagatgcctggaaaccttggctgctacaaggatcatggaaacccacctcctctcacgggcaccagtaaaacctctaacaagctcaccatacaaacctgtatcagcttctgtcggagtcagagattcaagtttgctgggatggagtcaggctatgcctgcttctgtgggaacaatcctgactactggaagcacggggaggcggccagcaccgagtgcaatagtgtctgcttcggggaccacacgcagccctgcggtggggacggcaggattatcctctttgacactctcgtgggcgcctgcggtgggaactactcagccatggcagccgtggtgtactcccctgacttccctgacacctacgccactggcagagtctgctactggaccatccgggttccaggagcctctcgcatccatttcaacttcaccctgtttgatatcagggactctgcagacatggtggagctgctggacggctacacccaccgcgtcctggtccggctcagtgggaggagccgcccgcctctgtctttcaatgtctctctggattttgtcattttgtatttcttctctgatcgcatcaatcaggcccagggatttgctgtgttgtaccaagccaccaaggaggaaccgccacaggagagacctgctgtcaaccagaccctggcagaggtgatcaccgagcaagccaacctcagtgtcagcgctgcccactcctccaaagtcctctatgtcatcacccccagccccagccaccctccgcagactgcccaggtagccattcctgggcaccgtcagttggggccaacagccacagagtggaaggatggactgtgtacggcctggcgaccctcctcatcctcacagtcacagcagttgtcgcaaagattcttctgcatgtcacatttaaatctcatcgagtccctgcatcaggagaccttagggactgtcgtcagcctggggcttctggagatatctggaccattttctatgaaccttccactacaatctccatctttaagaagaagctcaagggtcagagtcaacaagatgaccgcaatcccctcg

SEQ ID NO: 69 - protein, 470 aa, Mus sp.
MAPPAARLALLSAAALTLAARPAPGPRSGPECFTANGADYRGTQSWTALQGGKPCLFWNETFQHPYNTLKYPNGEGGLGEHNYCRNPDGDVSPWCYVAEHEDGVYWKYCEIPACQMPGNLGCYKDHGNPPPLTGTSKTSNKLTIQTCISFCRSQRFKFAGMESGYACFCGNNPDYWKHGEAASTECNSVCFGDHTQPCGGDGRIILFDTLVGACGGNYSAMAAVVYSPDFPDTYATGRVCYWTIRVPGASRIHFNFTLFDIRDSADMVELLDGYTHRVLVRLSGRSRPPLSFNVSLDFVILYFFSDRINQAQGFAVLYQATKEEPPQERPAVNQTLAEVITEQANLSVSAAHSSKVLYVITPSPSHPPQTAQVAIPGHRQLGPTATEWKDGLCTAWRPSSSSQSQQLSQRFFCMSHLNLIESLHQETLGTVVSLGLLEISGPFSMNLPLQSPSLRRSSRVRVNKMTAIPS

SEQ ID NO: 70 - protein, 760 aa, Mus sp.
MALPSLGQDSWSLLRVFFFQLFLLPSLPPASGTGGQGPMPRVKYHAGDGHRALSFFQQKGLRDFDTLLLSDDGNTLYVGARETVLALNIQNPGIPRLKNMIPWPASERKKTECAFKKKSNETQCFNFIRVLVSYNATHLYACGTFAFSPACTFIELQDSLLLPILIDKVMDGKGQSPLTLFTSTQAVLVDGMLYSGTMNNFLGSEPILMRTLGSHPVLKTDIFLRWLHADASFVAAIPSTQVVYFFFEETASEFDFFEELYISRVAQVCKNDVGGEKLLQKKWTTFLKAQLLCAQPGQLPFNIIRHAVLLPADSPSVSRIYAVFTSQWQVGGTRSSAVCAFSLTDIERVFKGKYKELNKETSRWTTYRGSEVSPRPGSCSMGPSSDKALTFMKDHFLMDEHVVGTPLLVKSGVEYTRLAVESARGLDGSSHVVMYLGTSTGPLHKAVVPQDSSAYLVEEIQLSPDSEPVRNLQLAPAQGAVFAGFSGGIWRVPRANCSVYESCVDCVLARDPHCAWDPESRLCSLLSGSTKPWKQDMERGNPEWVCTRGPMARSPRRQSPPQLIKEVLTVPNSILELRCPHLSALASYHWSHGRAKISEASATVYNGSLLLLPQDGVGGLYQCVATENGYSYPVVSYWVDSQDQPLALDPELAGVPRERVQVPLTRVGGGASMAAQRSYWPHFLIVTVLLAIVLLGVLTLLLASPLGALRARGKVQGCGMLPPREKAPLSRDQHLQPSKDHRTSASDVDADNNHLGAEVA

SEQ ID NO: 71 - DNA, 3046 bp, Mus sp.
ctcggacgcctgggttaggggtctgtactgctggggaaccatctggtgaccatctcaggctgaccatggccctaccatccctgggccaggactcatggagtctcctgcgtgtttttttcttccaactcttcctgctgccatcactgccacctgcttctgggactggtggtcaggggcccatgcccagagtcaaataccatgctggagacgggcacagggccctcagcttcttccaacaaaaaggcctccgagactttgacacgctgctcctgagtgacgatggcaacactctctatgtgggggctcgagagaccgtcctggccttgaatatccagaacccaggaatcccaaggctaaagaacatgataccctggccagccagtgagagaaaaaagaccgaatgtgcctttaagaagaagagcaatgagacacagtgtttcaacttcattcgagtcctggtctcttacaatgctactcacctctatgcctgtgggacctttgccttcagccctgcctgtaccttcattgaactccaagattccctcctgttgcccatcttgatagacaaggtcatggacgggaagggccaaagccctttgaccctgttcacaagcacacaagctgtcttggtcgatgggatgctttattccggcaccatgaacaacttcctgggcagcgagcccatcctgatgcggacactgggatcccatcctgttctcaagactgacatcttcttacgctggctgcacgcggatgcctccttcgtggcagccattccatccacccaggtcgtctatttcttctttgaggagacagccagcgagtttgacttctttgaagagctgtatatatccagggtggctcaagtctgcaagaacgacgtgggcggtgaaaagctgctgcagaagaagtggaccaccttcctcaaagcccagttgctctgcgctcagccagggcagctgccattcaacatcatccgccacgcggtcctgctgcccgccgattctccctctgtttcccgcatctacgcagtctttacctcccagtggcaggttggcgggaccaggagctcagcagtctgtgccttctctctcacggacattgagcgagtctttaaagggaagtacaaggagctgaacaaggagacctcccgctggaccacttaccggggctcagaggtcagcccgaggccaggcagttgctccatgggcccctcctctgacaaagccttgaccttcatgaaggaccattttctgatggatgagcacgtggtaggaacacccctgctggtgaagtctggtgtggagtacacacggcttgctgtggagtcagctcggggccttgatgggagcagccatgtggtcatgtatctgggtacctccacgggtcccctgcacaaggctgtggtgcctcaggacagcagtgcttatctcgtggaggagattcagctgagccctgactctgagcctgttcgaaacctgcagctggcccccgcccagggtgcagtgtttgcaggcttctctggaggcatctggagagttcccagggccaattgcagtgtctacgagagctgtgtggactgtgtgcttgccagggaccctcactgtgcctgggaccctgaatcaagactctgcagccttctgtctggctctaccaagccttggaagcaggacatggaacgcggcaacccggagtgggtatgcacccgtggccccatggccaggagcccccggcgtcagagcccccctcaactaattaaagaagtcctgacagtccccaactccatcctggagctgcgctgcccccacctgtcagcactggcctcttaccactggagtcatggccgagccaaaatctcagaagcctctgctaccgtctacaatggctccctcttgctgctgccgcaggatggtgtcgggggcctctaccagtgtgtggcgactgagaacggctactcataccctgtggtctcctattgggtagacagccaggaccagcccctggcgctggaccctgagctggcgggcgttccccgtgagcgtgtgcaggtcccgctgaccagggtcggaggcggagcttccatggctgcccagcggtcctactggccccattttctcatcgttaccgtcctcctggccatcgtgctcctgggagtgctcactctcctcctcgcttccccactgggggcgctgcgggctcggggtaaggttcagggctgtgggatgctgccccccagggaaaaggctccactgagcagggaccagcacctccagccctccaaggaccacaggacctctgccagtgacgtagatgccgacaacaaccatctgggcgccgaagtggcttaaacagggacacagatccgcagctgagcagagcaagccactggccttgttggctatgccaggcacagtgccactctgaccagggtaggaggctctcctgctaacgtgtgtcacctacagcacccagtaggtcctcccctgtgggactctcttctgcaagcacattgggctgtctccatacctgtacttgtgctgtgacaggaagagccagacaggtttctttgattttgattgacccaagagccctgcctgtaacaaacgtgctccaggagaccatgaaaggtgtggctgtctgggattctgtggtgacaaacctaagcatccgagcaagctggggctattcctgcaaactccatcctgaacgctgtcactctagaagcagctgctgctttgaacaccagcccaccctccttcccaagagtctctatggagttggccccttgtgtttcctttaccagtcgggccatactgtttgggaagtcatctctgaagtctaaccaccttccttcttggttcagtttggacagattgttattattgtctctgccctggctagaatgggggcataatctgagccttgttcccttgtccagtgtggctgacccttgacctcttccttcctcctccctttgttttgggattcagaaaactgcttgtcacagacaatttattttttattaaaaaagatataagctttaaag

SEQ ID NO: 72 - DNA, 2915 bp, Mus sp.
gtcgacccacgcgtccggccgcgcgtccttctgccggcttcagctcgtatccccggagtccacccgcccgtcccggggtgcggactggccctgagctggccgtacagcccggcttcggacggtcctcgctggagccatgggccgccggctcggcagggtggcggcgctgctgctcgggctgctagtggagtgcactgaggccaaaaaacattgctggtattttgaaggactctatcccacatactatatatgccgttcctatgaagactgctgtggctccaggtgctgtgtgagggccctttccatacagaggctgtggtatttttggttcctgctgatgatgggtgtgctgttctgctgtggtgccggtttcttcattcgccggcgcatgtatccgccaccactcattgaggagcccacattcaatgtgtcctataccaggcagccaccaaatcctgctccaggagcacagcaaatgggaccgccatattacaccgaccctggaggacccgggatgaatcctgttggcaataccatggctatggctttccaggtccagcccaattcacctcacggaggcacaacttacccaccccctccttcctactgcaacacgcctccacccccctatgaacaggtggtgaaggacaagtagcaagatgctacatcaaaggcaaagaggatggacaggcccttttgtttaccttcccatcctcaccgatacttgctgatagggtggtccaagggaaaacttggatattctcaaagcaagcccagctctctttcaagtcttttgtggaggacatttgaatccacactgtctcctctgttgcttctgtttctgatgtagtctgtgctctctgagagagtgtggcaacagtccctgagggttgatattcctagggtgtccagggtagatcctcgggagagaggctaaggggaaaggaaggcatagcctgtgtgttagggggcagataaagtggtcaggctgagataagactcacatgatgcagtagttggcagtgaacttcgaagagacactatccaccatcccagcccattctcctaatagaagctgtggggctgtgttgttgatgctctttggtctccactcacattttgaaaataggctttcctctgcaggaataggaaagacccaagtacatatttgcttccacttaaaaatgagggtcagaaccaggcctcagttggacatctatagttaaataaaggccattagagaggggaaatctttaagttaggggaaattctctaaatggagacattgcgttttatgaatcatcgtctggcttttcttttagtgcatgtattgaagtgagggtgtcctttgagatcagatggggagagtgaactctgcggggggtggggtgtctctactcagagggctccaacacccttttcttaggtagttctggtgatgggttttatgggcactatagagctgaggggcacattaggccgggtagttacattgacccttggagaggaagaggacagccaaagaaactcagcaaagcaagaccagcattgctgagttagagctagggttgtatgtgatcccaacagagatgtgctggcctcagaagaggggacgtttgtggatagagccgtgaaaacctacttagttgcacagatgacataatcaaaagtagagaaagaagtgtagttagagatgccatttcccaggtgagaatcagagctcatccatagatttacaagtagtggctggagttaacagtatggagttcttttcccttgcgtagttagtcacgttgatgtgtatttaaacccaggttgagaccttgtgtactaagagcaaggaagtatagctaagatgtctagattatttatatgtagtatggtggggagtggggctgcaaggaagggggctgacattgtaaatgagaaaatcagagccatttgataaactgttacttgttggatcaggcatccaaaagtgtctcttgagtggacattgagtattctttaccacctacaagaccaggaggcatggtgtcattctccattggggtatttatatgaggtagaggttcaggaatcgacagtagctgtgtgggcttagtttaaggactgaaagcatagggactggtagacagtttcataggaaactgcggggaaggaatggatacctttaaagacagtttgtggatgcagatgctgccacccatcattgagcacccttgtgtctctggcttcctgtcactggatccagtacccctccatgcttgggtccttgttttacataagacaacaaagcacaatgtctgctgtttacaatcaagacgactacatggtccaaacatttcttctctcttctatcacttgtggctttaacttccatttcctccgttcctttttaaaatcaagaagcacagtcagagctgcccctgggattgcatcagggaacggctgatcaaggcattcagtgtccatgactaaatcttatctttttgatagcaaatccttttaagaaactgaacaattgctaaggctcagcaattttatactccaatgtctgtgtaaggtaaattttgtttgccattgagcccacattggaattccttctgacgtcaacactgacaatgcctatggaaattgcacttctgggtatatgtcccagcatccttgttttcttatgtttggtgagtaaggctcaccccttccagcagctctacttctgtgtgctgaggtcctgtagagccggggcttgggcacagacatgaggcagacttgtgcatgctctttcttggcaacacttggctcatatttcttgttctcttttgatagagtcctgtttcctatgtatttaaaaaataataaaagtgaatttagtcaaaaaaaaaaaaaaaaaaaaaaaaaaaaagggcggccgc

SEQ ID NO: 73 - DNA, 516 bp, Mus sp.
atgggccgccggctcggcagggtggcggcgctgctgctcgggctgctagtggagtgcactgaggccaaaaaacattgctggtattttgaaggactctatcccacatactatatatgccgttcctatgaagactgctgtggctccaggtgctgtgtgagggccctttccatacagaggctgtggtatttttggttcctgctgatgatgggtgtgctgttctgctgtggtgccggtttcttcattcgccggcgcatgtatccgccaccactcattgaggagcccacattcaatgtgtcctataccaggcagccaccaaatcctgctccaggagcacagcaaatgggaccgccatattacaccgaccctggaggacccgggatgaatcctgttggcaataccatggctatggctttccaggtccagcccaattcacctcacggaggcacaacttacccaccccctccttcctactgcaacacgcctccacccccctatgaacaggtggtgaaggacaag

SEQ ID NO: 74 - protein, 172 aa, Mus sp.
MGRRLGRVAALLLGLLVECTEAKKHCWYFEGLYPTYYICRSYEDCCGSRCCVRALSIQRLWYFWFLLMMGVLFCCGAGFFIRRRMYPPPLIEEPTFNVSYTRQPPNPAPGAQQMGPPYYTDPGGPGMNPVGNTMAMAFQVQPNSPHGGTTYPPPPSYCNTPPPPYEQVVKDK

SEQ ID NO: 75 - protein, 398 aa, Homo sapiens
MWLLLTMASLISVLGTTHGLFGKLHPGSPEVTMNISQMITYWGYPNEEYEVVTEDGYILEVNRIPYGKKNSGNTGQRPVVFLQHGLLASATNWISNLPNNSLAFILADAGYDVWLGNSRGNTWARRNLYYSPDSVEFWAFSFDEMAKYDLPATIDFIVKKTGQKQLHYVGHSQGTTIGFIAFSTNPSLAKRIKTFYALAPVATVKYTKSLINKLRFVPQSLFKFIFGDKIFYPHNFFDQFLATEVCSREMLNLLCSNALFIICGFDSKNFNTSRLDVYLSHNPAGTSVQNMFHWTQAVKSGKFQAYDWGSPVQNRMHYDQSQPPYYNVTAMNVPIAVWNGGKDLLADPQDVGLLLPKLPNLIYHKEIPFYNHLDFIWAMDAPQEVYNDIVSMISEDKK

SEQ ID NO: 76 - protein, 760 aa, Mus sp.; identical to SEQ ID NO: 70

SEQ ID NO: 77 - DNA, 3046 bp, Mus sp.; identical to SEQ ID NO: 71

SEQ ID NO: 78 - protein, 1436 aa, Bos sp.
MALGRHLSLRGLCVLLLGTMVGGQALELRLKDGVHRCEGRVEVKHQGEWGTVDGYRWTLKDASVVCRQLGCGAAIGFPGGAYFGPGLGPIWLLYTSCEGTESTVSDCEHSNIKDYRNDGYNHGRDAGVVCSGFVRLAGGDGPCSGRVEVHSGEAWIPVSDGNFTLATAQIICAELGCGKAVSVLGHELFRESSAQVWAEEFRCEGEEPELWVCPRVPCPGGTCHHSGSAQVVCSAYSEVRLMTNGSSQCEGQVEMNISGQWRALCASHWSLANANVICRQLGCGVAISTPGGPHLVEEGDQILTARFHCSGAESFLWSCPVTALGGPDCSHGNTASVICSGNQIQVLPQCNDSVSQPTGSAASEDSAPYCSDSRQLRLVDGGGPCAGRVEILDQGSWGTICDDGWDLDDARVVCRQLGCGEALNATGSAHFGAGSGPIWLDNLNCTGKESHVWRCPSRGWGQHNCRHKQDAGVICSEFLALRMVSEDQQCAGWLEVFYNGTWGSVCRNPMEDITVSTICRQLGCGDSGTLNSSVALREGFRPQWVDRIQCRKTDTSLWQCPSDPWNYNSCSPKEEAYIWCADSRQIRLVDGGGRCSGRVEILDQGSWGTICDDRWDLDDARVVCKQLGCGEALDATVSSFFGTGSGPIWLDEVNCRGEESQVWRCPSWGWRQHNCNHQEDAGVICSGFVRLAGGDGPCSGRVEVHSGEAWTPVSDGNFTLPTAQVICAELGCGKAVSVLGHMPFRESDGQVWAEEFRCDGGEPELWSCPRVPCPGGTCLHSGAAQVVCSVYTEVQLMKNGTSQCEGQVEMKISGRWRALCASHWSLANANVVCRQLGCGVAISTPRGPHLVEGGDQISTAQFHCSGAESFLWSCPVTALGGPDCSHGNTASVICSGNHTQVLPQCNDFLSQPAGSAASEESSPYCSDSRQLRLVDGGGPCGGRVEILDQGSWGTICDDDWDLDDARVVCRQLGCGEALNATGSAHFGAGSGPIWLDDLNCTGKESHVWRCPSRGWGRHDCRHKEDAGVICSEFLALRMVSEDQQCAGWLEVFYNGTWGSVCRSPMEDITVSVICRQLGCGDSGSLNTSVGLREGSRPRWVDLIQCRKMDTSLWQCPSGPWKYSSCSPKEEAYISCEGRRPKSCPTAAACTDREKLRLRGGDSECSGRVEVWHNGSWGTVCDDSWSLAEAEVVCQQLGCGQALEAVRSAAFGPGNGSIWLDEVQCGGRESSLWDCVAEPWGQSDCKHEEDAGVRCSGVRTTLPTTTAGTRTTSNSLPGIFSLPGVLCLILGSLLFLVLVILVTQLLRWRAERRALSSYEDALAEAVYEELDYLLTQKEGLGSPDQMTDVPDENYDDAEEVPVPGTPSPSQGNEEEVPPEKEDGVRSSQTGSFLNFSREAANPGEGEESFWLLQGKKGDAGYDDVELSALGTSPVTFS

SEQ ID NO: 79 - DNA, 4308 bp, Bos sp.
atggctctgggcagacacctctccctgcggggactctgtgtcctcctcctcggcaccatggtgggtggtcaagctctggagctgaggttgaaggatggagtccatcgctgtgaggggagagtggaagtgaagcaccaaggagaatggggcacagtggatggttacaggtggacattgaaggatgcatctgtagtgtgcagacagctggggtgtggagctgccattggttttcctggaggggcttattttgggccaggacttggccccatttggcttttgtatacttcatgtgaagggacagagtcaactgtcagtgactgtgagcattctaatattaaagactatcgtaatgatggctataatcatggtcgggatgctggagtagtctgctcaggatttgtgcgtctggctggaggggatggaccctgctcagggcgagtagaagtgcattctggagaagcttggatcccagtgtctgatgggaacttcacacttgccactgcccagatcatctgtgcagagttgggttgtggcaaggctgtgtctgtcctgggacatgagctcttcagagagtccagtgcccaggtctgggctgaagagttcaggtgtgagggggaggagcctgagctctgggtctgccccagagtgccctgtccagggggcacgtgtcaccacagtggatctgctcaggttgtttgttcagcatactcagaagtccggctcatgacaaacggctcctctcagtgtgaagggcaggtggagatgaacatttctggacaatggagagcgctctgtgcctcccactggagtctggccaatgccaatgttatctgtcgtcagctcggctgtggagttgccatctccacccccggaggaccacacttggtggaagaaggtgatcagatcctaacagcccgatttcactgctctggggctgagtccttcctgtggagttgtcctgtgactgccctgggtggtcctgactgttcccatggcaacacagcctctgtgatctgctcaggaaaccagatccaggtgcttccccagtgcaacgactccgtgtctcaacctacaggctctgcggcctcagaggacagcgccccctactgctcagacagcaggcagctccgcctggtggacgggggcggtccctgcgccgggagagtggagatccttgaccagggctcctggggcaccatctgtgatgacggctgggacctggacgatgcccgcgtggtgtgcaggcagctgggctgtggagaagccctcaatgccacggggtctgctcacttcggggcaggatcagggcccatctggttggacaacttgaactgcacaggaaaggagtcccacgtgtggaggtgcccttcccggggctgggggcagcacaactgcagacacaagcaggacgcgggggtcatctgctcagagttcctggccctcaggatggtgagtgaggaccagcagtgtgctgggtggctggaagttttctacaatgggacctggggcagtgtctgccgtaaccccatggaagacatcactgtgtccacgatctgcagacagcttggctgtggggacagtggaaccctcaactcttctgttgctcttagagaaggttttaggccacagtgggtggatagaatccagtgtcggaaaactgacacctctctctggcagtgtccttctgacccttggaattacaactcatgctctccaaaggaggaagcctatatctggtgtgcagacagcagacagatccgcctggtggatggaggtggtcgctgctctgggagagtggagatccttgaccagggctcctggggcaccatctgtgatgaccgctgggacctggacgatgcccgtgtggtgtgcaagcagctgggctgtggagaagccctggacgccactgtctcttccttcttcgggacgggatcagggcccatctggctggatgaagtgaactgcagaggagaggagtcccaagtatggaggtgcccttcctggggatggcggcaacacaactgcaatcatcaagaagatgcaggagtcatctgctcaggatttgtgcgtctggctggaggagatggaccctgctcagggcgagtagaagtgcattctggagaagcctggaccccagtgtctgatggaaacttcacactccccactgcccaggtcatctgtgcagagctgggatgtggcaaggctgtgtctgtcctgggacacatgccattcagagagtccgatggccaggtctgggctgaagagttcaggtgtgatgggggggagcctgagctctggtcctgccccagagtgccctgtccaggaggcacatgtctccacagtggagctgctcaggttgtctgttcagtgtacacagaagtccagcttatgaaaaacggcacctctcaatgtgaggggcaggtggagatgaagatctctggacgatggagagcgctctgtgcctcccactggagtctggccaatgccaatgttgtctgtcgtcagctcggctgtggagtcgccatctccacccccagaggaccacacttggtggaaggaggtgatcagatctcaacagcccaatttcactgctcaggggctgagtccttcctgtggagttgtcctgtgactgccttgggtgggcctgactgttcccatggcaacacagcctctgtgatctgctcaggaaaccacacccaggtgctgccccagtgcaacgacttcctgtctcaacctgcaggctctgcggcctcagaggagagttctccctactgctcagacagcaggcagctccgcctggtggacgggggcggtccctgcggcgggagagtggagatccttgaccagggctcctggggcaccatctgtgatgatgactgggacctggacgatgcccgtgtggtgtgcaggcagctgggctgtggagaagccctcaatgccacggggtctgctcacttcggggcaggatcagggcccatctggctggacgacctgaactgcacaggaaaggagtcccacgtgtggaggtgcccttcccggggctgggggcggcacgactgcagacacaaggaggacgccggggtcatctgctcagagttcctggccctcaggatggtgagcgaggaccagcagtgtgctgggtggctggaggttttctacaacgggacctggggcagtgtctgccgcagccccatggaagatatcactgtgtccgtgatctgcagacagcttggatgtggggacagtggaagtctcaacacctctgttggtctcagggaaggttctagaccccggtgggtagatttaattcagtgtcggaaaatggatacctctctctggcagtgtccttctggcccatggaaatacagttcatgctctccaaaggaggaagcctacatctcatgtgaaggaagaagacccaagagctgtccaactgctgccgcctgcacagacagagagaagctccgcctcaggggaggagacagcgagtgctcagggcgggtggaggtgtggcacaacggctcctggggcaccgtgtgcgatgactcctggagcctggcagaggctgaggtggtgtgtcagcagctgggctgtggccaggccctggaagccgtgcggtctgcagcatttggccctggaaatgggagcatctggctggacgaggtgcagtgcgggggccgggagtcctccctgtgggactgtgttgcggagccctgggggcagagcgactgcaagcacgaggaggatgctggtgtgaggtgctctggtgtaaggacaacattgcccacgaccacagcagggaccagaacaacctcaaattctctccctggcatcttctccctgcctggggttctctgccttatcctggggtcgcttctcttcctggtcctcgtcatcctggtgactcagctactcagatggagagcagagcgcagagccttatccagctatgaagatgctcttgctgaagctgtgtatgaggagctcgattaccttctgacacagaaggaaggtctgggcagcccagatcagatgactgatgtccctgatgaaaattatgatgatgctgaagaagtaccagtgcctggaactccttctccctctcaggggaatgaggaggaagtgcccccagagaaggaggacggggtgaggtcctctcagacaggctctttcctgaacttctccagagaggcagctaatcctggggaaggagaagagagcttctggctgctccaggggaagaaaggggatgctgggtatgatgatgttgaactcagtgccctgggaacatccccagtgactttctcg